SEQUENCE LISTING (65 sequences)

SEQ ID NO: 1 - DNA, 1207 bp, Homo sapiens
tttctgtgaagcagaagtctgggaatcgatctggaaatcctcctaatttttactccctctccccgcgactcctgattcattgggaagtttcaaatcagctataactggagagtgctgaagattgatgggatcgttgccttatgcatttgttttggttttacaaaaaggaaacttgacagaggatcatgctgtacttaaaaaatacaacatcacagaggaagtagactgatattaacaatacttactaataataacgtgcctcatgaaataaagatccgaaaggaattggaataaaaatttcctgcatctcatgccaagggggaaacaccagaatcaagtgttccgcgtgattgaagacaccccctcgtccaagaatgcaaagcacatccaataaaatagctggattataactcctcttctttctctgggggccgtggggtgggagctggggcgagaggtgccgttggcccccgttgcttttcctctgggaaggatggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggctgaggccacaggtccgagatgcgggggttggagtgcgggtgggctcctggggcaatgggaggctgtggagccggcgaaataaaatcagagttgttgcta

SEQ ID NO: 2 - DNA, 4201 bp, Homo sapiens
ggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccatggctgatcagctgaccgaagaacagattgctgaattcaaggaagccttctccctatttgataaagatggcgatggcaccatcacaacaaaggaacttggaactgtcatgaggtcactgggtcagaacccaacagaagctgaattgcaggatatgatcaatgaagtggatgctgatggtaatggcaccattgacttccccgaatttttgactatgatggctagaaaaatgaaagatacagatagtgaagaagaaatccgtgaggcattccgagtctttgacaaggatggcaatggttatatcagtgcagcagaactacgtcacgtcatgacaaacttaggagaaaaactaacagatgaagaagtagatgaaatgatcagagaagcagatattgatggagacggacaagtcaactatgaagaattcgtacagatgatgactgcaaaatgaagacctactttcaactcctttttcccccctctagaagaatcaaattgaatcttttacttacctcttgcaaaaaaaagaaaaaagaaaaaagttcatttattcattctgtttctatatagcaaaactgaatgtcaaaagtaccttctgtccacacacacaaaatctgcatgtattggttggtggtcctgtcccctaaagatcaagctacacatcagttttacaatataaatacttgtactaccttaatgataaggactccttaaagttccatttgctaatgattaatacactgtttgggctggccagtttttcatgcatgcagcttgacgattgagcacagtcaggcctttgtattaaaaatgaaaaatgaaaaaacaaattcaaaacctattcaaatgggttctagttcaatttgtttagtataaattgtcatagctggtttactgaaaacaaacacatttaaaattggtttacctcaggatgacgtgcagaaaaatgggtgaaggataaaccgttgagacgtggccccactggtaggatggtcctcttgtacttcgtgtgctccgacccatggtgacgatgacacaccctggtggcatgcccgtgtatgttggtttagcgttgtctgcattgttctagagtgaaacaggtgtcaggctgtcactgttcacacaaatttttaataagaaacatttaccaagggagcatctttggactctctgtttttaaaaccttctgaaccatgacttggagccggcagagtaggctgtggctgtggacttcagcacaaccatcaacattgctgttcaaagaaattacagtttacgtccattccaagttgtaaatgctagtctttttttttttttttccaataaaaagaccattaacttaaagtggtgttaaatgctttgtaaagctgagatctaaatggggacaaggcaggtggaggggaggccagtgtacatgtaaatgcccacagcccagcattgggtttccctcccaaggccccagcaccaacctctgagcccaagaccttgcctgaaaacaagcagataccgattgcttcatcctatttatggacatgtaggtctagttgcattttcactggggggaggggggaaggtgaattatggtaacttttaatgatctattcaggcagtagagctcttaaggaaaaaaaaaaacccactttctctcaagcatgtatttaggggttgttctcaattgtgctgctgattacctgtcttatgtaactacttgagaccatctgcaagagacatgatttagtgtgtctgtaattcaatcttcgctgtgtgtggtagaagcagtagtcacttttgtaagccagtctcttcatgcctaaaagacactaccagtcacctttgattcgcgacttttaatttatgattatacttagcctcctcctcctttttttttttttcccaagttgacttgactttgcttttttccccccaagtagaactaatgctagcttccagcttgaaagtaaaactccagtgtggagtgaattttgtgtctaattataaacctgtaaccaaaactcagacatctggtactggtctttgcattgagattggtccctgtaaaaccccctttaaaagcatattgcatttagtacagagctcttttttgaaatgaaggctggagatgtgcatttttcacggtgttaactggttgtatcttattagcaaggagattggggttttgagtgtttgcgtgggtggtttcaatttgccagggaacagtggcaggctgctagcaaggcagtgagaagctcttggcagccaaatgggtgcattcagggctgatttatagagacccttggcttctccttctcctactccctgtctttctggcattttgtagcttgttagattttctgccagaggggtgggtcagagcagtggaggggagacatcgcccatgtgcttctgctactggtccttgggctgggtggttggtagaggagatgttgacactatgagctaagggttggcttttgtaattacctgaatctgaaaggaatgcctaaggttaccttggggtttctcttctggtgagatagggttcctggtttgagtaagttaatgtcctggatatttcttgtggcagggggtggtcaaagagcctgattgctgacccagtctcaggcctgtggtcgatgacctctcggtagtttcaaagggggctggagggggatatttgacttgttttttcgaaatgtagccttctaaccctcaagtctttagaagctgggtggactcttagtggtcctgcagcgtatcctaaaagactacctttgaaacaggattcttgtatggccaggatcctgtctgggaaccagaaaccctacaccctccccctccagggaatgctgagttccagttttgagcagaggtgaggcagaatccactgtagccttccgccctggtatttggggggatgaccagcccaggcgttgggtgttagtctgcatgagtttgtgagaggaaatagctgggtgtcctggcagtgcccttgaagttggttaggaccttcctgtaaactcttgcccctacttctaactactctataaatatatacatatatttatatataaagtgattagttgaactggcatcctgctttagcctgagacttgccataagaaactgctgagtacttggcaaaccctttcatagttttgttctccatctgtttggggtaggtgttgagcgaggcaaatggatctcgatatttcagatgggcttttgatgcactgttgccaaggaaggctttttctgattttttgacaaatgaatttttgcacactttcattggtgtctttcggcaacttacacacattgaaaatgagctattgtacatatttttatattctctttataaatgcatgtctgattgtacttgtaacaatattgtaatgaacggctgtgcagtaggcccagcgctgctgtgtctcgtcagaggaatagcttaccacgaacccctcagcatactgggaatctcttcctgaacaacgaatgtaaatttggtcaagtctactcttccgttcattcaattattttaagcatttgaattatttattgtatatcctaaatatatttctcctttggcagtgactagatttccactaatgtgtcttaatctatccctccagctggcagttactgtttttttaatcccctgaagttgtcctgtaggagacagaaattctttgctgtctgtatcccttggagtaagaaggtagtggcatgggtggagtgtgtgttctttctccaaatctattatgatgtttattaaacacttctgtagcaaagatggtggtagttcttttgttactgaagttgcccttcaccatggctatttgaaaaggagatgtacttggacgtttctgtaaatcttgagataaactgtttggagatttaaccacctctctgatgggggaccaactctatggaaattgtaaatacgttttatttataaacctggcactgtattcaataaacatttctgcagcctttcatctct

SEQ ID NO: 3 - DNA, 745 bp, Homo sapiens
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtac

SEQ ID NO: 4 - DNA, 2156 bp, Homo sapiens
ggcacgagcccagaaacaaagacttcacggacaaagtcccttggaaccagagagaagccgggatggaaactccaaacaccacagaggactatgacacgaccacagagtttgactatggggatgcaactccgtgccagaaggtgaacgagagggcctttggggcccaactgctgccccctctgtactccttggtatttgtcattggcctggttggaaacatcctggtggtcctggtccttgtgcaatacaagaggctaaaaaacatgaccagcatctacctcctgaacctggccatttctgacctgctcttcctgttcacgcttcccttctggatcgactacaagttgaaggatgactgggtttttggtgatgccatgtgtaagatcctctctgggttttattacacaggcttgtacagcgagatctttttcatcatcctgctgacgattgacaggtacctggccatcgtccacgccgtgtttgccttgcgggcacggaccgtcacttttggtgtcatcaccagcatcatcatttgggccctggccatcttggcttccatgccaggcttatacttttccaagacccaatgggaattcactcaccacacctgcagccttcactttcctcacgaaagcctacgagagtggaagctgtttcaggctctgaaactgaacctctttgggctggtattgcctttgttggtcatgatcatctgctacacagggattataaagattctgctaagacgaccaaatgagaagaaatccaaagctgtccgtttgatttttgtcatcatgatcatcttttttctcttttggaccccctacaatttgactatacttatttctgttttccaagacttcctgttcacccatgagtgtgagcagagcagacatttggacctggctgtgcaagtgacggaggtgatcgcctacacgcactgctgtgtcaacccagtgatctacgccttcgttggtgagaggttccggaagtacctgcggcagttgttccacaggcgtgtggctgtgcacctggttaaatggctccccttcctctccgtggacaggctggagagggtcagctccacatctccctccacaggggagcatgaactctctgctgggttctgactcagaccataggaggccaacccaaaataagcaggcgtgacctgccaggcacactgagccagcagcctggctctcccagccaggttctgactcttggcacagcatggagtcacagccacttgggatagagagggaatgtaatggtggcctggggcttctgaggcttctggggcttcagtcttttccatgaacttctcccctggtagaaagaagatgaatgagcaaaaccaaatattccagagactgggactaagtgtaccagagaagggcttggactcaagcaagatttcagatttgtgaccattagcatttgtcaacaaagtcacccacttcccactattgcttgcacaaaccaattaaacccagtagtggtgactgtgggctccattcaaagtgagctcctaagccatgggagacactgatgtatgaggaatttctgttcttccatcacctccccccccccgccaccctcccactgccaagaacttggaaatagtgatttccacagtgactccactctgagtcccagagccaatcagtagccagcatctgcctccccttcactcccaccgcaggatttgggctcttggaatcctggggaacatagaactcatgacggaagagttgagacctaacgagaaatagaaatgggggaactactgctggcagtggaactaagaaagcccttaggaagaatttttatatccactaaaatcaaacaattcagggagtgggctaagcacgggccatatgaataacatggtgtgcttcttaaaatagccataaaggggagggactcatcatttccatttacccttcttttctgactatttttcagaatctctcttcttttcaagttgggtgatatgttggtagattctaatggctttattgcagcgattaataacaggcaaaaggaagcagggttggtttcccttctttttgttcttcatctaagccttctggttttatgggtcagagttccgactgccatcttggacttgtcagcaaaaaaaaaaaaaaaaa

SEQ ID NO: 5 - DNA, 2232 bp, Homo sapiens
ggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataacattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttgctatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctaatttgccagtgggaactcctaaatcaaattggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgataaaag

SEQ ID NO: 6 - DNA, 1616 bp, Homo sapiens
gccaaggctggggcaggggagtcagcagaggcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctggggg

SEQ ID NO: 7 - DNA, 5731 bp, Homo sapiens
gagaagaaagccagtgcgtctctgggcgcaggggccagtggggctcggaggcacaggcaccccgcgacactccaggttccccgacccacgtccctggcagccccgattatttacagcctcagcagagcacggggcgggggcagaggggcccgcccgggagggctgctacttcttaaaacctctgcgggctgcttagtcacagccccccttgcttgggtgtgtccttcgctcgctccctccctccgtcttaggtcactgttttcaacctcgaataaaaactgcagccaacttccgaggcagcctcattgcccagcggaccccagcctctgccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccactttgatgagcactagtgctacagcaactgagacagcaaccaagaggcaagaaacctgggattggttttcatggttgtttctaccatcagagtcaaagaatcatcttcacacaacaacacaaatggctggtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtataacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgttttttttttttgacactgtccaaaggttttccatcctgtcctggaatcagagttggaagctgaggagcttcagcctcttttatggtttaatggccacctgttctctcctgtgaaaggctttgcaaagtcacattaagtttgcatgacctgttatccctggggccctatttcatagaggctggccctattagtgatttccaaaaacaatatggaagtgccttttgatgtcttacaataagagaagaagccaatggaaatgaaagagattggcaaaggggaaggatgatgccatgtagatcctgtttgacatttttatggctgtatttgtaaacttaaacacaccagtgtctgttcttgatgcagttgctatttaggatgagttaagtgcctggggagtccctcaaaaggttaaagggattcccatcattggaatcttatcaccagataggcaagtttatgaccaaacaagagagtactggctttatcctctaacctcatattttctcccacttggcaagtcctttgtggcatttattcatcagtcagggtgtccgattggtcctagaacttccaaaggctgcttgtcatagaagccattgcatctataaagcaacggctcctgttaaatggtatctcctttctgaggctcctactaaaagtcatttgttacctaaacttatgtgcttaacaggcaatgcttctcagaccacaaagcagaaagaagaagaaaagctcctgactaaatcagggctgggcttagacagagttgatctgtagaatatctttaaaggagagatgtcaactttctgcactattcccagcctctgctcctccctgtctaccctctcccctccctctctccctccacttcaccccacaatcttgaaaaacttcctttctcttctgtgaacatcattggccagatccattttcagtggtctggatttctttttattttcttttcaacttgaaagaaactggacattaggccactatgtgttgttactgccactagtgttcaagtgcctcttgttttcccagagatttcctgggtctgccagaggcccagacaggctcactcaagctctttaactgaaaagcaacaagccactccaggacaaggttcaaaatggttacaacagcctctacctgtcgccccagggagaaaggggtagtgatacaagtctcatagccagagatggttttccactccttctagatattcccaaaaagaggctgagacaggaggttattttcaattttattttggaattaaatacttttttccctttattactgttgtagtccctcacttggatatacctctgttttcacgatagaaataagggaggtctagagcttctattccttggccattgtcaacggagagctggccaagtcttcacaaacccttgcaacattgcctgaagtttatggaataagatgtattctcactcccttgatctcaagggcgtaactctggaagcacagcttgactacacgtcatttttaccaatgattttcaggtgacctgggctaagtcatttaaactgggtctttataaaagtaaaaggccaacatttaattattttgcaaagcaacctaagagctaaagatgtaatttttcttgcaattgtaaatcttttgtgtctcctgaagacttcccttaaaattagctctgagtgaaaaatcaaaagagacaaaagacatcttcgaatccatatttcaagcctggtagaattggcttttctagcagaacctttccaaaagttttatattgagattcataacaacaccaagaattgattttgtagccaacattcattcaatactgttatatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgttattgttactttgacttttcagagcacacccttcctctggtttttgtatatttattgatggatcaataataatgaggaaagcatgatatgtatattgctgagttgaaagcacttattggaaaatattaaaaggctaacattaaaagactaaaggaaacag

SEQ ID NO: 8 - DNA, 1480 bp, Homo sapiens
accagccttgtctcggccacctcaaggataatcactaaattctgccgaaaggactgaggaacggtgcctggaaaagggcaagaatatcacggcatgggcatgagtagcttgaaactgctgaagtatgtcctgtttttcttcaacttgctcttttggatctgtggctgctgcattttgggctttgggatctacctgctgatccacaacaacttcggagtgctcttccataacctcccctccctcacgctgggcaatgtgtttgtcatcgtgggctctattatcatggtagttgccttcctgggctgcatgggctctatcaaggaaaacaagtgtctgcttatgtcgttcttcatcctgctgctgattatcctccttgctgaggtgaccttggccatcctgctctttgtatatgaacagaagctgaatgagtatgtggctaagggtctgaccgacagcatccaccgttaccactcagacaatagcaccaaggcagcgtgggactccatccagtcatttctgcagtgttgtggtataaatggcacgagtgattggaccagtggcccaccagcatcttgcccctcagatcgaaaagtggagggttgctatgcgaaagcaagactgtggtttcattccaatttcctgtatatcggaatcatcaccatctgtgtatgtgtgattgaggtgttggggatgtcctttgcactgaccctgaactgccagattgacaaaaccagccagaccatagggctatgatctgcagtagttctgtggtgaagagacttgtttcatctccggaaatgcaaaaccatttatagcatgaagccctacatgatcactgcaggatgatcctcctcccatcctttccctttttaggtccctgtcttatacaaccagagaagtgggtgttggccaggcacatcccatctcaggcagcaagacaatctttcactcactgacggcagcagccatgtctctcaaagtggtgaaactaatatctgagcatcttttagacaagagaggcaaagacaaactggatttaatggcccaacatcaaagggtgaacccaggatatgaatttttgcatcttcccattgtcgaattagtctccagcctctaaataatgcccagtcttctccccaaagtcaagcaagagactagttgaagggagttctggggccaggctcactggaccattgtcacaaccctctgtttctctttgactaagtgccctggctacaggaattacacagttctctttctccaaagggcaagatctcatttcaatttctttattagagggccttattgatgtgttctaagtctttccagaaaaaaactatccagtgatttatatcctgatttcaaccagtcacttagctgataatcacagtaagaagacttctggtattatctctctatcagataagattttgttaatgtactattttactcttcaataaataaaacagtttattatctc

SEQ ID NO: 9 - DNA, 1424 bp, Homo sapiens
aggagccttaggaggtacggggagctcgcaaatactccttttggtttattcttaccaccttgcttctgtgttccttgggaatgctgctgtgcttatgcatctggtctctttttggagctacagtggacaggcatttgtgacagcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaataaaaaaaaaaaaaaaaaaaa

SEQ ID NO: 10 - DNA, 976 bp, Homo sapiens
gcgactgtctccgccgagcccccggggccaggtgtcccgggcgcgccacgatgcggccgcggctgtggctcctcttggccgcgcagctgacagttctccatggcaactcagtcctccagcagacccctgcatacataaaggtgcaaaccaacaagatggtgatgctgtcctgcgaggctaaaatctccctcagtaacatgcgcatctactggctgagacagcgccaggcaccgagcagtgacagtcaccacgagttcctggccctctgggattccgcaaaagggactatccacggtgaagaggtggaacaggagaagatagctgtgtttcgggatgcaagccggttcattctcaatctcacaagcgtgaagccggaagacagtggcatctacttctgcatgatcgtcgggagccccgagctgaccttcgggaagggaactcagctgagtgtggttgatttccttcccaccactgcccagcccaccaagaagtccaccctcaagaagagagtgtgccggttacccaggccagagacccagaagggccggcggaggagagcccggcttcgtttcatgaaacagcctcaaggggaaggtatatcaggaacctttgtcccccaatgcctgcatggatactacagcaatactacaacctcacagaagctgcttaacccatggatcctgaaaacataggcaagaagcacaggtcctgatgagtggatctttactacttttaccagattcctccctcgctcagagggatcctcccacacacccagcagagacttctctgccaccatcaaccccccaagtcatagggggctcaagttctgccctggtgagctgtggcccccatccttgtgaaccccaaagtgtcccccttgtggaaccaaatgtatccatcttgaataaatttgcccaaaatcctaaagaccgggaaaagggaaccattgagcttggcaaaaaaaaaaaaaaad

SEQ ID NO: 11 - DNA, 762 bp, Homo sapiens
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtataccataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatga

SEQ ID NO: 12 - DNA, 717 bp, Homo sapiens
agagcgatcatgtcgcacaaacaaatttactattcggacaaatacgacgacgaggagtttgagtatcgacatgtcatgctgcccaaggacatagccaagctggtccctaaaacccatctgatgtctgaatctgaatggaggaatcttggcgttcagcagagtcagggatgggtccattatatgatccatgaaccagaacctcacatcttgctgttccggcgcccactacccaagaaaccaaagaaatgaagctggcaagctacttttcagcctcaagctttacacagctgtccttacttcctaacatctttctgataacattattatgttgccttcttgtttctcactttgatatttaaaagatgttcaatacactgtttgaatgtgctggtaactgctttgcttcttgagtagagccaccaccaccatagcccagccagatgagtgctctgtggacccacagcctaagctgagtgtgaccccagaagccacgatgtgctctgtatccagaacacacttggcagatggaggaagcatctgagtttgagaccatggctgttacagggatcatgtaaacttgctgtttttgttttttctgccgggtgttgtatgtgtggtgacttgcggatttatgtttcagtgtactggaaactttccattttattcaagaaatctgttcatgttaaaagccttgattaaagaggaagtttttataat

SEQ ID NO: 13 - DNA, 3100 bp, Homo sapiens
actcgtctctggtaaagtctgagcaggacagggtggctgactggcagatccagaggttcccttggcagtccacgccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctgcactgctgtgctgtgcccaagttgtggtgctgacaaagcttggaagagcctgcaggtgccttggccgcgtgcatagcccagacacagaagaggctggttcttacgatggcacccagtgagcactcccaagtctacagagtgatagccttccgtaacccaactctcctggactgccttgaatatcccctcccagtcaccttgtgcaagcccctgcccatctgggaaaataccccatcattcatgctactgccaacctggggagccagggctatgggagcagcttttttttcccccctagaaacgtttggaacaatgtaaaactttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttc

SEQ ID NO: 14 - DNA, 1225 bp, Homo sapiens
cgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagactttttttttatacgataaataacttttttttaagttacacatttttcagatataaaagactgaccaatattgtacagtttttattgcttgttggatttttgtcttgtgtttctttagtttttgtg

SEQ ID NO: 15 - DNA, 1593 bp, Homo sapiens
ctgggagcgcggcgtaggtggctgccgagtcttttcctgtttagggtcttatcctggcattgagggcgccggactggcgcttttggccggcttggcattgggtgggcggcttcttgggacccacatgagccagtggcatcatccccgcagtggctggggccggagacgcgacttttcaggacgctcctcagccaagaagaagggcggaaaccacatccccgaaaggtggaaagactatctcccagttggacagcggatgcctgggactcgtttcattgctttcaaagttcctttgcaaaagagttttgaaaagaaacttgctccagaagaatgcttttcccctttggatctttttaacaaaatccgagaacaaaatgaagaacttggactgattattgatttaacatatactcaacgctattataaaccagaggatttgccagaaactgttccttacttaaaaatttttacagttggacatcaagtgcctgatgatgagactatttttaaattcaaacacgctgttaatgggtttttgaaagaaaataaagataatgataaacttattggtgtccactgtacccatggtttaaacaggactggctacctcatttgcagatatttgattgatgtagaaggcgtgaggccagatgatgcaattgaattattcaataggtgccggggacattgcttagaaagacaaaactacattgaagaccttcagaatggtcctatcagaaagaattggaattccagtgtacccaggtcaagtgattttgaagactcagcacatctcatgcaaccagtccacaataagcctgttaaacaaggacctaggtataatctacatcagatccagggtcactcagctcctcgacatttccacacccagacccaaagtttgcaacaatcagtcagaaaattttcagagaatccacatgtttaccagagacaccatctccctcctcctggtccccctggagaggactattcacacaggaggtattcttggaatgtgaagcccaatgccagtcgggcagcccaggatagaagaaggtggtatccttataattactccagactctcctatccagcctgttgggaatggacccagtgatacaaacctgtcctggaattctacctggagaccagagctggcctgaaaattactggtgtgacttttaattagttcaggtctaatcaggtttctttattgttcccttatgtattcaagcttaaggaaaaattgcattgctgtttacctctttgctgataaatttgcagtaattacagcattgcaggaaaaacaatctgttattccagtcttaaatttttctaaaagaagacaatattttagaactgaagcattgagaacttcccttgcaaattatttttaaaattctatcttgtttttctatgtatttctttctgactagacttgtgatatgcgtgtgtttatgtacagaaatttttagtgtttttgttatgttctgttattgacccaaaggccatctttattttctataactgttcaaaatttatattaaaatctacttaggagataatttctttagaaaaaaaaaaaaaaaaaaa

SEQ ID NO: 16 - DNA, 2755 bp, Homo sapiens
cctacccgcgcgcaggccaagttgctgaatcaatggagccctccccaacccgggcgttccccagcgaggcttccttcccatcctcctgaccaccggggcttttcgtgagctcgtctctgatctcgcgcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtaaaaaaaaaaaaaaaaaaaaaaaaaaaaaaaaaaaaaaaaaaaaaaaaaaaaaaaaaaaaaaaaaaa

SEQ ID NO: 17 - DNA, 2727 bp, Homo sapiens
ggggattttgggcgccgaccagacgcggcattttcggaaaatagcgccctgtgcagctgaagcgctttgtgtgtagcggggccgcgtcagcccggccgggtacgaggcgcctcgggtccccgcaccacctcctgctgccttcccgtcgccgctcccgaagcttttccagatgtccctggtagatttgggaaagaagcttttagaagcggcacgagcaggtcaagatgatgaagttcgtattttgatggcaaatggagctccctttactacagactggctgggaacttctccacttcatctagcagcacagtatggtcattattccaccacagaggtactgctgcgagctggtgtgagcagagatgccagaaccaaagtggaccgaacaccattacatatggcagcttctgagggccatgccagcatagtagaggttttacttaagcatggtgctgatgtcaatgcaaaggacatgttaaagatgacagctctccattgggccacagaacacaatcatcaagaggtggtggaacttttaatcaaatatggtgctgatgtacacacgcaaagtaaattttgtaaaactgcatttgatatttcaatagacaatggaaatgaagatttagcagagatattacagattgctatgcagaaccaaatcaacacaaacccagagagtcctgacactgtgacaatacatgctgcaacaccacagtttatcattggacctggaggggtggtgaacctaacaggtctggtatcttcagaaaattcatccaaggcaacagatgaaacgggtgtatctgctgttcagtttggaaactcttctacatcagtattagctacattagctgccttagctgaagcatctgctccattgtccaattcttcagaaactccagtagtggccacagaagaagtagttactgcagaatctgtggatggtgccattcagcaagtagttagttcagggggtcagcaagtcatcacaatagttacagatggaattcagcttggaaatttgcactctattccaaccagtggaattggtcagcccatcattgtgaccatgccagatggacaacaagtattaacagtaccagcaacagacattgctgaagaaactgttataagtgaagaaccaccagctaagagacaatgtatcgaaataattgaaaaccgggtggaatctgcagaaatagaagagagagaagctcttcagaaacagctggatgaagcaaatcgagaagcacaaaaatatcgacagcagctcctaaagaaagaacaggaagcagaggcctacagacagaagttggaagctatgactcgtcttcagactaataaagaagctgtttaattgaaatgaacatgtagtttgattttacttttggtcaagaaagaatacaatcttgaactgtacacaacaaaggtacagccatgggaatacagaatgatagaagagactacagatggataattggacttaagccatgagctctgagttcttgtaacataaaactttactttagaagttgtgaaatgtatttaaaactgaattctgtaaatagtttttttttttttacagttccaaatgagttgataaagattgttgaagagatccaaaaccagaataagccactgtttttgtgaattctttttgattttagtacaaaccttaatttctcagaaacggaacagttttaagggtgatcgttgttggttaggccaaatgttgtgtaataattatggtggactgatgctggaattactcctgtaggtataaacctctgtatgaagagaagatttctcccaggaaatctttgtacagctttaagttgtgtcagattctctgaaaacattttttagaaagcaaaatttttatatttgttcaatttcagctatacccaagtagatttacatgtatatgaagcaaatatttttaaaaatttctgtttgtacatattctgcatgttttataatttcaaaatgcatcacttacataggtatttctcccacagaaatgatgaaagtgaccagaaaaaaacaaaaacaaaacccctttactctgtaggtcattgaaacgaagtaagctggcagctggttttattggaatgacagtgttctcggaaggagcagcctacaagataacttgaatttgccaattctgcaaaatctgtgcttttttgaaaatttaagagtggggacgtgaaactgtattctgtgccttccatcatgatttccacatgaaagcactttaaggcactgattttaagataatgtttttggaaaacccaatgcatatgggtttctgaaatattttatggacttatttctccccaggaaacgattcttacggaaaaaaattgcttttgtatgtagaacaggaactttttgtattacagtgatgcaatagacatgtctaatgtaacttctacttttccttttgaaagctcagtgtctgtgctatgacttgctctcatcacaatattgttgaattccacaatgtatggacattaaacactggcagactgttcactttttctttttttttttttggtaaaatattacttcaaacccctttttcttgctttatttttcagtgttttattgctttatgaactgtttaaccctgaaatccctctaggttatctatactgtataaaaaagcaattacccttaaaactgtactctggcctacttttctattttgcaattaaatatcttttt

SEQ ID NO: 18 - DNA, 1378 bp, Homo sapiens
gctggcaccgaggcctgaccatggacgaggaatacgatgtgatcgtgctgggcaccggcctgacggaatgtatcctgtcaggtataatgtcagtgaatggcaagaaagttcttcatatggatcgaaacccttactacggaggagaaagtgcatctataacaccattggaagatttatacaaaagatttaaaataccaggatcaccacccgagtcaatggggagaggaagagactggaatgttgacttgattcccaagttccttatggctaatggtcagctggttaagatgctgctttatacagaggtaactcgctatctggattttaaagtgactgaagggagctttgtctataagggtggaaaaatctacaaggttccttccactgaagcagaagccctggcatctagcctaatgggattgtttgaaaaacgtcgcttcaggaaattcctagtgtatgttgccaacttcgatgaaaaagatccaagaacttttgaaggcattgatcctaagaagaccacaatgcgagatgtgtataagaaatttgatttgggtcaagacgttatagattttactggtcatgctcttgcactttacagaactgatgattacttagatcaaccgtgttatgaaaccattaatagaattaaactttacagtgaatctttggcaagatatggcaaaagcccatacctttatccactctatggccttggagaactgccccaaggatttgcaaggctaagtgctatttatggaggtacctatatgctgaataaacccattgaagaaatcattgtacagaatggaaaagtaattggtgtaaaatctgaaggagaaattgctcgctgtaagcagctcatctgtgaccccagctacgtaaaagatcgggtagaaaaagtgggccaggtgatcagagttatttgcatcctcagccaccccatcaagaacaccaatgatgccaactcctgccagatcattattccacagaaccaagtcaatcgaaagtcagatatctacgtctgcatgatctcctttgcgcacaatgtagcagcacaagggaagtacattgctatagttagtacaactgtggaaaccaaggagcctgagaaggaaatcagaccagctttggagctcttggaaccaattgaacagaaatttgttagcatcagtgacctcctggtaccaaaagacttgggaacagaaagccagatctttatttcccgcacatatgatgccaccactcattttgagacaacgtgtgatgacattaaaaacatctataagaggatgacaggatcagagtttgactttgaggaaatgaagcgcaagaagaatgacatctatggggaagactaacagcagtacatgttattatg

SEQ ID NO: 19 - DNA, 2581 bp, Homo sapiens
ctcgcctcagtctcctctgtcctctcccaggcaagaggaccggcggaggcacctctctcgagtcttaggctgcggaatctaagactcagcgagaggagcccgggaggagacagaactttccccttttttcccatcccttcttcttgctcagagaggcaagcaaggcgcggagctttagaaagttcttaagtggtcaggaaggtaggtgcttccctttttctcctcacaaggaggtgaggctgggacctccgggccagcttctcacctcatagggtgtacctttcccggctccagcagccaatgtgcttcggagccactctctgcagagccagagggcaggccggcttctcggtgtgtgcctaagaggatggatcggaggtcccgggctcagcagtggcgccgagctcgccataattacaacgacctgtgcccgcccataggccgccgggcagccaccgcgctcctctggctctcctgctccatcgcgctcctccgcgcccttgccacctccaacgcccgtgcccagcagcgcgcggctgcccaacagcgccggagcttccttaacgcccaccaccgctccggcgcccaggtattccctgagtcccccgaatcggaatctgaccacgagcacgaggaggcagaccttgagctgtccctccccgagtgcctagagtacgaggaagagttcgactacgagaccgagagcgagaccgagtccgaaatcgagtccgagaccgacttcgagaccgagcctgagaccgcccccaccactgagcccgagaccgagcctgaagacgatcgcggcccggtggtgcccaagcactccaccttcggccagtccctcacccagcgtctgcacgctctcaagttgcgaagccccgacgcctccccaagtcgcgcgccgcccagcactcaggagccccagagccccagggaaggggaggagctcaagcccgaggacaaagatccaagggaccccgaagagtcgaaggagcccaaggaggagaagcagcggcgtcgctgcaagccaaagaagcccacccgccgtgacgcgtccccggagtccccttccaaaaagggacccatccccatccggcgtcactaatggaggacgccgtccagattctccttgttttcatggattcaggtgctggagaatctggtaaaagcaccattgtgaagcagatgaggatcctgcatgttaatgggtttaatggagagggcggcgaagaggacccgcaggctgcaaggagcaacagcgatggcagtgagaaggcaaccaaagtgcaggacatcaaaaacaacctgaaagaggcgattgaaaccattgtggccgccatgagcaacctggtgccccccgtggagctggccaaccccgagaaccagttcagagtggactacatcctgagtgtgatgaacgtgcctgactttgacttccctcccgaattctatgagcatgccaaggctctgtgggaggatgaaggagtgcgtgcctgctacgaacgctccaacgagtaccagctgattgactgtgcccagtacttcctggacaagatcgacgtgatcaagcaggctgactatgtgccgagcgatcaggacctgcttcgctgccgtgtcctgacttctggaatctttgagaccaagttccaggtggacaaagtcaacttccacatgtttgacgtgggtggccagcgcgatgaacgccgcaagtggatccagtgcttcaacgatgtgactgccatcatcttcgtggtggccagcagcagctacaacatggtcatccgggaggacaaccagaccaaccgcctgcaggaggctctgaacctcttcaagagcatctggaacaacagatggctgcgcaccatctctgtgatcctgttcctcaacaagcaagatctgctcgctgagaaagtccttgctgggaaatcgaagattgaggactactttccagaatttgctcgctacactactcctgaggatgctactcccgagcccggagaggacccacgcgtgacccgggccaagtacttcattcgagatgagtttctgaggatcagcactgccagtggagatgggcgtcactactgctaccctcatttcacctgcgctgtggacactgagaacatccgccgtgtgttcaacgactgccgtgacatcattcagcgcatgcaccttcgtcagtacgagctgctctaagaagggaacccccaaatttaattaaagccttaagcacaattaattaaaagtgaaacgtaattgtacaagcagttaatcacccaccatagggcatgattaacaaagcaacctttcccttcccccgagtgattttgcgaaacccccttttcccttcagcttgcttagatgttccaaatttagaaagcttaaggcggcctacagaaaaaggaaaaaaggccacaaaagttccctctcactttcagtaaaaataaataaaacagcagcagcaaacaaataaaatgaaataaaagaaacaaatgaaataaatattgtgttgtgcagcattaaaaaaaatcaaaataaaaattaaatgtgagcaaagaatgaaaaaaaaaaaaaaaaaaaa

SEQ ID NO: 20 - DNA, 896 bp, Homo sapiens
gaattcggcagctccaaccagggcagccttcctgagaagatgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaggaacaggagccgacccagcagtttatccctgtgaaaagagccatcccccatccagcctataatcctaagaacttctccaatgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacattcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctactaactacaggaagcaaactaagcccccgctgtaatgaaacaccttctctggagccaagtccagatttacactgggagaggtgccagcaactgaataaatacctcttagctgagtgg

SEQ ID NO: 21 - DNA, 534 bp, Homo sapiens
cgactttcccgatcgccaggcaggagtttctctcggtgactactatcgctgtcatgtctggtcgtggcaagcaaggaggcaaggcccgcgccaaggccaagtcgcgctcgtcccgcgctggccttcagttcccggtagggcgagtgcatcgcttgctgcgcaaaggcaactacgcggagcgagtgggggccggcgcgcccgtctacatggctgcggtcctcgagtatctgaccgccgagatcctggagctggcgggcaacgcggctcgggacaacaagaagacgcgcatcatccctcgtcacctccagctggccatccgcaacgacgaggaactgaacaagctgctgggcaaagtcaccatcgcccagggcggcgtcttgcctaacatccaggccgtactgctccctaagaagacggagagtcaccacaaggcaaagggcaagtgaggctgacgtccggcccaagtgggcccagcccggcccgcgtctcgaaggggcacctgtgaactcaaaaggctcttttcagagccaccca

SEQ ID NO: 22 - DNA, 1700 bp, Homo sapiens
cgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaaggaaaaaa

SEQ ID NO: 23 - DNA, 1035 bp, Homo sapiens
atctccctccaggcagcccttggctggtccctgcgagcccgtggagactgccagagatgtcctctttcggttacaggaccctgactgtggccctcttcaccctgatctgctgtccaggatcggatgagaaggtattcgaggtacacgtgaggccaaagaagctggcggttgagcccaaagggtccctcgaggtcaactgcagcaccacctgtaaccagcctgaagtgggtggtctggagacctctctaaataagattctgctggacgaacaggctcagtggaaacattacttggtctcaaacatctcccatgacacggtcctccaatgccacttcacctgctccgggaagcaggagtcaatgaattccaacgtcagcgtgtaccagcctccaaggcaggtcatcctgacactgcaacccactttggtggctgtgggcaagtccttcaccattgagtgcagggtgcccaccgtggagcccctggacagcctcaccctcttcctgttccgtggcaatgagactctgcactatgagaccttcgggaaggcagcccctgctccgcaggaggccacagccacattcaacagcacggctgacagagaggatggccaccgcaacttctcctgcctggctgtgctggacttgatgtctcgcggtggcaacatctttcacaaacactcagccccgaagatgttggagatctatgagcctgtgtcggacagccagatggtcatcatagtcacggtggtgtcggtgttgctgtccctgttcgtgacatctgtcctgctctgcttcatcttcggccagcacttgcgccagcagcggatgggcacctacggggtgcgagcggcttggaggaggctgccccaggccttccggccatagcaaccatgagtggcatggccaccaccacggtggtcactggaactcagtgtgactcctcagggttgaggtccagccctggctgaaggactgtgacaggcagcagagacttgggacattgccttttctagcccgaatacaaacacctggactt

SEQ ID NO: 24 - DNA, 1190 bp, Homo sapiens
ggcacgaggctgaggtgtatttcgggtcttgctggggctgagagagaccacagccctttggggggtacaaacaagagttcagttgctgtgaattctgccactgtgcccagctctgaagcctcagctcttgccaaacagacccgagacccatgtcagccccactggatgccgccctccacgcccttcaggaggagcaggccagactcaagatgaggctgtgggacctgcagcagctgagaaaggagctcggggactcccccaaagacaaggtcccattttcagtgcccaagatccccctggtattccgaggacacacccagcaggacccggaagtgcctaagtctttagtttccaatttgcggatccactgccctctgcttgcgggctctgctctgatcacctttgatgaccccaaagtggctgagcaggtgctgcaacaaaaggagcacacgatcaacatggaggagtgccggctgcgggtgcaggtccagcccttggagctgcccatggtcaccaccatccaggtgatggtgtccagccagttgagtggccggagggtgttggtcactggatttcctgccagcctcaggctgagtgaggaggagctgctggacaagctagagatcttctttggcaagactaggaacggaggtggcgatgtggacgttcgggagctactgccagggagtgtcatgctggggtttgctagggatggagtggctcagcgtctgtgccaaatcggccagttcacagtgccactgggtgggcagcaagtccctctgagagtctctccgtatgtgaatggggagatccagaaggctgagatcaggtcgcagccagttccccgctcggtactggtgctcaacattcctgatatcttggatggcccggagctgcatgacgtcctggagatccacttccagaagcccacccgcgggggcggggaggtagaggccctgacagtcgtaccccaaggacagcagggcctagcagtcttcacctctgagtcaggctaggggcctccccttctcatcctccccacccccccgccaaggttctcacactggcctgggcttgggtgcccatataggaggtctgtatgttcaccaacagtgcggaggggtcacacattgcaaaacactgcccagaacagtaaaaagagcctgcatgccaaaaaaaaaaaaaaaaaa

SEQ ID NO: 25 - DNA, 1642 bp, Homo sapiens
ccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttctactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctataaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatatt

SEQ ID NO: 26 - DNA, 647 bp, Homo sapiens
gctcactgagcaccgtcccagcatccggacaccacagcggcccttcgctccacgcagaaaaccacacttctcataccttcactcaacacttccttccccaaagccagaagatgcacaaggaggaacatgaggtggctgtgctgggggcaccccccagcaccatccttccaaggtccaccgtgattaacatccacagcgagacctccgtgcccgaccatgtcgtctggtccctgttcaacaccctcttcttgaactggtgctgtctgggcttcatagcattcgcctactccgtaaagtctagggacaggaagatggttggcgacgtgaccggggcccaggcctatgcctccaccgccaagtgcctgaacatctgggccctgattctgggcatcctcatgaccattggattcatcctgttactggtattcggctctgtaacagtctaccatattatgttacagataatacaggaaaaacggggttactagtagccgcccatagcctgcaacctttgcactccactgtgcaatgctggccctgcacgctggggctgttgcccctgcccccttggtcctgcccctagatacagcagtttatacccacacacctgtctacagtgtcattcaataaagtgcacgtgcttgtga

SEQ ID NO: 27 - DNA, 1202 bp, Homo sapiens
tgtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgctgacgtcacatggagcacagaaatcaatgttagcagatagccagcccatacaagatcgtattgtattgtaggaggcatcgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttaaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgct

SEQ ID NO: 28 - DNA, 1451 bp, Homo sapiens
gaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatc

SEQ ID NO: 29 - DNA, 1770 bp, Homo sapiens
gaggagtggattacatattccaacagttgttattacattggtaaggaaagaagaacttgggaagaaagagtttgctggcctgtgcttcgaagaactctgatctgctttctatagataatgaggaagaaatggtatgtgtggggacttcccagttggctgtaagttgccatttgaactaaacgaaatagatcaggaactgaggacatatctaaattttctagttttatagaaggcttttatccacaagaatcaagatcttccctctctgagcaggaatcctttgtgcattgaagactttagattcctctctgcggtagacgtgcacttataagtatttgatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatctccattttttttctgctgcttcatcgctgtagccatgggaatccgtttcattattatggtagcaatatggagtgctgtattcctaaactcattattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacatacatctgcatgcaaaggactgtgtaaagatgatcaaccatctcaataaaagccaggaacagagaagagattacaccagcggtaacactgccaaccgagactaaaggaaacaaacaaaaacaggacaaaatgaccaaagactgtcagatttcttagactccacaggaccaaaccatagaacaatttcactgcaaacatgcatgattctccaagacaaaagaagagagatcctaaaggcaattcagatatccccaaggctgcctctcccaccacaagcccagagtggatgggctgggggaggggtgctgttttaatttctaaaggtaggaccaacacccaggggatcagtgaaggaagagaaggccagcagatcagtgagagtgcaaccccaccctccacaggaaattgcctcatgggcagggccacagcagagagacacagcatgggcagtgccttccctgcctgtgggggtcatgctgccacttttaatgggtcctccacccaacggggtcagggaggtggtgctgccccagtgggccatgattatcttaaaggcattattctccagccttaagatcttaggacgtttcctttgctatgatttgtacttgcttgagtcccatgactgtttctcttcctctctttcttccttttggaatagtaatatccatcctatgtttgtcccactattgtattttggaagcacataacttgtttggtttcacaggttcacagttaagaaggaattttgcctctgaataaatagaatcttgagtctcatgcaaaaaaaaaaaaaaaaaaaaaaaaa

SEQ ID NO: 30 - DNA, 1280 bp, Homo sapiens
atgccccaactttccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccagggtcacacccgtcacacatcccatcagatgtcaactccaaacaaggcatgtccagacatcctccagctcctgatatccctactttctatcccttgtctccgggtggtgttggacagatcaccccacctcttggttggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgtgctctgcttttctcctcccccatcccttcctcattccttcaaccccttcccctaaccaccaccaccaccaccttttaggaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctga

SEQ ID NO: 31 - DNA, 1518 bp, Homo sapiens
gaaactttcacgcgagcgggaaatatgggatgtataaaatcaaaagggaaagacagcttgagtgacgatggagtagatttgaagactcaaccagttccagaatctcagcttttacctggacagaggtttcaaactaaagatccagaggaacaaggagacattgtggtagccttgtacccctatgatggcatccacccggacgacttgtctttcaagaaaggagagaagatgaaagtcctggaggagcatggagaatggtggaaagcaaagtcccttttaacaaaaaaagaaggcttcatccccagcaactatgtggccaaactcaacaccttagaaacagaagagtggtttttcaaggatataaccaggaaggacgcagaaaggcagcttttggcaccaggaaatagcgctggagctttccttattagagaaagtgaaacattaaaaggaagcttctctctgtctgtcagagactttgaccctgtgcatggtgatgttattaagcactacaaaattagaagtctggataatgggggctattacatctctccacgaatcacttttccctgtatcagcgacatgattaaacattaccaaaagcaggcagatggcttgtgcagaagattggagaaggcttgtattagtcccaagccacagaagccatgggataaagatgcctgggagatcccccgggagtccatcaagttggtgaaaaggcttggcgctgggcagtttggggaagtctggatgggttactataacaacagtaccaaggtggctgtgaaaaccctgaagccaggaactatgtctgtgcaagccttcctggaagaagccaacctcatgaagaccctgcagcatgacaagctcgtgaggctctacgctgtggtcaccagggaggagcccatttacatcatcaccgagtacatggccaagggcagtttgctggatttcctgaagagcgatgaaggtggcaaagtgctgcttccaaagctcattgacttttctgctcagattgcagagggaatggcatacatcgagcggaagaactacattcaccgggacctgcgagcagctaatgttctggtctccgagtcactaatgtgcaaaattgcagattttggccttgctagagtaattgaagataatgagtacacagcaagggaaggtgctaagttccctattaagtggacggctccagaagcaatcaactttggatgtttcactattaagtctgatgtgtggtcctttggaatcctcctatacgaaattgtcacctatgggaaaattccctacccagggagaactaatgccgacgtgatgaccgccctgtcccagggctacaggatgccccgtgtggagaactgcccagatgagctctatgacattatgaaaatgtgctggaaagaaaaggcagaagagagaccaacgtttgactacttacagagcgtcctggatgatttctacacagccacggaagggcaataccagcagcagccttagagcacagggagacccgtc

SEQ ID NO: 32 - DNA, 396 bp, Homo sapiens
actccgccttccacgtgcacccactgcctcttcccttctcgcttgggaactctagtctcgcctcgggttgcaatggaccccaactgctcctgtgccgctggtgtctcctgcacctgcgccagctcctgcaagtgcaaagagtgcaaatgcacctcctgcaagaagagctgctgctcctgctgccctgtgggctgtgccaagtgtgcccaaggctgcatctgcaaaggggcatcggagaagtgcagctgctgcgcctgatgtcgggacagccctgctcccaagtacaaatagagtgacccgtaaaatctaggattttttgttttttgctacaatcttgacccctttgctacattcccttttttctgtgaaatatgtgaataataattaaacacttag

SEQ ID NO: 33 - DNA, 367 bp, Homo sapiens
ctccagtctcacctcggcttgcaatggaccccaactgctcctgcgaggctggtggctcctgcgcctgcgccggctcctgcaagtgcaaaaagtgcaaatgcacctcctgcaagaagagctgctgctcctgttgccccctgggctgtgccaagtgtgcccagggctgcatctgcaaaggggcgtcagagaagtgcagctgctgtgcctgatgtcgggacagccctgctgtcagatgaaaacagaatgacacgtaaaatccgaggttttttttttctacaactccgactcatttgctacattcctttttttctgtgaaatatgtgaataataattaaacacttagacttgaaaaaaaaaaaaaaaaaaa

SEQ ID NO: 34 - DNA, 2787 bp, Homo sapiens
agagcggaggccgcactccagcactgcgcagggaccgccttggaccgcagttgccggccaggaatcccagtgtcacggtggacacgcctccctcgcgcccttgccgcccacctgctcacccagctcaggggctttggaattctgtggccacactgcgaggagatcggttctgggtcggaggctacaggaagactcccactccctgaaatctggagtgaagaacgccgccatccagccaccattccaaggaggtgcaggagaacagctctgtgataccatttaacttgttgacattacttttatttgaaggaacgtatattagagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaaattaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgcggagccctgtctcctctctctgtaataaactcatttctagcccg

SEQ ID NO: 35 - DNA, 2682 bp, Homo sapiens
gggtagacccacgagtccgcccacgggtctgcatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggctgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctgaagactgttctgaggccctgggtgtgtgtgtatctgtctgcctgtccatgtacttctgccctgcctcctcctttcgttgtaggaggaatctgtgctctacttacctctcaattcctggagatgccaacttcacagacacgtctgcagcagctggacatcacatttcatgtcctgcatggaaccagtggctgtgagtggcatgtccacttgctggattatcagccaggacactatagaacaggaccagctgagactaagaaggaccagcagagccagctcagctctgagccattcacacatcttcaccctcagtttcctcacttgaggagtgggatggggagaacagagagtagctgtgtttgaatccctgtaggaaatggtgaagcatagctctgggtctcctgggggagaccaggcttggctgcgggagagctggctgttgctggactacatgctggccactgctgtgaccacgacactgctggggcagcttcttccacagtgatgcctactgatgcttcagtgcctctgcacaccgcccattccacttcctccttccccacagggcaggtggggaagcagtttggcccagcccaaggagaccccaccttgagccttatttcctaatgggtccacctctcatctgcatctttcacacctcccagcttctgcccaaccttcagcagtgacaagtccccaagagactcgcctgagcagcttgggctgcttttcatttccacctgtcaggatgcctgtggtcatgctctcagctccacctggcatgagaagggatcctggcctctggcatattcatcaagtatgagttctggggatgagtcactgtaatgatgtgagcagggagccttcctccctgggccacctgcagagagctttcccaccaactttgtaccttgattgccttacaaagttatttgtttacaaacagcgaccatataaaagcctcctgccccaaagcttgtgggcacatgggcacatacagactcacatacagacacacacatatatgtacagacatgtactctcacacacacaggcaccagcatacacacgtttttctaggtacagctcccaggaacagctaggtgggaaagtcccatcactgagggagcctaaccatgtccctgaacaaaaattgggcactcatctattccttttctcttgtgtccctactcattgaaaccaaactctggaaaggacccaatgtaccagtatttatacctctaatgaagcacagagagaggaagagagctgcttaaactcacacaacaatgaactgcagacacagctgttctctccctctctccttcccagagcaatttatactttaccctcaggctgtcctctggggagaaggtgccatggtcttaggtgtctgtgccccaggacagaccctaggaccctaaatccaatagaaaatgcatatctttgctccactttcagccaggctggagcaaggtaccttttcttaggatcttgggagggaatggatgcccctctctgcatgatcttgttgaggcatttagctgccatgcacctgtccccctttaatactgggcattttaaagccatctcaagaggcatcttctacatgttttgtacgcattaaaataatttcaaagatatctgagaaaagccgatatttgccattcttcctatatcctggaatatatcttgcatcctgagtttataataataaataatattctaccttggaaaaaaaaaaaaaaaaaaaaaaaaaa

SEQ ID NO: 36 - DNA, 1821 bp, Homo sapiens
cagagcccaacacatacagctgatacacgcagaccagatctggtcaggtcctcggaagctgagtccagagcgatgctgctgaagacagtgctcttgctgggacatgtggcccaggtgctgatgctggacaatgggctcctgcagacaccacccatgggctggctggcctgggaacgcttccgctgcaacattaactgtgatgaggacccaaagaactgcataagtgaacagctcttcatggagatggctgaccggatggcacaggatggatggcgggacatgggctacacatacctcaacattgatgactgctggatcggtggtcgcgatgccagtggccgcctgatgccggatcccaagcgcttccctcatggcattcctttcctggctgactacgttcactccctgggcctgaagttgggtatctacgcggacatgggcaacttcacctgcatgggttacccaggcaccacactggacaaggtggtccaggatgctcagaccttcgccgagtggaaggtagacatgctcaagctggatggctgcttctccacccccgaggagcgggcccaggggtaccccaagatggctgctgccctgaatgccacaggccgccccatcgccttctcctgcagctggccagcctatgaaggcggcctccccccaagggtgaactacagtctgctggcggacatctgcaacctctggcgtaactatgatgacatccaggactcctggtggagcgtgctctccatcctgaattggttcgtggagcaccaggacatactgcagccagtggccggccctgggcactggaatgaccctgacatgctgctcattgggaactttggtctcagcttagagcaatcccgggcccagatggccctgtggacggtgctggcagcccccctcttgatgtccacagacctgcgtaccatctccgcccagaacatggacattctgcagaatccactcatgatcaaaatcaaccaggatcccttaggcatccagggacgcaggattcacaaggaaaaatctctcatcgaagtgtacatgcggcctctgtccaacaaggctagcgccttagtcttcttcagctgcaggaccgatatgccttatcgctaccactcctcccttggccagctgaacttcaccgggtctgtgatatatgaggcccaggacgtctactcaggtgacatcatcagtggcctccgagatgaaaccaacttcacagtgatcatcaacccttcaggggtagtgatgtggtacctgtatcccatcaagaacctggagatgtcccagcagtgaggagctgggacatgtgacaggctgtggtggcaccactgagcctagaccatggagccttggcatgcccagggcaagtggggaggttctctgctccccaggcctgctcggtgactgaccccatcatacccaaagtgcaatctcacggccaggttctatgccctgtccaagcgtaaaccctcttggaaacttcttttggggcaattttcctgtggccttcctggcctctacttccatgtgcgcagccccacagacgttgctgagcaactcgccagcctcctgagctccatgcccatcaggactctagcctctgaccttgctgttgactctgaaatcaggatttggaagttttcgaattaggagtagagagatctgacctcttgccaggaatgcccatggatcatgtgattggcttttctacccatagagggccttgcagcctgataccacctgggagtgagggtcacaaaggagaccttggctccctcaggtcaccaataaacctgttctttaatc

SEQ ID NO: 37 - DNA, 1418 bp, Homo sapiens
aactgtgcgaaccagacccggcagccttgctcagttcagcatagcggagcggatccgatcggatcggagcacaccggagcaggctcatcgagaaggcgtctgcgagaccatggagaacggatacacctatgaagattataagaacactgcagaatggcttctgtctcatactaagcaccgacctcaagttgcaataatctgtggttctggattaggaggtctgactgataaattaactcaggcccagatctttgactacagtgaaatccccaactttcctcgaagtacagtgccaggtcatgctggccgactggtgtttgggttcctgaatggcagggcctgtgtgatgatgcagggcaggttccacatgtatgaagggtacccactctggaaggtgacattcccagtgagggttttccaccttctgggtgtggacaccctggtagtcaccaatgcagcaggagggctgaaccccaagtttgaggttggagatatcatgctgatccgtgaccatatcaacctacctggtttcagtggtcagaaccctctcagagggcccaatgatgaaaggtttggagatcgtttccctgccatgtctgatgcctacgaccggactatgaggcagagggctctcagtacctggaaacaaatgggggagcaacgtgagctacaggaaggcacctatgtgatggtggcaggccccagctttgagactgtggcagaatgtcgtgtgctgcagaagctgggagcagacgctgttggcatgagtacagtaccagaagttatcgttgcacggcactgtggacttcgagtctttggcttctcactcatcactaacaaggtcatcatggattatgaaagcctggagaaggccaaccatgaagaagtcttagcagctggcaaacaagctgcacagaaattggaacagtttgtctccattcttatggccagcattccactccctgacaaagccagttgacctgccttggagtcgtctggcatctcccacacaagacccaagtagctgctaccttctttggccccttgctggagtcatgtgcctctgtccttaggttgtagcagaaaggaaaagattcctgtccttcacctttcccactttcttctaccagacccttctggtgccagatcctcttctcaaagctgggattacaggtgtgagcatagtgagaccttggcgctacaaaataaagctgttctcattcctgttctttcttacacaagagctggagcccgtgccctaccacacatctgtggagatgcccaggatttgactcgggccttagaactttgcatagcagctgctactagctctttgagataatacattccgaggggctcagttctgccttatctaaatcaccagagaccaaacaaggactaatccaatacctcttgga

SEQ ID NO: 38 - DNA, 2525 bp, Homo sapiens
tcctgccccgcggcgctgccgcacgagccccacgagccgctcaccccgccgttctcagcgctgcccgaccccgctggcgcgccctcccgccgccagtcccggcagcgccctcagttgtcctccgactcgccctcggccttccgcgccagccgcagccacagccgcaacgccacccgcagccacagccacagccacagccccaggcatagccttcggcacagccccggctccggctcctgcggcagctcctctgggcaccgtccctgcgccgacatcctggaggttgggatgctcttgtccaaaatcaactcgcttgcccacctgcgcgccgcgccctgcaacgacctgcacgccaccaagctggcgcccggcaaggagaaggagcccctggagtcgcagtaccaggtgggcccgctactgggcagcggcggcttcggctcggtctactcaggcatccgcgtctccgacaacttgccggtggccatcaaacacgtggagaaggaccggatttccgactggggagagctgcctaatggcactcgagtgcccatggaagtggtcctgctgaagaaggtgagctcgggtttctccggcgtcattaggctcctggactggttcgagaggcccgacagtttcgtcctgatcctggagaggcccgagccggtgcaagatctcttcgacttcatcacggaaaggggagccctgcaagaggagctcgcccgcagcttcttctggcaggtgctggaggccgtgcggcactgccacaactgcggggtgctccaccgcgacatcaaggacgaaaacatccttatcgacctcaatcgcggcgagctcaagctcatcgacttcgggtcgggggcgctgctcaaagacaccgtctacacggacttcgatgggacccgagtgtatagccctccagagtggatccgctaccatcgctaccatggcaggtcggcggcagtctggtccctggggatcctgctgtatgatatggtgtgtggagatattcctttcgagcatgacgaagagatcatcaggggccaggttttcttcaggcagagggtctcttcagaatgtcagcatctcattagatggtgcttggccctgagaccatcagataggccaaccttcgaagaaatccagaaccatccatggatgcaagatgttctcctgccccaggaaactgctgagatccacctccacagcctgtcgccggggcccagcaaatagcagcctttctggcaggtcctcccctctcttgtcagatgcccgagggaggggaagcttctgtctccagcttcccgagtaccagtgacacgtctcgccaagcaggacagtgcttgatacaggaacaacatttacaactcattccagatcccaggcccctggaggctgcctcccaacagtgaggaagagtgactctccaggggtcctaggcctcaactcctcccatagatactctcttcttctcataggtgtccagcattgctggactgctgaaatatcccgggggtggggggtgggggtgggtcagaaccctgccatggaactgtttccttcatcatgagttctgctgaatgccgcgatgggtcaggtaggggggaaacaggttgggatgggataggactagcaccattttaagtccctgtcacctcttccgactctttctgagtgccttctgtggggactccggctgtgctgggagaaatacttgaacttgcctcttttacctgctgcttctccaaaaatctgcctgggttttgttccctatttttctctcctgtcctccctcaccccctccttcatatgaaaggtgccatggaagaggctacagggccaaacgctgagccacctgcccttttttctgcctcctttagtaaaactccgagtgaactggtcttcctttttggtttttacttaactgtttcaaagccaagacctcacacacagaaaaaatgcacaaacaatgcaatcaacagaaaagctgtaaatgtgtgtacagttggcatggtagtatacaaaaagattgtagtggatctaatttttcagaaattttgcctttaagttattttacctgtttttgtttcttgttttgaaagatgcgcattctaacctggaggtcaatgttatgtatttatttatttatttatttggttcccttcctattccaagcttccatagctgctgccctagttttctttcctcctttcctcctctgacttggggaccttttgggggagggctgcgacgcttgctctgtttgtggggtgacgggactcaggcgggacagtgctgcagctccctggcttctgtggggcccctcacctacttacccaggtgggtcccggctctgtgggtgatggggaggggcattgctgactgtgtatataggataattatgaaaagcagttctggatggtgtgccttccagatcctctctggggctgtgttttgagcagcaggtagcctggctggttttatctgagtgaaatactgtacaggggaataaaagagatcttattttt

SEQ ID NO: 39 - DNA, 2866 bp, Homo sapiens
gaggggaagaggatgtgatcatgcgaccgccagcagaagggaaattgtgaaagcgacctgctgtagaaaaggcggcccacagccacctcagcatgcagaggaggcccagctgctgagaggagttgcctgagagtgacctttgcatctgcctgtccagccagcatggaaccaaagcggatcagagagggctaccttgtgaagaaggggagcgtgttcaatacgtggaaacccatgtgggttgtattgttagaagatggaattgaattctataagaagaaaagtgacaacagccccaaaggaatgatcccgctgaaagggagcactctgactagcccttgtcaagactttggcaaaaggatgtttgtgtttaagatcactacgaccaaacagcaggaccacttcttccaggcagccttcctggaggagagagatgcctgggttcgggatatcaagaaggccattaaatgcattgaaggaggccagaaatttgccaggaaatctaccaggaggtccattcgactgccagaaaccattgacttaggtgccttatatttgtccatgaaagacactgaaaaaggaataaaagaactgaatctagagaaggacaagaagatttttaatcactgcttcacaggtaactgcgtcattgattggctggtatccaaccagtctgttaggaatcgccaggaaggcctcatgattgcttcatcgctgctcaatgaggggtatctgcagcctgctggagacatgtccaagagtgcagtggatggaactgctgaaaaccctttcctggacaaccctgatgccttctactactttccagacagtgggttcttctgtgaagagaattccagtgatgatgatgtgattctgaaagaagaattcagaggggtcattatcaagcagggatgtttactgaagcaggggcatagaaggaaaaactggaaagtgaggaagttcatcttgagagaagaccctgcctacctgcactactatgaccctgctggggcagaagatcccctgggagcaattcacttgagaggctgtgtggtgacttcagtggagagcaactcaaatggcaggaagagtgaggaagagaacctttttgagatcatcacagcagatgaagtgcactatttcttgcaagcagccacccccaaggagcgcacagagtggatcagagccatccagatggcctcccgaactgggaagtaaagagactcctgcattcctcctcccctcctgagggaagcccatggacaagctcagtccaggacctgtccacttctgtgacaaatcaacgggaaacagcccaggggtgggaagttttcatttgcaggggggtctgaatgtaactcaccatgtggtgtgcaaggttcccctgcattgtattgctcactgcagcccctctgcccctatccatgacccccaagcagatataacaagctgtgcagcctcagtaggctgcttgccctctccaggcctcagggcctcttctggaaaatgaagaaattcaactagtagattcctgaggtccccctagcttaaaaaaaaaaaaaatctgccccatgattctaacactcgcagtagtgatagtgtatctagttgttctgctggtgtccttccttggctaagtcttggccttcagttatcttcaaatgtaccagaacctgagccaacgcctccctgtgaaactgttgctgatctgtagtacagtaccaggaagaaacctcttttgttctctttagacatcttctacttgctcttggccttgagatcgtgtaacaaaatgaaggagggctctcttctttcttcctcatcctactcaaaaacttcccgagagcagtggtggttttgagggttttgacttctattacttttggcagcctggaaagttgtgtcttctgggaaagagacctggggaggccaggagtagctgagggtcctttctgtgcccttaaaccgcccagaggagccctattccactctggttttaggctgatctgagagggtctccctttgttcctttctggagcatttctctaacgtttattacaattaggagggggaccccacatctgtgagattctgtttcatttgaggtttacagaaaaaaaaaagtggccagatgtgttccccccatgggtgagaggcctgggcaactgcctggtgaatgtgtcttgcggcagctgcagcaagtggaggggctgaactactggccagctcactggatgatgggttaatacaacaactgcactgtaaggactcagagccacacagaacttctgagaggggctgttagcattgcgcagcatcttcagttctccagtaaatgatattgcgttcgtgcctcagctttaagcacaagtagcagcagctcctgcttgagttctgagggcatcatggccctatgattaaccagagtgatctaacctagactaaaattgggaacttatttgcaatttttgaccctgaccactaactagtgattcttctccaaaattgagaaagacagcacccattgaagcagatatgtgtgtgaaagtatatttttcaattccagatttttaattttaaggctccaggaaagaaaggagagtagaacatttttcctcattttatcaaatcctctcttgccctccctcaattcccctgtaacattcctgaagctgttcccactcccagatggttttatcaatagcctagaggtaaagaactgtctttttctctgattctttaataaattatctttatagaatatgcacaagtttttctacactcagtgttaaagtatttattaatgggaagtcaacttaatgttttgaaataaatatatgactctgtttaatgcaaaaaaaaaaaaaaa

SEQ ID NO: 40 - DNA, 996 bp, Homo sapiens
agcagaggactttttagctgctcactggccccgcttgtctgaccgactcatccgcccgcgacccctaatcccctctgcctgccccaagatgctgaagccagccctggagccccgagggggcttctccttcgagaactgccaaagaaatgcatcattggaacgcgtcctcccggggctcaaggtccctcacgcacgcaagaccgggaccaccatcgcgggcctggtgttccaagacggggtcattctgggcgccgatacgcgagccactaacgattcggtcgtggcggacaagagctgcgagaagatccacttcatcgcccccaaaatctactgctgtggggctggagtagccgcggacgccgagatgaccacacggatggtggcgtccaagatggagctacacgcgttatctacgggccgcgagccccgcgtggccacggtcactcgcatcctgcgccagacgctcttcaggtaccagggccacgtgggtgcatcgctgatcgtgggcggcgtagacctgactggaccgcagctctacggcgtgcatccccatggctcctacagccgtctgcccttcacagccctgggctctggtcaggacgcggccctggcggtgctagaagaccggttccagccgaacatgacgctggaggctgctcaggggctgctggtggaagccgtcaccgccgggatcttgggtgacctgggctccgggggcaatgtggacgcatgtgtgatcacaaagactggcgccaagctgctgcggacactgagctcacccacagagcccgtgaagaggtctggccgctaccactttgtgcctggaaccacagctgtcctgacccagacagtgaagccactaaccctggagctagtggaggaaactgtgcaggctatggaggtggagtaagctgaggcttagagcttggaacaagggggaataaacccagaaaatacagttaaaaaaaaaaaaaacaaaaaaaaaaaaaaaaaaaa

SEQ ID NO: 41 - DNA, 724 bp, Homo sapiens
ggaaaccagtgccccaggcggcgaaggagagcggtgccttgcagggatgctgcgggcgggagcaccaaccggggacttaccccgggcgggagaagtccacaccgggaccaccatcatggcagtggagtttgacgggggcgttgtgatgggttctgattcccgagtgtctgcaggcgaggcggtggtgaaccgagtgtttgacaagctgtccccgctgcacgagcgcatctactgtgcactctctggttcagctgctgatgcccaagccgtggccgacatggccgcctaccagctggagctccatgggatagaactggaggaacctccacttgttttggctgctgcaaatgtggtgagaaatatcagctataaatatcgagaggacttgtctgcacatctcatggtagctggctgggaccaacgtgaaggaggtcaggtatatggaaccctgggaggaatgctgactcgacagccttttgccattggtggctccggcagcacctttatctatggttatgtggatgcagcatataagccaggcatgtctcccgaggagtgcaggcgcttcaccacagacgctattgctctggccatgagccgggatggctcaagcgggggtgtcatctacctggtcactattacagctgccggtgtggaccatcgagtcatcttgggcaatgaactgccaaaattctatgatgagtgaaccttccccagacttctc

SEQ ID NO: 42 - DNA, 2048 bp, Homo sapiens
gctcggtgctgatggacggtgctggtggccagtggagaggcgctggccgcacttcccgtcggggagagagtgtaatatggcgaagacctacgattacctgttcaagctgctgctgatcggggactcgggggtggggaagacctgtgtcctgttccgcttctccgaggacgccttcaactccacttttatctccaccataggaattgactttaaaattaggaccatagagctcgatggcaagagaattaaactgcagatatgggacacagccggtcaggaacggtttcggacgatcacaacggcctactacaggggtgcaatgggcatcatgctggtctacgacatcaccaacgagaagtccttcgacaacatccggaactggattcgcaacattgaggagcacgcctctgcagacgtcgaaaagatgatactcgggaacaagtgtgatgtgaatgacaagagacaagtttccaaggaacggggagaaaagctggccctcgactatggaatcaagttcatggagaccagcgcgaaggccaacatcaatgtggaaaatgcatttttcactctcgccagagatatcaaagcaaaaatggacaaaaaattggaaggcaacagcccccaggggagcaaccagggagtcaaaatcacaccggaccagcagaagaggagcagctttttccgatgtgttcttctgtgaggaacaccgccttactctgagcctcgctcagcccagctgactgtgcctgttctgagtgagcccctcactcagccggggccctcccacctccaacgccccgcccacgccgcggccaccgggcccacggccaccagaatgcaattgagaaatcgtttattttagtaactgtctgatctttttcaactttggagatggaataagttaaaaatttgctatttttcctgtaacatctgctgaacgggcccacccacacgttgtatattcagagagagagagggagtcaaggtgtgaccgtcgaccacagccagtgtcaggcctctgcctctgggcctttgctttgtggcctcactgcaacacaaagctccaccaggaggctggttcacgtcccctaccacggaagcgaggtcccagaaggccagcggtggttccaggagcaacagctcccaaaccctgagcaaggcaaccgatcgccaggaccaggaagcatcacccaggagatttggcgcccacttccacctcttctctcagttttggacaagtgacaaaccattttgccccctcactcttcttttttaactgttaaaccaaaggaaagcacaaatgaaggaaatcctgtgtaaagcattgagaaggaaagaagcctggagcagcctctcctgtccacagccaggggttaggtctgcaggcccgtctgcggtccccatcgagcatcaaggggacgctgtgtgtgctgcaagtgaccccgaaaacaaccacagccgtcacatggtcctcctgaagttggggcaccctcctctcagcaccaaaatggcccccactccttcgtgtcctcccgctatctccaaatcggacgttctttctagctgagatttttatttttccacatctgtagtgccatgaagcgattctgtctttgacttccaatggcaaacctgggtgatcgggaacaagcacgttgtacccttggctggacatggccaagacacaaggcattccacggcggcaagctgaccgcacagcagtctggcttgattttcagccgtcatcattgggttctgttttgacagctctgctgtcccatagggactgcgactgggaccaggtcaaccacgccaggggggtgtcaccagccttttctttttttctttctttttttttttttcctccttaagctgctgtcaatccaaaccattggcatcatcgtttcttttcaattaaaaccaacatatcagcaatagtctgctctccccgggaatctctaacatgctctgtttacatcgataaatgcacttaaggaaaacaaacaaattaaagctcatcttaaagtccaaaaaaaaaaaaaaaaaaa

SEQ ID NO: 43 - DNA, 793 bp, Homo sapiens
atgcaggccatcaagtgtgtggtggtgggagatggggccgtgggcaagacctgccttctcatcagctacaccaccaacgcctttcccggagagtacatccccaccgtgtttgacaactattcagccaatgtgatggtggacagcaagccagtgaacctggggctgtgggacactgctgggcaggaggactacgaccgtctccggccgctctcctatccacagacggacgtcttcctcatctgcttctccctcgtcagcccagcctcttatgagaacgtccgcgccaagtggttcccagaagtgcggcaccactgccccagcacacccatcatcctggtgggcaccaagctggacctgcgggacgacaaggacaccatcgagaaactgaaggagaagaagctggctcccatcacctacccgcagggcctggcactggccaaggagattgactcggtgaaatacctggagtgctcagccctcacccagagaggcctgaaaaccgtgttcgacgaggccatccgggccgtgctgtgccctcagcccacgcggcagcagaagcgcgcctgcagcctcctctaggggttgcaccccagcgctcccacctagatgggtctgatccgccgccgccccaggcccgcgggctgctgacagagatacgcgccgtggtgcgcacggagcccttcacgggacgctacagcctgtgccgggccgggagctgggcagggggaaattagcagtggtgagaaaatgtataaagatagattctgggaaagaatttgctgcaaagttcatg

SEQ ID NO: 44 - DNA, 595 bp, Homo sapiens
gggcaaggctgggccgggaagggcgtgggttgaggagaggctccagacccgcacgccgcgcgcacagagctctcagcgccgctcccagccacagcctcccgcgcctcgctcagctccaacatggcaaaaatctccagccctacagagactgagcggtgcatcgagtccctgattgctgtcttccagaagtatgctggaaaggatggttataactacactctctccaagacagagttcctaagcttcatgaatacagaactagctgccttcacaaagaaccagaaggaccctggtgtccttgaccgcatgatgaagaaactggacaccaacagtgatggtcagctagatttctcagaatttcttaatctgattggtggcctagctatggcttgccatgactccttcctcaaggctgtcccttcccagaagcggacctgaggaccccttggccctggccttcaaacccaccccctttccttccagcctttctgtcatcatctccacagcccacccatcccctgagcacactaaccacctcatgcaggccccacctgccaatagtaataaagcaatgtcacttttttaaaacatgaaa

SEQ ID NO: 45 - DNA, 2324 bp, Homo sapiens
ctccctttgggcaaggacctgagacccttgtgctaagtcaagaggctcaatgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggcaagaaatccaagagaagtatgaatgacccatattaaatcgcccttggtgaaagaaaattcttggaatactaaaaatcatgagatcctttaaatccttccatgaaacgttttgtgtggtggcacctcctacgtcaaacatgaagtgtgtttccttcagtgcatctgggaagatttctacctgaccaacagttccttcagcttccatttcgcccctcatttatccctcaacccccagcccacaggtgtttatacagctcagctttttgtcttttctgaggagaaacaaataagaccataaagggaaaggattcatgtggaatataaagatggctgactttgctctttcttgactcttgttttcagtttcaattcagtgctgtacttgatgacagacacttctaaatgaagtgcaaatttgatacatatgtgaatatggactcagttttcttgcagatcaaatttcacgtcgtcttctgtatactgtggaggtacactcttatagaaagttcaaaaagtctacgctctcctttctttctaactccagtgaagtaatggggtcctgctcaagttgaaagagtcctatttgcactgtagcctcgccgtctgtgaattggaccatcctatttaactggcttcagcctccccaccttcttcagccacctctctttttcagttggctgacttccacacctagcatctcatgagtgccaagcaaaaggagagaagagagaaatagcctgcgctgttttttagtttgggggttttgctgtttccttttatgagacccattcctatttcttatagtcaatgtttcttttatcacgatattattagtaagaaaacatcactgaaatgctagctgcaagtgacatctctttgatgtcatatggaagagttaaaacaggtggagaaattccttgattcacaatgaaatgctctcctttcccctgcccccagaccttttatccacttacctagattctacatattctttaaatttcatctcaggcctccctcaaccccaccacttcttttataactagtcctttactaatccaacccatgatgagctcctcttcctggcttcttactgaaaggttaccctgtaacatgcaattttgcatttgaataaagcctgctttttaagtgttaa

SEQ ID NO: 46 - DNA, 2381 bp, Homo sapiens
agtgccgccgggactcttggcgggtgaaggtgtgtgtcagcttttgcgtcactcgagccctgggcgctgcttgctaaagagccgagcacgcgggtctgtcatcatgtcgcgttacgggcggtacggaggagaaaccaaggtgtatgttggtaacctgggaactggcgctggcaaaggagagttagaaagggctttcagttattatggtcctttaagaactgtatggattgcgagaaatcctccaggatttgcctttgtggaattcgaagatcctagagatgcagaagatgcagtacgaggactggatggaaaggtgatttgtggctcccgagtgagggttgaactatcgacaggcatgcctcggagatcacgttttgatagaccacctgcccgacgtccctttgatccaaatgatagatgctatgagtgtggcgaaaagggacattatgcttatgattgtcatcgttacagccggcgaagaagaagcaggtcacggtctagatcacattctcgatccagaggaaggcgatactctcgctcacgcagcaggagcaggggacgaaggtcaaggtcagcatctcctcgacgatcaagatctatctctcttcgtagatcaagatcagcttcactcagaagatctaggtctggttctataaaaggatcgaggtatttccaatccccgtcgaggtcaagatcaagatccaggtctatttcacgaccaagaagcagccgatcaaagtccagatctccatctccaaaaagaagtcgttccccatcaggaagtcctcgcagaagtgcaagtcctgaaagaatggactgaagctctcaagttcaccctttagggaaaagttattttgtttacattattataagggatttgtgatgtctgtaaagtgtaacctaggaaagataattcaaccatctaatcaaaatggatctggattactatgtaaattcacagcagtaagataatataaattttgttgaatgtattaacatcatatggtctgaaaatgtgggtttttatttggcacatttaaataaaatgtttctaactagatttttgatttgtgttcaatattaacacttcttaatttgatatatttgagagtcagacattataattgttaaccttattcatacatacctacattcagaattgaaaggtgttggttaagtcttgaacatcactattctatgcataaaacttggccaggatcttaagggactttgaaaattccatcttacccttgtagctctgggtaagatgacctgagtcccttatgatacagcctgaatgcatcatgacagatccttaagttagctaatccgtttgaagttggtgttagtaggtattgtatgatcagtggtgaagcaagtaggaccactgatgtgtctaaatgagcatgacaggaactaaacgaaactgattaaatgtatgagaaatagaaactgatttctggatgatctttatactaattgcagctttcaggctactaggtggcatagtgttaattaggactccccaagatatggggagttctactctcaatggtcttgtttctttgctttctacattagttaaccagttttataccaaaaaatgcatgtttgaggaattgtctgaaattgggacaaaacaccttcatgtaaaccagctttgcaaaattttccagcccagatactcttcatctattcaaatggattgtcttattctgagcaaagacctgttgttaatcttcaagctaggttttgcagttcccaaccacaacattcttctattttgccaggctggtgcaaagtaattaaagatgtcaatcagaaatgtcaatgagactaaagtggttttgtaaatctcagctatatttagcaacactccatgtagctaatattttttggtagcatctggtagaccttagaatgttacatagccagtaggttctttattcaaattttaagtatcttaagaatagtagggcagtaacagttacttttgagagttttctggtcaagcttttaccaggcattctctagccttggtacaaaaaaaaaaaaaacctgctggttgcgcagatacctaggcttgtccattttatgcatttcagcaaagtcattggatactattgcaacttgggaatactggtctgcatcaagtttattcggtagtttgaccgctagtatgttggaagttatttggattgtttttggaattttgactggctgaattatggttggtataaagttatgtgtataactggcaggcttatttatctgttgcacttggttagctttaattgttctgtattatttaaagataagtttactcaacaataaatctgcagagattgaacaagtaaaaaaaaaaaaaaaaaaaaaaaaaa

SEQ ID NO: 47 - DNA, 1879 bp, Homo sapiens
ctgaggcccacgcagggcctagggtgggaagatggcaggtgggggcggcgacctgagcaccaggaggctgaatgaatgtatttcaccagtagcaaatgagatgaaccatcttcctgcacacagccacgatttgcaaaggatgttcacggaagaccagggtgtagatgacaggctgctctatgacattgtattcaagcacttcaaaagaaataaggtggagatttcaaatgcaataaaaaagacatttccattcctcgagggcctccgtgatcgtgatctcatcacaaataaaatgtttgaagattctcaagattcttgtagaaacctggtccctgtacagagagtggtgtacaatgttcttagtgaactggagaagacatttaacctgccagttctggaagcactgttcagcgatgtcaacatgcaggaataccccgatttaattcacatttataaaggctttgaaaatgtaatccatgacaaattgcctctccaagaaagtgaagaagaagagagggaggagaggtctggcctccaactaagtcttgaacaaggaactggtgaaaactcttttcgaagcctgacttggccaccttcgggttccccatctcatgctggtacaaccccacctgaaaatggactctcagagcacccctgtgaaacagaacagataaatgcaaagagaaaagatacaaccagtgacaaagatgattcgctaggaagccaacaaacaaatgaacaatgtgctcaaaaggctgagccaacagagtcctgcgaacaaattgctgtccaagtgaataatggggatgctggaagggagatgccctgcccgttgccctgtgatgaagaaagcccagaggcagagctacacaaccatggaatccaaattaattcctgttctgtgcgactggtggatataaaaaaggaaaagccattttctaattcaaaagttgagtgccaagcccaagcaagaactcatcataaccaggcatctgacataatagtcatcagcagtgaggactctgaaggatccactgacgttgatgagcccttagaagtcttcatctcagcaccgagaagtgagcctgtgatcaataatgacaaccctttagaatcaaatgatgaaaaggagggccaagaagccacttgctcacgaccccagattgtaccagagcccatggatttcagaaaattatctacattcagagaaagttttaagaaaagagtgataggacaagaccacgacttttcagaatccagtgaggaggaggcgcccgcagaagcctcaagcggggcactgagaagcaagcatggtgagaaggctcctatgacttctagaagtacatctacttggagaatacccagcaggaagagacgtttcagcagtagtgacttttcagacctgagtaatggagaagagcttcaggaaacctgcagctcatccctaagaagagggtcaggtaaagaagattaggatgccaagacttggcctgcagaatgtcaggaatgtgaattaaaagctgctgtttccagacgctttttattctgagcaccttcactaccttgtatccagttcatctgggaactcctttttgcattttagaaaatggaaagaggcaggaaattatgataaactcatgtttaacagaaagagtttcactgactaaatgtatgtaattatattttgttgttgtagaagaaataaatagcaaatttgtggtattcttttttttaaacctgctctcattcctattaacactaagatcttagatttttatagtgataaatgggttgacatcattgtcgtttgtaattgtaaagcctcaaaagacaactgttcctactatgtaattatagacagaaataaaaacttcagatc

SEQ ID NO: 48 - DNA, 2320 bp, Homo sapiens
gaattccggtgcagtgagaggcatgcttccttagaccaggcaacatttcatttgttgcatttccagtttccaataccaactcacctacaaagattttaccaaaaaccttaggaccaataaatgtgaatgttggaccccaaatgattataagcacaccacagagactaaccagttcaggaagtgttctgattgggagtccatatacccctgcaccagcaatggttactcagacacacatagcagaagctactggctgggtccctggtgatagaaaacgggctagaaaatttatagactctgatttttcagaaagtaaacgaagcaaaaaaggagataaaaatgggaaaggcttgagacacttttcaatgaaagtgtgtgagaaagttcaacgaaaaggtacaacatcgtacaatgaagtcgctgatgagctggtgtcagagttcaccaattcaaataaccatttggctgctgattcgcaggcttatgatcagaagaacattaggcgaagagtttatgatgctttaaatgtgctaatggcaatgaacataatttcaaaggaaaaaaaagaaatcaagtggattggcctgcctaccaattctgctcaggaatgtcagaatctggagatagagaagcagaggcggatagaacggataaagcagaagcgggcccagctgcaagaacttctcctacagcaaatcgctttcaaaaacctggtacagagaaatcgacaaaatgagcagcaaaaccagggcccgccggctctgaactctaccattcagctgccattcataatcatcaatacaagcagaaaaacagtcatagattgcagcatctccagtgacaagtttgagtatcttttcaattttgacaacacctttgagatccatgatgacatagaagtactaaagcggatgggaatgtcgtttggcctggagtcaggcaaatgctctctggaggatctgaaacttgcgaaatccctggtgccaaaggctttagaaggttatatcacagatatctccacaggaccttcttggttaaatcagggactacttctgaactctacccaatcagtttcaaatttagacctgaccactggtgccaccttaccccagtcaagtgtaaaccaagggttatgcttggatgcagaagtggccttagcaactgggcagttcctggccccaaacagtcaccagtccagcagtgcggcctctcactgctccgagtcccgaggcgagaccccctgttcgttcaatgatgaagatgaggaagatgatgaggaggattcctcctccccagaataaagacaagagaaagcctacgtttcaatatgtgatgctcatgtgtgtttgtagtgtgagctcttgtttttgaaatgattgcttcagtctttgcctttgtttgcactgtgtgttcagtgaaaactagagaaacacaataagcaaatcacatgaaggctgagatgattgagatgaaagttaacatagtgtgaatgaaagtctggccaacagagcagtagatgatataacacagaaagcatgctttctgaggagatagtggggataatccctgaagagttggcagtgtgagtccaactcatttatcttctgtgaggtgggatttttgtgttccctcaccacccatgtaatattcttagaattgagattgctacacggagcagtccttgacgatcgtgtttctgacgtttctcttccttgcctggactgcctttgacaaggcagaaatgaacatgaaaagtccccaggagaggggagctgtgtagtgagggatactgagggcagtcctgtggctgaggggcacagattgaactgctgaactagttggaggtctagatgaggtgctttacgcatcagctgccttagacagcttctagaaaggagcgagcgctacttcttaagtacttaagtgacatttagataatttatagtaaaactgaaattattattagccaatgcattggtgcatagaatttactagggctacttctggaagcccccaatagaatagcatttccatgtgcattaaatactttgccagcactgcctttgccagcatcctaaatctggagttttaccaagaaggaaactgtatctttaggttaatccaagctatgcatttcatatagctttttcatttaaaacaaggcaaagaaacaaattcctatgaccaaattgcttgcctacagttccctgcagtaattgtatgatctcacccagtgtgcaatttgtggaaatgaaccaaatgtcattacttttgtcttaaagaaaagtctatttcaagaatatggacagcagtggaaaaa

SEQ ID NO: 49 - DNA, 4484 bp, Homo sapiens
tgcccaggaggagtaggagcaggagcagaagcagaagcggggtccggagctgcgcgcctacgcgggacctgtgtccgaaatgccggtgcgaggagaccgcgggtttccaccccggcgggagctgtcaggttggctccgcgccccaggcatggaagagctgatatgggaacagtacactgtgaccctacaaaaggattccaaaagaggatttggaattgcagtgtccggaggcagagacaacccccactttgaaaatggagaaacgtcaattgtcatttctgatgtgctcccgggtgggcctgctgatgggctgctccaagaaaatgacagagtggtcatggtcaatggcacccccatggaggatgtgcttcattcgtttgcagttcagcagctcagaaaaagtgggaaggtcgctgctattgtggtcaagaggccccggaaggtccaggtggccgcacttcaggccagccctcccctggatcaggatgaccgggcttttgaggtgatggacgagtttgatggcagaagtttccggagtggctacagcgagaggagccggctgaacagccatggggggcgcagccgcagctgggaggacagcccggaaagggggcgtccccatgagcgggcccggagccgggagcgggacctcagccgggaccggagccgtggccggagcctggagcggggcctggaccaagaccatgcgcgcacccgagaccgcagccgtggccggagcctggagcggggcctggaccacgactttgggccatcccgggaccgggaccgtgaccgcagccgcggccggagcattgaccaggactacgagcgagcctatcaccgggcctacgacccagactacgagcgggcctacagcccggagtacaggcgcggggcccgccacgatgcccgctctcggggaccccgaagccgcagccgcgagcacccgcactcacggagccccagccccgagcctagggggcggccggggcccatcggggtcctcctgatgaaaagcagagcgaacgaagagtatggtctccggcttgggagtcagatcttcgtaaaggaaatgacccgaacgggtctggcaactaaagatggcaaccttcacgaaggagacataattctcaagatcaatgggactgtaactgagaacatgtctttaacggatgctcgaaaattgatagaaaagtcaagaggaaaactacagctagtggtgttgagagacagccagcagaccctcatcaacatcccgtcattaaatgacagtgactcagaaatagaagatatttcagaaatagagtcaacccgatcattttctccagaggagagacgtcatcagtattctgattatgattatcattcctcaagtgagaagctgaaggaaaggccaagttccagagaggacacgccgagcagattgtccaggatgggtgcgacacccactccctttaagtccacaggggatattgcaggcacagttgtcccagagaccaacaaggaacccagataccaagaggaacccccagctcctcaaccaaaagcagccccgagaacttttcttcgtcctagtcctgaagatgaagcaatatatggccctaataccaaaatggtaaggttcaagaagggagacagcgtgggcctccggttggctggtggcaatgatgtcgggatatttgttgctggcattcaagaagggacctcggcggagcaggagggccttcaagaaggagaccagattctgaaggtgaacacacaggatttcagaggattagtgcgggaggatgccgttctctacctgttagaaatccctaaaggtgaaatggtgaccattttagctcagagccgagccgatgtgtatagagacatcctggcttgtggcagaggggattcgttttttataagaagccactttgaatgtgagaaggaaactccacagagcctggccttcaccagaggggaggtcttccgagtggtagacacactgtatgacggcaagctgggcaactggctggctgtgaggattgggaacgagttggagaaaggcttaatccccaacaagagcagagctgaacaaatggccagtgttcaaaatgcccagagagacaacgctggggaccgggcagatttctggagaatgcgtggccagaggtctggggtgaagaagaacctgaggaaaagtcgggaagacctcacagctgttgtgtctgtcagcaccaagttcccagcttatgagagggttttgctgcgagaagctggtttcaagagacctgtggtcttattcggccccatagctgatatagcaatggaaaaattggctaatgagttacctgactggtttcaaactgctaaaacggaaccaaaagatgcaggatctgagaaatccactggagtggtccggttaaataccgtgaggcaagttattgaacaggataagcatgcactactggatgtgactccgaaagctgtggacctgttgaattacacccagtggttctcaattgtgatttctttcacgccagactccagacaaggtgtcaacaccatgagacaaaggttagacccaacgtccaacaatagttctcgaaagttatttgatcacgccaacaagcttaaaaaaacgtgtgcacacctttttacagctacaatcaacctaaattcagccaatgatagctggtttggcagcttaaaggacactattcagcatcagcaaggagaagcggtttgggtctctgaaggaaagatggaagggatggatgatgaccccgaagaccgcatgtcctacttaactgccatgggcgcagactatctgagttgcgacagccgcctcatcagtgactttgaagacacggacggtgaaggaggcgcctacactgacaatgagctggatgagccagccgaggagccgctggtgtcgtccatcacccgctcctcggagccggtgcagcacgaggagagcataaggaaacccagcccagagccacgagctcagatgaggagggctgctagcagcgatcaacttagggacaatagcccgcccccagcattcaagccagagccgtccaaggccaaaacccagaacaaagaagaatcctatgacttctccaaatcctatgaatataagtcaaacccctctgccgttgctggtaatgaaactcctggggcatctaccaaaggttatcctcctcctgttgcagcaaaacctacctttgggcggtctatactgaagccctccactcccatccctcctcaagagggtgaggaggtgggagagagcagtgaggagcaagataatgctcccaaatcagtcctgggcaaagtcaaaatatttggagaagatggatcacaagggccagggttacaagagaatgcaggagctccaggaagcacagaatgcaaggatcgaaattgcccagaagcatcctgatatctatgcagttccaatcaaaacgcacaagccagaccctggcacgccccagcacacgagttccagaccccctgagccacagaaagctccttccagaccttatcaggataccagaggaagttatggcagtgatgccgaggaggaggagtaccgccagcagctgtcagaacactccaagcgcggttactatggccagtctgcccgataccgggacacagaattatagatgtctgagcacggactctcccaggcctgcctgcatggcatcagactagccactcctgccaggccgccgggatggttcttctccagttagaatgcaccatggagacgtggtgggactccagctcgtgtgtcctcatggagaacccaggggacagctggtgcaaattcagaactgagggctctgtttgtgggactgggttagaggagtctgtggctttttgttcagaattaagcagaacactgcagtcagatcctgttacttgcttcagtggaccgaaatctgtattctgtttgcgtacttgtaatatgtatattaagaagcaataactatttttcctcattaatagctgccttcaaggactgtttcagtgtgagtcagaatgtgaaaaaggaataaaaaatactgttgggctcaaactaaattcaaagaagtactttattgcaactcttttaagtgccttggatgagaagtgtcttaaattttcttcctttgaagctttaggcagagccataatggactaaaacattttgactaagtttttataccagcttaatagctgtagttttccctgcactgtgtcatcttttcaaggcatttgtctttgtaatattttccataaatttggactgtctatatcataactatacttgatagtttggctataagtgctcaatagcttgaagcccaagaagttggtatcgaaatttgttgtttgtttaaacccaagtgctgcacaaaagcagatacttgaggaaaacactatttccaaaagcacatgtattgacaacagttttataatttaataaaaaggaatacattgcaatccgt

SEQ ID NO: 50 - DNA, 2600 bp, Homo sapiens
ggatccaaaggagacctatagtgactcccaggagctcttagtgaccaagtgaaggtacctgtggggctcattgtgcccattgctctttcactgctttcaactggtagttgtgggttgaagcactggacaatgccacatactttgtggatggtgtgggtcttgggggtcatcatcagcctctccaaggaagaatcctccaatcaggcttctctgtcttgtgaccgcaatggtatctgcaagggcagctcaggatctttaaactccattccctcagggctcacagaagctgtaaaaagccttgacctgtccaacaacaggatcacctacattagcaacagtgacctacagaggtgtgtgaacctccaggctctggtgctgacatccaatggaattaacacaatagaggaagattctttttcttccctgggcagtcttgaacatttagacttatcctataattacttatctaatttatcgtcttcctggttcaagcccctttcttctttaacattcttaaacttactgggaaatccttacaaaaccctaggggaaacatctcttttttctcatctcacaaaattgcaaatcctgagagtgggaaatatggacaccttcactaagattcaaagaaaagattttgctggacttaccttccttgaggaacttgagattgatgcttcagatctacagagctatgagccaaaaagtttgaagtcaattcagaacgtaagtcatctgatccttcatatgaagcagcatattttactgctggagatttttgtagatgttacaagttccgtggaatgtttggaactgcgagatactgatttggacactttccatttttcagaactatccactggtgaaacaaattcattgattaaaaagtttacatttagaaatgtgaaaatcaccgatgaaagtttgtttcaggttatgaaacttttgaatcagatttctggattgttagaattagagtttgatgactgtacccttaatggagttggtaattttagagcatctgataatgacagagttatagatccaggtaaagtggaaacgttaacaatccggaggctgcatattccaaggttttacttattttatgatctgagcactttatattcacttacagaaagagttaaaagaatcacagtagaaaacagtaaagtttttctggttccttgtttactttcacaacatttaaaatcattagaatacttggatctcagtgaaaatttgatggttgaagaatacttgaaaaattcagcctgtgaggatgcctggccctctctacaaactttaattttaaggcaaaatcatttggcatcattggaaaaaaccggagagactttgctcactctgaaaaacttgactaacattgatatcagtaagaatagttttcattctatgcctgaaacttgtcagtggccagaaaagatgaaatatttgaacttatccagcacacgaatacacagtgtaacaggctgcattcccaagacactggaaattttagatgttagcaacaacaatctcaatttattttctttgaatttgccgcaactcaaagaactttatatttccagaaataagttgatgactctaccagatgcctccctcttacccatgttactagtattgaaaatcagtaggaatgcaataactacgttttctaaggagcaacttgactcatttcacacactgaagactttggaagctggtggcaataacttcatttgctcctgtgaattcctctccttcactcaggagcagcaagcactggccaaagtcttgattgattggccagcaaattacctgtgtgactctccatcccatgtgcgtggccagcaggttcaggatgtccgcctctcggtgtcggaatgtcacaggacagcactggtgtctggcatgtgctgtgctctgttcctgctgatcctgctcacgggggtcctgtgccaccgtttccatggcctgtggtatatgaaaatgatgtgggcctggctccaggccaaaaggaagcccaggaaagctcccagcaggaacatctgctatgatgcatttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgctttctgaaaactttgtgaagagtgagtggtgcaagtatgaactggacttctcccatttccgtctttttgaagagaacaatgatgctgccattctcattcttctggagcccattgagaaaaaagccattccccagcgcttctgcaagctgcggaagataatgaacaccaagacctacctggagtggcccatggacgaggctcagcgggaaggattttgggtaaatctgagagctgcgataaagtcctaggttcccatatttaagaccagtctttgtctagttgggatctttatgtcactagttatagttaagttcattcagacataattatataaaaactacgtggatgtaccgtcatttgagga

SEQ ID NO: 51 - DNA, 597 bp, Homo sapiens
agctgaagttgaggatctcttactctctaagccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtgggtactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctgggtgaaatataccaaattctgcatctccagaggaaaataagaaataaagatgaattgttgcaactcttaaaaaaa

SEQ ID NO: 52 - DNA, 2058 bp, Homo sapiens
gcacgaggaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaactgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacgtgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgataccaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaattatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgc

SEQ ID NO: 53 - DNA, 1145 bp, Homo sapiens
gctccggccagccgcggtccagagcgcgcgaggttcggggagctccgccaggctgctggtacctgcgtccgcccggcgagcaggacaggctgctttggtttgtgacctccaggcaggacggccatcctctccagaatgaagatcttcttgccagtgctgctggctgcccttctgggtgtggagcgagccagctcgctgatgtgcttctcctgcttgaaccagaagagcaatctgtactgcctgaagccgaccatctgctccgaccaggacaactactgcgtgactgtgtctgctagtgccggcattgggaatctcgtgacatttggccacagcctgagcaagacctgttccccggcctgccccatcccagaaggcgtcaatgttggtgtggcttccatgggcatcagctgctgccagagctttctgtgcaatttcagtgcggccgatggcgggctgcgggcaagcgtcaccctgctgggtgccgggctgctgctgagcctgctgccggccctgctgcggtttggcccctgaccgcccagaccctgtcccccgatcccccagctcaggaaggaaagcccagccctttctggatcccacagtgtatgggagcccctgactcctcacgtgcctgatctgtgcccttggtcccaggtcaggcccaccccctgcacctccacctgccccagcccctgcctctgcccaagtgggccagctgccctcacttctggggtggatgatgtgaccttccttgggggactgcggaagggacgagggttccctggagtcttacggtccaacatcagaccaagtcccatggacatgctgacagggtccccagggagaccgtgtcagtagggatgtgtgcctggctgtgtacgtgggtgtgcagtgcacgtgagagcacgtggcggcttctgggggccatgtttggggagggaggtgtgccagcagcctggagagcctcagtccctgtagccccctgccctggcacagctgcatgcacttcaagggcagcctttgggggttggggtttctgccacttccgggtctaggccctgcccaaatccagccagtcctgccccagcccacccccacattggagccctcctgctgctttggtgcctcaaataaatacagatgtcccc

SEQ ID NO: 54 - DNA, 1826 bp, Homo sapiens
agtctgcactggagctgcctggtgaccagaagtttggagtccgctgacgtcgccgcccagatggcctccaggctgaccctgctgaccctcctgctgctgctgctggctggggatagagcctcctcaaatccaaatgctaccagctccagctcccaggatccagagagtttgcaagacagaggcgaagggaaggtcgcaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttggagagtcattcaacagaggccgtgttgggggatgctttggtagatttctccctgaagctctaccacgccttctcagcaatgaagaaggtggagaccaacatggccttttccccattcagcatcgccagcctccttacccaggtcctgctcggggctgggcagaacaccaaaacaaacctggagagcatcctctcttaccccaaggacttcacctgtgtccaccaggccctgaagggcttcacgaccaaaggtgtcacctcagtctctcagatcttccacagcccagacctggccataagggacacctttgtgaatgcctctcggaccctgtacagcagcagccccagagtcctaagcaacaacagtgacgccaacttggagctcatcaacacctgggtggccaagaacaccaacaacaagatcagccggctgctagacagtctgccctccgatacccgccttgtcctcctcaatgctatctacctgagtgccaagtggaagacaacatttgatcccaagaaaaccagaatggaaccctttcacttcaaaaactcagttataaaagtgcccatgatgaatagcaagaagtaccctgtggcccatttcattgaccaaactttgaaagccaaggtggggcagctgcagctctcccacaatctgagtttggtgatcctggtaccccagaacctgaaacatcgtcttgaagacatggaacaggctctcagcccttctgttttcaaggccatcatggagaaactggagatgtccaagttccagcccactctcctaacactaccccgcatcaaagtgacgaccagccaggatatgctctcaatcatggagaaattggaattcttcgatttttcttatgaccttaacctgtgtgggctgacagaggacccagatcttcaggtttctgcgatgcagcaccagacagtgctggaactgacagagactggggtggaggcggctgcagcctccgccatctctgtggcccgcaccctgctggtctttgaagtgcagcagcccttcctcttcgtgctctgggaccagcagcacaagttccctgtcttcatggggcgagtatatgaccccagggcctgagacctgcaggatcaggttagggcgagcgctacctctccagcctcagctctcagttgcagccctgctgctgcctgcctggacttgcccctgccacctcctgcctcaggtgtccgctatccaccaaaagggctcctgagggtctgggcaagggacctgcttctattagcccttctccatggccctgccatgctctccaaaccactttttgcagctttctctagttcaagttcaccagactctataaataaaacctgacagaccat

SEQ ID NO: 55 - DNA, 1919 bp, Homo sapiens
ctgaagaacaaatcagcctggtcaccagcttttcggaacagcagagacacagagggcagtcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggaggataggaagacagggggcccaacctgggattgctgagcagggaagctttgcatgttgctctaaggtacatttttaaagagttgttttttggccgggcgcagtgctcatgcctgtaatcccagaactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagagagccaagattgcg

SEQ ID NO: 56 - DNA, 634 bp, Homo sapiens
cggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagattggcgtgcacgccttccagcagcgtctggctgtccacccgagcggtgtggcgctgcaggacagggtcccccttgccagccagggcctgggccctggcagcacggtcctgctggtggtggacaaatgcgacgaacctctgagcatcctggtgaggaataacaagggccgcagcagcacctacgaggtccggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaagagaat

SEQ ID NO: 57 - DNA, 1742 bp, Homo sapiens
tctttgaagcttcaaggctgctgaataatttccttctcccattttgtgcctgcctagctatccagacagagcagctaccctcagctctagctgatactacagacagtacaacagatcaagaagtatggcagtgacaactcgtttgacatggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcagaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctgctgctgggtccaattggagctgggaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcgaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaataataataatttttcttggatttatgttctgtatctgtgaaaaaataaatttcttataaaactcgggtctaaaaaaaaaaaaaaaaaa

SEQ ID NO: 58 - DNA, 325 bp, Homo sapiens
ccccctagactatcagatgctcaagtaggagcatgaggtggctgtgctgggggcaccccacaactctgctcccccgaggtccaccgtgatccacatccacagcgagacctctgtgcccaaccatgttgtctggtccctgttcaacaccctcttcatgaactcctgctgcctgggcttcatagcagtcacctactccatgaagtctagggacaggaagatggttggcgacctaaccgggggccaggcctgtgcctccaccgccaagtgcctgaacgtctgggccctggttctgggcatcctcatgagcattctgctcatcttcatc

SEQ ID NO: 59 - DNA, 482 bp, Homo sapiens
ggcatgtgtgactgtttcagcgactgcggagtctgtctctgtggcacattttgtttcccgtgccttgggtgtcaagttgcagctgatatgaatgaatgctgtctgtgtggaacaagcgtcgcaatgaggactctctacaggacccgatatggcatccctggatctatttgtgatgactatatggcaactctttgctgtcctcattgtactctttgccaaatcaagagagatatcaacagaaggagagccatgcgtactttctaaaaactgatggtgaaaagctcttaccgaagcaacaaaattcagcagacacctcttcagcttgagttcttcaccatcttttgcaactgaaatatgatggatatgcttaagtacaactgatggcatgaaaaaaatcaaatttttgatttattataaatgaatgttgtccctgaacttagctaaatggtgcaacttagtttctccttgctttcatattatcg

SEQ ID NO: 60 - DNA, 3380 bp, Homo sapiens
gcgcgccggcctgagagccctgtggacaacctcgtcattgtcaggcacagagcggtagaccctgcttctctaagtgggcagcggacagcggcacgcacatttcacctgtcccgcagacaacagcaccatctgcttgggagaaccctctcccttctctgagaaagaaagatgtcgaatgggtattccacagacgagaatttccgctatctcatctcgtgcttcagggccagggtgaaaatgtacatccaggtggagcctgtgctggactacctgacctttctgcctgcagaggtgaaggagcagattcagaggacagtcgccacctccgggaacatgcaggcagttgaactgctgctgagcaccttggagaagggagtctggcaccttggttggactcgggaattcgtggaggccctccggagaaccggcagccctctggccgcccgctacatgaaccctgagctcacggacttgccctctccatcgtttgagaacgctcatgatgaatatctccaactgctgaacctccttcagcccactctggtggacaagcttctagttagagacgtcttggataagtgcatggaggaggaactgttgacaattgaagacagaaaccggattgctgctgcagaaaacaatggaaatgaatcaggtgtaagagagctactaaaaaggattgtgcagaaagaaaactggttctctgcatttctgaatgttcttcgtcaaacaggaaacaatgaacttgtccaagagttaacaggctctgattgctcagaaagcaatgcagagattgagaatttatcacaagttgatggtcctcaagtggaagagcaacttctttcaaccacagttcagccaaatctggagaaggaggtctggggcatggagaataactcatcagaatcatcttttgcagattcttctgtagtttcagaatcagacacaagtttggcagaaggaagtgtcagctgcttagatgaaagtcttggacataacagcaacatgggcagtgattcaggcaccatgggaagtgattcagatgaagagaatgtggcagcaagagcatccccggagccagaactccagctcaggccttaccaaatggaagttgcccagccagccttggaagggaagaatatcatcatctgcctccctacagggagtggaaaaaccagagtggctgtttacattgccaaggatcacttagacaagaagaaaaaagcatctgagcctggaaaagttatagttcttgtcaataaggtactgctagttgaacagctcttccgcaaggagttccaaccatttttgaagaaatggtatcgtgttattggattaagtggtgatacccaactgaaaatatcatttccagaagttgtcaagtcctgtgatattattatcagtacagctcaaatccttgaaaactccctcttaaacttggaaaatggagaagatgctggtgttcaattgtcagacttttccctcattatcattgatgaatgtcatcacaccaacaaagaagcagtgtataataacatcatgaggcattatttgatgcagaagttgaaaaacaatagactcaagaaagaaaacaaaccagtgattccccttcctcagatactgggactaacagcttcacctggtgttggaggggccacgaagcaagccaaagctgaagaacacattttaaaactatgtgccaatcttgatgcatttactattaaaactgttaaagaaaaccttgatcaactgaaaaaccaaatacaggagccatgcaagaagtttgccattgcagatgcaaccagagaagatccatttaaagagaaacttctagaaataatgacaaggattcaaacttattgtcaaatgagtccaatgtcagattttggaactcaaccctatgaacaatgggccattcaaatggaaaaaaaagctgcaaaaaaaggaaatcgcaaagaacgtgtttgtgcagaacatttgaggaagtacaatgaggccctacaaattaatgacacaattcgaatgatagatgcgtatactcatcttgaaactttctataatgaagagaaagataagaagtttgcagtcatagaagatgatagtgatgagggtggtgatgatgagtattgtgatggtgatgaagatgaggatgatttaaagaaacctttgaaactggatgaaacagatagatttctcatgactttattttttgaaaacaataaaatgttgaaaaggctggctgaaaacccagaatatgaaaatgaaaagctgaccaaattaagaaataccataatggagcaatatactaggactgaggaatcagcacgaggaataatctttacaaaaacacgacagagtgcatatgcgctttcccagtggattactgaaaatgaaaaatttgctgaagtaggagtcaaagcccaccatctgattggagctggacacagcagtgagttcaaacccatgacacagaatgaacaaaaagaagtcattagtaaatttcgcactggaaaaatcaatctgcttatcgctaccacagtggcagaagaaggtctggatattaaagaatgtaacattgttatccgttatggtctcgtcaccaatgaaatagccatggtccaggcccgtggtcgagccagagctgatgagagcacctacgtcctggttgctcacagtggttcaggagttatcgaacatgagacagttaatgatttccgagagaagatgatgtataaagctatacattgtgttcaaaatatgaaaccagaggagtatgctcataagattttggaattacagatgcaaagtataatggaaaagaaaatgaaaaccaagagaaatattgccaagcattacaagaataacccatcactaataactttcctttgcaaaaactgcagtgtgctagcctgttctggggaagatatccatgtaattgagaaaatgcatcacgtcaatatgaccccagaattcaaggaactttacattgtaagagaaaacaaagcactgcaaaagaagtgtgccgactatcaaataaatggtgaaatcatctgcaaatgtggccaggcttggggaacaatgatggtgcacaaaggcttagatttgccttgtctcaaaataaggaattttgtagtggttttcaaaaataattcaacaaagaaacaatacaaaaagtgggtagaattacctatcacatttcccaatcttgactattcagaatgctgtttatttagtgatgaggattagcacttgattgaagattcttttaaaatactatcagttaaacatttaatatgattatgattaatgtattcattatgctacagaactgacataagaatcaataaaatgattgttttactctgaaaaaaaaaaaaaaa

SEQ ID NO: 61 - DNA, 491 bp, Homo sapiens
  misc_feature (314)..(314): n is a, c, g, or t
ggcggcaatagtttcagcttcagcagcgccagcagtcttagtagcagcagcaccagtgcgggttgcgccagcagccttggcggcggcggcgcctcggagcttctccctgcaacacagcccacagccagcagcgctcccaaaagccccgagccagcccaaggcgcgcttggctgcttatagactgtactagggcggaggggatccgggccttgcgtgcagcctcccaaccatgggctgggttttgtgcttactgtatgttggcgacttggtagggcaggagacgcagcgtggagcctacctcccgacattcacgnttcgccccacgctgctccgactggctgcagcggacactgcccaaagcagaggggagtctcagtgtcctgctagccagccgaacacttctctccggaagcaggctggttcgactgtgaggtgtttgactaaactgtttctctgactcgccccagaggtcgtggctcaaaggcacttag

SEQ ID NO: 62 - DNA, 1610 bp, Homo sapiens
cccagagcagcgctcgccacctccccccggcctgggcagcgctcgcccggggagtccagcggtgtcctgtggagctgccgccatggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatgaaactcggggaaaccagcccagctaagtccggagtgaaggagcctctctgctttagctaaagacgactgagaagaggtgcaaggaagcgggctccaggagcaagctcaccaggcctctcagaagtcccagcaggatctcacggactgccgggtcggcgcctcctgcgcgagggagcaggttctccgcattcccatgggcaccacctgcctgcctgtcgtgccttggacccagggcccagcttcccaggagagaccaaaggcttctgagcaggatttttatttcattacagtgtgagctgcctggaatacatgtggtaatgaaataaaaaccctgccccgaatcttccgtccctcatcctaacttgcagttcacagagaaaagtgacatacccaaagctctctgtcaattacaaggcttctcctggcgtgggagacgtctacagggaagacaccagcgtttgggcttctaaccaccctgtctccagctgctctgcacacatggacagggacctgggaaaggtgggagagatgctgagcccagcgaatcctctccattgaaggattcaggaagaagaaaactcaactcagtgccattttacgaatatatgcgtttatatttatacttccttgtctattatatctatacattatatattatttgtattttgacattgtaccttgtataaacaaaataaaacatctattttcaatatttttaaaatgca

SEQ ID NO: 63 - DNA, 2544 bp, Homo sapiens
ggcacgagggcggcggagccgactcgtcgcggccgaggcgcacgcggtccgcgccggcgtcagtctgggattggccggcccgcgacttcctccgccccctgccaatcgccggggacgacttccgtgggtttttccggctctcccgcgtcgctaaggagcgacgggctgtcggccagaccccgagttctcggtgcgctcagcggccgccgacgctaggaggccgcgctccgcccccgctaccatgaggccccggaaagccttcctgctcctgctgctcttggggctggtgcagctgctggccgtggcgggtgccgagggcccggacgaggattcttctaacagagaaaatgccattgaggatgaagaggaggaggaggaggaagatgatgatgaggaagaagacgacttggaagttaaggaagaaaatggagtcttggtcctaaatgatgcaaactttgataattttgtggctgacaaagacacagtgctgctggagttttatgctccatggtgtggacattgcaagcagtttgctccggaatatgaaaaaattgccaacatattaaaggataaagatcctcccattcctgttgccaagatcgatgcaacctcagcgtctgtgctggccagcaggtttgatgtgagtggctaccccaccatcaagatccttaagaaggggcaggctgtagactacgagggctccagaacccaggaagaaattgttgccaaggtcagagaagtctcccagcccgactggacgcctccaccagaagtcacgcttgtgttgaccaaagagaactttgatgaagttgtgaatgatgcagatatcattctggtggagttttatgccccatggtgtggacactgcaagaaacttgcccccgagtatgagaaggccgccaaggagctcagcaagcgttctcctccaattcccctggcaaaggtcgacgccaccgcagaaacagacctggccaagaggtttgatgtctctggctatcccaccctgaaaattttccgcaaaggaaggccttatgactacaacggcccacgagaaaaatatggaatcgttgattacatgatcgagcagtccgggcctccctccaaggagattctgaccctgaagcaggtccaggagttcctgaaggatggagacgatgtcatcatcatcggggtctttaagggggagagtgacccagcctaccagcaataccaggatgccgctaacaacctgagagaagattacaaatttcaccacactttcagcacagaaatagcaaagttcttgaaagtctcccaggggcagttggttgtaatgcagcctgagaaattccagtccaagtatgagccccggagccacatgatggacgtccagggctccacccaggactcggccatcaaggacttcgtgctgaagtacgccctgcccctggttggccaccgcaaggtgtcaaacgatgctaagcgctacaccaggcgccccctggtggtcgtctactacagtgtggacttcagctttgattacagagctgcaactcagttttggcggagcaaagtcctagaggtggccaaggacttccctgagtacacctttgccattgcggacgaagaggactatgctggggaggtgaaggacctggggctcagcgagagtggggaggatgtcaatgccgccatcctggacgagagtgggaagaagttcgccatggagccagaggagtttgactctgacaccctccgcgagtttgtcactgctttcaaaaaaggaaaactgaagccagtcatcaaatcccagccagtgcccaagaacaacaagggacccgtcaaggtcgtggtgggaaagacctttgactccattgtgatggaccccaagaaggacgtcctcatcgagttctacgcaccatggtgcgggcactgcaagcagctagagcccgtgtacaacagcctggccaagaagtacaagggccaaaagggcctggtcatcgccaagatggacgccactgccaacgacgtccccagcgaccgctataaggtggagggcttccccaccatctacttcgcccccagtggggacaaaaagaacccagttaaatttgagggtggagacagagatctggagcatttgagcaagtttatagaagaacatgccacaaaactgagcaggaccaaggaagagctttgaaggcctgaggtctgcggaaggtgggaggaggcagacaccctgcgtggcccatggtcggggcgtccacgccgaggccggcaacaaacgacagtatctcggattccttttttttttttttttaattttttatactttggtgtttcacttcatgctctgaatactgaataaccatgaatgactgaatagtttagtccagatttttacagaggatacatctatttttatcattatttggggtttgaaaaatttttttttacaccttctaatttctttatttctcaaagcagataattcttctgtgtgaaaatgttttctttttttaatttaaggtttaaaattcctttgccaaaaaaaaaaaaaaaaaa

SEQ ID NO: 64 - DNA, 544 bp, Homo sapiens
gactactttctatgcaagactgagatatggaatagataggaagagatatgtactgctgggtacatggacagtaagtgtgttttcagatggagtaccagcaccgaaaatgggttgagggaggatgggttgtatgtatgtttctgcccactaattttgagcagccatattatgaattaaatcgtcacagccaagtaataacccaagaatggtatgagtttcatgtgtaatagctcaaatggaataagcatgaatgctggagtggaccattatcctcaaatattctatgtcacttctcatttaaagactcttgttatgaactattagaaactttaggcaaaatcaaaagtatttgcggcaaaataaaggcctattctactcttatttaaagtgaaacactgtatacttgtttctctccaaagcgaaattaagtatttataatttcaattgcctcgataagtttccaagtcactgaaatctgctgaaggttttactgtattgttgcacaactttaagataatttttgtctcaatgtcaacttttttca

SEQ ID NO: 65 - DNA, 272 bp, Homo sapiens
  misc_feature (66)..(66): n is a, c, g, or t
aggttgcagtgtggtgagatcatgccactacactccagcctggcgacagagcgagacttggtttcnaaaaaaaaaaaaaaaaaaacttcagtaagtacgtgttatttttttcaataaaattctattacagtatgtcatgtttgctgtagtgctcatatttattgttgtttttgttttagtactcacttgtttcataatatcaagattactaaaaatgggggaaaggacttctaatctttttttcataatatctttgacacatattacagaag

(19) United States
(12) Patent Application Publication
(10) Pub. No.: US 2009/0325176 A1
(43) Pub. Date: Dec. 31, 2009

(54) Gene Expression Profiles Associated with Asthma Exacerbation Attacks

(75) Inventors: Margot Mary O'Toole, Newton, MA (US); Frederick William Immermann, Suffern, NY (US); Padmalatha Sunkara Reddy, Lexington, MA (US); Andrew Arthur Hill, Cambridge, MA (US); John Louis Ryan, Philadelphia, PA (US); Andrew Joseph Dorner, Lexington, MA (US); Cristina Ileana Csimma, Lincoln, MA (US); Charlotte Marie McKee, Newton, MA (US); Wei Liu, Lexington, MA (US); Divya Chaudhary, Andover, MA (US); Matthew Ren Silver, Rockport, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/478,940

(22) Filed: Jun. 5, 2009

Related U.S. Application Data
(60) Provisional application No. 61/059,153, filed on Jun. 5, 2008.
(60) Provisional application No. 61/084,787, filed on Jul. 30, 2008.
(60) Provisional application No. 61/111,917, filed on Nov. 6, 2008.

(51) Int. Cl. C12Q 1/68; G01N 33/53; C40B 40/08; C40B 40/10

(52) U.S. Cl. 435/6; 435/7.1; 506/17; 506/18

(57) ABSTRACT

The present invention provides methods for the assessment, diagnosis, or prognosis of asthma exacerbation, by assessing the level of expression of asthma exacerbation gene products in a sample derived from a patient. The markers of the present invention can be used in methods to identify or evaluate agents capable of modulating marker expression levels in subjects with asthma.

This application claims priority to U.S. Provisional Application Nos. 61/059,153, filed on Jun. 5, 2008; 61/084,787, filed on Jul. 30, 2008 and 61/111,917, filed Nov. 6, 2008 respectively, and which are incorporated herein by reference in their entirety.

### BACKGROUND

The present invention relates to markers of acute asthma exacerbation and methods of using the same for the prediction, diagnosis and prognosis of acute asthma exacerbation.

Asthma is a chronic inflammatory disease of the airways that is characterized by recurrent episodes of reversible airway obstruction and airway hyperresponsiveness (AHR). Typical clinical manifestations of acute asthma exacerbation (also known as asthma attack) include shortness of breath, wheezing, coughing and chest tightness that can become life threatening or fatal. Despite the considerable progress that has been made in elucidating the pathophysiology of asthma, the prevalence, morbidity, and mortality of the disease has increased during the past two decades. In 1995, in the United States alone, nearly 1.8 million emergency room visits, 466,000 hospitalizations and 5,429 deaths were directly attributed to acute asthma exacerbation.

It is generally accepted that allergic asthma is initiated by an inappropriate inflammatory reaction to airborne allergens. The lungs of asthmatics demonstrate an intense infiltration of lymphocytes, mast cells and eosinophils. A large body of evidence has demonstrated this immune response to be driven by CD4⁺ T-cells expressing a T_{H2} cytokine profile. Four major pathophysiological responses seen in human asthma include upregulation of serum IgE (atopy), eosinophilia, excessive mucus secretion, and AHR.

Current therapy for asthma includes use of bronchodilators, corticosteroids and leukotriene inhibitors. The treatments share the same therapeutic goal of bronchodilation, reducing inflammation and facilitating expectoration. Many of such treatments, however, include undesired side effects and lose effectiveness after being used for a period of time. Additionally, only limited agents for therapeutic intervention are available for decreasing the airway remodeling process that occurs in asthmatics. Therefore, there remains a need for an increased molecular understanding of asthma, and a need for the identification of novel therapeutic strategies to combat these complex diseases.

### SUMMARY

In one aspect, the invention provides a method for determining the molecular signature of asthma exacerbation attack of a subject, comprising the steps of determining the level of at least one biomarker in said subject prior to an exacerbation attack; determining the level of the at least one biomarker in said subject during an asthma exacerbation attack; and ascertaining the difference between the level of the biomarker prior to the attack and the level during the attack. The difference in the level of a particular biomarker or plurality of biomarkers (i.e., a change in expression of one or more biomarkers) indicates the molecular signature, which in turn indicates the type of asthma exacerbation attack. In some embodiments, the levels of biomarkers are determined from a sample obtained from the subject. In one embodiment, the sample is a blood sample comprising peripheral blood mononuclear cells (PBMCs). In some embodiments, the type of asthma exacerbation attack is one of innate immunity (subgroup X), as indicated e.g. by a change in expression of one or more biomarkers listed in Tables 4, 6 and 9. In some embodiments, the type of asthma exacerbation attack is one of cognate immunity (subgroup Y), as indicated e.g. by a change in expression of one or more biomarkers listed in Tables 5 and 10. In some embodiments, the type of asthma exacerbation attack is coextensive with an airway infection, as indicated e.g. by a change in expression of one or more biomarkers listed in Tables 11 and 12. In yet other embodiments, the type of asthma exacerbation attack does not involve an airway infection, as indicated by a change in expression of biomarkers selected from a group comprising interferon induced with helicase C domain 1 (IFIH1; e.g. SEQ ID NO:60), leukotriene A4 hydrolase (LTA4H; e.g. SEQ ID NO:61) and open reading frame number 25 of human chromosome 6 (C6ORF25; SEQ ID NO:62). In some embodiments, the biomarkers are nucleic acids. In other embodiments, the biomarkers are polypeptides.

In another aspect, the invention provides a method for selecting a treatment for asthma exacerbation in a patient, comprising the steps of determining the type of asthma exacerbation based on the molecular signature in the patient (supra), then selecting a treatment corresponding to the type of asthma exacerbation. In some embodiments, the therapies are tailored to stopping T and/or B cell cognate immunity, innate immunity and/or airway infection. In some embodiments, the blood of the patient is monitored to ascertain a change in the levels of one or more biomarkers to assess the effectiveness of treatment and to revise therapy as indicated.

In one aspect, the invention provides a method for identifying individuals at risk for asthma, by identifying an individual who does not yet exhibit symptoms of asthma, measuring the level of at least one product in a sample obtained from the individual, comparing that level to a reference level of the product, and optionally providing the result of the comparison to a user. The product is the product of at least one gene that is differentially expressed in individuals having an acute exacerbation of asthma versus those not having an acute exacerbation of asthma. A difference between the reference level and the level of the product indicates that the individual is at risk for asthma.

In another aspect, the invention provides a method for identifying individuals at risk for asthma exacerbation, by identifying an individual who is a known asthmatic, measuring the level of at least one product in a sample obtained from the individual, comparing that level to a reference level of the product, and optionally providing the result of the comparison to a user. The product is the product of at least one gene that is differentially expressed in individuals having an acute exacerbation of asthma versus those not having an acute exacerbation of asthma. A difference between the reference level and the level of the product indicates that the individual is at risk for acute exacerbation of asthma.

In some embodiments, the invention provides a method of identifying individuals at risk for asthma or asthma exacerbation, comprising: (a) identifying an individual who does not exhibit symptoms of asthma; (b) measuring the level of at least one product in a sample obtained from the individual, wherein the product is produced from a gene which is differentially expressed during asthma exacerbation; and (c) comparing said level of step (a) to a reference level of said product, wherein a difference between said level of step (a) and the reference level indicates that the individual is at risk for asthma or asthma exacerbation. In one embodiment, the individual has exhibited one or more symptoms of asthma previously. In another embodiment, the individual has not exhibited one or more symptoms of asthma previously.

In another aspect, the invention provides an array for use in assessing the risk for asthma or asthma exacerbation in a patient, comprising a plurality of discrete regions or addresses, each of which comprises a target molecule disposed thereon, wherein a subset of the plurality of discrete regions has disposed thereon target molecules that can specifically detect a marker of asthma exacerbation. In some embodiments, the subset of the plurality of discrete regions that can specifically detect a marker of asthma exacerbation is at least 5%, 15%, 20%, 25%, 30%, 40%, 45%, 50%, 55%, 60%, 65%, 70%, 75%, 80%, 90%, 95%, or 99% of the total discrete regions on the array. In some embodiments, the target molecules are single stranded polynucleotides that hybridize to polynucleotides obtained from a sample. In other embodiments, the target molecules are peptide recognition moieties, such as for example antibodies or antibody fragments, aptamers, cognate ligands or receptors, and the like. In some embodiments, the sample is obtained from an individual. In some embodiments, the sample from the individual is a blood sample which contains peripheral blood mononuclear cells.

In some embodiments of the aforementioned aspects, the genes or markers that are differentially expressed during asthma exacerbation are depicted in Tables 2-6 and 8-12. In other embodiments, the genes or markers are involved in interleukin-15 (IL-15) signaling, (B-cell receptor) BCR signaling, toll-like receptor (TLR) signaling, interferon (IFN) signaling and/or interferon regulatory factor (IRFs) pathways.

In any one or more of the foregoing aspects, the reference levels of gene products that are differentially expressed during asthma exacerbation are levels of those gene products that are expressed in individuals free of asthma symptoms or in an asthma quiet period. In some embodiments, the reference level is an average of levels obtained from symptom free or asthma quiet individuals. In other embodiments, the reference level is obtained during an asthma quiet period from the same individual who is being tested.

In another aspect, the invention provides a combination of polynucleotides comprising at least 2 or more, or at least 10 substantially purified and isolated polynucleotides, wherein each polynucleotide comprises at least 22 contiguous nucleotides of a gene selected from the group comprising the genes set forth in Table 2, Table 3, Table 4, Table 5, Table 6, Table 8, Table 9, Table 10, Table 11, Table 12 and SEQ ID NOs: 1-77, or the complements and fragments thereof. In one embodiment, the combination of polynucleotides is attached to a substrate to form an array.

In another aspect, the invention provides a kit comprising a detection reagent which binds to the gene product of one or more genes that are differentially expressed in a sample obtained from an individual having an asthma exacerbation versus a sample obtained from an individual having an asthma quiet period. In some embodiments, the one or more genes are selected from the group consisting of the genes set forth in Tables 1-6 and 8-12, and SEQ ID NOs:1-59. In some embodiments, the sample obtained from an individual having an asthma exacerbation is a blood sample. In some embodiments, the gene product comprises a polypeptide and the detection reagent comprises an antibody or an aptamer. In other embodiments, the gene product comprises a polynucleotide and the detection reagent comprises an oligonucleotide or a polynucleotide.

In any one or more of the foregoing aspects, the samples obtained from individuals can be any cell, tissue or fluid. In some embodiments, the sample is a blood sample, which contains peripheral blood mononuclear cells (PBMCs). In other embodiments, the sample is serum.

In another aspect, the invention provides a method of discovering a compound that is effective for treating asthma exacerbation, comprising: providing a candidate compound; determining whether said compound inhibits IL-15 activity, wherein inhibition of IL-15 activity indicates that said compound is effective for treating acute exacerbation of asthma.

In one embodiment, the methods for determining the molecular signature of asthma exacerbation comprise combining a sample from a patient with one or more agents capable of reacting with one or more markers in the sample, and detecting a reaction.

### DETAILED DESCRIPTION

### Asthma Exacerbation Markers

The present invention provides a new class of markers that are differentially expressed in acute exacerbation of asthma, particularly in peripheral blood mononuclear cells and/or serum. Specifically, the markers of the present invention upregulate or downregulate their expression in individuals having an asthma attack. The present invention provides methods for assessing the state-of-health as it relates to asthma in an individual by comparing the expression level of one or more markers with a reference expression level of the one or more markers. The present invention also provides methods for asthma diagnosis, prognosis, or assessment in which the expression level of one or more markers of the present invention is compared to a reference level of the one or more markers.

A study was conducted to investigate the transcriptomics and proteomics of asthma exacerbation. The study was intended to identify potential new targets and/or markers for asthma, particularly asthma exacerbation. The approach to the answers to these questions involved seeking to identify differences between the asthma quiet and asthma exacerbation phenotypes at the molecular level.

The inventors have discovered that particular sets of genes are differentially expressed in individuals during asthma exacerbation as compared to during an asthma quiet time. A subset of those genes that are differentially expressed during exacerbation versus quiet, and which have a false discovery rate of less than 0.05 are listed in Table 2. The study individuals were clustered into three subgroups, based upon their exacerbation molecular profile: subgroup X, subgroup Y and subgroup Z.

For subgroup X individuals, Table 4 depicts 1081 genes having an exacerbation versus quiet expression differential with a false discovery rate of less than 0.05. The subgroup X differentially expressed genes include many well-defined interferon (IFN)-inducible genes and transcription factors, such as IFNα, IFNβ, ISGF3G, IRF7, IRF1, SP100, OAS1, OAS2, MX1, MX2, ISG15, IFITM1, NM1, IR27, IR6, IR30, GBP1, GBP2, SP110, IRF4, IFITM2, and IFI16. The subgroup X differentially expressed genes also include genes linked to IFN, such as for example FGL2, LGALS, IL23A, ARTS-1, STAT1, STAT2, IRF1, IRF4, IRF7, ISGF3G, and the like. The subgroup X differentially expressed genes also include those genes driven by interferon regulatory factors (IRFs), such as OAS2, STAT2, IL15, TAP1, CTSS, IFIT3, OAS1, EIF2AK2, PSMB10, CYBB, CASP7, BCL2, STAT1, PSMB9, CASP8, CDKN1A, CASP1, HLA-G, VIL2, GATA3, GBP1, CXCR4, MS4A1, DNASE2, CCL5, TAP2, TEGT, PLSCR1, ISG15, and TNFSF10. The subgroup X differentially expressed genes also include those genes regulated by interleukin-15 (IL-15), which are listed for example in Table 6.

The differential expression of one or more subgroup X differentially expressed genes in a sample of an individual comprises a molecular profile of an asthma exacerbation that indicates that the asthma exacerbation involves innate immunity. The innate immune system is generally known in the art to be involved in the recruitment of immune cells to sites of infection and inflammation through the production of cytokines, the activation of the complement cascade, the identification and removal of foreign substances by leukocytes, and the activation of the adaptive (cognate) immune system via antigen presentation. For subgroup Y individuals, Table 5 depicts 574 genes having an exacerbation versus quiet expression differential with a false discovery rate of less than 0.05. The B-cell receptor (BCR) pathway was identified as a canonical pathway specific to subgroup Y, which includes genes CD72, CD19, CD79B, Syk, BLNK, Rac/Cdc42, MEKKs, and IKK. For subgroup Z individuals, the Toll-like receptor—Toll-IL-1 receptor domain-containing adaptor inducing interferon-β (TLR-TRIF)-induced intracellular signaling pathway was identified as a canonical pathway specific to subgroup Z genes.

The differential expression of one or more subgroup Y differentially expressed genes in a sample of an individual comprises a molecular profile of an asthma exacerbation that indicates that the asthma exacerbation involves cognate immunity. Cognate (adaptive) immunity is generally known in the art to involve the generation and/or elicitation of a specific B-cell (antibody) and T-cell (T-cell receptor) response to antigens and is triggered when a pathogen or other foreign agent evades the innate immune system and generates a threshold level of antigen. Activation of the cognate system integrates with the innate system through antigen presenting cells.

“Asthma exacerbation,” “acute exacerbation of asthma” “exacerbation attack” and “asthma attack” are phrases that are used interchangeably. “Asthma quiet,” “asthma quiet period,” “quiet asthma period,” and “quiet visits,” are phrases that are used interchangeably and generally refer to asthma symptomless periods. In some cases, the air passages of individuals with asthma are inflamed during a quiet period.

The terms “molecular signature,” “expression profile” and “gene expression profile” refer to two or more genes or gene products which represent a particular state of health of an individual. Alternatively, the molecular signature represents a collection of expression values for a plurality (e.g., at least two, but frequently about 10, about 100, about 1000, or more) of members of a library of genes or gene products. In some embodiments, the molecular signature represents the expression pattern for all of the nucleotide sequences in a library or array of nucleotide sequences or genes. Alternatively, the molecular signature represents the expression pattern for one or more subsets of a library of genes or gene products. In some embodiments, the molecular signature indicates the asthma status of an individual, such as e.g. a quiet period or an exacerbation. In some embodiments, the molecular signature is a molecular signature of asthma exacerbation for an individual with asthma, which indicates the type of exacerbation. Types of exacerbation include e.g. exacerbation involving innate immunity, exacerbation involving cognate or adaptive immunity, exacerbation associated with an infection and exacerbation not associated with any infection.

Various aspects of the invention are described in further detail in the following subsections. The use of subsections is not meant to limit the invention. Each subsection may apply to any aspect of the invention.

### Identification of Asthma Exacerbation Markers

As discussed earlier, expression level of markers of the present invention can be used as an indicator and/or predictor of asthma exacerbation. Detection and measurement of the relative amount of an asthma-associated gene, marker or gene product (polynucleotide or polypeptide) of the invention (generally referred to as “marker” or “biomarker”) can be by any method known in the art.

Methodologies for peptide detection include protein extraction from a cell or tissue sample, followed by binding of an antibody specific for the target protein to the protein sample, and detection of the antibody. Antibodies are generally detected by the use of a labeled secondary antibody. The label can be a radioisotope, a fluorescent compound, an enzyme, an enzyme co-factor, or ligand. Such methods are well understood in the art.

Detection of specific polynucleotide molecules may be assessed by gel electrophoresis, column chromatography, or direct sequencing, quantitative PCR, RT-PCR, or nested PCR among many other techniques well known to those skilled in the art.

Detection of the presence or number of copies of all or part of a marker as defined by the invention may be performed using any method known in the art. It is convenient to assess the presence and/or quantity of a DNA or cDNA by Southern analysis, in which total DNA from a cell or tissue sample is extracted, is hybridized with a labeled probe (i.e., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as would be understood by one skilled in the art.

Methodologies for detection of a transcribed polynucleotide can include RNA extraction from a cell or tissue sample, followed by hybridization of a labeled probe (i.e., a complementary polynucleotide molecule) specific for the target RNA to the extracted RNA and detection of the probe (e.g., Northern blotting).

### Diagnosis, Prognosis, and Assessment of Asthma Exacerbation

The markers disclosed in the present invention can be employed in the prediction, diagnosis and/or prognosis of asthma exacerbation comprising the steps of (a) detecting an expression level of an asthma exacerbation marker in a patient; (b) comparing that expression level to a reference expression level of the same asthma exacerbation marker; (c) and diagnosing a patient has having asthma or an asthma exacerbation event, based upon the comparison made. This can be achieved by comparing the expression profile of one or more asthma exacerbation markers in a subject of interest to at least one reference expression profile of the asthma exacerbation markers. The reference expression profile(s) can include an average expression profile or a set of individual expression profiles each of which represents the gene expression of the asthma exacerbation markers in a particular asthma patient during a quiet period or in a disease-free individual.

In many embodiments, one or more asthma exacerbation markers, which are selected from any one or more of Tables 2-6 and 8-12 and SEQ ID NOs:1-77, can be used for asthma diagnosis or disease monitoring. In one embodiment, each asthma exacerbation marker has a p-value of less than 0.01, 0.005, 0.001, 0.0005, 0.0001, or less. In another embodiment, the asthma exacerbation marker comprises a gene having a log 2 difference between asthma exacerbation and asthma quiet of ≧|0.25| (absolute value of 0.25).

The asthma exacerbation markers of the present invention can be used alone, or in combination with other clinical tests, for asthma diagnosis, prognosis or monitoring. Conventional methods for detecting or diagnosing asthma include, but are not limited to, blood tests, chest X-ray, biopsies, skin tests, mucus tests, urine/excreta sample testing, physical exam, or any and all related clinical examinations known to the skilled artisan. Any of these methods, as well as any other conventional or non-conventional method, can be used, in addition to the methods of the present invention, to improve the accuracy of asthma diagnosis, prognosis or monitoring.

The expression profile of a patient of interest (which by definition comprises the level of at least one marker in a sample obtained from an individual) can be compared to one or more reference expression profiles. The reference expression profiles (which by definition comprise a reference level of the marker) can be determined concurrently with the expression profile of the patient of interest. The reference expression profiles can also be predetermined or prerecorded in electronic or other types of storage media.

The reference expression profiles can include average expression profiles, or individual profiles representing gene expression patterns in particular patients. In one embodiment, the reference expression profiles used for a prediction or diagnosis of asthma exacerbation include an average expression profile of the marker(s) in tissue samples, such as peripheral blood samples, of healthy volunteers or individuals during an asthma quiet period. In one embodiment, the reference expression profiles include an average expression profile of the marker(s) in tissue samples, such as peripheral blood samples, of reference asthma patients who have known or determinable disease status. Any averaging method may be used, such as arithmetic means, harmonic means, average of absolute values, average of log-transformed values, or weighted average. In one example, the reference asthma patients have the same disease assessment. In another example, the reference patients are healthy volunteers used in a diagnostic method. In another example, the reference asthma patients can be divided into at least two classes, each class of patients having a different respective disease assessment. The average expression profile in each class of patients constitutes a separate reference expression profile, and the expression profile of the patient of interest is compared to each of these reference expression profiles.

Other types of reference expression profiles can also be used in the present invention. In yet another embodiment, the present invention uses a numerical threshold as a control level. The numerical threshold may comprise a ratio, including, but not limited to, the ratio of the expression level of a marker in an asthma patient in relation to the expression level of the same marker in a healthy or asthma quiet individual; or the ratio between the expression levels of the marker in an asthma patient both before and after an exacerbation event. The numerical threshold may also by a ratio of marker expression levels between patients with differing disease assessments.

The expression profile of the patient of interest and the reference expression profile(s) can be constructed in any form. In one embodiment, the expression profiles comprise the expression level of each marker used in outcome prediction. The expression levels can be absolute, normalized, or relative levels. Suitable normalization procedures include, but are not limited to, those used in nucleic acid array gene expression analyses or those described in Hill, et al., (Hill (2001) Genome Biol. 2:research0055.1-0055.13). In one example, the expression levels are normalized such that the mean is zero and the standard deviation is one. In another example, the expression levels are normalized based on internal or external controls, as appreciated by those skilled in the art. In still another example, the expression levels are normalized against one or more control transcripts with known abundances in blood samples. In many cases, the expression profile of the patient of interest and the reference expression profile(s) are constructed using the same or comparable methodologies.

In another embodiment, each expression profile being compared comprises one or more ratios between the expression levels of different markers. An expression profile can also include other measures that are capable of representing gene expression patterns or protein levels.

### Samples

The peripheral blood samples used in the present invention can be either whole blood samples, samples comprising enriched PBMCs, or serum. In one example, the peripheral blood samples used for preparing the reference expression profile(s) comprise enriched or purified PBMCs, and the peripheral blood sample used for preparing the expression profile of the patient of interest is a whole blood sample. In another example, all of the peripheral blood samples employed in outcome prediction comprise enriched or purified PBMCs. In many cases, the peripheral blood samples are prepared from the patient of interest and reference patients using the same or comparable procedures.

Other types of blood samples can also be employed in the present invention, such as serum, which contains protein biomarkers; and the gene or protein expression profiles in these blood samples are statistically significantly correlated with patient outcome.

### Assays

Construction of the expression profiles typically involves detection of the expression level of each marker used in the prediction, diagnosis, prognosis or monitoring of asthma exacerbation. Numerous methods are available for this purpose. For instance, the expression level of a gene can be determined by measuring the level of the RNA transcript(s) of the gene(s). Suitable methods include, but are not limited to, quantitative RT-PCR, Northern blot, in situ hybridization, slot-blotting, nuclease protection assay, and nucleic acid array (including bead array). The expression level of a gene can also be determined by measuring the level of the polypeptide(s) encoded by the gene. Suitable methods include, but are not limited to, immunoassays (such as ELISA, RIA, FACS, or Western blot), 2-dimensional gel electrophoresis, mass spectrometry, or protein arrays.

In one aspect, the expression level of a marker is determined by measuring the RNA transcript level of the gene in a tissue sample, such as a peripheral blood sample. RNA can be isolated from the peripheral blood or tissue sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR (RT-PCR), isothermal amplification, ligase chain reaction, and Q-beta replicase.

In one embodiment, the amplification protocol employs reverse transcriptase. The isolated mRNA can be reverse transcribed into cDNA using a reverse transcriptase, and a primer consisting of oligo (dT) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TaqMan, ABI) is used for detecting or comparing the RNA transcript level of a marker of interest. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a marker of interest. The nucleic acid arrays can be commercial oligonucleotide or cDNA arrays. They can also be custom arrays comprising concentrated probes for the markers of the present invention. In many examples, at least 5%, 10%, 15%, 20%, 25%, 30%, 35%, 40%, 45%, 50%, or more of the total probes on a custom array of the present invention are probes for asthma exacerbation markers. These probes can hybridize under stringent or nucleic acid array hybridization conditions to the RNA transcripts, or the complements thereof, of the corresponding markers.

“Nucleic acid array hybridization conditions” refer to the temperature and ionic conditions that are normally used in nucleic acid array hybridization. These conditions include 16-hour hybridization at 45° C., followed by at least three 10-minute washes at room temperature. The hybridization buffer comprises 100 mM MES, 1 M Na⁺, 20 mM EDTA, and 0.01% Tween 20. The pH of the hybridization buffer preferably is between 6.5 and 6.7. The wash buffer is 6×SSPET, which contains 0.9 M NaCl, 60 mM NaH₂PO₄, 6 mM EDTA, and 0.005% Triton X-100. Under more stringent nucleic acid array hybridization conditions, the wash buffer can contain 100 mM MES, 0.1 M Na⁺, and 0.01% Tween 20.

As used herein, “stringent conditions” are at least as stringent as, for example, conditions G-L shown in Table 7. “Highly stringent conditions” are at least as stringent as conditions A-F shown in Table 7. Hybridization is carried out under the hybridization conditions (Hybridization Temperature and Buffer) for about four hours, followed by two 20-minute washes under the corresponding wash conditions (Wash Temp. and Buffer).

In one example, a nucleic acid array of the present invention includes at least 2, 5, 10, or more different probes. Each of these probes is capable of hybridizing under stringent or nucleic acid array hybridization conditions to a different respective marker of the present invention. Multiple probes for the same marker can be used on the same nucleic acid array. The probe density on the array can be in any range.

The probes for a marker of the present invention can be a nucleic acid probe, such as, DNA, RNA, PNA (peptide nucleic acid), or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the markers can be stably attached to discrete regions on a nucleic acid array. By “stably attached,” it means that a probe maintains its position relative to the attached discrete region during hybridization and signal detection. The position of each discrete region on the nucleic acid array can be either known or determinable. All of the methods known in the art can be used to make the nucleic acid arrays of the present invention. Hybridization probes or amplification primers for the markers of the present invention can be prepared by using any method known in the art.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

In one embodiment, the probes/primers for a marker significantly diverge from the sequences of other markers. This can be achieved by checking potential probe/primer sequences against a human genome sequence database, such as the Entrez database at the U.S. National Center for Biotechnology Information (“NCBI”). One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by those skilled in the art.

In another embodiment, the probes for markers can be polypeptide in nature, such as, antibody probes. The expression levels of the markers of the present invention are thus determined by measuring the levels of polypeptides encoded by the markers. Methods suitable for this purpose include, but are not limited to, immunoassays such as ELISA, RIA, FACS, dot blot, Western Blot, immunohistochemistry, and antibody-based radio-imaging. In addition, high-throughput protein sequencing, 2-dimensional SDS-polyacrylamide gel electrophoresis, mass spectrometry, or protein arrays can be used.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can be detected. Detection can be achieved by the addition of a second antibody which is specific for the target proteins and is linked to a detectable label. Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying ELISA, the samples suspected of containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, ¹²⁵I. In one embodiment, a fixed concentration of ¹²⁵I-labeled polypeptide is incubated with a series of dilution of an antibody specific to the polypeptide. When the unlabeled polypeptide is added to the system, the amount of the ¹²⁵I-polypeptide that binds to the antibody is decreased. A standard curve can therefore be constructed to represent the amount of antibody-bound ¹²⁵I-polypeptide as a function of the concentration of the unlabeled polypeptide. From this standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (e.g., such as those which inhibit dimer formation) can also be used. Methods for preparing these antibodies are well known in the art. In one embodiment, the antibodies of the present invention can bind to the corresponding marker gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the markers. Methods for making protein arrays or biochips are well known in the art. In many embodiments, a substantial portion of probes on a protein array of the present invention are antibodies specific for the marker products. For instance, at least 5%, 10%, 15%, 20%, 25%, 30%, 35%, 40%, 45%, 50%, or more probes on the protein array can be antibodies specific for the marker gene products.

In yet another aspect, the expression levels of the markers are determined by measuring the biological functions or activities of these genes. Where a biological function or activity of a gene is known, suitable in vitro or in vivo assays can be developed to evaluate the function or activity. These assays can be subsequently used to assess the level of expression of the marker.

After the expression level of each marker is determined, numerous approaches can be employed to compare expression profiles. Comparison of the expression profile of a patient of interest to the reference expression profile(s) can be conducted manually or electronically. In one example, comparison is carried out by comparing each component in one expression profile to the corresponding component in a reference expression profile. The component can be the expression level of a marker, a ratio between the expression levels of two markers, or another measure capable of representing gene expression patterns. The expression level of a gene can have an absolute or a normalized or relative value. The difference between two corresponding components can be assessed by fold changes, absolute differences, or other suitable means.

Comparison of the expression profile of a patient of interest to the reference expression profile(s) can also be conducted using pattern recognition or comparison programs, such as the k-nearest-neighbors algorithm as described in Armstrong, et al., (Armstrong (2002) Nature Genetics 30:41-47), or the weighted voting algorithm as described below. In addition, the serial analysis of gene expression (SAGE) technology, the GEMTOOLS gene expression analysis program (Incyte Pharmaceuticals), the GeneCalling and Quantitative Expression Analysis technology (Curagen), and other suitable methods, programs or systems can be used to compare expression profiles.

Multiple markers can be used in the comparison of expression profiles. For instance, 2, 4, 6, 8, 10, 12, 14, or more markers can be used. In addition, the marker(s) used in the comparison can be selected to have relatively small p-values (e.g., two-sided p-values). In many examples, the p-values indicate the statistical significance of the difference between gene expression levels in different classes of patients. In many other examples, the p-values suggest the statistical significance of the correlation between gene expression patterns and clinical outcome. In one embodiment, the markers used in the comparison have p-values of no greater than 0.05, 0.01, 0.001, 0.0005, 0.0001, or less. Markers with p-values of greater than 0.05 can also be used. These genes may be identified, for instance, by using a relatively small number of blood samples.

Similarity or difference between the expression profile of a patient of interest and a reference expression profile is indicative of the class membership of the patient of interest. Similarity or difference can be determined by any suitable means. The comparison can be qualitative, quantitative, or both.

In one example, a component in a reference profile is a mean value, and the corresponding component in the expression profile of the patient of interest falls within the standard deviation of the mean value. In such a case, the expression profile of the patient of interest may be considered similar to the reference profile with respect to that particular component. Other criteria, such as a multiple or fraction of the standard deviation or a certain degree of percentage increase or decrease, can be used to measure similarity.

In another example, at least 50% (e.g., at least 60%, 70%, 80%, 90%, or more) of the components in the expression profile of the patient of interest are considered similar to the corresponding components in a reference profile. Under these circumstances, the expression profile of the patient of interest may be considered similar to the reference profile. Different components in the expression profile may have different weights for the comparison. In some cases, lower percentage thresholds (e.g., less than 50% of the total components) are used to determine similarity.

The marker(s) and the similarity criteria can be selected such that the accuracy of the diagnostic determination or the outcome prediction (the ratio of correct calls over the total of correct and incorrect calls) is relatively high. For instance, the accuracy of the determination or prediction can be at least 50%, 60%, 70%, 80%, 90%, or more.

### Screening Methods

The invention also provides methods (also referred to herein as “screening assays”) for identifying agents capable of modulating marker expression (“modulators”), i.e., candidate or test compounds or agents comprising therapeutic moieties (e.g., peptides, peptidomimetics, peptoids, polynucleotides, small molecules or other drugs) which (a) bind to a marker gene product or (b) have a modulatory (e.g., upregulation or downregulation; stimulatory or inhibitory; potentiation/induction or suppression) effect on the activity of a marker gene product or, more specifically, (c) have a modulatory effect on the interactions of the marker gene product with one or more of its natural substrates, or (d) have a modulatory effect on the expression of the marker. Such assays typically comprise a reaction between the marker gene product and one or more assay components. The other components may be either the test compound itself, or a combination of test compound and a binding partner of the marker gene product.

The test compounds of the present invention are generally either small molecules or biomolecules. Small molecules include, but are not limited to, inorganic molecules and small organic molecules. Biomolecules include, but are not limited to, naturally-occurring and synthetic compounds that have a bioactivity in mammals, such as polypeptides, polysaccharides, and polynucleotides. In one embodiment, the test compound is a small molecule. In another embodiment, the test compound is a biomolecule. One skilled in the art will appreciate that the nature of the test compound may vary depending on the nature of the protein encoded by the marker of the present invention.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckerman et al. (Zuckerman (1994) J. Med. Chem. 37:2678-85); spatially addressable parallel solid phase or solution phase libraries; synthetic library methods requiring deconvolution; the “one-bead, one-compound” library method; and synthetic library methods using affinity chromatography selection. The biological library and peptoid library approaches are applicable to peptide, non-peptide oligomers or small molecule libraries of compound (Lam (1997) Anticancer Drug Des. 12:145).

The invention provides methods of screening test compounds for inhibitors of the marker gene products of the present invention. The method of screening comprises obtaining samples from subjects diagnosed with or suspected of having asthma, contacting each separate aliquot of the samples with one or more of a plurality of test compounds, and comparing expression of one or more marker gene products in each of the aliquots to determine whether any of the test compounds provides a substantially decreased level of expression or activity of a marker gene product relative to samples with other test compounds or relative to an untreated sample or control sample. In addition, methods of screening may be devised by combining a test compound with a protein and thereby determining the effect of the test compound on the protein.

In addition, the invention is further directed to a method of screening for test compounds capable of modulating with the binding of a marker gene product and a binding partner, by combining the test compound, the marker gene product, and binding partner together and determining whether binding of the binding partner and the marker gene product occurs. The test compound may be either a small molecule or a biomolecule.

Modulators of marker gene product expression, activity or binding ability are useful as therapeutic compositions of the invention. Such modulators (e.g., antagonists or agonists) may be formulated as pharmaceutical compositions, as described herein below. Such modulators may also be used in the methods of the invention, for example, to diagnose, treat, or prognose asthma.

The invention provides methods of conducting high-throughput screening for test compounds capable of inhibiting activity or expression of a marker gene product of the present invention. In one embodiment, the method of high-throughput screening involves combining test compounds and the marker gene product and detecting the effect of the test compound on the marker gene product.

A variety of high-throughput functional assays well-known in the art may be used in combination to screen and/or study the reactivity of different types of activating test compounds. Since the coupling system is often difficult to predict, a number of assays may need to be configured to detect a wide range of coupling mechanisms. A variety of fluorescence-based techniques is well-known in the art and is capable of high-throughput and ultra high throughput screening for activity, including but not limited to BRET™ (bioluminescence resonance energy transfer) or FRET™ (fluorescence resonance energy transfer) (both by Packard Instrument Co., Meriden, Conn.). The ability to screen a large volume and a variety of test compounds with great sensitivity permits for analysis of the therapeutic targets of the invention to further provide potential inhibitors of asthma. The BIACORE™ system (a plasmon resonance system) may also be manipulated to detect binding of test compounds with individual components of the therapeutic target, to detect binding to either the encoded protein or to the ligand.

Therefore, the invention provides for high-throughput screening of test compounds for the ability to inhibit activity of a protein encoded by the marker gene products listed in Tables 2, 3, 4, 5, 6, 8, 9, 10, 11, 12 and/or SEQ ID NOs:1-77, by combining the test compounds and the protein in high-throughput assays such as BIACORE™, or in fluorescence-based assays such as FRET or BRET™. In addition, high-throughput assays may be utilized to identify specific factors which bind to the encoded proteins, or alternatively, to identify test compounds which prevent binding of the receptor to the binding partner. In the case of orphan receptors, the binding partner may be the natural ligand for the receptor. Moreover, the high-throughput screening assays may be modified to determine whether test compounds can bind to either the encoded protein or to the binding partner (e.g., substrate or ligand) which binds to the protein.

In one embodiment, the high-throughput screening assay detects the ability of a plurality of test compounds to bind to a marker gene product selected from the group consisting of the markers listed in Tables 2, 3, 4, 5, 6, 8, 9, 10, 11, 12 and/or SEQ ID NOs:1-77. In another specific embodiment, the high-throughput screening assay detects the ability of a plurality of a test compound to inhibit a binding partner (such as a ligand) to bind to a marker gene product selected from the group consisting of the markers listed in Tables 2, 3, 4, 5, 6, 8, 9, 10, 11, 12 and/or SEQ ID NOs:1-77. In yet another specific embodiment, the high-throughput screening assay detects the ability of a plurality of a test compounds to modulate signaling through a marker gene product selected from the group consisting of the markers listed in Tables 2, 3, 4, 5, 6, 8, 9, 10, 11, 12 and/or SEQ ID NOs:1-77.

### Nucleic Acid Arrays

Polynucleotide probes that correspond to the genes/markers of the present invention can be used to make nucleic acid arrays. A typical nucleic acid array includes at least one substrate support. The substrate support includes a plurality of discrete regions or addresses. The location of each discrete region is either known or determinable. The discrete regions can be organized in various forms or patterns. For instance, the discrete regions can be arranged as an array of regularly spaced areas on the surface of the substrate. Other patterns, such as linear, concentric or spiral patterns, can be used. In one embodiment, a nucleic acid array of the present invention is a bead array which includes a plurality of beads stably associated with the polynucleotide probes of the present invention.

Polynucleotide probes can be stably attached to their respective discrete regions through covalent and/or non-covalent interactions. By “stably attached” or “stably associated,” it means that during nucleic acid array hybridization the polynucleotide probe maintains its position relative to the discrete region to which the probe is attached. Any suitable method can be used to attach polynucleotide probes to a nucleic acid array substrate. In one embodiment, the attachment is achieved by first depositing the polynucleotide probes to their respective discrete regions and then exposing the surface to a solution of a cross-linking agent, such as glutaraldehyde, borohydride, or other bifunctional agents. In another embodiment, the polynucleotide probes are covalently bound to the substrate via an alkylamino-linker group or by coating the glass slides with polyethylenimine followed by activation with cyanuric chloride for coupling the polynucleotides. In yet another embodiment, the polynucleotide probes are covalently attached to a nucleic acid array through polymer linkers. The polymer linkers may improve the accessibility of the probes to their purported targets.

In addition, the polynucleotide probes can be stably attached to a nucleic acid array substrate through non-covalent interactions. In one embodiment, the polynucleotide probes are attached to the substrate through electrostatic interactions between positively charged surface groups and the negatively charged probes. In another embodiment, the substrate is a glass slide having a coating of a polycationic polymer on its surface, such as a cationic polypeptide. The probes are bound to these polycationic polymers. In yet another embodiment, the methods described in U.S. Pat. No. 6,440,723, which is incorporated herein by reference, are used to attach the probes to the nucleic acid array substrate(s).

Various materials can be used to make the substrate support. Suitable materials include, but are not limited to, glasses, silica, ceramics, nylons, quartz wafers, gels, metals, and papers. The substrates can be flexible or rigid. In one embodiment, they are in the form of a tape that is wound up on a reel or cassette. Two or more substrate supports can be used in the same nucleic acid array.

The surfaces of the substrate support can be smooth and substantially planar. The surfaces of the substrate can also have a variety of configurations, such as raised or depressed regions, trenches, v-grooves, mesa structures, and other irregularities. The surfaces of the substrate can be coated with one or more modification layers. Suitable modification layers include inorganic and organic layers, such as metals, metal oxides, polymers, or small organic molecules. In one embodiment, the surface(s) of the substrate is chemically treated to include groups such as hydroxyl, carboxyl, amine, aldehyde, or sulfhydryl groups.

The discrete regions on the substrate can be of any size, shape and density. For instance, they can be squares, ellipsoids, rectangles, triangles, circles, other regular or irregular geometric shapes, or any portion or combination thereof. In one embodiment, each of the discrete regions has a surface area of less than 10⁻¹ cm², such as less than 10⁻², 10⁻³, 10⁻⁴, 10⁻⁵, 10⁻⁶, or 10⁻⁷ cm². In another embodiment, the spacing between each discrete region and its closest neighbor, measured from center-to-center, is in the range of from about 10 to about 400 μm. The density of the discrete regions may range, for example, between 50 and 50,000 regions/cm².

All of the methods known in the art can be used to make the nucleic acid arrays of the present invention. For instance, the probes can be synthesized in a step-by-step manner on the substrate, or can be attached to the substrate in pre-synthesized forms. Algorithms for reducing the number of synthesis cycles can be used. In one embodiment, a nucleic acid array of the present invention is synthesized in a combinational fashion by delivering monomers to the discrete regions through mechanically constrained flowpaths. In another embodiment, a nucleic acid array of the present invention is synthesized by spotting monomer reagents onto a substrate support using an ink jet printer. In yet another embodiment, polynucleotide probes are immobilized on a nucleic acid array of the present invention by using photolithography techniques.

The nucleic acid arrays of the present invention can also be bead arrays which comprise a plurality of beads. Polynucleotide probes can be stably attached to each bead using any of the above-described methods.

In one embodiment, a substantial portion of all polynucleotide probes on a nucleic acid array of the present invention can hybridize under stringent or nucleic acid array hybridization conditions (Table 7) to genes that are differentially expressed in samples from individuals having asthma exacerbation versus an asthma quiet period. In some embodiments, at least 5%, 10%, 15%, 20%, 25%, 35%, 45%, 50%, 55%, 60%, 65%, 70%, 75%, 80%, 85%, 90%, 95%, 99% or more of all polynucleotide probes on the nucleic acid array can hybridize to asthma exacerbation differentially expressed genes. The probes for these genes can be concentrated on one substrate support. They can also be attached to two or more substrate supports, such as in the bead arrays.

Any number of polynucleotide probes can be included in a nucleic acid array of the present invention. For instance, the nucleic acid array can include at least 2, 5, 10, 20, 30, 40, 50, 100, 200, 300, 400, 500, 1,000 or more different probes, and each probe can hybridize under stringent or nucleic acid array hybridization conditions to a different respective gene selected from asthma exacerbation genes. In one embodiment, a nucleic acid array of the present invention includes a first set of probes which are capable of hybridizing under stringent or nucleic acid array hybridization conditions to different respective asthma exacerbation genes. In yet another embodiment, a nucleic acid array of the present invention includes at least 2, 5, 10, 20, 30, 40, 50, 100, 200, 300, 400, 500, 1,000, 2,000, 3,000, 4,000, 5,000, or more different probes, and each probe can hybridize under stringent or nucleic acid array hybridization conditions to a different respective target sequence selected from any one or more of Tables 2-6 and 8-12, and SEQ ID NOs:1-77, or the complement thereof.

Multiple probes can be included in the nucleic acid arrays of the present invention for detecting the same target sequence. For instance, at least 2, 5, 10, 15, 20, 25, 30 or more different probes can be used for detecting the same target sequence selected from any one or more of Tables 2-6 and 8-12, and SEQ ID NOs:1-77. In one embodiment, a nucleic acid array of the present invention includes at least 30, 40, 50, or 60 different probes for each target sequence of interest. In another embodiment, a nucleic acid array of the present invention includes 25-39 probes for each target sequence of interest.

Each probe can be attached to a different respective discrete region on a nucleic acid array. Alternatively, two or more different probes can be attached to the same discrete region. The concentration of one probe with respect to the other probe or probes in the same region may vary according to the objectives and requirements of the particular experiment. In one embodiment, different probes in the same region are present in approximately equimolar ratio.

In some embodiments, probes for different tiling or target sequences are attached to different discrete regions on a nucleic acid array. In some applications, probes for different tiling or target sequences are attached to the same discrete region.

The length of each probe on a nucleic acid array of the present invention can be selected to achieve the desirable hybridization effects. For instance, each probe can include or consist of 15, 20, 25, 30, 35, 40, 45, 50, 60, 70, 80, 90, 100 or more consecutive nucleotides. In one embodiment, each probe consists of 25 consecutive nucleotides.

The nucleic acid arrays of the present invention can also include control probes which can hybridize under stringent or nucleic acid array hybridization conditions to respective control sequences, or the complements thereof.

### Kits for Prognosis, Diagnosis, or Selection of Treatment of Asthma

In addition, the present invention features kits useful for the diagnosis or selection of treatment of asthma. Each kit includes or consists essentially of at least one probe for an asthma exacerbation marker. Reagents or buffers that facilitate the use of the kit can also be included. Any type of probe can be used in the present invention, such as hybridization probes, amplification primers, antibodies, or any and all other probes commonly used and known to the skilled artisan. In one embodiment, the asthma exacerbation markers are selected from Table 2, Table 3, Table 4, Table 5, Table 6, Table 8, Table 9, Table 10, Table 11, Table 12 and/or SEQ ID NOs:1-77.

In one embodiment, a kit of the present invention includes or consists essentially of at least 1, 2, 3, 4, 5, 6, 7, 8, 9, 10, or more polynucleotide probes or primers. Each probe/primer can hybridize under stringent conditions or nucleic acid array hybridization conditions to a different respective asthma exacerbation marker. As used herein, a polynucleotide can hybridize to a gene if the polynucleotide can hybridize to an RNA transcript, or complement thereof, of the gene. In another embodiment, a kit of the present invention includes one or more antibodies, each of which is capable of binding to a polypeptide encoded by a different respective asthma prognostic or disease gene/marker.

In one example, a kit of the present invention includes or consists essentially of probes (e.g., hybridization or PCR amplification probes or antibodies) for at least 1, 2, 3, 4, 5, 10, 15, 20, 25, 30, 35, 40, 45, 50, 55, 60, 65, 70, 75, or more genes selected from Tables 2, 3, 4, 5, 6, 8, 9, 10, 11, 12 and/or SEQ ID NOs:1-77. In another embodiment, the kit can contain nucleic acid probes and antibodies to 1, 2, 3, 4, 5, 10, 15, 20, 25, 30, 35, 40, 45, 50, 55, 60, 65, 70, 75, or more genes selected from Tables 2, 3, 4, 5, 6, 8, 9, 10, 11, 12 and/or SEQ ID NOs:1-77.

The probes employed in the present invention can be either labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The kits of the present invention can also have containers containing buffer(s) or reporter means. In addition, the kits can include reagents for conducting positive or negative controls. In one embodiment, the probes employed in the present invention are stably attached to one or more substrate supports. Nucleic acid hybridization or immunoassays can be directly carried out on the substrate support(s). Suitable substrate supports for this purpose include, but are not limited to, glasses, silica, ceramics, nylons, quartz wafers, gels, metals, papers, beads, tubes, fibers, films, membranes, column matrices, or microtiter plate wells. The kits of the present invention may also contain one or more controls, each representing a reference expression level of a marker detectable by one or more probes contained in the kits.

It should be understood that the above-described embodiments and the following examples are given by way of illustration, not limitation. Various changes and modifications within the scope of the present invention will become apparent to those skilled in the art from the present description.

### Example 1

### Human Subjects and Study Design

Adult subjects age 18 years or older with confirmed diagnosis of mild, moderate or severe persistent asthma were enrolled in a prospective 12-month non-interventional study of gene expression associated with asthma. Enrollment was stratified by severity of asthma as defined by NIH 1997 guidelines (NIH Publication No. 07-4051, originally printed July 1997).

A prospective, multi-center, non-interventional study, which included subjects having asthma, was conducted in five countries (Australia, Iceland, Ireland, U.K., and USA). Three types of study visits were conducted: (a) exacerbation visits, defined as taking place during exacerbation attacks and within 14 days of attack onset; (b) follow-up visits, defined as taking place within 14 days after cessation of exacerbation attack; and (c) quiet visits, defined as taking place during stable disease at approximately 3 month intervals.

Blood samples were collected for gene expression analyses from each subject at each visit. Samples were collected into Vacutainer™ cell preparation tubes (CPT, Becton Dickinson). Samples were shipped overnight and cell differential counts taken using a Pentra 5™ (Horiba ABX). Peripheral blood mononuclear cells (“PBMCs”) were purified according to manufacturer's instructions. Isolated PBMC pellets were stored at −80° C. pending RNA purification. RLT lysis buffer (with 0.1% β-mercaptoethanol; Qiagen) was added to the frozen pellets. RNA was isolated from the lysate using RNeasy™ Mini Kit (Qiagen Catalog #74104) and DNase treated (Qiagen RNase-free DNase Kit Catalog #79254). The DNase treated RNA preparation was further purified using a Phase Lock Gel™ column (Brinkman). RNA quality was assessed as acceptable by Agilent Bioanalyzer™ gel (Model 2100), and quantified using SpectraMax™ (Molecular Devices).

Exacerbation Visit Samples: From the total of 357 enrolled subjects, at least one evaluable exacerbation visit sample was collected from each of 118 (59 severe, 51 moderate, and 8 mild) subjects. A total of 166 exacerbation visits samples were collected from these 118 subjects. Of these, 25% were collected on the day of exacerbation attack onset, 16% one day post onset, 18% two days post onset, 37% between 3 and 9 days post onset, and the remaining 4% between 10 and 14 days post-onset. 161 of the exacerbation samples were collected while the subjects were experiencing one or more of the following symptoms: wheezing, chest tightness, and/or shortness of breath, (with concomitant symptom of cough reported for 48% of samples). For the other 5 exacerbation samples, for which neither wheezing, chest tightness nor shortness of breath were reported, cough attributed by the physician to an exacerbation attack was noted. Symptoms of upper respiratory infections associated with exacerbation attack were reported for 23% of 166 exacerbation visit samples.

Follow-up Visit Samples: A total of 125 evaluable follow-up samples from 102 subjects were collected from the 118 exacerbation visit subjects.

Quiet Visit Samples: A total of 393 evaluable quiet visit samples were collected from the 118 subjects used in the comparison of quiet and exacerbation visits. A total of 345 evaluable quiet visit samples were collected from the 102 subjects used in analyses relating to follow-up visits.

### Example 2

### Determination of Gene Expression Levels

Gene expression levels in samples were determined using the U133A Affymetrix GENECHIP Array®. Quality control acceptance criteria are shown in Table 1. Samples that did not pass these quality control criteria were re-run, and samples that failed twice were excluded from analyses. A sample was considered evaluable if (a) GENECHIP quality control acceptance criteria were met, and (b) at least one exacerbation visit sample and at least one quiet visit sample was available from the same subject. Labeled target for oligonucleotide arrays were prepared using 2 μg of total RNA according to the Affymetrix protocol. Biotinylated cRNA was hybridized to the HG-U133A Affymetrix GENECHIP Array®. Raw intensity values were processed using Affymetrix MAS 5.0 software, which calculated signal expression levels and present/absent calls for each probe set.

Of the 22,283 probe sets present on the U133A array, a subset of 9,696 probe sets, which met the following two criteria, were analyzed: (a) those probe sets detected as being present in at least 10% of the samples; and (b) those probe sets having a signal of at least 50 in at least 10% of the samples.

The clinical and gene expression databases were merged using SAS version 9.1. Correct association of GENECHIP data with sample donor was verified by determining that gender specific expression patterns correctly reflected the donor's gender, and by a consistent expression pattern of HLA marker genes in samples collected at different times from the same donor.

Analysis of covariance (ANCOVA) methods were used to adjust for covariates when testing for differences in expression levels between visit types. The ANCOVA models used log₂-transformed MAS 5.0 signal as the dependent variable and included terms for visit type, asthma severity defined by NIH guidelines, sex, age (18-39, 40-59, or 60-83), race, sample processing lab, maximum corticosteroid exposure (a 4-level variable reflecting corticosteroid exposure at time of visit, with systemic>inhaled>intranasal>no corticosteroid exposure), an indicator for use of leukotriene antagonist at time of visit, bactin-GAPDH ratio (an indicator of RNA quality), and monocyte/lymphocyte ratio. The visit type factor was limited to quiet visits and exacerbation visits in some analyses, while in others it included follow-up visits. Some analyses included an additional 3-level factor for exacerbation subgroup. In some analyses, pairwise contrasts were run between specific levels of factors with more than two levels. In such cases, the contrasts were performed using two-sided t-tests, with the denominator of the t-statistics derived from the ANCOVA error term. Separate ANCOVAs were run for each probe set. To adjust for the multiplicity of testing, false discovery rates were calculated across all probe sets, separately for each term in the ANCOVA model or pairwise contrast. All ANCOVAs and false discovery rate (“FDR”; Benjamini and Hochberg, J. of the Royal Statistical Society, Series B, 57:289-3001995) adjustments for multiplicity of testing calculations were run using SAS version 9.1.

Complete linkage hierarchical analysis (SPOTFIRE version 8.1) was used to identify exacerbation visit samples with similar exacerbation associated patterns of gene expression. The difference between the log 2 expression level during each individual exacerbation visit and the mean log 2 expression level of quiet visits for the same subject was calculated for each of the 166 exacerbation visits for each probe set with an association with exacerbation P<0.05. These log ratios were ordered by spectral bi-clustering analysis to organize the expression profiles of each exacerbation visit according to their level of similarity to each other. Based on the heterogeneity observed, iterative K-means clustering was used to assess the evidence for distinct visit subgroups (see, e.g., Hartigan and Wong, Applied Statistics 1979, 28:100-108.) Clustering of visits was executed for K=2, 3, 4, and 8 clusters. For each clustering, the strength of the clustering was assessed by two complementary methods. First, the silhouette statistic (SW) was calculated for each cluster (subgroup) and the overall clustering (see, e.g., Rousseeuw P, J Comput Appl Math 1987, 20:53-65). Second, typical levels of gaussian experimental noise were injected into the expression data, 100 realizations of this noisy data were each clustered, and the weighted sum of the fraction of realizations where the same groups of visits were co-clustered was calculated to generate a robustness index, (R), which is closely related to the measures described in McShane, 2002 (McShane et al., Bioinformatics 2002, 18 (11):1462-1469). For K=2 clusters, there was a clear and robust separation into two clusters (SW=0.19, R=0.998). For K=3 clusters, robust groupings were also found (SW=0.08, R=0.88). Beyond K=3 clusters, the SW and R measures declined further, indicating little support for more than 3 subgroups. Based on these results, K means clustering using 3 clusters was used to segregate exacerbation visit samples into three subgroups designated as X, Y and Z. Analyses using data for all 9,696 probe sets were then conducted to compare subgroup exacerbation visit expression levels to quiet visit expression levels. Probe sets showing a within-subgroup exacerbation association of FDR<0.05 and average fold change with exacerbation >1.2 were defined as meeting the criteria for association with exacerbation.

In order to track the biological pathways and functional networks implicated in exacerbation attacks, genes associated with exacerbation attack were analyzed using Ingenuity Pathway Analysis (IPA 3.1 release, Ingenuity® Systems, www.Ingenuity.com; see e.g., Calvano et al., 2005 Nature 437:1032-1037) to reveal relationships between them.

### Example 3

### Nucleic Acid Methods

Conversion of 2 μg of total RNA from the above preparations to cDNA was accomplished using the Applied Biosystems High Capacity cDNA Archive Kit (Applied Biosystems Catalog #4322171) was performed according to the manufacturer's instructions. Pre-validated, QC tested gene specific primer-probe pairs, optimized for use on any ABI PRISM™ sequence detection system, were purchased from Applied Biosystems (ABI). Real-time quantitative gene expression assay kits were obtained from Applied Biosystems. The genes assayed were IFNα1 (assay Hs00256882_s1), IFNβ1 (assay Hs00277188_s1), IFNγ (assay Hs00174143_m1), IL18 (assay Hs00155517_m1), IL13 (assay Hs00174379_m1), and the endogenous normalizer control, ZNF592 (assay Hs00206029_m1). All study samples were normalized to ZNF592 levels to determine relative concentration values.

Using the Taqman Assay-On-Demand (“AOD”) product insert volume recommendations, a master mix was prepared using Taqman™ Universal PCR Master Mix (Catalog #4304437) and aliquoted into a 96 well plate (ABI Catalog #N801-0560 and caps #N801-0935) for a final volume of 50 μl/well. Duplicate wells for serially diluted standards and cDNA samples (50 ng/well) were assayed on an ABI PRISM 7700 Sequence detector (Sequence Detector Software v1.7) using universal thermal cycling conditions of 50° C. for 2 minutes, 95° C. for 10 minutes and 40 cycles of 95° C. for 15 seconds, 60° C. for 1 minute.

Relative quantification of RNA transcript levels was performed following the guidelines described in ABI PRISM 7700 Sequence Detection System User Bulletin #2 using the relative standard curve method. Specifically, standard curves are calculated for target standards and endogenous control, input values determined for target and endogenous control using standard curves' slope and y-intercept, and target input values are normalized to endogenous control. Fold change is calculated using the 50 ng standard as a calibrator and relative concentration of sample is obtained by multiplying fold change by calibrator, then averaged. To utilize the standard curve method for RNA quantification, a tissue empirically determined to express the target gene was identified using Applied Biosystems Taqman AOD™. Standard curve tissue sources were: cervix tumor from Ambion for INFα, activated human monocyte for IFNβ, activated human PBMC for INFγ and IL18, and thymus from Ambion for IL13. Cycle threshold (Ct) values of >35 were considered below the limits of detection. For standard curve development, the goal was to achieve a Ct value between 18 and 25 for 100 ng of cDNA. This allowed for appropriate standard curve dynamic range. Standard curves consisted of two-fold serial dilutions of total cDNA from 100 ng/well to 1.5 ng/well. Standard curves were performed on each plate for every assay and were used for sample quantification and assay performance monitoring. Inter-plate % CV for standard curve points were <4.5% for IFNα and IL-13 and <3% for interferon-b1, IFNβ and IL-18.

AOD for single exon gene targets (IFNα1 and IFNβ1) can produce inaccurate transcript expression values if the RNA preparations used for cDNA conversion contain genomic DNA. The following strategy was developed and employed to determine which cDNA samples contained genomic DNA.

Genomic sequence analysis was performed in the area of the human KIAA0644 gene product (accession #NM_{—}014817) to determine predicted mRNA sequences using the Ensembl Gene Browser (see Fernandez Suarez and Schuster, “Using the Ensembl Genome Server to Browse Genomic Sequence Data,” UNIT 1.15 in Current Protocols in Bioinformatics, Supplement 16, January 2007; and also http://www.ensembl.org/index.html). A Taqman primer/probe pair was designed (ABI Primer Express) from a predicted nontranslated sequence located approximately 1.5 Kb 3′ of the KIAA0644 single exon gene product open reading frame on chromosome 7. In a Taqman assay, this primer/probe pair was shown to produce a strong signal, Ct value of 24.14, using a human genomic DNA preparation (Clontech catalog #6550-1) and no signal (Ct value of 40) using a commercially available purified RNA preparation from Ambion (human kidney, catalog #7976). Taqman analysis of all AOS cDNA preparations was performed using this primer/probe pair. Samples producing a Ct value of 35 or greater were determined to not be contaminated with genomic DNA, while samples producing a Ct value of less than 35 were considered to be contaminated with genomic DNA. For single exon gene target results, samples containing genomic DNA were not included in the statistical analysis (12% of AOS samples).

Statistical analyses were conducted to compare gene expression for 5 preselected genes (IFNα1, IFNβ1, IFNγ, IL13, and IL18) for exacerbated and quiet asthma periods. For each subgroup of patients (defined by K-means analysis described above), a mixed model analysis of variance was fit to the expression data to compare expression between exacerbation and quiet periods at the five percent significance level. The model included a fixed effect for visit type (quiet or exacerbated) and a random effect for patient to account for multiple visits per patient.

Gene expression from the quiet periods were then combined for the two subgroups to estimate the between and within subject variability in expression for asthma patients during quiet periods. These variance components were also estimated for a set of 28 healthy volunteers.

### Example 4

### PBMC Gene Expression Associated with Exacerbation

By ANCOVA analysis of the 118 subjects comparing quiet (393 samples) and exacerbation (166 samples), 78 probe sets had changes in expression that were associated with exacerbation, based on a criterion of FDR<0.05. The significance of the association with exacerbation ranged from 5.16E-5 to 4.6E-2 (Table 2). A listing and annotation of these 78 probe sets and the significance of association is given in Table 2.

The comparison between exacerbation and quiet visits that identified the 78 sequences was based on 118 subjects, and for 16 of these subjects no evaluable follow-up visit samples were available. The ANCOVA comparing quiet and exacerbation visit gene expression was rerun on visits only from the 102 subjects that also had follow-up visit data. The significant differences between quiet and exacerbation in the comparison based on 118 subjects also trend towards significance in the comparison based on 102 subjects, although, as predictable given the loss of statistical power, there are fewer probe sets with FDR<0.05 in the analysis based on 102 donors (Table 2).

The ANCOVA comparing mean expression levels in quiet and follow-up genes indicated that gene expression levels associated with exacerbation had returned to quiet visit levels at follow-up visit (Table 2).

### Example 5

### Asthma Subgroups

To examine in more detail expression patterns associated with exacerbation, spectral bi-clustering analysis was performed using the difference between the log 2 expression levels during each individual exacerbation visit and the mean log 2 expression level of quiet visits for each of the 166 exacerbation visits. This analysis revealed significant heterogeneity between the expression profiles of exacerbation visits, and suggested that sub-grouping of visits might increase our power to detect transcripts that were differentially expressed only within specific subgroups. It was determined that K-means clustering using 3 clusters defined three relatively distinct and robust exacerbation associated gene expression patterns (see Methods section). K-means clustering was therefore used to assign each exacerbation sample to one of three subgroups (or clusters) designated as X (30 visits), Y (64 visits) and Z (72 visits). ANCOVA was performed on all 9,696 probe sets to compare mean expression levels in each exacerbation subgroup with mean quiet visit expression levels, thereby identifying sub-group specific expression profiles that might have been masked by heterogeneity when data from all exacerbation visits were lumped together.

Since the subgroups (or clusters) were defined based on minimizing variability among members of the same subgroup, the p-values and FDR values observed for specific exacerbation subgroup versus quiet visit comparisons for any given probe set can not be interpreted as numerically equivalent to p-values and FDRs obtained in the earlier analysis of quiet versus exacerbation expression levels. Rather the “within subgroup” FDR values are used primarily to rank the probe sets in terms of significance of differences between exacerbation and quiet expression levels. Therefore, FDR values generated from within subgroup analyses were designated as “comparative FDRs”. Therefore, in practical terms, a probe set with a comparative within subgroup X FDR of <1E-15 is much more likely to be associated with exacerbation than the probe sets with comparative FDRs of >0.05, but the overall probability of association with exacerbation can not be stated to be FDR<1E-15.

Within subgroup X, 1,081 probe sets had differences between exacerbation and quiet visit expression levels, as defined by comparative FDR<0.05 and absolute average fold change >1.2, and 48% of these 1,081 probe sets had comparative FDR<1E-3. Table 4 lists these probes sets along with their gene annotations and the strength of association with exacerbation as determined by comparative FDR. These findings indicate a very robust exacerbation associated gene expression profile within subgroup X. Analyses were then performed to determine the differences between quiet and follow-up visits within subgroup X. Of the 30 subgroup X exacerbation visits, evaluable follow-up samples were available for 22, resulting in 8 (26.6%) fewer samples in the analysis comparing quiet to follow-up visits within subgroup X. Even with this smaller sample size, ANCOVA comparing expression in quiet visits and 22 exacerbation visits for which there was a corresponding follow-up visit, showed that 793 (74%) of the 1,081 exacerbation associated probes sets retained a comparative FDR<0.05, indicating that a robust exacerbation associated expression profile was detectable even with a 26.6% decrease in sample size. In stark contrast, the ANCOVA comparing quiet visits and 22 follow-up visits of subgroup X exacerbation visits for all 9,696 probe sets identified only 36 differences with FCR<0.05, indicating that, unlike exacerbation samples, follow-up samples are very similar to quiet visit samples. Of the 793 probe sets significantly associated with exacerbation in the 22 visit analysis, only 2 had a significant difference between quiet and follow-up visits.

Many of the 1,081 exacerbation-associated subgroup X probe sets did not show even a slight trend towards association with exacerbation in subgroup Y, with 26% having a subgroup Y association FDR >0.5 (50%). These data indicate significant qualitative gene expression differences in subgroup X and Y exacerbations. Overlap between subgroups was also observed, however, with 21% of the subgroup X probe sets showing an association with exacerbation (comparative FDR<0.05) within subgroup Y.

ANCOVA comparing exacerbation and quiet visit expression levels within sub-group Y identified 574 probe sets associated with exacerbation. For subgroup Y, there were 64 exacerbation visits in the analyses comparing quiet and exacerbation, and 51 in the analyses that included follow-up visits. As was seen in the both the conglomerate and subgroup X analyses, for most probe sets subgroup Y expression levels had returned to quiet visit levels by follow-up. The list of the subgroup Y probe sets together with the metrics for association with exacerbation is given in Table 5. Of the probes sets associated with exacerbation in subgroup Y, 24% overlapped with subgroup X probe sets. In addition, subgroup Y probe sets include 39% that did not show even a slight trend with exacerbation (comparative FDR >0.5) in subgroup X. These data confirm the striking difference between subgroups X and Y exacerbations.

Subgroup Z contained the largest number of exacerbation visits (72) and the analyses that included follow-up visits contained 52 samples. The total number of exacerbation association probe sets in subgroup Z was 211, and the lowest relative FDR observed was 0.0004. No probe sets were identified in subgroup Z that did not also show a significant association with exacerbation in subgroup X and/or Y, indicating that subgroup Z does not represent a third qualitatively distinct exacerbation associated profile. Rather the data show that subgroup Z contains the visits that differ the least from quiet visits, and suggest that visits with weak to absent exacerbation associated profiles were assigned by the K-means algorithm to this group.

Chi-square tests or ANOVAs were performed to determine if clinical or technical parameters could be identified that had a significant association with subgroup assignment. A significant association between body mass index (BMI) and subgroup assignment was identified. Mean BMI was statistically significantly lower (p=0.006) in subgroup X than subgroup Y, and was statistically suggestively lower (p=0.0501) in subgroup Z than subgroup Y. Mean BMI was 28.4, 32.4, and 30.2 in subgroups X, Y and Z, respectively. Additionally, subgroup Y samples were somewhat less likely (p=0.042) to be from fasting subjects at time of visit, with 30%, 22% and 29% fasting samples in subgroup X, Y and Z respectively. Subgroup Y samples tended to be from older patients (mean age 46.0 years) than those in subgroup X (mean age 39.1) or Z (mean age 43.5), and this difference was significant in the comparison of subgroups X and Y (p=0.03).

No evidence was found for association between subgroup assignments and any of the following parameters: sex, race, country, disease severity, atopy status, respiratory infection, systemic, inhaled, intra-nasal corticosteroid or leukotriene inhibitor use, histamine H2 antagonist or PPI use, medical history of acid reflux, time between onset of exacerbation and sample collection, or sample processing lab. Also no evidence was found for association with FEV1 or IgE, but many values were missing in this analysis.

The mean number of days between quiet and exacerbation visits was significantly smaller for subgroup X (48.4 days) than for subgroup Y (62.7 days) and or Z (79.6 days). (The p-value of 0.03 is for the overall test comparing the means for the 3 subgroups; the p-value for X vs. Z was 0.014; the p-value for X vs. Y was >0.05). While not wishing to be bound by theory, given the study design, this difference possibly indicates that subgroup X samples were more likely to be collected from subjects who sought medical attention due to symptoms of attack, whereas the samples in the other subgroups were more likely to include some samples collected during a scheduled visit whose exacerbation attack symptoms had not triggered the patient to come in for an exacerbation visit. Other explanations for this observed difference are also possible, including that the different types of exacerbation visits may just have different frequencies of occurrence. The associations between subgroup assignment and clinical parameters therefore suggest that exacerbations with the most acute attack symptoms (as defined by prompting the subject to seek more immediate medical attention) tended to be in subgroup X. Those with exacerbations (and asthma) associated the high BMI tended to be in subgroup Y, perhaps providing a molecular signature for the previously observed link between higher BMI and symptoms of asthma. Those in subgroup Z tended to display more mild form of exacerbation profile as apparently reflected in the significantly longer intervals between seeking medical attention and the very much less robust molecular signature.

### Example 6

### Genes/Markers Associated with Asthma Exacerbation

To determine the biological pathways and functional networks implicated in exacerbation by gene expression patterns, genes were analyzed using Ingenuity Pathway Analysis. Various canonical pathways are specific to subgroups X and Y, respectively. For example, subgroup X canonical signaling pathways include e.g. natural killer cell, antigen presentation, leukocyte extravasation, JAK/Stat, interferon, GM-CSF, T cell receptor, toll-like receptor and IL-10 signaling. Subgroup Y canonical signaling pathways include e.g. IL-4, B cell receptor, death receptor, SAPK/JNK, IL-2, PTEN, circadian rhythm, IGF-1, actin cytoskeleton, PI3K/Akt and insulin receptor signaling. Many IFN-inducible genes were noted in subgroup X. These include the interferon regulatory factors (IRFs) that are known to drive the transcription of various IFN-inducible genes. IRF1, IRF7, IRF9 are upregulated in exacerbation while IRF4 is down in exacerbation.

Networks were built (using the Connect Tool) around these IRFs using the subgroup X associated genes and expression and/or transcription as the connectivity from IRFs to the subgroup X associated genes. Subgroup X associated genes for IRF1, IRF7 and IRF9 are also upregulated in exacerbation suggesting that these IRFs drive the expression of these exacerbation genes. Likewise, exacerbation genes in the IRF4 hub are down and so is IRF4 in exacerbation. A network centered around IL15 was also highly significant, suggesting that IL15 could be regulating the expression of several exacerbation genes. All subgroup X associated genes were connected to IL15 based on information available in IPA for IL15 regulation of gene expression using the Connect tool (Table 6).

Subgroup Y showed a robust signature for the canonical pathway for B cell signaling. While subgroup Z did not have a robust signature, pathway analysis identified TLR pathway as well represented among the genes that passed the significance filter in this subgroup.

Since interferon response elements were so strongly identified with exacerbations, and IFNγ is so strongly identified with a Th1 type response and IFNα and β more consistent with the Th2 response classically associated with asthma, TAQMAN analysis of a subset of samples from subgroups X and Y was performed to assess the association of each of these genes with subgroups X and Y. As shown in Table 3, the results indicate that elevated levels of IFNα and β were associated with subgroup X exacerbations. IFNγ did not differ significantly between quiet and exacerbation samples.

Comparison of quiet and exacerbation visit gene expression profiles identified significant exacerbation associated changes in gene expression levels. Expression levels had returned to quiet visit levels two weeks after the attack. Clustering algorithms identified three relatively distinct exacerbation phenotypes defined by PBMC gene expression profiles, and analysis showed that gene expression patterns identified by ANCOVA performed within subgroups had also returned to quiet visit levels two weeks following an exacerbation, confirming that the within subgroup analysis did, indeed, identify genes significantly associated with exacerbation.

Pathway analysis for the three subgroups identified distinct pathways active within subgroups X, Y and Z. Many IFN-inducible genes such as OAS1, OAS3, MX1, IFITM3, IFIT3, IFI27, IFI35, IFIT1, et cetera are observed in subgroup X. These include interferon regulatory factors (IRFs), a family of transcription factors involved in the regulation of the interferon response. Of the nine known IRFs, IRF1, IRF7 and IRF9 are up-regulated in exacerbation, while IRF4 is down-regulated in exacerbation. The majority of the subgroup X genes that are regulated by IRF1 and IRF7 are also up-regulated in exacerbation. The majority of the subgroup X genes regulated by IRF4, such as CXCR4, MS4A, VIL2 and GATA3 are also down-regulated in exacerbation. IFN response in subgroup X is likely regulated by these IRFs and maybe either a Type I IFN (IFNα/IFNβ and others, such as IFN-ω, -ε, and -κ) or a Type II IFN (IFNγ) response. Taqman data indicates that the subgroup X IFN pathway is driven by IFNα and IFNβ. Data analysis indicates that the IFN pathway activation observed in the instant exacerbation samples are not attributable to respiratory infections, and that samples in this subgroup tend to have come from patients with normal BMI.

Another likely player in subgroup X exacerbations is IL15. IL15 is a TH1 cytokine that activates T-cells in a T-cell receptor independent manner. TCR a, TCR z and CD3D, which is associated with TCR, are down-regulated in exacerbation along with CD8B, a co-receptor for MHC class I as well as downstream signaling proteins such as ITK, PLCg1, TEC, SOS2, PIK3R1 and CALM1. IL15 is up-regulated in exacerbation and so is IL2RG, the shared signaling component of IL15R. So likely subgroup X type exacerbations involve IL15 activation of T-cells in a TCR-independent manner. IRF1 induces IL15, and IFNs may activate CD8T-cells via IL15.

TLRs trigger IFN-responses. TLR-signal transduction occurs either in a MYD-88 dependent manner through the recruitment of IRAK1/4, TRAF6, TAB1/2, TAK1 or in a MYD-88 independent manner that involves TRAM, TRIF, TBK-1, IKK-e and other signaling molecules. TLR3 and TLR4 are the only Toll receptors that utilize the MYD-88 independent signaling pathway. TLR1, TLR2, TLR4 are all expressed at significantly higher levels in exacerbation as well as MYD88, MD-2, CD14 and a downstream kinase EIF2AK1

MDA5/IFIH, which is a cytosolic receptor for intracellular viral RNAs and synthetic dsRNAs, and which mediates TLR-independent induction of type I IFN genes, is also upregulated in subgroup X suggesting that both TLR-dependent and independent pathways are activated in subgroup X.

Additional pathways regulated in subgroup X include, for example, the NK-cell signaling pathway and the antigen presentation pathway.

The NK-cell signaling pathway is common to subgroup Y as well. Subgroup Y genes involved in NK activation such as FCER1 and FCGR3 are expressed at higher levels in exacerbation, as well as the downstream signaling molecules LCK, SYK, LAT, RAC and RRAS, but not PIK3C1 and PIK3RA1. On the NK-inhibition side, receptors LILRB1, LAIR1, AIRM1, as well some downstream signaling molecules, are up-regulated in exacerbation, suggesting compensatory mechanisms in place for NK signaling. Some parallels and some differences in both arms of NK signaling can be noted for subgroup X. Actin-cytoskeletal structural genes such as ARPC5, PFN, CYFIP1, ARPC1B, but not VIL2, are upregulated in Subgroup Y. Some of these trend in the opposite direction for Subgroup X.

The expression levels of TLRs, IRFs, IL15 do not significantly change in subgroup Y compared to the quiets. Few genes common to the TLR, IFN, IL15 pathways in subgroup X such as for example MDA5, IFI35, ICAM2, CCR2, and IL2RG are also seen in Subgroup Y, and almost all trend in the same direction.

Additionally, different genes with similar functions showed sub-group specificity. For example, phopholipase scramblase 1 (PSCR1) is elevated in subgroup X (FDR=7.13E-13) but not in sub-group Y (FDR=0.509), whereas phopholipase scramblase 3 (PSCR3) is elevated in sub-group Y (FDR=0.003) but not in subgroup X (FDR=0.99).

The following tables, which are referenced in the foregoing description, are herein incorporated in their entirety.

### Example 7

### Serum Markers of Exacerbation

Plasma samples from asthmatic donors during either previously scheduled or random exacerbation visits, and healthy volunteer donors were analyzed by ELISA for the presence of various cytokines, sST2 protein, which is the soluble form of ST2, an IL-1 receptor family member and cognate receptor for IL-33 (see Sanada et al., J. Clin. Invest., 117:1538-1548, 2007, which is incorporated herein by reference), and chitinase 3-like 1 protein (YKL-40, CHI3L1) (see Table 8.) CHI3L1 showed a significant difference is expression in the sera of asthmatics versus healthy volunteers, indicating its usefulness as an asthma-associated biomarker.

Serum sST2 concentrations were found to be significantly higher in (a) asthmatics versus healthy donors (p<0.05); (b) asthmatics during exacerbation versus asthmatics during scheduled visits (p<0.05); and (c) asthmatics during exacerbation versus healthy volunteers (p<0.0005). Specifically, the concentration of sST2 in sera was observed to be elevated upon exacerbation (90 pg/mL) relative to normal controls (55 pg/ml) (p value<0.0001). It was further observed that, upon asthma exacerbation, males have higher sST2 concentration in the sera (126 pg/mL) relative to females (78 pg/mL) (p value<0.01).

The question of whether sST2 is induced in response to G-protein coupled receptor (GPCR) activation was examined in a human mast cell line (HMC-1; see Versluis et al., Int. Immunopharmacol., 8:866-873, 2008.) We observed strong induction of sST2 mRNA and protein expression upon cell activation with asthma associated anaphylatoxin C5a and adenosine analog NECA, that activate GPCR signaling via C5a and adenosine receptor, respectively. Thus, sST2 is a useful asthma and exacerbation biomarker for the clinic.

### Example 8

### Intra-Subject Variability of Biomarkers

We have shown that there are significant differences in PBMC gene expression profiles of asthma exacerbation subjects and asthma quiet or healthy subjects. In this example, we have shown that the expression level of many asthma associated genes can vary over time (e.g. between visits separated by time) within a subject, and can range from close to healthy to very different from healthy, and that differences between subjects are not necessarily greater than differences within subjects. The result of such an analysis will enable the selection of more optimal asthma and asthma exacerbation biomarker candidates that have higher incidences of deviation from healthy and quiet, respectively, on a per visit basis, as well as lower intra-subject deviations. (See copending U.S. Patent Application No. 60/879,994, which is herein incorporated by reference.) Non-limiting examples of such more optimal biomarkers for exacerbation include BLVRA (biliverdin reductase A), CSE1L (chromosome segregation 1-like), CTSC (cathepsin C), FCN1 (ficolin 1), GRN (granulin), LAMP2 (lysosomal-associated membrane protein 2), PECAM1 (platelet/endothelial cell adhesion molecule-1), S100A9 (S100 calcium binding protein A9) and SP110 (SP110 nuclear body protein). Exacerbation biomarkers having low intra-subject variability and high deviation from quiet or healthy are also shown in Table 9 and Table 10 for cluster X and cluster Y subgroups, respectively. These markers can be used to predict an exacerbation event in asthma sufferers.

To demonstrate this intra-subject variability, a first analysis was run on GeneChips from the first visit for each subject and a second analysis was run on GeneChips from the second visit for each subject (subsequent analysis looked at later visits). Using all subjects and analyzing data from all visits analysis, 438 probesets, which were significantly associated with asthma, were selected. For each probeset, the log 2 fold change was calculated for each asthma sample (including exacerbation asthma samples) over average healthy (all subjects, all visits). A quantitative scale was devised, which indicates the “distance” between an individual asthma (asthma exacerbation) profile and the mean healthy profile. Then the range of distance of asthma or asthma exacerbation from healthy was analyzed on a subject-by-subject basis.

The first and second visit analyses gave the same results, including the same cluster structure, same asthma genes, and almost the same fold change in expression level. However, it was noted that the subjects move between a subcluster that is very different from healthy and a subcluster that is close to healthy, showing that some asthma-associated and exacerbation-associated genes vary within a subject over time.

### Example 9

### Biomarkers for Inflammatory Diseases

The 438 probesets used for asthma profile (supra) were examined for their association with other inflammatory diseases. Approximately 155 of those markers were significantly associated with asthma and not with multiple sclerosis (MS) or inflammatory bowel disease (IBD). 164 were associated with asthma and MS, with an additional 112 at least trending to significance in MS. 16 markers were associated with asthma and Crohn's disease, 10 of which did not also associated with MS. Nine (9) markers were associated with asthma and ulcerative colitis (UC).

The majority of genes common to MS and asthma changed in the same direction relative to normal or healthy in both diseases, with the following exceptions: IL21R (interleukin 21 receptor) was up in MS, down in asthma, and down more in severe asthma; CUTL1 (Cut-like 1, CCAAT displacement protein) was up in MS, down in asthma, down more in severe asthma; DGKD (Diacylglycerol kinase, delta 130 kDa) was up in MS, down in asthma, down more in severe asthma; and KIAA0528 (hypothetical protein LOC9847) was up in MS, down in asthma, and down more in severe asthma.

### Example 10

### Exacerbation During Respiratory Infections

Of the 166 exacerbation samples, 39 occurred during a respiratory system infection and 127 occurred with out symptoms of infection. To identify probe sets that showed association with exacerbation only in the presence of infection, an ANCOVA was performed comparing the 39 samples collected during infection with the quiet visits from the same patients. 54 probesets were identified with FDR<0.05 (Table 11) Of note among the 54 were 16 of the 54 probe sets showed an association with exacerbation in the presence of infection, but did not show a significant association in the analysis comparing the mixed group of 166 exacerbations (with and without infection) and quiet samples (Table 12). Consistent with this finding, none of these 16 was significantly associated with exacerbations in the absence of infection. These data indicate that there were some probe sets whose association with exacerbation was detectable only in the presence of a concomitant infection.

### Example 11

### Exacerbation in the Absence of Infection

At least three probe sets were observed to be associated with exacerbation in the absence of infection (i.e. not associated with exacerbation in the presence of infection). Those probes sets include: (a) interferon induced with helicase C domain 1 (IFIH1; e.g. SEQ ID NO:60), (b) leukotriene A4 hydrolase (LTA4H; e.g. SEQ ID NO:61) and (c) open reading frame number 25 of human chromosome 6 (C6ORF25; SEQ ID NO:62). These probe sets can serve as biomarkers of exacerbation triggered by inert non-infectious agents.

TABLE 1
Quality Control Criteria for Inclusion of GENECHIP in Analysis

| | | |
|---|---|---|
| 1 | Defect on visual inspection | |
| 2 | Bactin Gapdh Freq Avg Exp | >0.6 |
| 3 | Genechip Raw Q Exp | <7 |
| 4 | Qc P Prob Freq Exp | <20 |
| 5 | Qc P Prob Avg Diff Exp | <205 |
| 6 | Qc Sensitivity Exp | <6.1 |
| 7 | Scale Factor Exp | <4 and >0.25 |

1 Defect on visual inspection: Patterns in chip fluorescence visible after the chip has been run that reveal scratches, uneven staining or other defects.
2 Ratio of signal portion of the gene. A measure of the integrity of the RNA sample.
3 Raw Q: measure of the noise level of the array, it is the degree of pixel-to-pixel variation among the probe cells used to calculate the background.
4 QCP probability average difference: signal value for which there is a 70% probability of a Present call.
5 QCP probability frequency: QCP probability average difference expressed in ppm units.
6 Chip sensitivity: concentration level, in ppm, at which there is a 70% probability of obtaining a Present call.
7 Scale factor: the value required to obtain a trimmed mean intensity indicated by the target value. For all data in this study, the target value was set to a value of 100 and the scale factor was determined by dividing the trimmed mean of all probe sets by the target value.

TABLE 2
Exacerbation Genes and Metrics

| AFFYMETRIX | | | Exemplar | FDR Quiet | FDR Quiet | Mean Δlog2 |
|---|---|---|---|---|---|---|
| HG-U133A | | | Entrez | v. Exacer'n, | v. Exacer'n | Quiet v. |
| Probe set ID | Gene Name | Gene Description | Gene ID: | N = 118 | N = 102 | Exacer'n |
| 200057_s_at | NONO | non-POU domain containing, | 4841 | 2.97E−03 | 1.15E−04 | 0.32 |
| | | octamer-binding | | | | |
| 200661_at | CTSA | cathepsin A | 5476 | 2.97E−03 | 1.64E−04 | 0.86 |
| 200962_at | RPL31 | ribosomal protein L31 | 6160 | 2.43E−02 | 3.39E−04 | 0.47 |
| 200986_at | SERPING1 | serpin peptidase inhibitor, clade | 710 | 5.40E−04 | 1.33E−03 | 0.37 |
| | | G (C1 inhibitor), member 1, | | | | |
| | | (angioedema, hereditary) | | | | |
| 201064_s_at | PABPC4 | poly(A) binding protein, | 8761 | 4.61E−02 | 1.33E−03 | 0.28 |
| | | cytoplasmic 4 (inducible form) | | | | |
| 201256_at | COX7A2L | cytochrome c oxidase subunit | 9167 | 4.05E−02 | 1.33E−03 | −0.15 |
| | | VIIa polypeptide 2 like | | | | |
| 201315_x_at | IFITM2 | interferon induced | 10581 | 1.79E−02 | 1.33E−03 | 0.34 |
| | | transmembrane protein 2 (1-8D) | | | | |
| 201600_at | PHB2 | prohibitin 2 | 11331 | 4.22E−02 | 1.47E−03 | 0.41 |
| 201601_x_at | IFITM1 | interferon induced | 8519 | 4.52E−04 | 1.59E−03 | 0.30 |
| | | transmembrane protein 1 (9-27) | | | | |
| 201649_at | UBE2L6 | ubiquitin-conjugating enzyme | 9246 | 4.38E−02 | 1.66E−03 | 0.21 |
| | | E2L 6 | | | | |
| 201762_s_at | PSME2 | proteasome (prosome, | 5721 | 9.51E−03 | 1.69E−03 | −0.14 |
| | | macropain) activator subunit 2 | | | | |
| | | (PA28 beta) | | | | |
| 201939_at | PLK2 | polo-like kinase 2 (Drosophila) | 10769 | 3.29E−02 | 3.39E−03 | 0.22 |
| 202086_at | MX1 | myxovirus (influenza virus) | 4599 | 1.56E−03 | 3.67E−03 | 0.46 |
| | | resistance 1, interferon- | | | | |
| | | inducible protein p78 (mouse) | | | | |
| 202087_s_at | CTSL1 | cathepsin L1 | 1514 | 4.17E−02 | 3.67E−03 | 0.34 |
| 202145_at | LY6E | lymphocyte antigen 6 complex, | 4061 | 7.62E−04 | 3.67E−03 | −0.15 |
| | | locus E | | | | |
| 202374_s_at | RAB3GAP2 | RAB3 GTPase activating | 25782 | 4.63E−02 | 3.98E−03 | 0.16 |
| | | protein subunit 2 (non-catalytic) | | | | |
| 202411_at | IFI27 | interferon, alpha-inducible | 3429 | 8.17E−05 | 1.75E−02 | −0.11 |
| | | protein 27 | | | | |
| 202503_s_at | KIAA0101 | KIAA0101 | 9768 | 4.38E−02 | 1.80E−02 | −0.11 |
| 202589_at | TYMS | thymidylate synthetase | 7298 | 2.75E−02 | 1.80E−02 | −0.10 |
| 203153_at | IFIT1 | interferon-induced protein with | 3434 | 4.61E−02 | 2.02E−02 | 0.30 |
| | | tetratricopeptide repeats 1 | | | | |
| 204043_at | TCN2 | transcobalamin II; macrocytic | 6948 | 1.01E−04 | 2.04E−02 | −0.14 |
| | | anemia | | | | |
| 204415_at | IFI6 | interferon, alpha-inducible | 2537 | 1.47E−04 | 2.52E−02 | 0.07 |
| | | protein 6 | | | | |
| 204698_at | ISG20 | interferon stimulated | 3669 | 6.28E−03 | 2.52E−02 | 0.20 |
| | | exonuclease gene 20 kDa | | | | |
| 204747_at | IFIT3 | interferon-induced protein with | 3437 | 2.97E−03 | 2.52E−02 | 0.24 |
| | | tetratricopeptide repeats 3 | | | | |
| 204858_s_at | ECGF1 | endothelial cell growth factor 1 | 1890 | 2.02E−02 | 2.52E−02 | 0.27 |
| | | (platelet-derived) | | | | |
| 204972_at | OAS2 | 2′-5′-oligoadenylate synthetase | 4939 | 2.75E−02 | 2.52E−02 | 0.15 |
| | | 2, 69/71 kDa | | | | |
| 205055_at | ITGAE | integrin, alpha E (antigen | 3682 | 1.54E−02 | 2.52E−02 | 0.30 |
| | | CD103, human mucosal | | | | |
| | | lymphocyte antigen 1; alpha | | | | |
| | | polypeptide) | | | | |
| 205483_s_at | ISG15 | ISG15 ubiquitin-like modifier | 9636 | 7.24E−05 | 2.52E−02 | 0.23 |
| 205552_s_at | OAS1 | 2′,5′-oligoadenylate synthetase | 4938 | 2.30E−02 | 2.58E−02 | 0.25 |
| | | 1, 40/46 kDa | | | | |
| 205660_at | OASL | 2′-5′-oligoadenylate synthetase- | 8638 | 2.38E−03 | 3.39E−02 | 0.17 |
| | | like | | | | |
| 206111_at | RNASE2 | ribonuclease, RNase A family, 2 | 6036 | 2.75E−02 | 3.50E−02 | 0.26 |
| | | (liver, eosinophil-derived | | | | |
| | | neurotoxin) | | | | |
| 206332_s_at | IFI16 | interferon, gamma-inducible | 3428 | 2.98E−03 | 3.50E−02 | 0.15 |
| | | protein 16 | | | | |
| 206513_at | AIM2 | absent in melanoma 2 | 9447 | 4.63E−02 | 3.50E−02 | 0.16 |
| 208436_s_at | IRF7 | interferon regulatory factor 7 | 3665 | 2.68E−02 | 4.94E−02 | 0.36 |
| 208631_s_at | HADHA | hydroxyacyl-Coenzyme A | 3030 | 1.12E−02 | 4.98E−02 | 0.33 |
| | | dehydrogenase/3-ketoacyl- | | | | |
| | | Coenzyme A thiolase/enoyl- | | | | |
| | | Coenzyme A hydratase | | | | |
| | | (trifunctional protein), alpha | | | | |
| | | subunit | | | | |
| 208805_at | PSMA6 | proteasome (prosome, | 5687 | 4.71E−02 | 5.23E−02 | 0.25 |
| | | macropain) subunit, alpha type, 6 | | | | |
| 208966_x_at | IFI16 | interferon, gamma-inducible | 3428 | 1.36E−02 | 5.23E−02 | 0.16 |
| | | protein 16 | | | | |
| 209009_at | ESD | esterase D/formylglutathione | 2098 | 1.04E−02 | 5.98E−02 | 0.10 |
| | | hydrolase | | | | |
| 209207_s_at | SEC22B | SEC22 vesicle trafficking | 9554 | 2.43E−02 | 5.98E−02 | 0.15 |
| | | protein homolog B (S. cerevisiae) | | | | |
| 209313_at | XAB1 | XPA binding protein 1, GTPase | 11321 | 4.61E−02 | 5.98E−02 | 0.20 |
| 209417_s_at | IFI35 | interferon-induced protein 35 | 3430 | 2.75E−02 | 6.05E−02 | −0.11 |
| 209684_at | RIN2 | Ras and Rab interactor 2 | 54453 | 7.66E−03 | 7.35E−02 | −0.11 |
| 209906_at | C3AR1 | complement component 3a | 719 | 2.75E−02 | 7.85E−02 | −0.09 |
| | | receptor 1 | | | | |
| 210027_s_at | APEX1 | APEX nuclease (multifunctional | 328 | 4.09E−03 | 8.27E−02 | −0.12 |
| | | DNA repair enzyme) 1 | | | | |
| 210797_s_at | OASL | 2′-5′-oligoadenylate synthetase- | 8638 | 4.09E−03 | 9.32E−02 | 0.18 |
| | | like | | | | |
| 210873_x_at | APOBEC3A | apolipoprotein B mRNA editing | 200315 | 5.88E−04 | 9.32E−02 | 0.16 |
| | | enzyme, catalytic polypeptide- | | | | |
| | | like 3A | | | | |
| 211937_at | EIF4B | eukaryotic translation initiation | 1975 | 1.24E−03 | 9.48E−02 | 0.15 |
| | | factor 4B | | | | |
| 211938_at | EIF4B | eukaryotic translation initiation | 1975 | 2.54E−04 | 1.11E−01 | 0.21 |
| | | factor 4B | | | | |
| 211954_s_at | RANBP5 | RAN binding protein 5 | 3843 | 2.75E−02 | 1.21E−01 | 0.18 |
| 212063_at | CD44 | CD44 molecule (Indian blood | 960 | 1.54E−02 | 1.21E−01 | 0.21 |
| | | group) | | | | |
| 212145_at | MRPS27 | mitochondrial ribosomal protein | 23107 | 2.43E−02 | 1.21E−01 | 0.13 |
| | | S27 | | | | |
| 212203_x_at | IFITM3 | interferon induced | 10410 | 5.17E−06 | 1.21E−01 | 0.23 |
| | | transmembrane protein 3 (1-8U) | | | | |
| 212658_at | LHFPL2 | lipoma HMGIC fusion partner- | 10184 | 4.61E−02 | 1.21E−01 | −0.08 |
| | | like 2 | | | | |
| 213293_s_at | TRIM22 | tripartite motif-containing 22 | 10346 | 1.27E−02 | 1.24E−01 | 0.17 |
| 213294_at | HUMPEIF2A | P1/eIF-2a protein kinase | NP_002750.1 | 4.24E−02 | 1.24E−01 | 0.12 |
| 214022_s_at | IFITM1 | interferon induced | 8519 | 6.01E−03 | 1.36E−01 | 0.30 |
| | | transmembrane protein 1 (9-27) | | | | |
| 214290_s_at | HIST2H2AA3 | histone cluster 2, H2aa3 /// | 723790 /// | 2.98E−02 | 1.55E−01 | −0.11 |
| | | histone cluster 2, H2aa4 | 8337 | | | |
| 214442_s_at | PIAS2 | protein inhibitor of activated | 9063 | 4.54E−02 | 1.72E−01 | 0.24 |
| | | STAT, 2 | | | | |
| 214453_s_at | IFI44 | interferon-induced protein 44 | 10561 | 1.65E−04 | 1.72E−01 | 0.19 |
| 216565_x_at | LOC391020 | interferon induced | 391020 | 6.59E−05 | 1.76E−01 | −0.12 |
| | | transmembrane protein | | | | |
| | | pseudogene | | | | |
| 216598_s_at | CCL2 | chemokine (C-C motif) ligand 2 | 6347 | 2.43E−02 | 1.76E−01 | 0.22 |
| 216950_s_at | FCGR1A | Fc fragment of IgG, high affinity | 2209 | 4.63E−02 | 1.76E−01 | −0.09 |
| | | Ia, receptor (CD64) | | | | |
| 217719_at | EIF3EIP | eukaryotic translation initiation | 51386 | 1.82E−04 | 1.76E−01 | 0.61 |
| | | factor 3, subunit E interacting | | | | |
| | | protein | | | | |
| 217846_at | QARS | glutaminyl-tRNA synthetase | 5859 | 4.36E−03 | 1.87E−01 | 0.07 |
| 218232_at | C1QA | complement component 1, q | 712 | 9.51E−03 | 2.14E−01 | 0.54 |
| | | subcomponent, A chain | | | | |
| 218280_x_at | HIST2H2AA3 | histone cluster 2, H2aa3 /// | 723790 /// | 4.61E−02 | 2.17E−01 | −0.07 |
| | | histone cluster 2, H2aa4 | 8337 | | | |
| 218400_at | OAS3 | 2′-5′-oligoadenylate synthetase | 4940 | 9.51E−03 | 2.23E−01 | 0.18 |
| | | 3, 100 kDa | | | | |
| 218458_at | GMCL1 | germ cell-less homolog 1 | 64395 | 2.74E−02 | 2.30E−01 | 0.14 |
| | | (Drosophila) | | | | |
| 219014_at | PLAC8 | placenta-specific 8 | 51316 | 2.43E−02 | 2.33E−01 | −0.07 |
| 219209_at | IFIH1 | interferon induced with helicase | 64135 | 2.76E−02 | 2.33E−01 | −0.09 |
| | | C domain 1 | | | | |
| 219863_at | HERC5 | hect domain and RLD 5 | 51191 | 9.51E−03 | 2.33E−01 | −0.12 |
| 221476_s_at | RPL15 | ribosomal protein L15 | 6138 | 2.62E−03 | 2.44E−01 | 0.16 |
| 221726_at | RPL22 | ribosomal protein L22 | 6146 | 1.29E−03 | 2.52E−01 | −0.08 |
| 221741_s_at | YTHDF1 | YTH domain family, member 1 | 54915 | 4.61E−02 | 2.52E−01 | −0.10 |
| 221875_x_at | HLA-F | major histocompatibility | 3134 | 2.75E−02 | 2.68E−01 | 0.14 |
| | | complex, class I, F | | | | |
| 222154_s_at | DNAPTP6 | viral DNA polymerase- | 26010 | 3.42E−02 | 2.84E−01 | −0.07 |
| | | transactivated protein 6 | | | | |
| 33304_at | ISG20 | interferon stimulated | 3669 | 9.51E−03 | 3.10E−01 | −0.24 |
| | | exonuclease gene 20 kDa | | | | |
| 44673_at | SIGLEC1 | sialic acid binding Ig-like lectin | 6614 | 1.65E−04 | 3.30E−01 | 0.16 |
| | | 1, sialoadhesin | | | | |

TABLE 3
Results of Mixed Model Analysis Comparing Exacerbation vs
Quiet Visits

| | | | | | 95% Confidence | |
|---|---|---|---|---|---|---|
| | | | Standard | | Interval | |
| Subgroup | Gene | Estimate¹ | Error | P Value | Lower | Upper |
| X | IFNα1 | 16.97 | 5.82 | 0.0047* | 5.37 | 28.57 |
| | IFNβ1 | 2.51 | 0.82 | 0.0031* | 0.88 | 4.15 |
| | IFNγ | −0.19 | 0.15 | 0.2310 | −0.49 | 0.12 |
| | IL13 | −11.01 | 6.89 | 0.1133 | −24.69 | 2.67 |
| | IL18 | −100.34 | 148.52 | 0.5010 | −395.33 | 194.64 |
| Y | IFNα1 | −6.40 | 8.51 | 0.4537 | −23.27 | 10.47 |
| | IFNβ1 | −0.04 | 0.24 | 0.8502 | −0.51 | 0.42 |
| | IFNγ | −0.01 | 0.21 | 0.9514 | −0.42 | 0.40 |
| | IL13 | −4.98 | 3.54 | 0.1626 | −11.99 | 2.03 |
| | IL18 | 162.40 | 84.82 | 0.0576 | −5.34 | 330.26 |

¹Estimated Difference for Exacerbation Expression − Quiet Expression.
*Gene expression for exacerbation visit is statistically different from that observed at quiet visits at 5% significance level.

TABLE 4
Subgroup X Genes and Metrics

| | | FDR | FDR | FDR | | |
|---|---|---|---|---|---|---|
| | | Exacerbation | Exacerbation | Follow-up | Average log2 | |
| AFFYMETRIX | | versus quiet | versus quiet, | Versus Quiet | difference | |
| HG-U133A | IPA-Gene | 30 visit | 22 visit | 22 visit | exacerbation | |
| Probe set ID | Symbol | analysis | analysis | analysis | versus quiet | SEQ ID NO: |
| 202411_at | IFI27 | 1.00E−13 | 1.00E−13 | 0.314342283 | 3.2264742 | SEQ ID NO: 51 |
| 218943_s_at | DDX58 | 1.00E−13 | 2.28786E−10 | 0.418656737 | 1.257131388 | |
| 216950_s_at | FCGR1A | 1.00E−13 | 4.37509E−11 | 0.543105461 | 0.868556644 | |
| 219014_at | PLAC8 | 1.00E−13 | 1.00E−13 | 0.547460786 | 0.87127678 | |
| 211938_at | EIF4B | 1.00E−13 | 1.00E−13 | 0.576368551 | −0.723426408 | |
| 201762_s_at | PSME2 | 1.00E−13 | 4.12266E−12 | 0.576624724 | 0.870211608 | |
| 221476_s_at | RPL15 | 1.00E−13 | 1.23025E−13 | 0.62004893 | −0.463678293 | |
| 205552_s_at | OAS1 | 1.00E−13 | 2.71458E−12 | 0.625905427 | 1.698977288 | |
| 203153_at | IFIT1 | 1.00E−13 | 9.56864E−13 | 0.632763895 | 3.237554236 | |
| 208012_x_at | SP110 | 1.00E−13 | 6.0713E−10 | 0.663534687 | 0.72768352 | |
| 204698_at | ISG20 | 1.00E−13 | 3.3066E−11 | 0.677299865 | 0.894249544 | |
| 214511_x_at | FCGR1A | 1.00E−13 | 1.03296E−09 | 0.713883189 | 0.85734853 | |
| 210797_s_at | OASL | 1.00E−13 | 9.01233E−13 | 0.726031892 | 1.112482705 | |
| 202270_at | GBP1 | 1.00E−13 | 5.25108E−13 | 0.745247946 | 1.568665304 | |
| 216565_x_at | | 1.00E−13 | 1.00E−13 | 0.764947366 | 1.456897986 | |
| 217846_at | QARS | 1.00E−13 | 9.56864E−13 | 0.770211009 | −0.49716941 | |
| 217719_at | EIF3EIP | 1.00E−13 | 1.00E−13 | 0.804966785 | −0.585342786 | |
| 212203_x_at | IFITM3 | 1.00E−13 | 1.00E−13 | 0.804966785 | 1.398071666 | |
| 201649_at | UBE2L6 | 1.00E−13 | 1.00E−13 | 0.804966785 | 0.981987226 | |
| 200887_s_at | STAT1 | 1.00E−13 | 8.19311E−10 | 0.806943262 | 0.871530247 | |
| 213294_at | | 1.00E−13 | 1.00E−13 | 0.808403299 | 1.06592408 | |
| 219209_at | IFIH1 | 1.00E−13 | 3.10885E−09 | 0.809600216 | 0.985007302 | |
| 211937_at | EIF4B | 1.00E−13 | 1.00E−13 | 0.816104227 | −0.689405283 | |
| 218986_s_at | FLJ20035 | 1.00E−13 | 2.26626E−13 | 0.82330521 | 1.291531957 | |
| 204994_at | MX2 | 1.00E−13 | 1.00E−13 | 0.823670503 | 0.979758523 | |
| 205241_at | SCO2 | 1.00E−13 | 1.00E−13 | 0.8250555 | 1.126907874 | |
| 208966_x_at | IFI16 | 1.00E−13 | 3.40484E−11 | 0.825177207 | 0.687857558 | |
| 33304_at | ISG20 | 1.00E−13 | 1.23025E−13 | 0.826503176 | 0.708565248 | |
| 213293_s_at | TRIM22 | 1.00E−13 | 1.00E−13 | 0.833074897 | 0.863567736 | |
| 209762_x_at | SP110 | 1.00E−13 | 7.81859E−11 | 0.838299238 | 0.688848248 | |
| 209417_s_at | IFI35 | 1.00E−13 | 1.00E−13 | 0.844024603 | 1.340312324 | |
| 204929_s_at | VAMP5 | 1.00E−13 | 9.01233E−13 | 0.845163449 | 0.767890787 | |
| 206332_s_at | IFI16 | 1.00E−13 | 3.3066E−11 | 0.864992216 | 0.746245403 | |
| 217933_s_at | LAP3 | 1.00E−13 | 2.26626E−13 | 0.87331579 | 0.904969621 | |
| 205660_at | OASL | 1.00E−13 | 1.00E−13 | 0.89701828 | 1.452000093 | |
| 218400_at | OAS3 (includes | 1.00E−13 | 1.00E−13 | 0.899088585 | 1.773759512 | |
| | EG: 4940) | | | | | |
| 204439_at | IFI44L | 1.00E−13 | 1.00E−13 | 0.903665108 | 3.343964203 | SEQ ID NO: 52 |
| 202145_at | LY6E (includes | 1.00E−13 | 1.00E−13 | 0.906012249 | 2.022298855 | SEQ ID NO: 53 |
| | EG: 4061) | | | | | |
| 218543_s_at | PARP12 | 1.00E−13 | 1.00E−13 | 0.906722412 | 0.769313779 | |
| 204415_at | IFI6 | 1.00E−13 | 1.00E−13 | 0.911573276 | 1.766313456 | |
| 200628_s_at | WARS | 1.00E−13 | 8.81825E−09 | 0.920178258 | 0.674972037 | |
| 202086_at | MX1 | 1.00E−13 | 1.00E−13 | 0.92335789 | 1.933302682 | |
| 204858_s_at | ECGF1 | 1.00E−13 | 2.26626E−13 | 0.926416412 | 1.091960902 | |
| 202269_x_at | GBP1 | 1.00E−13 | 1.77141E−09 | 0.926416412 | 1.522330967 | |
| 200986_at | SERPING1 | 1.00E−13 | 1.00E−13 | 0.926817552 | 2.389898415 | SEQ ID NO: 54 |
| 204747_at | IFIT3 | 1.00E−13 | 1.00E−13 | 0.932963506 | 2.237939985 | SEQ ID NO: 55 |
| 201601_x_at | IFITM1 | 1.00E−13 | 1.00E−13 | 0.932963506 | 1.073969376 | |
| 202869_at | OAS1 | 1.00E−13 | 7.4694E−12 | 0.940129298 | 1.738328761 | |
| 219352_at | HERC6 | 1.00E−13 | 1.11447E−11 | 0.940468793 | 1.37694416 | |
| 44673_at | SIGLEC1 | 1.00E−13 | 1.00E−13 | 0.940613622 | 1.921070997 | |
| 201641_at | BST2 | 1.00E−13 | 3.80484E−11 | 0.947786651 | 0.866641592 | |
| 202688_at | TNFSF10 | 1.00E−13 | 1.72681E−10 | 0.971768475 | 1.107047024 | |
| 214022_s_at | IFITM1 | 1.00E−13 | 1.00E−13 | 0.977627636 | 0.916009634 | |
| 208436_s_at | IRF7 | 1.00E−13 | 1.00E−13 | 0.977971618 | 1.584740885 | |
| 210873_x_at | APOBEC3A | 1.00E−13 | 1.00E−13 | 0.978184927 | 1.449174452 | |
| 202748_at | GBP2 (includes | 1.00E−13 | 1.67719E−08 | 0.978184927 | 0.591465092 | |
| | EG: 2634) | | | | | |
| 217502_at | IFIT2 | 1.00E−13 | 1.33546E−09 | 0.979892081 | 1.189237002 | |
| 200629_at | WARS | 1.00E−13 | 7.81759E−09 | 0.983061504 | 0.815198536 | |
| 205483_s_at | ISG15 | 1.00E−13 | 1.00E−13 | 0.9845525 | 2.458789526 | SEQ ID NO: 56 |
| 219863_at | HERC5 | 1.00E−13 | 1.00E−13 | 0.989252635 | 1.340107229 | |
| 214453_s_at | IFI44 | 1.00E−13 | 1.00E−13 | 0.989523303 | 2.06603506 | SEQ ID NO: 57 |
| 221726_at | RPL22 | 1.00E−13 | 4.30589E−13 | 0.99162365 | −0.517692133 | |
| 53720_at | FLJ11286 | 1.00E−13 | 2.35906E−10 | 0.99162365 | 0.597380083 | |
| 222154_s_at | DNAPTP6 | 1.00E−13 | 1.00E−13 | 0.992470596 | 1.268255203 | |
| 212145_at | MRPS27 | 1.00E−13 | 1.00E−13 | 0.994302012 | −0.62736433 | |
| 204972_at | OAS2 | 1.00E−13 | 1.23025E−13 | 0.994302012 | 1.128075814 | |
| 202687_s_at | TNFSF10 | 1.00E−13 | 2.18312E−08 | 0.994302012 | 0.98900227 | |
| 203964_at | NMI | 1.26644E−13 | 4.2913E−10 | 0.652660658 | 0.669128866 | |
| 211623_s_at | FBL | 1.87213E−13 | 3.31222E−13 | 0.833074897 | −0.60033015 | |
| 204211_x_at | EIF2AK2 | 4.30589E−13 | 3.02612E−08 | 0.804966785 | 1.594408461 | |
| 204043_at | TCN2 | 9.09695E−13 | 9.227E−10 | 0.980726521 | 0.690121385 | |
| 202446_s_at | PLSCR1 | 1.01667E−12 | 2.64986E−08 | 0.85177202 | 0.755042505 | |
| 219062_s_at | ZCCHC2 | 1.5336E−12 | 8.91807E−09 | 0.896710842 | 0.687393544 | |
| 202307_s_at | TAP1 | 2.50207E−12 | 3.13346E−06 | 0.921239504 | 0.536758483 | |
| 203052_at | C2 | 3.55953E−12 | 1.7915E−09 | 0.992470596 | 0.857425826 | |
| 208751_at | NAPA | 4.13592E−12 | 1.06049E−08 | 0.906012249 | 0.580955305 | |
| 219403_s_at | HPSE | 5.4243E−12 | 5.89108E−09 | 0.915587288 | 0.80562162 | |
| 201315_x_at | IFITM2 | 7.23169E−12 | 2.60589E−09 | 0.748460361 | 0.836283869 | |
| 216598_s_at | CCL2 | 7.90321E−12 | 8.87013E−12 | 0.896257222 | 3.396641489 | |
| 213716_s_at | SECTM1 | 1.50706E−11 | 5.90667E−09 | 0.947058156 | 0.84862382 | |
| 214470_at | KLRB1 | 2.81743E−11 | 1.91211E−10 | 0.439510344 | −0.793264263 | |
| 217497_at | ECGF1 | 3.50772E−11 | 3.28619E−07 | 0.897429484 | 0.781032998 | |
| 205055_at | ITGAE | 4.17619E−11 | 2.31436E−09 | 0.825177207 | −0.539147903 | |
| 202863_at | SP100 | 4.59295E−11 | 7.7357E−09 | 0.77858286 | 0.430558471 | |
| 203258_at | DRAP1 | 4.69595E−11 | 9.72445E−08 | 0.669240413 | 0.589634478 | |
| 202430_s_at | PLSCR1 | 6.83435E−11 | 1.79462E−07 | 0.972269431 | 0.845088924 | |
| 202087_s_at | CTSL1 | 7.25567E−11 | 6.54854E−12 | 0.594216566 | 1.306864024 | |
| 206133_at | BIRC4BP | 9.81058E−11 | 2.19014E−07 | 0.662172655 | 1.636633862 | |
| 203882_at | ISGF3G | 1.18436E−10 | 5.965E−08 | 0.69958278 | 0.561582554 | |
| 221816_s_at | PHF11 | 1.32526E−10 | 5.58325E−08 | 0.978029446 | 0.400769775 | |
| | (includes | | | | | |
| | EG: 51131) | | | | | |
| 201030_x_at | LDHB | 1.36369E−10 | 8.5811E−09 | 0.995153496 | −0.459647176 | |
| 201064_s_at | PABPC4 | 1.3938E−10 | 1.77234E−07 | 0.377556356 | −0.375919864 | |
| 206491_s_at | NAPA | 1.82885E−10 | 4.51507E−08 | 0.654429654 | 0.527768347 | |
| 200705_s_at | EEF1B2 | 2.58537E−10 | 4.91623E−10 | 0.933418803 | −0.45534209 | |
| 214329_x_at | TNFSF10 | 3.11293E−10 | 8.01708E−06 | 0.846591986 | 1.081146781 | |
| 210027_s_at | APEX1 | 3.68378E−10 | 8.83764E−08 | 0.842242847 | −0.426855876 | |
| 209969_s_at | STAT1 | 3.7661E−10 | 1.00035E−05 | 0.996581247 | 1.22371884 | |
| 204279_at | PSMB9 | 5.56075E−10 | 3.43766E−06 | 0.633489787 | 0.723472351 | |
| 208631_s_at | HADHA | 5.71422E−10 | 1.03033E−08 | 0.968104648 | −0.439593818 | |
| 212657_s_at | IL1RN | 6.59662E−10 | 1.25404E−06 | 0.980673033 | 1.117091191 | |
| 211729_x_at | BLVRA | 9.49683E−10 | 1.679E−05 | 0.913332358 | 0.395647321 | |
| 201637_s_at | FXR1 | 1.08247E−09 | 5.18658E−07 | 0.867793855 | −0.394212413 | |
| 201798_s_at | FER1L3 | 1.12058E−09 | 3.50361E−06 | 0.978012719 | 0.804547413 | |
| 202659_at | PSMB10 | 1.31884E−09 | 5.16183E−05 | 0.570133912 | 0.514936934 | |
| 206513_at | AIM2 | 1.32014E−09 | 9.75118E−08 | 0.577947358 | 0.874116153 | |
| 204224_s_at | GCH1 | 1.43325E−09 | 3.05898E−06 | 0.958976925 | 0.458470138 | |
| 214167_s_at | RPLP0 | 1.47044E−09 | 4.94913E−09 | 0.977971618 | −0.545742349 | |
| | (includes | | | | | |
| | EG: 6175) | | | | | |
| 201812_s_at | TOMM7 | 1.81354E−09 | 2.44475E−07 | 0.997185403 | −0.378798248 | |
| 213361_at | TDRD7 | 2.49844E−09 | 1.26415E−07 | 0.974654709 | 0.437970793 | |
| 209124_at | MYD88 | 2.53766E−09 | 1.00782E−05 | 0.899359693 | 0.344411265 | |
| 208697_s_at | EIF3E | 2.611E−09 | 3.04635E−07 | 0.985020691 | −0.359281275 | |
| 209761_s_at | SP110 | 3.12069E−09 | 1.19836E−06 | 0.865251659 | 0.758063399 | |
| 205875_s_at | TREX1 | 3.44688E−09 | 4.30558E−05 | 0.668804606 | 0.542376727 | |
| | (includes | | | | | |
| | EG: 11277) | | | | | |
| 34689_at | TREX1 | 3.56006E−09 | 1.04345E−06 | 0.706296724 | 0.40831158 | |
| | (includes | | | | | |
| | EG: 11277) | | | | | |
| 203582_s_at | RAB4A | 5.80722E−09 | 1.85329E−09 | 0.882049407 | −0.455069678 | |
| 201669_s_at | MARCKS | 5.99094E−09 | 6.23664E−08 | 0.896681815 | 0.887496769 | |
| | (includes | | | | | |
| | EG: 4082) | | | | | |
| 200036_s_at | RPL10A | 1.01645E−08 | 5.22692E−08 | 0.899991236 | −0.439060404 | |
| | (includes | | | | | |
| | EG: 4736) | | | | | |
| 217988_at | CCNB1IP1 | 1.06098E−08 | 2.21844E−07 | 0.784207096 | −0.476825911 | |
| 213762_x_at | RBMX | 1.06768E−08 | 3.28619E−07 | 0.971652444 | −0.350958082 | |
| 206584_at | LY96 | 1.07795E−08 | 8.54545E−07 | 0.770211009 | 0.740360256 | |
| 201600_at | PHB2 | 1.3624E−08 | 6.23664E−08 | 0.708968475 | −0.346901246 | |
| 204834_at | FGL2 | 1.45774E−08 | 8.12098E−06 | 0.519109042 | 1.288816531 | |
| 200089_s_at | RPL4 | 1.46276E−08 | 1.65355E−07 | 0.988678861 | −0.505446846 | |
| 203236_s_at | LGALS9 | 1.65314E−08 | 5.33164E−06 | 0.542658561 | 0.655449754 | |
| 200937_s_at | RPL5 | 1.69382E−08 | 1.45556E−07 | 0.991325836 | −0.506636182 | |
| 209193_at | PIM1 | 1.77548E−08 | 2.40667E−07 | 0.971652444 | 0.452796841 | |
| 218232_at | C1QA | 1.78536E−08 | 3.20582E−06 | 0.970304109 | 0.827307093 | |
| 219356_s_at | CHMP5 | 2.08519E−08 | 2.44506E−06 | 0.940578006 | 0.637083293 | |
| 201670_s_at | MARCKS | 2.08619E−08 | 1.72623E−07 | 0.926350227 | 1.064785892 | |
| | (includes | | | | | |
| | EG: 4082) | | | | | |
| 200005_at | EIF3D | 2.67828E−08 | 3.00878E−07 | 0.553496047 | −0.343060011 | |
| 211710_x_at | RPL4 | 2.67828E−08 | 3.08059E−07 | 0.857260432 | −0.428953368 | |
| 211666_x_at | RPL3 | 2.67828E−08 | 1.90854E−07 | 0.9722812 | −0.440596971 | |
| 200814_at | PSME1 | 2.80502E−08 | 0.000272302 | 0.996581247 | 0.310736438 | |
| 200094_s_at | EEF2 | 3.0418E−08 | 2.24843E−05 | 0.676993566 | −0.37012599 | |
| 201154_x_at | RPL4 | 3.06216E−08 | 2.38122E−07 | 0.995153496 | −0.428958782 | |
| 208805_at | PSMA6 | 3.07928E−08 | 5.3051E−06 | 0.996581247 | 0.334500935 | |
| 204533_at | CXCL10 | 3.13289E−08 | 0.000169945 | 0.977971618 | 0.649918318 | |
| 213418_at | HSPA6 | 3.34632E−08 | 1.33215E−08 | 0.916817284 | 0.645613395 | |
| 219590_x_at | DPH5 | 3.54892E−08 | 3.39148E−07 | 0.984183933 | −0.56634902 | |
| 205992_s_at | IL15 | 3.65239E−08 | 5.65213E−06 | 0.920139527 | 0.693000634 | |
| 211395_x_at | FCGR2C | 3.79402E−08 | 2.64289E−05 | 0.949368864 | 0.504597766 | |
| 218660_at | DYSF | 3.82874E−08 | 8.90393E−07 | 0.525380174 | 0.858852687 | |
| 212659_s_at | IL1RN | 4.02669E−08 | 5.84398E−06 | 0.884660098 | 0.794747033 | |
| 204006_s_at | FCGR3A | 4.05855E−08 | 1.00186E−05 | 0.926817552 | 1.139105262 | |
| 203595_s_at | IFIT5 | 4.15916E−08 | 0.000196146 | 0.927054575 | 0.462431585 | |
| 218168_s_at | CABC1 | 4.35065E−08 | 7.99186E−06 | 0.712482556 | −0.434659676 | |
| 213564_x_at | LDHB | 4.51081E−08 | 2.25956E−07 | 0.997185403 | −0.338018026 | |
| 221875_x_at | HLA-F | 4.51302E−08 | 6.87174E−05 | 0.843903188 | 0.252103695 | |
| 207040_s_at | ST13 | 4.6487E−08 | 5.74755E−06 | 0.806943262 | −0.317357338 | |
| 215963_x_at | RPL3 | 5.43326E−08 | 5.22692E−08 | 0.739399013 | −0.54170032 | |
| 204204_at | SLC31A2 | 5.58708E−08 | 4.86643E−05 | 0.899088585 | 0.503456523 | |
| 208892_s_at | DUSP6 | 5.80686E−08 | 2.25956E−07 | 0.745326368 | 0.931549428 | |
| 200660_at | S100A11 | 6.19255E−08 | 2.55682E−05 | 0.884660098 | 0.780649063 | |
| | (includes | | | | | |
| | EG: 6282) | | | | | |
| 218253_s_at | LGTN | 6.58812E−08 | 1.4878E−06 | 0.954990264 | −0.390068742 | |
| 204232_at | FCER1G | 8.70985E−08 | 2.37802E−05 | 0.845163449 | 0.515516462 | |
| 213214_x_at | ACTG1 | 1.10815E−07 | 1.88018E−06 | 0.909279827 | −0.286450562 | |
| 216243_s_at | IL1RN | 1.10815E−07 | 4.71896E−05 | 0.932963506 | 0.733657533 | |
| 217969_at | C11ORF2 | 1.27636E−07 | 1.66268E−06 | 0.793520318 | −0.468285993 | |
| 209009_at | ESD | 1.62581E−07 | 3.86107E−05 | 0.983024519 | −0.329837339 | |
| 201592_at | EIF3H | 1.68399E−07 | 8.5382E−06 | 0.66341159 | −0.347008103 | |
| 200086_s_at | COX4I1 | 1.7144E−07 | 1.60503E−06 | 0.254883603 | −0.324425641 | |
| 218495_at | UXT | 1.74894E−07 | 4.70865E−07 | 0.971652444 | −0.389750956 | |
| 201400_at | PSMB3 | 1.85558E−07 | 0.000145002 | 0.978029446 | 0.306473442 | |
| 207614_s_at | CUL1 | 2.07029E−07 | 0.000251413 | 0.971652444 | 0.336153832 | |
| 221345_at | FFAR2 | 2.13477E−07 | 6.27001E−06 | 0.96226171 | 1.56455422 | |
| 201761_at | MTHFD2 | 2.40953E−07 | 7.61713E−05 | 0.971652444 | 0.456828545 | |
| 210470_x_at | NONO | 2.54095E−07 | 7.97249E−05 | 0.572703468 | −0.297512722 | |
| 204205_at | APOBEC3G | 2.56556E−07 | 3.28891E−05 | 0.570133912 | 0.538643341 | |
| 210146_x_at | LILRB2 | 2.6892E−07 | 2.08727E−05 | 0.806943262 | 0.634455458 | |
| 208912_s_at | CNP | 2.90992E−07 | 0.001420257 | 0.97607782 | 0.346507294 | |
| 208717_at | OXA1L | 3.17515E−07 | 5.33164E−06 | 0.627940295 | −0.39144339 | |
| 201256_at | COX7A2L | 3.48182E−07 | 1.35286E−06 | 0.920178258 | −0.313875053 | |
| 201786_s_at | ADAR | 3.61686E−07 | 0.000160373 | 0.983540219 | 0.332746832 | |
| 212761_at | TCF7L2 | 3.91925E−07 | 0.000507052 | 0.991516688 | 0.461601921 | |
| 212063_at | CD44 | 4.13799E−07 | 1.35286E−06 | 0.757136645 | −0.328548578 | |
| 212995_x_at | FAM128B | 4.43613E−07 | 7.62784E−07 | 0.980673033 | −0.4525389 | |
| 208771_s_at | LTA4H | 4.56734E−07 | 8.85437E−07 | 0.818684405 | −0.505919359 | |
| 219528_s_at | BCL11B | 5.16386E−07 | 5.54335E−06 | 0.931474938 | −0.613739228 | |
| 221827_at | RBCK1 | 5.1805E−07 | 0.000500307 | 0.937972084 | 0.265058447 | |
| 201647_s_at | SCARB2 | 5.5928E−07 | 6.43719E−05 | 0.949462591 | 0.394796339 | |
| 209684_at | RIN2 | 6.1967E−07 | 4.20656E−05 | 0.996581247 | 0.615141074 | |
| 210992_x_at | FCGR2C | 6.24826E−07 | 0.000516537 | 0.933364008 | 0.540639219 | |
| 208959_s_at | TXNDC4 | 6.27267E−07 | 8.01708E−06 | 0.989523303 | 0.358056192 | |
| 208796_s_at | CCNG1 | 6.30959E−07 | 0.000137277 | 0.998249748 | −0.356027629 | |
| 214042_s_at | RPL22 | 6.56996E−07 | 4.9363E−06 | 0.940129298 | −0.408753498 | |
| 200030_s_at | SLC25A3 | 6.59369E−07 | 6.65459E−05 | 0.514325032 | −0.251827666 | |
| 206111_at | RNASE2 | 6.80154E−07 | 3.4304E−07 | 0.103154853 | 0.900316586 | |
| 213166_x_at | FAM128A | 7.36947E−07 | 2.52156E−06 | 0.972604469 | −0.459478297 | |
| 48531_at | TNIP2 | 7.59908E−07 | 2.64623E−05 | 0.948066734 | 0.304570703 | |
| 200715_x_at | RPL13A | 8.12603E−07 | 1.679E−05 | 0.976541414 | −0.325439068 | |
| 204563_at | SELL | 8.35783E−07 | 9.86081E−06 | 0.542658561 | 0.570840007 | |
| 218429_s_at | FLJ11286 | 8.44404E−07 | 0.000290242 | 0.542658561 | 0.422610209 | |
| 209593_s_at | TOR1B | 8.44404E−07 | 0.005088536 | 0.957267847 | 0.308269962 | |
| 218271_s_at | PARL | 1.09568E−06 | 2.93579E−07 | 0.644078042 | −0.358246135 | |
| 221475_s_at | RPL15 | 1.22492E−06 | 7.99186E−06 | 0.85740335 | −0.426401885 | |
| 205819_at | MARCO | 1.22492E−06 | 0.000149568 | 0.913862795 | 1.305945932 | |
| 204007_at | FCGR3B | 1.24312E−06 | 5.36671E−05 | 0.460110071 | 1.328602783 | |
| 205170_at | STAT2 | 1.24744E−06 | 2.64623E−05 | 0.896710842 | 0.625304165 | |
| 208540_x_at | S100A11 | 1.25006E−06 | 0.000145182 | 0.858338088 | 0.486038424 | |
| | (includes | | | | | |
| | EG: 6282) | | | | | |
| 213002_at | MARCKS | 1.33961E−06 | 1.91357E−05 | 0.980673033 | 0.602740512 | |
| | (includes | | | | | |
| | EG: 4082) | | | | | |
| 208698_s_at | NONO | 1.3502E−06 | 0.000153504 | 0.891670708 | −0.261819767 | |
| 216032_s_at | ERGIC3 | 1.87633E−06 | 7.13495E−06 | 0.611156155 | −0.371879627 | |
| 208826_x_at | HINT1 | 1.99657E−06 | 8.10441E−06 | 0.824385407 | −0.355922859 | |
| 218854_at | DSE | 2.11257E−06 | 9.63401E−08 | 0.741625882 | 0.834549374 | |
| 213988_s_at | SAT1 | 2.21005E−06 | 1.12606E−05 | 0.913332358 | 0.635144779 | |
| 207721_x_at | HINT1 | 2.4571E−06 | 6.05064E−05 | 0.842053841 | −0.450360691 | |
| 201892_s_at | IMPDH2 | 2.52815E−06 | 1.33532E−05 | 0.504375257 | −0.519690211 | |
| 218154_at | GSDMDC1 | 2.55311E−06 | 0.001157149 | 0.958976925 | 0.515052858 | |
| 212085_at | SLC25A6 | 2.55512E−06 | 2.12978E−05 | 0.893024231 | −0.295997965 | |
| 221691_x_at | NPM1 | 2.89278E−06 | 2.76878E−05 | 0.952102861 | −0.547195243 | |
| | (includes | | | | | |
| | EG: 4869) | | | | | |
| 208819_at | RAB8A | 3.01224E−06 | 0.002328471 | 0.913292392 | 0.294688001 | |
| 207697_x_at | LILRB2 | 3.01224E−06 | 0.000198214 | 0.998828056 | 0.42477032 | |
| 201368_at | ZFP36L2 | 3.49162E−06 | 0.00385991 | 0.554763569 | −0.339565447 | |
| 218599_at | REC8 | 4.17798E−06 | 2.51425E−05 | 0.649255083 | 0.387711796 | |
| 217807_s_at | GLTSCR2 | 4.57745E−06 | 2.60872E−05 | 0.985775064 | −0.321527374 | |
| 209620_s_at | ABCB7 | 4.59573E−06 | 3.71364E−05 | 0.838839885 | −0.49253984 | |
| 207713_s_at | RBCK1 | 5.56529E−06 | 0.001230539 | 0.9722812 | 0.485489106 | |
| 207574_s_at | GADD45B | 5.64844E−06 | 2.33291E−05 | 0.658621166 | 0.584857315 | |
| 200024_at | RPS5 | 5.74376E−06 | 2.32942E−05 | 0.980264971 | −0.371099528 | |
| 212018_s_at | RSL1D1 | 5.75524E−06 | 5.2651E−05 | 0.852763334 | −0.374756011 | |
| 211345_x_at | EEF1G | 5.87551E−06 | 2.63616E−05 | 0.878694832 | −0.332288405 | |
| 218561_s_at | LYRM4 | 6.35001E−06 | 0.000178513 | 0.927054575 | −0.383313694 | |
| 209861_s_at | METAP2 | 6.61524E−06 | 0.000438021 | 0.950173591 | −0.400293647 | |
| 214290_s_at | HIST2H2AA3 | 6.69398E−06 | 1.11005E−05 | 0.570133912 | 0.545713891 | |
| 200823_x_at | RPL29 | 7.27556E−06 | 1.00186E−05 | 0.959247226 | −0.438585583 | |
| | (includes | | | | | |
| | EG: 6159) | | | | | |
| 221494_x_at | EIF3K | 7.52644E−06 | 1.96128E−05 | 0.949368864 | −0.313036928 | |
| 201922_at | TINP1 | 7.53268E−06 | 0.000111159 | 0.993864266 | −0.304975325 | |
| 217436_x_at | HLA-A | 7.54331E−06 | 0.000811135 | 0.926350227 | 0.288688983 | |
| 203561_at | FCGR2A | 7.58179E−06 | 0.00140219 | 0.989523303 | 0.541054572 | |
| 203771_s_at | BLVRA | 8.2961E−06 | 0.014081568 | 0.997185403 | 0.40479609 | |
| 200678_x_at | GRN | 8.45692E−06 | 0.000536107 | 0.997185403 | 0.477018079 | |
| 222218_s_at | PILRA | 8.80032E−06 | 0.000475117 | 0.811796229 | 0.50513639 | |
| 218280_x_at | HIST2H2AA3 | 9.23142E−06 | 1.12089E−05 | 0.504375257 | 0.537868991 | |
| 200661_at | CTSA | 9.23142E−06 | 0.000204583 | 0.774402907 | 0.364971499 | |
| 219505_at | CECR1 | 9.40966E−06 | 0.00042371 | 0.997185403 | 0.57028576 | |
| 207181_s_at | CASP7 | 9.80392E−06 | 0.000222785 | 0.890400749 | 0.453533094 | |
| 219690_at | TMEM149 | 9.82898E−06 | 0.003896087 | 0.726031892 | 0.254732155 | |
| 206420_at | IGSF6 | 1.02608E−05 | 0.00042371 | 0.884660098 | 0.741966662 | |
| 215262_at | OXNAD1 | 1.03082E−05 | 7.9003E−05 | 0.915759954 | −0.779468561 | |
| 210592_s_at | SAT1 | 1.03377E−05 | 9.85604E−05 | 0.870341928 | 0.304373382 | |
| 213958_at | CD6 | 1.19573E−05 | 0.000206884 | 0.558360244 | −0.546783187 | |
| 218773_s_at | MSRB2 | 1.26553E−05 | 0.000282964 | 0.42752641 | 0.653901617 | |
| 214097_at | RPS21 | 1.27079E−05 | 2.78036E−05 | 0.957907274 | −0.510530358 | |
| 214084_x_at | NCF1 | 1.30587E−05 | 0.000589823 | 0.480845338 | 0.634088365 | |
| 32069_at | N4BP1 | 1.32096E−05 | 1.50662E−05 | 0.83826875 | 0.548797525 | |
| 218919_at | ZFAND1 | 1.35464E−05 | 2.78026E−05 | 0.941532908 | −0.440976806 | |
| 200962_at | RPL31 | 1.38143E−05 | 0.008278452 | 0.815013718 | −0.228873204 | |
| | (includes | | | | | |
| | EG: 6160) | | | | | |
| 201217_x_at | RPL3 | 1.43294E−05 | 2.23558E−05 | 0.918228194 | −0.365069349 | |
| 219938_s_at | PSTPIP2 | 1.46256E−05 | 0.000335844 | 0.603006862 | 0.50344177 | |
| 201369_s_at | ZFP36L2 | 1.46256E−05 | 0.007035026 | 0.745247946 | −0.594951593 | |
| 211072_x_at | TUBA1B | 1.48847E−05 | 0.000500307 | 0.957267847 | 0.287212702 | |
| 217752_s_at | CNDP2 | 1.54096E−05 | 0.001296308 | 0.739399013 | 0.501512566 | |
| 216342_x_at | Need to update | 1.55326E−05 | 2.32942E−05 | 0.706296724 | −0.323819243 | |
| | annotation | | | | | |
| 211336_x_at | LILRB1 | 1.60186E−05 | 0.001507031 | 0.922570226 | 0.447319539 | |
| 201433_s_at | PTDSS1 | 1.66951E−05 | 0.000199218 | 0.849443057 | −0.298448249 | |
| 211284_s_at | GRN | 1.74918E−05 | 0.002054495 | 0.904036627 | 0.455123958 | |
| 204119_s_at | ADK | 1.88846E−05 | 0.002815734 | 0.926817552 | −0.257696817 | |
| 202864_s_at | SP100 | 1.94278E−05 | 0.000673806 | 0.957267847 | 0.400984735 | |
| 201272_at | AKR1B1 | 1.97498E−05 | 8.5382E−06 | 0.824385407 | −0.380485363 | |
| 220755_s_at | C6ORF48 | 2.00312E−05 | 0.006348967 | 0.813784664 | −0.252143599 | |
| 200093_s_at | HINT1 | 2.00617E−05 | 8.29478E−05 | 0.927054575 | −0.359178969 | |
| 216041_x_at | GRN | 2.0389E−05 | 0.001563684 | 0.957267847 | 0.497052621 | |
| 205896_at | SLC22A4 | 2.04806E−05 | 8.71593E−05 | 0.964319867 | 0.478927514 | |
| | (includes | | | | | |
| | EG: 6583) | | | | | |
| 200063_s_at | NPM1 | 2.05577E−05 | 1.679E−05 | 0.933418803 | −0.380986943 | |
| | (includes | | | | | |
| | EG: 4869) | | | | | |
| 207104_x_at | LILRB1 | 2.05577E−05 | 0.008495265 | 0.952168804 | 0.492361467 | |
| 209616_s_at | CES1 (includes | 2.0569E−05 | 0.000196146 | 0.839175678 | 0.528248527 | |
| | EG: 1066) | | | | | |
| 200689_x_at | EEF1G | 2.0569E−05 | 4.93084E−05 | 0.847283605 | −0.334904567 | |
| 210501_x_at | EIF3K | 2.15942E−05 | 0.000207377 | 0.976541414 | −0.275759712 | |
| 216035_x_at | TCF7L2 | 2.27983E−05 | 0.016007945 | 0.937972084 | 0.356351135 | |
| 208918_s_at | NADK | 2.35932E−05 | 0.002522694 | 0.558360244 | 0.494520309 | |
| 222163_s_at | SPATA5L1 | 2.35932E−05 | 0.000290242 | 0.885856306 | 0.352402229 | |
| 214280_x_at | HNRPA1 | 2.35932E−05 | 0.000589823 | 0.906012249 | −0.334912016 | |
| 216570_x_at | RPL29 | 2.39804E−05 | 7.1509E−05 | 0.921230073 | −0.446641707 | |
| | (includes | | | | | |
| | EG: 6159) | | | | | |
| 201033_x_at | RPLP0 | 2.39804E−05 | 5.68087E−05 | 0.948783771 | −0.319753176 | |
| | (includes | | | | | |
| | EG: 6175) | | | | | |
| 218223_s_at | PLEKHO1 | 2.41851E−05 | 0.004700829 | 0.978184927 | 0.370133687 | |
| 218754_at | NOL9 | 2.42684E−05 | 0.001020355 | 0.722442937 | −0.336999354 | |
| 219599_at | EIF4B | 2.50822E−05 | 0.000438687 | 0.958855347 | −0.562792124 | |
| 202469_s_at | CPSF6 | 2.51361E−05 | 5.23645E−05 | 0.983164326 | −0.32609193 | |
| 211984_at | CALM1 | 2.79959E−05 | 2.98467E−05 | 0.667944101 | −0.359804924 | |
| 205269_at | LCP2 | 2.91093E−05 | 0.000106144 | 0.826503176 | 0.464073202 | |
| 217995_at | SQRDL | 2.91093E−05 | 0.008300582 | 0.862781884 | 0.28577528 | |
| | (includes | | | | | |
| | EG: 58472) | | | | | |
| 211073_x_at | RPL3 | 2.96591E−05 | 0.000214153 | 0.959098102 | −0.298765595 | |
| 213261_at | LBA1 | 3.11947E−05 | 0.005806222 | 0.896710842 | 0.276333078 | |
| 200652_at | SSR2 | 3.15124E−05 | 0.000150797 | 0.749293634 | −0.282063207 | |
| 209282_at | PRKD2 | 3.15124E−05 | 0.002011765 | 0.967981598 | 0.343096169 | |
| 212313_at | CHMP7 | 3.15124E−05 | 0.000708686 | 0.996387193 | −0.398559843 | |
| 203538_at | CAMLG | 3.28125E−05 | 0.002274258 | 0.72033594 | −0.258987523 | |
| 220942_x_at | C3ORF28 | 3.28713E−05 | 0.002357652 | 0.99162365 | −0.318433955 | |
| 204959_at | MNDA | 3.55057E−05 | 0.00099468 | 0.556263799 | 0.725578329 | |
| 220933_s_at | ZCCHC6 | 3.85292E−05 | 0.000113929 | 0.793788265 | 0.55076101 | |
| 212690_at | DDHD2 | 4.01316E−05 | 0.000121671 | 0.845163449 | −0.385897611 | |
| 200942_s_at | HSBP1 | 4.08133E−05 | 0.000566984 | 0.777646896 | 0.366713414 | |
| 211967_at | TMEM123 | 4.08133E−05 | 0.00123082 | 0.989523303 | 0.317676844 | |
| 218746_at | TAPBPL | 4.15837E−05 | 0.005350708 | 0.570133912 | 0.413987811 | |
| 203492_x_at | CEP57 | 4.2409E−05 | 0.000535638 | 0.870852621 | −0.325547339 | |
| 200008_s_at | GDI2 | 4.29585E−05 | 1.61347E−05 | 0.974685401 | −0.375970405 | |
| 201422_at | IFI30 | 4.64093E−05 | 0.006089172 | 0.857260432 | 0.377761352 | |
| 117_at | HSPA6 | 4.91495E−05 | 0.003228567 | 0.87331579 | 0.513120601 | |
| 200877_at | CCT4 | 4.91495E−05 | 0.000267502 | 0.9722812 | −0.320444714 | |
| 218747_s_at | TAPBPL | 5.04997E−05 | 0.005239299 | 0.820704131 | 0.411214137 | |
| 206881_s_at | LILRA3 | 5.07001E−05 | 0.000727893 | 0.968104648 | 0.688073893 | |
| 203773_x_at | BLVRA | 5.14119E−05 | 0.012307101 | 0.99162365 | 0.339155772 | |
| 211927_x_at | EEF1G | 5.23546E−05 | 0.000714064 | 0.803159361 | −0.287856256 | |
| 204780_s_at | FAS | 5.23546E−05 | 0.000227842 | 0.971652444 | 0.488565671 | |
| 208856_x_at | RPLP0 | 5.27196E−05 | 3.78677E−05 | 0.939374883 | −0.32035963 | |
| | (includes | | | | | |
| | EG: 6175) | | | | | |
| 205098_at | CCR1 | 5.34222E−05 | 7.90801E−05 | 0.708968475 | 0.904349461 | |
| 201939_at | PLK2 | 5.54204E−05 | 6.94986E−05 | 0.975695805 | −0.991234778 | |
| 35254_at | TRAFD1 | 5.55383E−05 | 0.012001139 | 0.706089088 | 0.275742178 | |
| 200826_at | SNRPD2 | 5.58734E−05 | 8.96776E−05 | 0.989523303 | −0.352157943 | |
| 201924_at | AFF1 | 5.68138E−05 | 0.003760437 | 0.525380174 | 0.294218003 | |
| 201743_at | CD14 | 5.80196E−05 | 0.000927887 | 0.926027044 | 0.532529524 | |
| 202646_s_at | CSDE1 | 5.85627E−05 | 0.001267314 | 0.703685034 | −0.267474933 | |
| 211955_at | RANBP5 | 6.19023E−05 | 0.000269924 | 0.994302012 | −0.361149345 | |
| 204745_x_at | MT1G | 6.24714E−05 | 0.000535638 | 0.99162365 | 0.482758396 | |
| 208965_s_at | IFI16 | 6.26238E−05 | 0.00099468 | 0.921230073 | 0.685450957 | |
| 204187_at | GMPR | 6.46454E−05 | 0.001633158 | 0.899991236 | 0.580973366 | |
| 218680_x_at | HYPK | 6.51351E−05 | 0.003141727 | 0.741625882 | 0.244004498 | |
| 215693_x_at | DDX27 | 6.55698E−05 | 0.000150797 | 0.827347393 | −0.34011042 | |
| 212348_s_at | AOF2 | 6.56031E−05 | 0.007108849 | 0.463553935 | −0.257602593 | |
| 202592_at | BLOC1S1 | 6.7862E−05 | 0.001686828 | 0.994302012 | 0.286307236 | |
| 208581_x_at | MT1X | 7.09165E−05 | 0.00100511 | 0.963348318 | 0.73046362 | |
| 212185_x_at | MT2A | 7.09565E−05 | 0.000440646 | 0.97215811 | 0.667478886 | |
| 208891_at | DUSP6 | 7.35402E−05 | 4.45417E−05 | 0.741625882 | 0.924214277 | |
| 203042_at | LAMP2 | 7.35402E−05 | 0.005146761 | 0.749123103 | 0.413530001 | |
| 208594_x_at | LILRA6 | 7.42589E−05 | 0.009107667 | 0.970304109 | 0.274201604 | |
| 211971_s_at | LRPPRC | 7.5771E−05 | 0.007283007 | 0.94123881 | −0.251164739 | |
| 208664_s_at | TTC3 | 7.63081E−05 | 0.001537804 | 0.838371349 | −0.769757056 | |
| 204493_at | BID | 7.77238E−05 | 0.003938812 | 0.884660098 | 0.331998151 | |
| 208073_x_at | TTC3 | 8.21711E−05 | 0.000149568 | 0.908091808 | −0.300142872 | |
| 220299_at | SPATA6 | 8.28224E−05 | 0.002267971 | 0.825527416 | −0.337259357 | |
| 203416_at | CD53 | 8.3029E−05 | 0.000753618 | 0.776697234 | 0.337815779 | |
| 201324_at | EMP1 | 8.32129E−05 | 2.78036E−05 | 0.932963506 | 1.028302244 | |
| 208804_s_at | SFRS6 | 8.48247E−05 | 0.002747678 | 0.737884754 | −0.235945586 | |
| 209201_x_at | CXCR4 | 8.48738E−05 | 0.009918127 | 0.554763569 | −0.352992299 | |
| 218764_at | PRKCH | 8.51435E−05 | 2.78011E−05 | 0.651124512 | −0.509978449 | |
| 212807_s_at | SORT1 | 8.57713E−05 | 0.002522694 | 0.905358421 | 0.344965524 | |
| 203044_at | CHSY1 | 8.74059E−05 | 0.000335844 | 0.906012249 | 0.465640594 | |
| 216037_x_at | TCF7L2 | 9.17002E−05 | 0.018088923 | 0.927054575 | 0.293147345 | |
| 200096_s_at | ATP6V0E1 | 9.37476E−05 | 0.00437166 | 0.808788579 | 0.328205791 | |
| 200663_at | CD63 | 9.56515E−05 | 0.000223886 | 0.85740335 | 0.328258707 | |
| 210176_at | TLR1 | 9.76332E−05 | 0.000113929 | 0.922570226 | 0.597335658 | |
| 212560_at | C11ORF32 | 9.76332E−05 | 3.86107E−05 | 0.957267847 | −0.405581852 | |
| 222010_at | TCP1 | 9.81152E−05 | 0.000773739 | 0.899088585 | −0.308461921 | |
| 204683_at | ICAM2 | 0.000103459 | 0.003026185 | 0.725120606 | 0.384609385 | |
| 208072_s_at | DGKD | 0.000103459 | 0.002560028 | 0.9404858 | −0.411104014 | |
| 217906_at | KLHDC2 | 0.000104391 | 0.007884053 | 0.837193604 | −0.300351283 | |
| 201456_s_at | BUB3 | 0.000105142 | 0.001148959 | 0.782350826 | −0.417697398 | |
| 216841_s_at | SOD2 | 0.000121579 | 0.002479609 | 0.460110071 | 0.593305775 | |
| 202193_at | LIMK2 | 0.000129311 | 0.002641311 | 0.927054575 | 0.732622819 | |
| 208798_x_at | GOLGA8A | 0.000130254 | 0.003361757 | 0.9845525 | −0.36990332 | |
| 205270_s_at | LCP2 | 0.000133398 | 0.005239299 | 0.980264971 | 0.304580978 | |
| 215399_s_at | OS-9 | 0.000135232 | 0.04826413 | 0.976541414 | 0.192838334 | |
| 209575_at | IL10RB | 0.000136707 | 0.004491629 | 0.903665108 | 0.391467267 | |
| 203396_at | PSMA4 | 0.000139905 | 0.006264569 | 0.922570226 | 0.246413994 | |
| 212820_at | DMXL2 | 0.000141003 | 0.010230962 | 0.921230073 | 0.370167057 | |
| 213969_x_at | RPL29 | 0.000142041 | 0.000342023 | 0.997185403 | −0.300406883 | |
| | (includes | | | | | |
| | EG: 6159) | | | | | |
| 205842_s_at | JAK2 | 0.000144633 | 0.004506325 | 0.93869607 | 0.462990735 | |
| 217989_at | HSD17B11 | 0.000145778 | 2.32942E−05 | 0.951263967 | −0.345467249 | |
| 207610_s_at | EMR2 | 0.000150007 | 0.001371535 | 0.901541785 | 0.52062863 | |
| 204804_at | TRIM21 | 0.000151208 | 0.012126009 | 0.770211009 | 0.238585468 | |
| 208630_at | HADHA | 0.000151208 | 3.78677E−05 | 0.932963506 | −0.419726556 | |
| 204102_s_at | EEF2 | 0.000153539 | 0.01306258 | 0.910685215 | −0.237231825 | |
| 212039_x_at | RPL3 | 0.000156078 | 0.000516537 | 0.942974617 | −0.312659646 | |
| 216336_x_at | MT1M | 0.000156831 | 0.001761164 | 0.808230912 | 0.515017334 | |
| 201947_s_at | CCT2 | 0.000158973 | 0.006761486 | 0.993864266 | −0.263439476 | |
| 204070_at | RARRES3 | 0.00016176 | 0.050940897 | 0.933418803 | 0.238144405 | |
| 208646_at | RPS14 | 0.000167286 | 0.000269924 | 0.852171473 | −0.49321732 | |
| 200017_at | RPS27A | 0.000167286 | 0.000106786 | 0.86861163 | −0.389483016 | |
| 208638_at | PDIA6 | 0.000169202 | 0.022203936 | 0.921230073 | 0.216927173 | |
| 200000_s_at | PRPF8 | 0.000173098 | 0.001316885 | 0.710647678 | −0.301065526 | |
| 213101_s_at | ACTR3 | 0.000173683 | 0.000126297 | 0.554763569 | 0.367744698 | |
| 221680_s_at | ETV7 | 0.000176285 | 0.008422522 | 0.809600216 | 0.422233187 | |
| 200811_at | CIRBP | 0.000182308 | 0.000342023 | 0.576368551 | −0.290815817 | |
| 214567_s_at | XCL2 | 0.000191679 | 6.73554E−05 | 0.906722412 | −0.778832174 | |
| 201569_s_at | SAMM50 | 0.000192531 | 0.001774795 | 0.906722412 | −0.342912382 | |
| 211954_s_at | RANBP5 | 0.000193131 | 0.00119833 | 0.861456705 | −0.324324521 | |
| 218366_x_at | METT11D1 | 0.000193131 | 0.002021656 | 0.916817284 | −0.400617253 | |
| 205686_s_at | CD86 | 0.000193853 | 0.019468497 | 0.69202515 | 0.292707371 | |
| 212953_x_at | CALR | 0.000196693 | 0.002853479 | 0.913292392 | 0.24789854 | |
| 210644_s_at | LAIR1 | 0.000197548 | 0.001737269 | 0.811796229 | 0.414456955 | |
| 218380_at | NLRP1 | 0.000197548 | 0.006418624 | 0.971652444 | −0.237882256 | |
| 204961_s_at | NCF1 | 0.000198079 | 0.006735956 | 0.493067373 | 0.645196061 | |
| 201090_x_at | TUBA1B | 0.000201993 | 0.007154623 | 0.995153496 | 0.246651583 | |
| 211135_x_at | LILRB2 | 0.000203553 | 0.005359237 | 0.82604375 | 0.604255867 | |
| 208763_s_at | TSC22D3 | 0.000205683 | 0.011915365 | 0.721257498 | −0.265245905 | |
| 207023_x_at | KRT10 | 0.000220135 | 0.001523636 | 0.972604469 | −0.282689951 | |
| 202524_s_at | SPOCK2 | 0.000224915 | 0.003256107 | 0.493067373 | −0.424843553 | |
| 208886_at | H1F0 | 0.000229618 | 4.32561E−05 | 0.570133912 | 0.624500357 | |
| 206968_s_at | NFRKB | 0.000232267 | 0.007671535 | 0.574996104 | −0.252565265 | |
| 203113_s_at | EEF1D | 0.00023918 | 0.000721381 | 0.989523303 | −0.4416568 | |
| 208787_at | MRPL3 | 0.000240465 | 0.00093572 | 0.925734912 | −0.36424026 | |
| 201653_at | CNIH | 0.000249558 | 0.001369281 | 0.957267847 | −0.371051819 | |
| 200941_at | HSBP1 | 0.00025073 | 0.000857478 | 0.653680011 | 0.336300609 | |
| 204089_x_at | MAP3K4 | 0.000252114 | 0.025031513 | 0.460110071 | −0.289870986 | |
| 214150_x_at | ATP6V0E1 | 0.00027072 | 0.020501576 | 0.806943262 | 0.23805218 | |
| 221985_at | KLHL24 | 0.00027072 | 0.046675751 | 0.824385407 | −0.274959819 | |
| 201356_at | SF3A1 | 0.000279263 | 0.002747678 | 0.436770009 | −0.275540687 | |
| 210069_at | CPT1B | 0.000285211 | 0.002170519 | 0.408299445 | 0.355909262 | |
| 221641_s_at | ACOT9 | 0.000285211 | 0.002306028 | 0.710824574 | 0.348957648 | |
| 215838_at | LILRA5 | 0.000285211 | 0.003027785 | 0.838371349 | 0.689814945 | |
| 221756_at | PIK3IP1 | 0.000287807 | 0.010073144 | 0.717703472 | −0.45540524 | |
| 201646_at | SCARB2 | 0.000295746 | 0.005641363 | 0.884660098 | 0.294088056 | |
| 204249_s_at | LMO2 | 0.000299566 | 0.028108844 | 0.955997488 | 0.274672756 | |
| 218809_at | PANK2 | 0.00030108 | 0.013167345 | 0.275116383 | 0.226774944 | |
| 219243_at | GIMAP4 | 0.000302454 | 0.015295263 | 0.957267847 | 0.491082072 | |
| 211919_s_at | CXCR4 | 0.000303358 | 0.048977002 | 0.647246593 | −0.284135188 | |
| 212647_at | RRAS | 0.000303358 | 0.000756774 | 0.874249756 | 0.898538883 | |
| 205099_s_at | CCR1 | 0.000303358 | 0.006752633 | 0.980264971 | 0.997306148 | |
| 211058_x_at | TUBA1B | 0.000305393 | 0.006761486 | 0.978991398 | 0.236121092 | |
| 214686_at | ZNF266 | 0.000319692 | 0.003137784 | 0.591222948 | −0.347543601 | |
| 219049_at | CHGN | 0.000336807 | 0.039771173 | 0.511801929 | −0.284541914 | |
| 217868_s_at | METTL9 | 0.000339692 | 0.002020053 | 0.922570226 | −0.357567284 | |
| 205681_at | BCL2A1 | 0.000344351 | 0.003364282 | 0.968649563 | 0.609541704 | |
| 212406_s_at | PCMTD2 | 0.000348623 | 0.001710167 | 0.998328856 | −0.322403539 | |
| 211133_x_at | LILRB2 | 0.000360302 | 0.009405926 | 0.908091808 | 0.446173357 | |
| 216559_x_at | HNRPA1 | 0.000361214 | 0.000451118 | 0.97607782 | −0.366507064 | |
| 200022_at | RPL18 | 0.000366884 | 0.000607336 | 0.942959418 | −0.318836404 | |
| 216640_s_at | PDIA6 | 0.000383384 | 0.008422522 | 0.907894589 | 0.263013219 | |
| 219371_s_at | KLF2 | 0.000387198 | 0.008146602 | 0.952102861 | −0.285987047 | |
| 221123_x_at | ZNF395 | 0.00039184 | 0.01465184 | 0.439510344 | −0.350035271 | |
| 212380_at | KIAA0082 | 0.000394564 | 7.82029E−05 | 0.82604375 | 0.460014641 | |
| 202180_s_at | MVP | 0.000394683 | 0.098664757 | 0.839137004 | 0.23899631 | |
| 212737_at | GM2A | 0.000396843 | 0.00426447 | 0.804966785 | 0.349031287 | |
| 213102_at | ACTR3 | 0.000406755 | 0.000847512 | 0.493067373 | 0.38533818 | |
| 204446_s_at | ALOX5 | 0.000409964 | 0.010730062 | 0.517633091 | 0.353559732 | |
| 214394_x_at | EEF1D | 0.000411744 | 0.000885874 | 0.852969982 | −0.362814581 | |
| 218458_at | GMCL1 | 0.000411744 | 0.018155896 | 0.927054575 | −0.293518292 | |
| 202068_s_at | LDLR | 0.000411744 | 0.000886343 | 0.976077352 | 0.563864068 | |
| 216945_x_at | PASK | 0.000418874 | 0.040273221 | 0.926027044 | −0.339768876 | |
| 203470_s_at | PLEK | 0.000427191 | 0.005442512 | 0.991325836 | 0.529418531 | |
| 219646_at | FLJ20186 | 0.000433149 | 0.002781205 | 0.645055666 | −0.330686962 | |
| 201298_s_at | MOBKL1B | 0.000435123 | 0.000463884 | 0.739477563 | 0.446942833 | |
| 218149_s_at | ZNF395 | 0.000435888 | 0.017767982 | 0.39977166 | −0.343534668 | |
| 215346_at | CD40 | 0.000444998 | 0.00482122 | 0.943566129 | 0.484897905 | |
| 205898_at | CX3CR1 | 0.000454627 | 0.021988609 | 0.888078708 | 0.747111855 | |
| 202833_s_at | SERPINA1 | 0.000472262 | 0.024006884 | 0.811796229 | 0.348410728 | |
| 222217_s_at | SLC27A3 | 0.000472285 | 0.00752454 | 0.9404858 | 0.420872519 | |
| 55692_at | ELMO2 | 0.00048374 | 0.001905065 | 0.968649563 | 0.367590053 | |
| 209304_x_at | GADD45B | 0.000490046 | 0.001112052 | 0.740342948 | 0.564383956 | |
| 211799_x_at | HLA-C | 0.000490046 | 0.01330827 | 0.959098102 | 0.234282769 | |
| 212757_s_at | CAMK2G | 0.000490046 | 0.000803479 | 0.998615467 | −0.296177222 | |
| 206461_x_at | MT1H | 0.000492013 | 0.001066545 | 0.980726521 | 0.737329152 | |
| 200803_s_at | TEGT | 0.0005025 | 0.012797193 | 0.891670708 | 0.25065271 | |
| 217202_s_at | GLUL | 0.000503252 | 0.000493023 | 0.997185403 | 0.631358384 | |
| 200042_at | C22ORF28 | 0.00051179 | 0.082077787 | 0.711933676 | 0.182344332 | |
| 217794_at | PRR13 | 0.000513337 | 0.007100635 | 0.761083429 | 0.291184619 | |
| 212462_at | Need to update | 0.000513337 | 0.010760912 | 0.811796229 | −0.349376398 | |
| | annotation | | | | | |
| 216383_at | HCG 2040224 | 0.000513337 | 0.000552261 | 0.846751596 | −0.543196644 | |
| 201952_at | ALCAM | 0.000513337 | 0.017218526 | 0.973104905 | −0.257642142 | |
| 213646_x_at | TUBA1B | 0.000521447 | 0.009552139 | 0.939653852 | 0.242315436 | |
| 200810_s_at | CIRBP | 0.000521834 | 0.004276434 | 0.689887873 | −0.280606824 | |
| 210425_x_at | GOLGA8B | 0.000527301 | 0.084397141 | 0.9404858 | −0.25737113 | |
| 212014_x_at | CD44 | 0.000537985 | 0.004518647 | 0.404804753 | −0.386035136 | |
| 209933_s_at | CD300A | 0.000562336 | 0.004506325 | 0.974685401 | 0.484834372 | |
| 218987_at | ATF7IP | 0.000575666 | 0.002021169 | 0.570133912 | −0.337529831 | |
| 209835_x_at | CD44 | 0.000577792 | 0.001774795 | 0.233883948 | −0.31988032 | |
| 212665_at | TIPARP | 0.000577792 | 0.005350708 | 0.463553935 | −0.337663556 | |
| 218535_s_at | RIOK2 | 0.000579363 | 0.006704494 | 0.622352164 | −0.400136904 | |
| 203041_s_at | LAMP2 | 0.000579363 | 0.004440578 | 0.947792157 | 0.413824286 | |
| 204706_at | INPP5E | 0.000583831 | 0.01306258 | 0.921387833 | −0.244364287 | |
| 219108_x_at | DDX27 | 0.000588644 | 0.004037506 | 0.893325545 | −0.324986959 | |
| 210633_x_at | KRT10 | 0.000591061 | 0.000516537 | 0.849563735 | −0.415728601 | |
| 200666_s_at | DNAJB1 | 0.000606799 | 0.01454404 | 0.621038766 | −0.357251555 | |
| 212495_at | JMJD2B | 0.000606945 | 0.001458413 | 0.906722412 | −0.450191108 | |
| 221483_s_at | ARPP-19 | 0.000613195 | 0.009953383 | 0.511801929 | −0.248035301 | |
| 200858_s_at | RPS8 | 0.000613195 | 0.000327341 | 0.938509968 | −0.369135153 | |
| 201576_s_at | GLB1 | 0.000623374 | 0.011973966 | 0.835997143 | 0.342339064 | |
| 216705_s_at | ADA | 0.000632586 | 0.002781205 | 0.943771576 | 0.306990949 | |
| 211456_x_at | MT1P2 | 0.000639141 | 0.001401757 | 0.997185403 | 0.744016347 | |
| 214442_s_at | PIAS2 | 0.000642096 | 0.011798434 | 0.006848914 | −0.253731909 | |
| 200002_at | RPL35 | 0.00064429 | 0.001057167 | 0.980264971 | −0.355531622 | |
| 221988_at | C19ORF42 | 0.000656502 | 0.000174071 | 0.980754763 | −0.523975286 | |
| 213503_x_at | ANXA2 | 0.000659784 | 0.026353097 | 0.811796229 | 0.332837372 | |
| 203028_s_at | CYBA | 0.000694018 | 0.006166017 | 0.676993566 | 0.331149854 | |
| 203410_at | AP3M2 | 0.000696278 | 0.038963638 | 0.806943262 | −0.295470157 | |
| 200973_s_at | TSPAN3 | 0.000696278 | 0.011435535 | 0.9845525 | −0.334248544 | |
| 210427_x_at | ANXA2 | 0.000706529 | 0.020819979 | 0.833074897 | 0.336200928 | |
| 200664_s_at | DNAJB1 | 0.000718995 | 0.014988565 | 0.481710884 | −0.373826408 | |
| 201590_x_at | ANXA2 | 0.000722606 | 0.02939767 | 0.749123103 | 0.316243552 | |
| 218085_at | CHMP5 | 0.000723341 | 0.012687139 | 0.652660658 | 0.363761991 | |
| 211594_s_at | MRPL9 | 0.000751237 | 0.007394863 | 0.740342948 | −0.399938201 | |
| 203454_s_at | ATOX1 | 0.00075348 | 0.039264525 | 0.994302012 | 0.38634657 | |
| 202644_s_at | TNFAIP3 | 0.000755493 | 0.045075126 | 0.706296724 | −0.282282288 | |
| 221622_s_at | TMEM126B | 0.000755957 | 0.003952573 | 0.563830701 | 0.33832929 | |
| 204924_at | TLR2 | 0.000763579 | 0.000631869 | 0.981976319 | 0.609317622 | |
| 218344_s_at | RCOR3 | 0.000765713 | 0.0274525 | 0.826503176 | −0.310921835 | |
| 200965_s_at | ABLIM1 | 0.000767558 | 0.000210718 | 0.932963506 | −0.418271022 | |
| 203276_at | LMNB1 | 0.000767975 | 0.004082556 | 0.890400749 | 0.580225782 | |
| 201760_s_at | WSB2 | 0.000767975 | 0.023975743 | 0.994302012 | 0.248294941 | |
| 210225_x_at | LILRB2 | 0.00076903 | 0.023630979 | 0.894898588 | 0.462366549 | |
| 202907_s_at | NBN | 0.000769354 | 0.001521011 | 0.799693669 | 0.35193611 | |
| 201581_at | TXNDC13 | 0.000796894 | 0.00875001 | 0.83681131 | −0.251541541 | |
| 213588_x_at | RPL14 | 0.000821928 | 0.003026707 | 0.937972084 | −0.293914008 | |
| 210385_s_at | ARTS-1 | 0.000851116 | 0.076481332 | 0.739726094 | 0.263386249 | |
| 203471_s_at | PLEK | 0.000855203 | 0.00920277 | 0.921964349 | 0.37671753 | |
| 211725_s_at | BID | 0.000867216 | 0.013142031 | 0.717703472 | 0.316370725 | |
| 214315_x_at | CALR | 0.000868206 | 0.037858967 | 0.892303998 | 0.260644273 | |
| 215001_s_at | GLUL | 0.000868206 | 0.010361012 | 0.966599128 | 0.29235252 | |
| 219033_at | PARP8 | 0.000873331 | 0.018530669 | 0.804614382 | −0.239294084 | |
| 202230_s_at | CHERP | 0.000874383 | 0.007871094 | 0.493067373 | −0.317436342 | |
| 38241_at | BTN3A3 | 0.000880045 | 0.045193355 | 0.654429654 | 0.299200503 | |
| 221221_s_at | KLHL3 | 0.00088145 | 0.019547173 | 0.992470596 | −0.397613329 | |
| | (includes | | | | | |
| | EG: 26249) | | | | | |
| 203922_s_at | CYBB | 0.000887 | 0.031820023 | 0.890400749 | 0.320794193 | |
| 209251_x_at | TUBA1C | 0.000887316 | 0.011718661 | 0.614354446 | 0.282059782 | |
| 207275_s_at | ACSL1 | 0.000887316 | 0.001906965 | 0.940021472 | 0.65843976 | |
| 207224_s_at | SIGLEC7 | 0.000891971 | 0.00920277 | 0.809600216 | 0.476452984 | |
| 206983_at | CCR6 | 0.000898256 | 0.012660545 | 0.825177207 | −0.482482474 | |
| 209787_s_at | HMGN4 | 0.000898256 | 0.025538134 | 0.952168804 | 0.254826309 | |
| 210825_s_at | PEBP1 | 0.000898256 | 0.002930074 | 0.969968852 | −0.313529462 | |
| 200888_s_at | RPL23 | 0.000904895 | 0.001710167 | 0.985775064 | −0.301386866 | |
| 201607_at | PWP1 | 0.000925757 | 0.001341171 | 0.993864266 | −0.324177277 | |
| 202405_at | TIAL1 | 0.000930351 | 0.04826413 | 0.82693078 | −0.24098632 | |
| 218654_s_at | MRPS33 | 0.000938023 | 0.001170572 | 0.882218308 | −0.395220925 | |
| 213241_at | PLXNC1 | 0.000945389 | 0.016609627 | 0.518649529 | 0.477531003 | |
| 210582_s_at | LIMK2 | 0.000945389 | 0.010429417 | 0.980192601 | 0.536975779 | |
| 210113_s_at | NLRP1 | 0.000949494 | 0.003695966 | 0.933418803 | −0.332340611 | |
| 201172_x_at | ATP6V0E1 | 0.000962774 | 0.007953975 | 0.989523303 | 0.26719857 | |
| 208864_s_at | TXN | 0.000967186 | 0.011435535 | 0.726166985 | 0.292113103 | |
| 211250_s_at | SH3BP2 | 0.000970367 | 0.000635992 | 0.996581247 | 0.414609016 | |
| 200843_s_at | EPRS | 0.000988133 | 0.003026185 | 0.649255083 | −0.303517149 | |
| 203494_s_at | CEP57 | 0.001009764 | 0.000516499 | 0.903665108 | −0.354629675 | |
| 201241_at | DDX1 | 0.001013717 | 0.002084072 | 0.784412478 | −0.414030979 | |
| 204019_s_at | SH3YL1 | 0.001030022 | 0.003361757 | 0.574996104 | −0.389856408 | |
| 209901_x_at | AIF1 | 0.001030022 | 0.016164027 | 0.891454344 | 0.379496405 | |
| 207387_s_at | GK | 0.001030022 | 0.021186181 | 0.932963506 | 0.654457685 | |
| 200074_s_at | RPL14 | 0.00103668 | 0.001703809 | 0.97793068 | −0.329340666 | |
| 221666_s_at | PYCARD | 0.00105502 | 0.018530669 | 0.885856306 | 0.379709728 | |
| 211429_s_at | SERPINA1 | 0.001065102 | 0.019119967 | 0.903665108 | 0.313891813 | |
| 208195_at | TTN | 0.001085516 | 0.014960098 | 0.997537915 | −0.303979146 | |
| 217826_s_at | UBE2J1 | 0.001091763 | 0.00869776 | 0.971652444 | 0.295813149 | |
| 216511_s_at | TCF7L2 | 0.001092487 | 0.04316627 | 0.99162365 | 0.33019635 | |
| 202367_at | CUTL1 | 0.001117484 | 0.041002541 | 0.377556356 | 0.276851206 | |
| 218357_s_at | TIMM8B | 0.001117484 | 0.035658714 | 0.570645923 | 0.224141838 | |
| 202020_s_at | LANCL1 | 0.001117484 | 0.010158376 | 0.68704615 | −0.300078637 | |
| 218476_at | POMT1 | 0.001117484 | 0.003925287 | 0.989523303 | −0.452181221 | |
| 210070_s_at | CPT1B | 0.001146661 | 0.003947253 | 0.892303998 | 0.306480967 | |
| 202414_at | ERCC5 | 0.001170122 | 0.004276434 | 0.969487152 | −0.306685713 | |
| 200701_at | NPC2 | 0.001202659 | 0.032221551 | 0.655141739 | 0.216264415 | |
| 218298_s_at | C14ORF159 | 0.00122103 | 0.010766065 | 0.525380174 | 0.468319482 | |
| 204998_s_at | ATF5 | 0.00122103 | 0.116529465 | 0.806943262 | 0.228359114 | |
| 208680_at | PRDX1 | 0.001246093 | 0.019133444 | 0.849784409 | 0.285755256 | |
| 212859_x_at | MT1E | 0.00125981 | 0.00428389 | 0.9404858 | 0.496034508 | |
| 209536_s_at | EHD4 | 0.001271367 | 0.000862146 | 0.602107351 | 0.500016127 | |
| 209733_at | LOC286440 | 0.001294607 | 0.005972081 | 0.66764821 | −0.389101037 | |
| 201470_at | GSTO1 | 0.001298045 | 0.003675484 | 0.959247226 | 0.277751923 | |
| 204175_at | ZNF593 | 0.001312859 | 0.008383113 | 0.570133912 | 0.273324989 | |
| 211985_s_at | CALM1 | 0.001332095 | 0.002522694 | 0.957267847 | −0.32859565 | |
| 208944_at | TGFBR2 | 0.001345505 | 0.028000613 | 0.920178258 | −0.261960571 | |
| 208822_s_at | DAP3 | 0.001354808 | 0.004585775 | 0.716959241 | −0.294784118 | |
| 212285_s_at | AGRN | 0.001356035 | 0.014751333 | 0.896710842 | 0.365172385 | |
| 219316_s_at | FLVCR2 | 0.001376113 | 0.007295947 | 0.704384868 | 0.488143001 | |
| 213018_at | GATAD1 | 0.001386955 | 0.004923299 | 0.798376788 | −0.355469919 | |
| 200932_s_at | DCTN2 | 0.00141155 | 0.001035731 | 0.913292392 | −0.324444916 | |
| 202767_at | ACP2 | 0.001430865 | 0.003957353 | 0.658726886 | 0.314860329 | |
| 203428_s_at | ASF1A | 0.001430865 | 0.013167345 | 0.909501667 | −0.395078596 | |
| 212440_at | RY1 | 0.00143871 | 0.000380111 | 0.460110071 | −0.414104645 | |
| 213534_s_at | PASK | 0.001446238 | 0.041171417 | 0.755657054 | −0.354090552 | |
| 202912_at | ADM | 0.001457959 | 0.001675882 | 0.995153496 | 0.709207627 | |
| 219439_at | C1GALT1 | 0.001467726 | 0.008550305 | 0.570133912 | 0.335781258 | |
| 203127_s_at | SPTLC2 | 0.001467726 | 0.080807694 | 0.940578006 | 0.204545861 | |
| | (includes | | | | | |
| | EG: 9517) | | | | | |
| 209647_s_at | SOCS5 | 0.001481468 | 0.003633372 | 0.86219885 | −0.364123725 | |
| 218734_at | NAT11 | 0.001499671 | 0.018530669 | 0.658621166 | −0.246005267 | |
| 203610_s_at | TRIM38 | 0.001499671 | 0.009107667 | 0.741625882 | 0.355751974 | |
| 217165_x_at | MT1F | 0.001582759 | 0.011718661 | 0.87331579 | 0.468426853 | |
| 219315_s_at | C16ORF30 | 0.001584536 | 0.013085216 | 0.884660098 | −0.257539313 | |
| 202910_s_at | CD97 | 0.001584536 | 0.005547328 | 0.931474938 | 0.356334383 | |
| 202250_s_at | WDR42A | 0.001623004 | 0.038995938 | 0.931474938 | −0.265826992 | |
| 202122_s_at | M6PRBP1 | 0.001632577 | 0.01173034 | 0.647302266 | 0.274962463 | |
| 219343_at | CDC37L1 | 0.001640474 | 0.05270375 | 0.679835233 | −0.338595411 | |
| 202832_at | GCC2 | 0.001640474 | 0.052684705 | 0.911573276 | −0.283445314 | |
| 213095_x_at | AIF1 | 0.001640474 | 0.019258047 | 0.995153496 | 0.349424159 | |
| 204568_at | KIAA0831 | 0.00165125 | 0.00948745 | 0.608791366 | −0.31832152 | |
| 201326_at | CCT6A | 0.001700329 | 0.020501576 | 0.626064956 | −0.29412661 | |
| 205831_at | CD2 | 0.001700329 | 0.027031678 | 0.933615651 | −0.286425887 | |
| 204793_at | GPRASP1 | 0.001730888 | 0.038034197 | 0.926027044 | −0.353562469 | |
| 204794_at | DUSP2 | 0.001744444 | 0.004369135 | 0.496888495 | −0.474603942 | |
| 203642_s_at | COBLL1 | 0.001744444 | 0.006424855 | 0.717703472 | −0.422705467 | |
| 222139_at | KIAA1466 | 0.001744444 | 0.007001124 | 0.957267847 | 0.550595052 | |
| 212206_s_at | H2AFV | 0.001752486 | 0.06884886 | 0.670488531 | −0.242099204 | |
| 202179_at | BLMH | 0.001807502 | 0.002599745 | 0.884660098 | −0.670098186 | |
| 209305_s_at | GADD45B | 0.001808823 | 0.005641363 | 0.861984472 | 0.458240739 | |
| 218611_at | IER5 | 0.001808823 | 0.038758876 | 0.867588284 | 0.266027709 | |
| 218999_at | TMEM140 | 0.001831038 | 0.032642302 | 0.745326368 | 0.299391846 | |
| 201237_at | CAPZA2 | 0.001831038 | 0.01085208 | 0.811151638 | 0.276258879 | |
| 200692_s_at | HSPA9 | 0.001831038 | 0.011175217 | 0.952271823 | −0.340807584 | |
| 209185_s_at | IRS2 | 0.001844782 | 0.021416843 | 0.974685401 | −0.336365573 | |
| 208885_at | LCP1 | 0.001847763 | 0.028151767 | 0.963348318 | 0.247481673 | |
| 210784_x_at | LILRB2 | 0.001864354 | 0.034519224 | 0.927054575 | 0.430331821 | |
| 213615_at | MBOAT5 | 0.001867904 | 0.036924797 | 0.884660098 | −0.256709194 | |
| 205321_at | EIF2S3 | 0.001867904 | 0.005997244 | 0.997185403 | −0.535802439 | |
| 208018_s_at | HCK | 0.001906005 | 0.036087451 | 0.613727458 | 0.443368869 | |
| 203814_s_at | NQO2 | 0.001931351 | 0.019119967 | 0.658726886 | 0.482908465 | |
| 46665_at | SEMA4C | 0.00193228 | 0.044723957 | 0.158947458 | −0.32734474 | |
| 202447_at | DECR1 | 0.001942864 | 0.027841001 | 0.852019395 | 0.306751151 | |
| 218231_at | NAGK | 0.001949758 | 0.048981958 | 0.670889982 | 0.330588322 | |
| 200648_s_at | GLUL | 0.00198354 | 0.00527049 | 0.913332358 | 0.697474204 | |
| 213122_at | TSPYL5 | 0.00198354 | 0.022687088 | 0.945676894 | −0.277246833 | |
| 205126_at | VRK2 | 0.001984886 | 0.018685204 | 0.983352528 | 0.274539756 | |
| 204494_s_at | C15ORF39 | 0.001988327 | 0.028151767 | 0.806943262 | 0.26199721 | |
| 212812_at | SERINC5 | 0.002021718 | 0.101514284 | 0.929312703 | −0.2760828 | |
| 208893_s_at | DUSP6 | 0.002036658 | 0.006319223 | 0.937164918 | 1.281497985 | |
| 208982_at | PECAM1 | 0.002048249 | 0.079270267 | 0.997185403 | 0.224737326 | |
| 210949_s_at | EIF3C | 0.002067967 | 0.000975446 | 0.999684232 | −0.360444341 | |
| 208816_x_at | ANX2P2 | 0.002083728 | 0.007890896 | 0.83681131 | 0.323780056 | |
| 209751_s_at | TRAPPC2 | 0.002100409 | 0.018977796 | 0.878184512 | −0.309223681 | |
| 208623_s_at | VIL2 | 0.002112139 | 0.017267896 | 0.82330521 | −0.377393449 | |
| 204098_at | RBMX2 | 0.002115591 | 0.019160876 | 0.994302012 | −0.30492075 | |
| 201380_at | CRTAP | 0.002180282 | 0.007726118 | 0.949315993 | −0.304642718 | |
| 213607_x_at | NADK | 0.002249929 | 0.038312345 | 0.827347393 | 0.377743985 | |
| 212578_x_at | RPS17 | 0.002249929 | 0.021857135 | 0.994302012 | −0.252639434 | |
| | (includes | | | | | |
| | EG: 6218) | | | | | |
| 206687_s_at | PTPN6 | 0.002295934 | 0.012698387 | 0.748460361 | 0.275839516 | |
| 211893_x_at | CD6 | 0.002328916 | 0.076173873 | 0.684438087 | −0.42156473 | |
| 211048_s_at | PDIA4 | 0.002374482 | 0.079390663 | 0.614354446 | 0.254948869 | |
| 213527_s_at | ZNF688 | 0.002375008 | 0.046069362 | 0.992470596 | 0.238402052 | |
| 216199_s_at | MAP3K4 | 0.00237884 | 0.021520834 | 0.940578006 | −0.30913149 | |
| 203567_s_at | TRIM38 | 0.00244585 | 0.007856926 | 0.92632367 | 0.510133358 | |
| 212467_at | DNAJC13 | 0.002463344 | 0.021954471 | 0.339878792 | 0.412169546 | |
| 202906_s_at | NBN | 0.002463344 | 0.008603033 | 0.895350322 | 0.47762565 | |
| 204781_s_at | FAS | 0.002463344 | 0.002374667 | 0.9546428 | 0.331757378 | |
| 212675_s_at | CEP68 | 0.002508306 | 0.061221458 | 0.976541414 | −0.317506099 | |
| 208074_s_at | AP2S1 | 0.002512384 | 0.025202689 | 0.906722412 | 0.243247711 | |
| 211900_x_at | CD6 | 0.002555068 | 0.099688256 | 0.627940295 | −0.380349746 | |
| 210046_s_at | IDH2 | 0.002558945 | 0.131639009 | 0.963348318 | 0.179725917 | |
| 202747_s_at | ITM2A | 0.002580928 | 0.07366103 | 0.882599341 | −0.314112044 | |
| 207001_x_at | TSC22D3 | 0.002612245 | 0.071884369 | 0.775565942 | −0.423736994 | |
| 213274_s_at | CTSB | 0.002632035 | 0.027841001 | 0.937972084 | 0.33677359 | |
| 201850_at | CAPG | 0.002695412 | 0.018295566 | 0.708363394 | 0.381728667 | |
| 209207_s_at | SEC22B | 0.002713692 | 0.005415056 | 0.717703472 | 0.333034778 | |
| 206492_at | FHIT | 0.002713692 | 0.020576696 | 0.989523303 | −0.288495472 | |
| 219817_at | C12ORF47 | 0.002720658 | 0.007459361 | 0.970166679 | −0.304423811 | |
| 214909_s_at | DDAH2 | 0.002764927 | 0.024531252 | 0.964319867 | 0.27355178 | |
| 221757_at | PIK3IP1 | 0.002779628 | 0.058129054 | 0.756328383 | −0.321192532 | |
| 202523_s_at | SPOCK2 | 0.002783903 | 0.02715706 | 0.463523306 | −0.34716225 | |
| 205382_s_at | CFD | 0.002809191 | 0.102540247 | 0.87331579 | 0.268032323 | |
| 203413_at | NELL2 | 0.002818871 | 0.027815279 | 0.980673033 | −0.379895837 | |
| 200766_at | CTSD | 0.002839836 | 0.00340538 | 0.544104436 | 0.353066091 | |
| 202374_s_at | RAB3GAP2 | 0.002839836 | 0.160685179 | 0.864671393 | −0.176697263 | |
| 207857_at | LILRA2 | 0.00284801 | 0.051395894 | 0.735106887 | 0.278419117 | |
| 200782_at | ANXA5 | 0.00284801 | 0.021627048 | 0.96226171 | 0.299232848 | |
| 218494_s_at | SLC2A4RG | 0.002864755 | 0.009115983 | 0.862432274 | −0.319185279 | |
| 205256_at | ZBTB39 | 0.002901176 | 0.002930074 | 0.677299865 | −0.32847357 | |
| 219055_at | SRBD1 | 0.002939152 | 0.044631413 | 0.613727458 | 0.310356543 | |
| 205237_at | FCN1 | 0.002977119 | 0.026100589 | 0.92335789 | 0.316019186 | |
| 221011_s_at | LBH | 0.002980455 | 0.026851131 | 0.903552974 | −0.300333828 | |
| 208862_s_at | CTNND1 | 0.003019451 | 0.090262707 | 0.090242763 | −0.311168415 | |
| 218026_at | CCDC56 | 0.003116567 | 0.026774511 | 0.525711859 | 0.289130576 | |
| 203140_at | BCL6 | 0.003116567 | 0.017549829 | 0.903325227 | 0.327171653 | |
| 217118_s_at | C22ORF9 | 0.003171274 | 0.051652279 | 0.902614556 | 0.322645076 | |
| 209155_s_at | NT5C2 | 0.003171274 | 0.137127736 | 0.915663453 | 0.207641141 | |
| 205129_at | NPM3 | 0.003189118 | 0.083885368 | 0.931474938 | −0.201727885 | |
| 215051_x_at | AIF1 | 0.003220648 | 0.042501273 | 0.978012719 | 0.263344748 | |
| 202610_s_at | MED14 | 0.003228283 | 0.009000926 | 0.940578006 | −0.35183311 | |
| 219788_at | PILRA | 0.003234559 | 0.071289231 | 0.582517878 | 0.353600361 | |
| 213227_at | PGRMC2 | 0.003249496 | 0.083818883 | 0.554763569 | −0.276589537 | |
| 205568_at | AQP9 | 0.003318349 | 0.01209333 | 0.94466883 | 0.660912235 | |
| 213570_at | EIF4E2 | 0.003370592 | 0.006232883 | 0.400416235 | −0.300252785 | |
| 209375_at | XPC | 0.00337838 | 0.066025213 | 0.846591986 | −0.266873767 | |
| 209906_at | C3AR1 | 0.003419893 | 0.002321226 | 0.933418803 | 0.819839706 | |
| 205633_s_at | ALAS1 | 0.003421631 | 0.003633372 | 0.945733557 | 0.357107375 | |
| 217379_at | Need to update | 0.00348547 | 0.003301103 | 0.929312703 | −0.395568974 | |
| | annotation | | | | | |
| 204651_at | NRF1 | 0.003562633 | 0.002034121 | 0.825177207 | −0.343940015 | |
| 213348_at | CDKN1C | 0.00361125 | 0.359989751 | 0.339878792 | 0.17004092 | |
| 206335_at | GALNS | 0.003616738 | 0.009972197 | 0.997185403 | 0.341076825 | |
| 202387_at | BAG1 | 0.003666577 | 0.011175217 | 0.932963506 | 0.375692854 | |
| 211100_x_at | LILRA2 | 0.003758821 | 0.046711074 | 0.937686659 | 0.295017549 | |
| 210212_x_at | MTCP1 | 0.003770255 | 0.065330152 | 0.971652444 | −0.242830116 | |
| 208919_s_at | NADK | 0.003770388 | 0.015298913 | 0.815777859 | 0.443867875 | |
| 210119_at | KCNJ15 | 0.003788475 | 0.010539381 | 0.460110071 | 1.090969266 | |
| 215905_s_at | WDR57 | 0.003796022 | 0.034214155 | 0.946102625 | −0.266456759 | |
| | (includes | | | | | |
| | EG: 9410) | | | | | |
| 201963_at | ACSL1 | 0.003829226 | 0.018963938 | 0.98230595 | 0.439072522 | |
| 220054_at | IL23A | 0.003897951 | 0.081029058 | 0.706822729 | −0.266216156 | |
| 209788_s_at | ARTS-1 | 0.003939996 | 0.102540247 | 0.814313307 | 0.405643872 | |
| 219700_at | PLXDC1 | 0.003939996 | 0.083844234 | 0.992470596 | −0.26720345 | |
| 206631_at | PTGER2 | 0.003963935 | 0.025563375 | 0.937598763 | 0.365669176 | |
| 205068_s_at | ARHGAP26 | 0.004000498 | 0.012736339 | 0.896710842 | 0.380960494 | |
| 209407_s_at | DEAF1 | 0.004015257 | 0.053343511 | 0.903665108 | −0.245833031 | |
| 217977_at | SEPX1 | 0.004077343 | 0.058629219 | 0.706822729 | 0.556329571 | |
| 215332_s_at | CD8B | 0.004103592 | 0.026501749 | 0.711933676 | −0.494046928 | |
| 214112_s_at | CXORF40A | 0.004151672 | 0.253490893 | 0.803159361 | −0.145135989 | |
| 209162_s_at | PRPF4 | 0.004278233 | 0.003695966 | 0.689887873 | −0.49923349 | |
| 206296_x_at | MAP4K1 | 0.004289789 | 0.001681474 | 0.903665108 | −0.74579783 | |
| 212696_s_at | RNF4 | 0.004309434 | 0.02939767 | 0.652902586 | −0.376513628 | |
| 212669_at | CAMK2G | 0.004309434 | 0.046716858 | 0.913332358 | −0.266009209 | |
| 212068_s_at | KIAA0515 | 0.004343314 | 0.035073983 | 0.777646896 | −0.501169346 | |
| 209357_at | CITED2 | 0.004351868 | 0.018723142 | 0.893518013 | −0.387064354 | |
| 205329_s_at | SNX4 | 0.004376197 | 0.037305878 | 0.828414098 | −0.25956829 | |
| 205599_at | TRAF1 | 0.004377463 | 0.071289231 | 0.855380438 | −0.261039405 | |
| 219053_s_at | VPS37C | 0.004423914 | 0.001959571 | 0.92335789 | 0.37798312 | |
| 218432_at | FBXO3 | 0.004423914 | 0.024609768 | 0.957288918 | −0.361399133 | |
| 211750_x_at | TUBA1C | 0.004450762 | 0.032221551 | 0.576624724 | 0.272705076 | |
| 204992_s_at | PFN2 | 0.004534971 | 0.04006978 | 0.823670503 | −0.251134207 | |
| 218315_s_at | CDK5RAP1 | 0.004605511 | 0.017641904 | 0.570645923 | −0.291155276 | |
| 216274_s_at | SEC11A | 0.004605511 | 0.000949277 | 0.825177207 | −0.378159692 | |
| 209422_at | PHF20 | 0.004605511 | 0.017998525 | 0.935442363 | −0.385257882 | |
| 36829_at | PER1 | 0.004625639 | 0.194358982 | 0.591499146 | −0.236059027 | |
| 38340_at | HIP1R | 0.004625681 | 0.048615893 | 0.899088585 | −0.285830801 | |
| 212178_s_at | POM121 | 0.004729829 | 0.045193355 | 0.174921132 | −0.250353794 | |
| 212602_at | WDFY3 | 0.004735822 | 0.022252697 | 0.8856292 | 0.475289863 | |
| 214219_x_at | MAP4K1 | 0.00474476 | 0.001157259 | 0.937164918 | −0.954745175 | |
| 209177_at | C3ORF60 | 0.004748968 | 0.093442772 | 0.504375257 | 0.269283722 | |
| 211596_s_at | LRIG1 | 0.004762462 | 0.052606873 | 0.710647678 | −0.306502009 | |
| 218329_at | PRDM4 | 0.004765228 | 0.025023243 | 0.557396992 | −0.288865493 | |
| 202332_at | CSNK1E | 0.004778822 | 0.056814203 | 0.906012249 | −0.31847663 | |
| 221769_at | SPSB3 | 0.004874618 | 0.000873824 | 0.989523303 | −0.473608464 | |
| 211210_x_at | SH2D1A | 0.004944449 | 0.050940897 | 0.947459717 | −0.328965475 | |
| | (includes | | | | | |
| | EG: 4068) | | | | | |
| 218346_s_at | SESN1 | 0.004968862 | 0.182658265 | 0.982927437 | −0.1925237 | |
| 206126_at | BLR1 | 0.004993954 | 0.10315335 | 0.932963506 | −0.392408445 | |
| 203341_at | CEBPZ | 0.005031823 | 0.021988609 | 0.800382255 | −0.382726185 | |
| 209803_s_at | PHLDA2 | 0.005038526 | 0.040009654 | 0.896257222 | 0.485277576 | |
| 217823_s_at | UBE2J1 | 0.005109744 | 0.032982357 | 0.438252502 | 0.307047069 | |
| 209448_at | HTATIP2 | 0.005147167 | 0.039224073 | 0.557025701 | 0.391415598 | |
| 221484_at | B4GALT5 | 0.005160466 | 0.000422525 | 0.970304109 | 0.576135181 | |
| 203593_at | CD2AP | 0.005170123 | 0.044631413 | 0.793788265 | 0.380848049 | |
| 219183_s_at | PSCD4 | 0.005170123 | 0.024712694 | 0.837193604 | 0.278800417 | |
| 208754_s_at | NAP1L1 | 0.005174786 | 0.008597407 | 0.504375257 | −0.327955166 | |
| 210836_x_at | PDE4D | 0.005177082 | 0.054963257 | 0.944102219 | −0.278719955 | |
| 213497_at | ABTB2 | 0.005198329 | 0.080653538 | 0.983164326 | 0.273717046 | |
| 202703_at | DUSP11 | 0.005242435 | 0.018496832 | 0.542658561 | −0.267153293 | |
| 202981_x_at | SIAH1 | 0.005244418 | 0.030827921 | 0.807579104 | −0.329243885 | |
| 206877_at | MXD1 | 0.005244418 | 0.027139794 | 0.989523303 | 0.420763274 | |
| 201386_s_at | DHX15 | 0.005265833 | 0.100367382 | 0.594216566 | −0.214370956 | |
| 208760_at | UBE2I | 0.005299734 | 0.040716879 | 0.932963506 | −0.248417224 | |
| 210031_at | CD247 | 0.005306398 | 0.009376411 | 0.376725352 | −0.393876014 | |
| 218927_s_at | CHST12 | 0.005343153 | 0.05488334 | 0.933809558 | 0.409880547 | |
| 212658_at | LHFPL2 | 0.005343153 | 1.12606E−05 | 0.966121414 | 0.481980455 | |
| 201897_s_at | CKS1B | 0.005355825 | 0.029128323 | 0.552322903 | 0.268726061 | |
| 210190_at | STX11 | 0.005358298 | 0.076481332 | 0.807579104 | 0.339889768 | |
| 204404_at | SLC12A2 | 0.005395374 | 0.026529648 | 0.833458294 | −0.278528948 | |
| 212510_at | GPD1L | 0.005395374 | 0.001992216 | 0.974685401 | −0.548493813 | |
| 205312_at | SPI1 | 0.005444162 | 0.011756501 | 0.952168804 | 0.465937265 | |
| 213848_at | Need to update | 0.005469295 | 0.03290968 | 0.906012249 | 0.289533766 | |
| | annotation | | | | | |
| 211665_s_at | SOS2 | 0.005639289 | 0.080720349 | 0.62004893 | −0.223059239 | |
| 208981_at | PECAM1 | 0.005654459 | 0.14859908 | 0.9404858 | 0.195110953 | |
| 202206_at | ARL4C | 0.005746516 | 0.13182953 | 0.089058602 | −0.242175972 | |
| 204346_s_at | RASSF1 | 0.005746516 | 0.00567005 | 0.679835233 | −0.316787126 | |
| 212218_s_at | FASN | 0.005782194 | 0.032829207 | 0.654429654 | −0.271838734 | |
| 210948_s_at | LEF1 | 0.005850693 | 0.053343511 | 0.848214323 | −0.323253165 | |
| 203665_at | HMOX1 | 0.005926028 | 0.006435669 | 0.558360244 | 0.501438949 | |
| 207677_s_at | NCF4 | 0.005990637 | 0.04118555 | 0.518206857 | 0.444466999 | |
| 210423_s_at | SLC11A1 | 0.006072736 | 0.005255478 | 0.978029446 | 0.461627396 | |
| 218878_s_at | SIRT1 | 0.006099797 | 0.048486242 | 0.711933676 | −0.252398797 | |
| 209782_s_at | DBP | 0.006100528 | 0.002478215 | 0.926817552 | −0.492805369 | |
| 206976_s_at | HSPH1 | 0.006112857 | 0.053406191 | 0.83948366 | −0.386970539 | |
| 201367_s_at | ZFP36L2 | 0.006168213 | 0.200691332 | 0.99162365 | −0.53621983 | |
| 209682_at | CBLB | 0.006171548 | 0.014751333 | 0.480690341 | −0.365162018 | |
| 210139_s_at | PMP22 | 0.006171548 | 0.053657896 | 0.558786143 | −0.648429059 | |
| 221742_at | CUGBP1 | 0.006171548 | 0.229039457 | 0.906722412 | −0.234867174 | |
| 205821_at | KLRK1 | 0.006240933 | 0.005483071 | 0.932963506 | −0.365286687 | |
| 211806_s_at | KCNJ15 | 0.006269218 | 0.042359673 | 0.711933676 | 0.353421684 | |
| 204158_s_at | TCIRG1 | 0.00630195 | 0.024128392 | 0.843903188 | 0.283716907 | |
| 217987_at | ASNSD1 | 0.006347825 | 0.043738517 | 0.724798293 | −0.247609028 | |
| 220175_s_at | CBWD5 | 0.006472153 | 0.262405096 | 0.824385407 | 0.23079063 | |
| 217914_at | TPCN1 | 0.006507875 | 0.020195013 | 0.911573276 | −0.302077637 | |
| 220066_at | NOD2 | 0.006560891 | 0.014162131 | 0.844024603 | 0.414790172 | |
| 218312_s_at | ZSCAN18 | 0.006645837 | 0.037558025 | 0.793788265 | −0.249159845 | |
| 201477_s_at | RRM1 | 0.006657771 | 0.152929649 | 0.925560488 | −0.197192664 | |
| 205239_at | AREG | 0.006662718 | 0.137330159 | 0.793788265 | −0.561714888 | |
| 201529_s_at | RPA1 | 0.006677051 | 0.005239299 | 0.939374883 | −0.319790627 | |
| 204550_x_at | GSTM1 | 0.00668529 | 0.006539863 | 0.519363959 | −0.306251806 | |
| 210754_s_at | LYN | 0.006743374 | 0.102540247 | 0.712482556 | 0.233277739 | |
| 214157_at | GNAS | 0.006783131 | 0.180486724 | 0.937236314 | −0.193755563 | |
| 202232_s_at | EIF3M | 0.006851658 | 0.007992314 | 0.915587288 | −0.350202481 | |
| 203640_at | MBNL2 | 0.006862456 | 0.025241455 | 0.973104905 | −0.375480454 | |
| 200646_s_at | NUCB1 | 0.006865251 | 0.044355722 | 0.846488521 | 0.289261951 | |
| 210660_at | LILRA1 | 0.006865251 | 0.152193981 | 0.904447339 | 0.342439177 | |
| 208926_at | NEU1 | 0.006936416 | 0.01609353 | 0.660094786 | 0.309506084 | |
| 220370_s_at | USP36 | 0.006946096 | 0.036211188 | 0.558360244 | −0.434914018 | |
| 202207_at | ARL4C | 0.007130826 | 0.08975223 | 0.281969259 | −0.211644383 | |
| 212706_at | RASA4 | 0.007130826 | 0.00204532 | 0.873092938 | −0.340570127 | |
| 205049_s_at | CD79A | 0.007212892 | 0.035658714 | 0.85740335 | −0.327567934 | |
| 209240_at | OGT | 0.007263415 | 0.015292598 | 0.838371349 | −0.319554053 | |
| 219374_s_at | ALG9 | 0.007273424 | 0.090701484 | 0.462774646 | −0.245867392 | |
| 212914_at | CBX7 | 0.007273424 | 0.124767142 | 0.705393437 | −0.212077587 | |
| 201635_s_at | FXR1 | 0.007287471 | 0.062042268 | 0.926350227 | −0.325358861 | |
| 207079_s_at | MED6 | 0.007339646 | 0.037804294 | 0.587867787 | −0.343744921 | |
| 209602_s_at | GATA3 | 0.007371787 | 0.008525014 | 0.838839885 | −0.495283623 | |
| 212589_at | SCP2 | 0.007495618 | 0.175147105 | 0.97607782 | −0.315811594 | |
| 205353_s_at | PEBP1 | 0.007518218 | 0.015956229 | 0.926966603 | −0.311886694 | |
| 215719_x_at | FAS | 0.007558593 | 0.008422522 | 0.938509968 | 0.457875965 | |
| 218017_s_at | HGSNAT | 0.00756576 | 0.039403383 | 0.838839885 | −0.370381753 | |
| 207005_s_at | BCL2 | 0.00760915 | 0.126644969 | 0.8856292 | −0.251095968 | |
| 211339_s_at | ITK | 0.00770117 | 0.127563619 | 0.845553812 | −0.230103899 | |
| 210264_at | GPR35 | 0.00775976 | 0.002887818 | 0.82604375 | 0.36415151 | |
| 211530_x_at | HLA-G | 0.00780468 | 0.039928129 | 0.906722412 | 0.347381355 | |
| 203182_s_at | SRPK2 | 0.007822228 | 0.007725788 | 0.824385407 | −0.319818652 | |
| 202085_at | TJP2 | 0.007894163 | 0.019684146 | 0.968262217 | 0.367585114 | |
| 219806_s_at | C11ORF75 | 0.007934301 | 0.13556187 | 0.87331579 | 0.326283676 | |
| 209501_at | CDR2 | 0.007957413 | 0.073840644 | 0.971652444 | −0.259508834 | |
| 212319_at | RUTBC1 | 0.007967542 | 0.039008412 | 0.570133912 | −0.262148486 | |
| 201360_at | CST3 | 0.007967542 | 0.071289231 | 0.77858286 | 0.283221018 | |
| 221206_at | PMS2 | 0.007994284 | 0.014642114 | 0.753411615 | −0.386445278 | |
| 208622_s_at | VIL2 | 0.008035758 | 0.178519882 | 0.518649529 | −0.280919204 | |
| 206978_at | CCR2 | 0.008035758 | 0.057311059 | 0.792060926 | 0.303512327 | |
| 211744_s_at | CD58 | 0.008081567 | 0.007582799 | 0.906012249 | 0.431050257 | |
| 207723_s_at | KLRC3 | 0.008091871 | 0.00099468 | 0.749123103 | −0.519195904 | |
| 212851_at | DCUN1D4 | 0.008222755 | 0.101868562 | 0.879699508 | −0.218403278 | |
| 202988_s_at | RGS1 | 0.008232385 | 0.011435535 | 0.793788265 | −0.815780929 | |
| 204769_s_at | TAP2 | 0.008273972 | 0.026353097 | 0.939653852 | 0.276805843 | |
| 211366_x_at | CASP1 | 0.008295672 | 0.182714573 | 0.725581316 | 0.218033461 | |
| 209476_at | TXNDC1 | 0.008307494 | 0.106919075 | 0.676993566 | 0.225606616 | |
| 213017_at | ABHD3 | 0.008358718 | 0.023223945 | 0.460110071 | 0.475746215 | |
| 200649_at | NUCB1 | 0.008407797 | 0.057311059 | 0.684376681 | 0.309553242 | |
| 221558_s_at | LEF1 | 0.008470173 | 0.022687088 | 0.927054575 | −0.287620386 | |
| 204749_at | NAP1L3 | 0.008529966 | 0.017767982 | 0.765886782 | −0.364788658 | |
| 204951_at | RHOH | 0.008749703 | 0.125931231 | 0.801876388 | −0.252265007 | |
| 202838_at | FUCA1 | 0.008837332 | 0.112301886 | 0.460110071 | 0.209117006 | |
| 203725_at | GADD45A | 0.009040951 | 0.217913452 | 0.686191702 | −0.225484265 | |
| 209569_x_at | D4S234E | 0.009092541 | 0.017130768 | 0.983729727 | −0.322718467 | |
| 209555_s_at | CD36 | 0.009208966 | 0.089960889 | 0.706296724 | 0.395342739 | |
| 207008_at | IL8RB | 0.009246353 | 0.036211188 | 0.576624724 | 0.71152798 | |
| 202589_at | TYMS | 0.009284264 | 0.080653538 | 0.014796277 | 0.384958451 | |
| 207270_x_at | CD300C | 0.009349473 | 0.023705752 | 0.844024603 | 0.38303717 | |
| 207540_s_at | SYK | 0.009362913 | 0.080653538 | 0.493618819 | 0.715540031 | |
| 203068_at | KLHL21 | 0.009415367 | 0.018151619 | 0.314342283 | −0.616327633 | |
| 214366_s_at | ALOX5 | 0.00945614 | 0.047352479 | 0.726031892 | 0.327830344 | |
| 218718_at | PDGFC | 0.009582838 | 0.02613039 | 0.717703472 | −0.32514312 | |
| 206472_s_at | TLE3 | 0.009602301 | 0.022004396 | 0.81186408 | 0.381170905 | |
| 208190_s_at | LSR | 0.009612752 | 0.177315023 | 0.970304109 | −0.476797951 | |
| 201690_s_at | TPD52 | 0.009659332 | 0.023223945 | 0.242802241 | −0.321170027 | |
| 203505_at | ABCA1 | 0.009659332 | 0.003633372 | 0.940578006 | 0.778682584 | |
| 217234_s_at | VIL2 | 0.009779784 | 0.099668492 | 0.686973252 | −0.329869246 | |
| 209970_x_at | CASP1 | 0.0098609 | 0.204521053 | 0.613727458 | 0.214014925 | |
| 204957_at | ORC5L | 0.009879341 | 0.059327291 | 0.937261364 | −0.26845207 | |
| 213357_at | GTF2H5 | 0.010110733 | 0.041397161 | 0.086816858 | 0.3978722 | |
| 201879_at | ARIH1 | 0.010263909 | 0.35973853 | 0.480690341 | −0.139471305 | |
| 211138_s_at | KMO | 0.010276714 | 0.088723342 | 0.809600216 | 0.290804418 | |
| 212439_at | IHPK1 | 0.010525524 | 0.127493222 | 0.677299865 | −0.275597596 | |
| 214438_at | HLX | 0.010594965 | 0.04826413 | 0.970304109 | 0.2904212 | |
| 214039_s_at | LAPTM4B | 0.010716792 | 0.028151767 | 0.924278945 | −0.287201922 | |
| 218636_s_at | MAN1B1 | 0.010849901 | 0.325367749 | 0.996248165 | −0.242056143 | |
| 209369_at | ANXA3 | 0.010875364 | 0.032995797 | 0.01525423 | 0.640563446 | |
| 201749_at | ECE1 | 0.011154306 | 0.005050996 | 0.861456705 | 0.403999243 | |
| 202393_s_at | KLF10 | 0.011226627 | 0.084689941 | 0.922862728 | 0.303796756 | |
| 213278_at | MTMR9 | 0.011228946 | 0.023880247 | 0.793788265 | −0.287287315 | |
| 204294_at | AMT (includes | 0.011246251 | 0.106919075 | 0.884660098 | −0.218131032 | |
| | EG: 275) | | | | | |
| 220086_at | IKZF5 | 0.011526475 | 0.12360589 | 0.671515228 | −0.22770966 | |
| 215731_s_at | MPHOSPH9 | 0.011630638 | 0.00529338 | 0.867793855 | −0.704486948 | |
| 204562_at | IRF4 | 0.011634495 | 0.088670705 | 0.454165885 | −0.253054268 | |
| 203723_at | ITPKB | 0.011634495 | 0.071289231 | 0.853800604 | −0.340859709 | |
| 217957_at | C16ORF80 | 0.011680989 | 0.176732619 | 0.745247946 | −0.306153713 | |
| 212447_at | KBTBD2 | 0.011756298 | 0.047553128 | 0.757749921 | −0.261216593 | |
| 211991_s_at | HLA-DPA1 | 0.011756298 | 0.371927664 | 0.947786651 | 0.142001843 | |
| 204622_x_at | NR4A2 | 0.011798072 | 0.051467095 | 0.721821849 | −0.408511875 | |
| 221826_at | ANGEL2 | 0.012244732 | 0.143126266 | 0.846591986 | −0.229164026 | |
| 201301_s_at | ANXA4 | 0.012247414 | 0.049001016 | 0.570133912 | 0.858211651 | |
| 204336_s_at | RGS19 | 0.01228945 | 0.190075069 | 0.906722412 | 0.191451565 | |
| 213539_at | CD3D | 0.012364628 | 0.006230433 | 0.937164918 | −0.337874427 | |
| 213672_at | MARS | 0.012445306 | 0.038312345 | 0.850755602 | −0.431723776 | |
| | (includes | | | | | |
| | EG: 4141) | | | | | |
| 210766_s_at | CSE1L | 0.012544794 | 0.079390663 | 0.906012249 | −0.253562708 | |
| 208890_s_at | PLXNB2 | 0.012572426 | 0.42023393 | 0.600791844 | 0.157960485 | |
| 211883_x_at | CEACAM1 | 0.012603414 | 0.060483745 | 0.54126922 | 0.312243791 | |
| 208880_s_at | PRPF6 | 0.012603414 | 0.016609627 | 0.921230073 | −0.407193108 | |
| 213145_at | FBXL14 | 0.012736544 | 0.015214306 | 0.643328545 | −0.300748427 | |
| 207419_s_at | RAC2 | 0.012755037 | 0.100111799 | 0.554763569 | 0.272077943 | |
| 215210_s_at | DLST | 0.012789654 | 0.182456166 | 0.770047411 | −0.221021784 | |
| 209773_s_at | RRM2 | 0.012867343 | 0.120778597 | 0.403965698 | 0.30763863 | |
| 206255_at | BLK | 0.012961723 | 0.163040248 | 0.570133912 | −0.43735596 | |
| 212346_s_at | MXD4 | 0.01304429 | 0.191875661 | 0.504375257 | −0.227045717 | |
| 210818_s_at | BACH1 | 0.013249311 | 0.005483071 | 0.77678859 | 0.340649029 | |
| 202100_at | RALB | 0.013424653 | 0.13940172 | 0.717703472 | 0.270826629 | |
| 221920_s_at | SLC25A37 | 0.013424653 | 0.039008412 | 0.825177207 | 0.499252449 | |
| 209845_at | MKRN1 | 0.013424653 | 0.055782899 | 0.970304109 | 0.37410074 | |
| 221569_at | AHI1 | 0.013470953 | 0.137381622 | 0.903665108 | −0.202496009 | |
| 222026_at | RBM3 | 0.01350236 | 0.007642761 | 0.570133912 | −0.450979832 | |
| 214455_at | HIST1H2BC | 0.013533356 | 0.035658714 | 0.262239522 | 0.564730366 | |
| 202426_s_at | RXRA | 0.013568315 | 0.058293142 | 0.554763569 | 0.330293309 | |
| 219089_s_at | ZNF576 | 0.013634698 | 0.081784947 | 0.878689864 | −0.228182027 | |
| 201302_at | ANXA4 | 0.013697532 | 0.08084446 | 0.422140415 | 0.40111778 | |
| 218645_at | ZNF277P | 0.01375991 | 0.009000926 | 0.932963506 | −0.332320974 | |
| 203678_at | MTMR15 | 0.014018481 | 0.014288107 | 0.809600216 | −0.418966858 | |
| 208744_x_at | HSPH1 | 0.014027881 | 0.247709375 | 0.927054575 | −0.435330989 | |
| 217967_s_at | FAM129A | 0.014091343 | 0.346939576 | 0.82604375 | 0.214696528 | |
| 213204_at | PARC | 0.014314457 | 0.126756122 | 0.884660098 | −0.359478603 | |
| 207568_at | CHRNA6 | 0.01431841 | 0.010429417 | 0.830719817 | −0.325429211 | |
| 209930_s_at | NFE2 | 0.01441225 | 0.034636644 | 0.891670708 | 0.400069337 | |
| 210224_at | MR1 | 0.014421369 | 0.20627165 | 0.649409254 | 0.218885928 | |
| 212231_at | FBXO21 | 0.014440182 | 0.108656399 | 0.567915232 | −0.232315789 | |
| 214326_x_at | JUND | 0.014553654 | 0.236385207 | 0.891953978 | −0.251108764 | |
| 206118_at | STAT4 | 0.01470608 | 0.049001016 | 0.689229599 | −0.266212673 | |
| 222115_x_at | N-PAC | 0.014716395 | 0.051170918 | 0.819456917 | −0.310590511 | |
| 213674_x_at | IGHD | 0.014911014 | 0.039938777 | 0.695073101 | −0.501844766 | |
| 219777_at | GIMAP6 | 0.015043577 | 0.144407805 | 0.570133912 | 0.326848663 | |
| 221601_s_at | FAIM3 | 0.015047614 | 0.027336598 | 0.885856306 | −0.305777039 | |
| 209118_s_at | TUBA1A | 0.015056809 | 0.128749695 | 0.962540421 | 0.233704948 | |
| 201695_s_at | NP | 0.015304243 | 7.1509E−05 | 0.978029446 | 0.504320958 | |
| 214945_at | LOC202134 | 0.015493442 | 0.232239839 | 0.885856306 | −0.237166546 | |
| 210629_x_at | LST1 | 0.015774191 | 0.183606534 | 0.906722412 | 0.197284912 | |
| 212569_at | SMCHD1 | 0.015774191 | 0.087852473 | 0.940595209 | 0.30797767 | |
| 205822_s_at | HMGCS1 | 0.015852397 | 0.059327291 | 0.997185403 | −0.304518474 | |
| 209567_at | RRS1 | 0.015886509 | 0.083999076 | 0.980264971 | −0.348378346 | |
| 219534_x_at | CDKN1C | 0.015943545 | 0.463007545 | 0.866775981 | 0.200589944 | |
| 218309_at | CAMK2N1 | 0.015984508 | 0.009605583 | 0.320340002 | −0.400982962 | |
| 201566_x_at | ID2 | 0.016142401 | 0.057909009 | 0.922875053 | 0.359282319 | |
| 201701_s_at | PGRMC2 | 0.016207876 | 0.02715706 | 0.902614556 | −0.38800159 | |
| 205997_at | ADAM28 | 0.016219595 | 0.251515075 | 0.885856306 | −0.255301646 | |
| 202201_at | BLVRB | 0.016264535 | 0.040866126 | 0.90587437 | 0.387417159 | |
| 213839_at | KIAA0500 | 0.01628162 | 0.039648402 | 0.905763968 | −0.396441068 | |
| 206011_at | CASP1 | 0.016827593 | 0.12414678 | 0.60494547 | 0.286452892 | |
| 221748_s_at | TNS1 | 0.016871581 | 0.017998525 | 0.973546798 | 0.488992364 | |
| 208621_s_at | VIL2 | 0.016908109 | 0.078950734 | 0.631444377 | −0.34500341 | |
| 211367_s_at | CASP1 | 0.016963127 | 0.346529064 | 0.717703472 | 0.220770374 | |
| 202595_s_at | LEPROTL1 | 0.017055141 | 0.134342766 | 0.584976844 | −0.246822942 | |
| 218517_at | PHF17 | 0.01729529 | 0.126277973 | 0.853800604 | −0.229045499 | |
| 211902_x_at | TRA@ | 0.017446858 | 0.022184928 | 0.865251659 | −0.380605249 | |
| 204860_s_at | NAIP | 0.017450484 | 0.136220241 | 0.824328734 | 0.302867609 | |
| 211963_s_at | ARPC5 | 0.01776995 | 0.098966874 | 0.652902586 | 0.249707545 | |
| 213353_at | ABCA5 | 0.01776995 | 0.158870096 | 0.884660098 | −0.23474082 | |
| 210116_at | SH2D1A | 0.01776995 | 0.065442642 | 0.936853005 | −0.33592137 | |
| | (includes | | | | | |
| | EG: 4068) | | | | | |
| 222251_s_at | GMEB2 | 0.017802241 | 0.102730031 | 0.376725352 | −0.234073959 | |
| 202626_s_at | LYN | 0.017816929 | 0.211441079 | 0.552322903 | 0.195225713 | |
| 202943_s_at | NAGA | 0.018177789 | 0.186012025 | 0.981360886 | 0.206473896 | |
| 203923_s_at | CYBB | 0.018351402 | 0.168111748 | 0.552322903 | 0.303131083 | |
| 207075_at | NLRP3 | 0.018351402 | 0.013167345 | 0.915663453 | 0.591472979 | |
| 214744_s_at | RPL23 | 0.018351402 | 0.011123203 | 0.992169204 | −0.334250799 | |
| 205745_x_at | ADAM17 | 0.018388943 | 0.005935136 | 0.827347393 | 0.374950593 | |
| 221485_at | B4GALT5 | 0.018388943 | 0.005379129 | 0.932963506 | 0.555017947 | |
| 201487_at | CTSC | 0.018628959 | 0.151874357 | 0.943771576 | 0.249401114 | |
| 221653_x_at | APOL2 | 0.018694504 | 0.169194339 | 0.629434643 | 0.504841584 | |
| 212769_at | TLE3 | 0.01897878 | 0.039008412 | 0.846488521 | 0.342859284 | |
| 218130_at | C17ORF62 | 0.019043437 | 0.133051813 | 0.893435169 | 0.472265875 | |
| 206214_at | PLA2G7 | 0.019147471 | 0.442198685 | 0.803159361 | 0.190235824 | |
| 215786_at | RSF1 | 0.019151091 | 0.49890241 | 0.899991236 | −0.188609275 | |
| 210792_x_at | SIVA1 | 0.019180223 | 0.070955978 | 0.45663747 | −0.258975831 | |
| 203751_x_at | JUND | 0.019180223 | 0.297742427 | 0.680969676 | −0.210364435 | |
| 216942_s_at | CD58 | 0.019188421 | 0.042713549 | 0.940468793 | 0.361798864 | |
| 205173_x_at | CD58 | 0.019311744 | 0.032221551 | 0.907359477 | 0.412717957 | |
| 202104_s_at | SPG7 | 0.019426762 | 0.187298695 | 0.933418803 | −0.229675114 | |
| 207490_at | TUBA4 | 0.019451966 | 0.128275331 | 0.864671393 | 0.236332987 | |
| 222315_at | Need to update | 0.019489922 | 0.004351803 | 0.845163449 | −0.65783036 | |
| | annotation | | | | | |
| 210356_x_at | MS4A1 | 0.019789714 | 0.087296713 | 0.636658025 | −0.363890781 | |
| 204655_at | CCL5 | 0.01983136 | 0.002547452 | 0.940129298 | −0.435049716 | |
| 211560_s_at | ALAS2 | 0.01989019 | 0.026441636 | 0.980673033 | 0.946356737 | |
| 205292_s_at | HNRPA2B1 | 0.019939501 | 0.159812536 | 0.404804753 | 0.256446785 | |
| 203845_at | PCAF | 0.020259923 | 0.067897502 | 0.775176914 | 0.269051192 | |
| 218522_s_at | MAP1S | 0.020393984 | 0.013344213 | 0.613727458 | 0.328985626 | |
| 201780_s_at | RNF13 | 0.020532722 | 0.102540247 | 0.86194884 | 0.274624257 | |
| 221234_s_at | BACH2 | 0.020667873 | 0.062697542 | 0.923176795 | −0.267014013 | |
| 203313_s_at | TGIF1 | 0.020895853 | 0.366896505 | 0.668753123 | −0.165433985 | |
| 211101_x_at | LILRA2 | 0.020895853 | 0.10139137 | 0.996581247 | 0.250662434 | |
| 204821_at | BTN3A3 | 0.021021782 | 0.362114581 | 0.811510087 | 0.214935123 | |
| 210895_s_at | CD86 | 0.021148346 | 0.109013424 | 0.634508876 | 0.25389383 | |
| 202284_s_at | CDKN1A | 0.02176265 | 0.108969489 | 0.436770009 | 0.264668049 | |
| 205987_at | CD1C | 0.02176265 | 0.078348501 | 0.800382255 | −0.503826416 | |
| 213475_s_at | ITGAL | 0.02176265 | 0.53362722 | 0.808230912 | 0.124232857 | |
| 203092_at | TIMM44 | 0.02176265 | 0.223418785 | 0.859477285 | −0.349971753 | |
| 217142_at | Need to update | 0.022030272 | 0.006421353 | 0.804966785 | −0.614240987 | |
| | annotation | | | | | |
| 36564_at | IBRDC3 | 0.022043675 | 0.042251555 | 0.985411674 | 0.318898723 | |
| 214992_s_at | DNASE2 | 0.0221327 | 0.244921515 | 0.710824574 | 0.192600667 | |
| 207686_s_at | CASP8 | 0.022251637 | 0.293295305 | 0.761190158 | −0.216609555 | |
| 217418_x_at | MS4A1 | 0.022292129 | 0.102810003 | 0.689284686 | −0.381015062 | |
| 203535_at | S100A9 | 0.022328545 | 0.081536361 | 0.543044503 | 0.276794159 | |
| 218153_at | CARS2 | 0.022470077 | 0.368914376 | 0.646290307 | 0.150673741 | |
| 204445_s_at | ALOX5 | 0.022497073 | 0.160127618 | 0.542658561 | 0.224758249 | |
| 208782_at | FSTL1 | 0.022858433 | 0.174757062 | 0.983352528 | −0.228315866 | |
| 216609_at | TXN | 0.023172694 | 0.099122148 | 0.741144916 | 0.354826262 | |
| 210379_s_at | TLK1 | 0.023300636 | 0.044336207 | 0.93197518 | −0.678196701 | |
| 221210_s_at | NPL | 0.023711476 | 0.082081555 | 0.654429654 | 0.429266523 | |
| 204168_at | MGST2 | 0.023788349 | 0.030439266 | 0.854833543 | −0.379724773 | |
| 214790_at | SENP6 | 0.024264859 | 0.074582758 | 0.710647678 | −0.2848028 | |
| 205639_at | AOAH | 0.024264859 | 0.152121537 | 0.884660098 | 0.238715067 | |
| 209066_x_at | UQCRB | 0.024355364 | 0.051844029 | 0.994302012 | −0.34577675 | |
| | (includes | | | | | |
| | EG: 7381) | | | | | |
| 201560_at | CLIC4 | 0.024378822 | 0.284723127 | 0.922875053 | 0.183824393 | |
| 209344_at | TPM4 | 0.024492869 | 0.018168174 | 0.954560682 | 0.35789861 | |
| 217580_x_at | ARL6IP2 | 0.024590772 | 0.032151111 | 0.99363955 | −0.32492921 | |
| 201662_s_at | ACSL3 | 0.024628161 | 0.022571085 | 0.708542801 | 0.378306535 | |
| 218404_at | SNX10 | 0.024763042 | 0.173031608 | 0.613727458 | 0.257277393 | |
| 202789_at | PLCG1 | 0.024827653 | 0.08903096 | 0.927054575 | −0.254047489 | |
| 210424_s_at | GOLGA8A | 0.025087642 | 0.028000613 | 0.809600216 | −0.426691788 | |
| 205715_at | BST1 | 0.025114072 | 0.150950206 | 0.804966785 | 0.307260943 | |
| 202599_s_at | NRIP1 | 0.025357862 | 0.019222851 | 0.966794706 | 0.486469213 | |
| 206765_at | KCNJ2 | 0.02570662 | 0.059497978 | 0.613727458 | 0.581853261 | |
| 201070_x_at | SF3B1 | 0.026391529 | 0.294935988 | 0.995153496 | −0.221864528 | |
| 214244_s_at | ATP6V0E1 | 0.026394427 | 0.063783265 | 0.741625882 | 0.329307349 | |
| 213183_s_at | CDKN1C | 0.026539963 | 0.274313528 | 0.726031892 | 0.226276402 | |
| 201348_at | GPX3 | 0.026686091 | 0.082081555 | 0.666831456 | −0.365530362 | |
| 202861_at | PER1 | 0.026726313 | 0.2482877 | 0.658726886 | −0.242651891 | |
| 202636_at | RNF103 | 0.027112829 | 0.293009431 | 0.706296724 | −0.218783657 | |
| 215223_s_at | SOD2 | 0.027124769 | 0.100361826 | 0.927054575 | 0.329399145 | |
| 212894_at | SUPV3L1 | 0.027172687 | 0.073524055 | 0.460110071 | −0.268038645 | |
| 214148_at | Need to update | 0.027444722 | 0.178491981 | 0.955997488 | −0.212981318 | |
| | annotation | | | | | |
| 204520_x_at | BRD1 | 0.027460368 | 0.189992666 | 0.460110071 | −0.255287237 | |
| 209671_x_at | TRA@ | 0.027947129 | 0.150767177 | 0.857260432 | −0.308300427 | |
| 216015_s_at | NLRP3 | 0.028162825 | 0.086398294 | 0.633838349 | 0.436473324 | |
| 202746_at | ITM2A | 0.028309925 | 0.232580835 | 0.980264971 | −0.216105082 | |
| 214582_at | PDE3B | 0.028389236 | 0.054662177 | 0.926914122 | −0.303819563 | |
| 202901_x_at | CTSS | 0.029554042 | 0.413823405 | 0.885856306 | 0.187087835 | |
| 206130_s_at | ASGR2 | 0.029889904 | 0.263030666 | 0.631444377 | 0.208922768 | |
| 205689_at | PCNXL2 | 0.029929092 | 0.069781211 | 0.980808943 | −0.311434983 | |
| | (includes | | | | | |
| | EG: 80003) | | | | | |
| 209161_at | PRPF4 | 0.029963806 | 0.215112626 | 0.716868136 | −0.206140316 | |
| 213370_s_at | SFMBT1 | 0.030436666 | 0.111978848 | 0.944102219 | −0.273940987 | |
| 204642_at | EDG1 | 0.030530732 | 0.109209362 | 0.863302136 | −0.333674522 | |
| 209561_at | THBS3 | 0.0307313 | 0.25458731 | 0.996581247 | −0.205332375 | |
| 203148_s_at | TRIM14 | 0.03079373 | 0.15413446 | 0.493618819 | 0.369444372 | |
| 206700_s_at | JARID1D | 0.03079373 | 0.03290968 | 0.974320691 | −0.510194162 | |
| 211368_s_at | CASP1 | 0.031088222 | 0.351223201 | 0.587867787 | 0.194796989 | |
| 206715_at | TFEC | 0.031692073 | 0.182305616 | 0.878184512 | 0.262716165 | |
| 220386_s_at | EML4 | 0.032071275 | 0.240072037 | 0.703685034 | −0.232496456 | |
| 218152_at | HMG20A | 0.032071275 | 0.025023243 | 0.87331579 | −0.331804799 | |
| 221428_s_at | TBL1XR1 | 0.032071275 | 0.218882785 | 0.926879061 | −0.219118973 | |
| 205685_at | CD86 | 0.03213621 | 0.133433486 | 0.766023636 | 0.288434558 | |
| 203081_at | CTNNBIP1 | 0.032230779 | 0.070955978 | 0.955860316 | −0.300167763 | |
| 215087_at | C15ORF39 | 0.032462237 | 0.137330159 | 0.929077542 | 0.249938638 | |
| 211702_s_at | USP32 | 0.032740923 | 0.019939423 | 0.944102219 | 0.329211319 | |
| 218711_s_at | SDPR | 0.032753899 | 0.082301542 | 0.83826875 | −0.307944714 | |
| 218963_s_at | KRT23 | 0.032909054 | 0.171437026 | 0.964140878 | 0.590754653 | |
| 219007_at | NUP43 | 0.033280508 | 0.040928464 | 0.542658561 | −0.409889901 | |
| 213291_s_at | UBE3A | 0.033740756 | 0.217913452 | 0.72033594 | −0.228700873 | |
| 204326_x_at | MT1X | 0.033740756 | 0.073126948 | 0.916817284 | 0.297075777 | |
| 217739_s_at | PBEF2 | 0.033740756 | 0.097661052 | 0.938509968 | 0.314295378 | |
| 217755_at | HN1 | 0.034086039 | 0.318045852 | 0.904036627 | 0.182399518 | |
| 217985_s_at | BAZ1A | 0.03466727 | 0.206293395 | 0.83681131 | 0.546330304 | |
| 220939_s_at | DPP8 | 0.034720358 | 0.118168782 | 0.90275246 | −0.283309897 | |
| 201313_at | ENO2 | 0.034816907 | 0.103747652 | 0.940613622 | −0.263537736 | |
| 215533_s_at | UBE4B | 0.034875223 | 0.071884369 | 0.966527259 | −0.305565485 | |
| 204820_s_at | BTN3A3 | 0.035010794 | 0.234492658 | 0.570645923 | 0.233894373 | |
| 209604_s_at | GATA3 | 0.035010794 | 0.189352776 | 0.773450053 | −0.343302452 | |
| 216153_x_at | RECK | 0.035010794 | 0.242887888 | 0.92934451 | 0.256080351 | |
| 202643_s_at | TNFAIP3 | 0.035347675 | 0.288366984 | 0.636658025 | −0.218475172 | |
| 218592_s_at | CECR5 | 0.035395293 | 0.026647711 | 0.856352566 | −0.324706701 | |
| 209128_s_at | SART3 | 0.035427345 | 0.786783631 | 0.544104436 | −0.060928286 | |
| 201297_s_at | MOBKL1B | 0.035484934 | 0.191007054 | 0.613727458 | 0.235466714 | |
| 218918_at | MAN1C1 | 0.035919274 | 0.126282967 | 0.937164918 | −0.253876962 | |
| 219073_s_at | OSBPL10 | 0.036604792 | 0.093148343 | 0.33827973 | −0.342811873 | |
| 202481_at | DHRS3 | 0.036854609 | 0.244931292 | 0.915663453 | −0.218874569 | |
| 221060_s_at | TLR4 | 0.036961275 | 0.069577792 | 0.734889789 | 0.365264223 | |
| 205488_at | GZMA | 0.037325756 | 0.294992587 | 0.460110071 | 0.337484953 | |
| 218532_s_at | FAM134B | 0.037325756 | 0.226426426 | 0.690696537 | −0.243620882 | |
| 202742_s_at | PRKACB | 0.03742914 | 0.092656285 | 0.496888495 | −0.295415741 | |
| 221698_s_at | CLEC7A | 0.037452387 | 0.057086696 | 0.922190084 | 0.297635075 | |
| 204618_s_at | GABPB2 | 0.037591714 | 0.381825503 | 0.896710842 | −0.218042909 | |
| 210972_x_at | TRA@ | 0.037637249 | 0.109209362 | 0.885856306 | −0.286575049 | |
| 203046_s_at | TIMELESS | 0.03767595 | 0.11248276 | 0.840058778 | 0.32358318 | |
| 201921_at | GNG10 | 0.037687753 | 0.252397888 | 0.630710872 | 0.224649839 | |
| 214152_at | CCPG1 | 0.037744945 | 0.162408905 | 0.74501549 | 0.276041521 | |
| 206267_s_at | MATK | 0.037846145 | 0.047433804 | 0.711933676 | −0.31260423 | |
| 214091_s_at | GPX3 | 0.038059023 | 0.22479257 | 0.710824574 | −0.294139182 | |
| 212764_at | Need to update | 0.03856123 | 0.195211631 | 0.921230073 | −0.397047724 | |
| | annotation | | | | | |
| 47560_at | LPHN1 | 0.039036499 | 0.40178772 | 0.745384696 | −0.164877039 | |
| 204148_s_at | ZP3 | 0.039246736 | 0.144924218 | 0.867793855 | −0.286257858 | |
| 204116_at | IL2RG | 0.039543072 | 0.144407805 | 0.761190158 | 0.314711363 | |
| 214995_s_at | APOBEC3F | 0.039953312 | 0.239878061 | 0.710647678 | 0.271497898 | |
| 212533_at | WEE1 | 0.040140137 | 0.115441586 | 0.846591986 | −0.427512664 | |
| 201930_at | MCM6 | 0.040202104 | 0.135773151 | 0.878689864 | −0.303067431 | |
| 204491_at | PDE4D | 0.040240856 | 0.353176062 | 0.543105461 | −0.18749664 | |
| 211764_s_at | UBE2D1 | 0.040536755 | 0.195360834 | 0.80941741 | 0.221343709 | |
| 210837_s_at | PDE4D | 0.040834283 | 0.123837945 | 0.631444377 | −0.283111624 | |
| 202030_at | BCKDK | 0.040834283 | 0.117548398 | 0.881158079 | 0.266327295 | |
| 216387_x_at | Need to update | 0.040910517 | 0.024403587 | 0.970304109 | −0.472198243 | |
| | annotation | | | | | |
| 200596_s_at | EIF3A | 0.041091878 | 0.076632972 | 0.998154228 | −0.365016425 | |
| 202205_at | VASP | 0.041506462 | 0.045384808 | 0.830933046 | 0.366499287 | |
| 202594_at | LEPROTL1 | 0.041603383 | 0.220538122 | 0.663534687 | −0.215771141 | |
| 211575_s_at | UBE3A | 0.041691347 | 0.224064374 | 0.702016627 | −0.274002243 | |
| 209206_at | SEC22B | 0.041816139 | 0.090689696 | 0.294369359 | 0.319067462 | |
| 203504_s_at | ABCA1 | 0.042178608 | 0.011718661 | 0.826503176 | 0.550771084 | |
| 213649_at | SFRS7 | 0.042304017 | 0.136220241 | 0.884660098 | −0.259117862 | |
| 202723_s_at | FOXO1 | 0.042304017 | 0.21264104 | 0.927625491 | −0.292781981 | |
| 209818_s_at | HABP4 | 0.042636263 | 0.33203729 | 0.945676094 | −0.311322134 | |
| 208792_s_at | CLU | 0.042931149 | 0.144123259 | 0.726166985 | 0.276378538 | |
| 216894_x_at | CDKN1C | 0.042970438 | 0.297130477 | 0.814313307 | 0.293525643 | |
| 204572_s_at | PIN4 | 0.043371704 | 0.088869941 | 0.800734652 | −0.294104374 | |
| 201909_at | RPS4Y1 | 0.04363416 | 0.663971605 | 0.896681815 | −0.100566885 | |
| 204411_at | KIF21B | 0.043854222 | 0.07765649 | 0.570133912 | −0.427487501 | |
| 212239_at | PIK3R1 | 0.044194887 | 0.213451154 | 0.670488531 | −0.287257475 | |
| 212484_at | FAM89B | 0.044430061 | 0.06670052 | 0.852171473 | 0.344627775 | |
| 203542_s_at | KLF9 | 0.044646893 | 0.331308769 | 0.971652444 | −0.218842169 | |
| 203005_at | LTBR | 0.045172875 | 0.112342861 | 0.809600216 | 0.286047045 | |
| 203589_s_at | TFDP2 | 0.045666747 | 0.66361192 | 0.658726886 | −0.107291109 | |
| 212003_at | C1ORF144 | 0.045996858 | 0.038203947 | 0.947479541 | 0.422374051 | |
| 202531_at | IRF1 | 0.04657947 | 0.196576193 | 0.997185403 | 0.23465268 | |
| 205147_x_at | NCF4 | 0.046735336 | 0.122418354 | 0.745384696 | 0.300343046 | |
| 210164_at | GZMB | 0.046934401 | 0.479543269 | 0.939653852 | 0.183105831 | |
| 216248_s_at | NR4A2 | 0.047462798 | 0.123672921 | 0.708968475 | −0.342837018 | |
| 201486_at | RCN2 | 0.047614506 | 0.189705454 | 0.980264971 | −0.246724101 | |
| 41577_at | PPP1R16B | 0.047627651 | 0.212046833 | 0.63027463 | −0.278401921 | |
| 214257_s_at | SEC22B | 0.048396634 | 0.17845009 | 0.281969259 | 0.247587803 | |
| 205019_s_at | VIPR1 | 0.048510692 | 0.168196372 | 0.943771576 | −0.241780228 | |
| 206934_at | SIRPB1 | 0.049103457 | 0.213729478 | 0.761190158 | 0.366059397 | |
| 212309_at | CLASP2 | 0.049281475 | 0.261467089 | 0.983164326 | −0.222347475 | |
| 202637_s_at | ICAM1 | 0.049617325 | 0.186713735 | 0.778166718 | 0.324877483 | |
| 201876_at | PON2 | 0.049717196 | 0.094257318 | 0.911573276 | −0.285845859 | |
| 203408_s_at | SATB1 | 0.049974155 | 0.206310608 | 0.998615467 | −0.239211025 | |

TABLE 5
Subgroup Y Genes and Metrics

| | | | | FDR | |
|---|---|---|---|---|---|
| AFFYMETRIX | | FDR | FDR | Exacer'n v | |
| HG-U133A | | Exacer'n v Quiet, | Exacer'n v Quiet, | Follow-up, | Mean Δ log2 |
| Probe set ID | Gene | N = 64 | N = 51 | N = 51 | Exacer'n v Quiet |
| 205267_at | POU2AF1 | 6.09889E−06 | 4.91347E−05 | 0.989449741 | 0.506602032 |
| 204571_x_at | PIN4 | 9.20878E−06 | 0.000269021 | 0.989449741 | 0.32332882 |
| 202546_at | VAMP8 | 9.20878E−06 | 0.000469175 | 0.989449741 | 0.271926692 |
| 204683_at | ICAM2 | 1.11155E−05 | 0.00029751 | 0.989449741 | 0.332416528 |
| 208680_at | PRDX1 | 1.11155E−05 | 0.000390432 | 0.989449741 | 0.296295802 |
| 213620_s_at | ICAM2 | 1.23038E−05 | 0.000307743 | 0.989449741 | 0.322945755 |
| 203259_s_at | HDDC2 | 1.56333E−05 | 0.000169818 | 0.989449741 | 0.296766769 |
| 200046_at | DAD1 | 1.78084E−05 | 0.000469175 | 0.989449741 | 0.273165135 |
| 201726_at | ELAVL1 | 1.78084E−05 | 0.00029751 | 0.989449741 | 0.345713605 |
| 208818_s_at | COMT | 1.8168E−05 | 0.000725864 | 0.989449741 | 0.328690658 |
| 206111_at | RNASE2 | 1.8168E−05 | 0.000931464 | 0.989449741 | 0.445855302 |
| 201746_at | TP53 | 1.8168E−05 | 0.000228137 | 0.989449741 | 0.309465491 |
| 218747_s_at | TAPBPL | 2.31671E−05 | 0.000269021 | 0.989449741 | 0.377510405 |
| 219505_at | CECR1 | 2.36519E−05 | 0.000713756 | 0.989449741 | 0.398952771 |
| 201240_s_at | SPCS2 | 2.57282E−05 | 0.000228137 | 0.989449741 | 0.470592216 |
| 210427_x_at | ANXA2 | 2.73645E−05 | 0.000937591 | 0.989449741 | 0.328866724 |
| 212577_at | SMCHD1 | 3.31201E−05 | 0.000689198 | 0.994302976 | −0.319379373 |
| 204116_at | IL2RG | 3.33315E−05 | 0.000206001 | 0.989449741 | 0.521996243 |
| 212827_at | IGHM | 3.50226E−05 | 0.001238391 | 0.989449741 | 0.349524739 |
| 208858_s_at | FAM62A | 3.91712E−05 | 0.00029751 | 0.989449741 | 0.487341826 |
| 209374_s_at | IGHM | 3.91712E−05 | 0.001424129 | 0.989449741 | 0.360281972 |
| 200661_at | CTSA | 4.21575E−05 | 0.001190689 | 0.989449741 | 0.233015986 |
| 213603_s_at | RAC2 | 4.21575E−05 | 0.000269021 | 0.989449741 | 0.283090983 |
| 203828_s_at | IL32 | 4.52678E−05 | 0.000635413 | 0.989449741 | 0.760887517 |
| 212175_s_at | AK2 | 4.68725E−05 | 0.000469175 | 0.989449741 | 0.281226543 |
| 201590_x_at | ANXA2 | 5.18784E−05 | 0.001415219 | 0.989449741 | 0.312862095 |
| 210644_s_at | LAIR1 | 5.18784E−05 | 0.000689198 | 0.989449741 | 0.321130978 |
| 32209_at | FAM89B | 6.26777E−05 | 0.000760418 | 0.989449741 | 0.295826718 |
| 213503_x_at | ANXA2 | 6.47265E−05 | 0.001632865 | 0.989449741 | 0.317729923 |
| 216984_x_at | IGL@ | 7.43565E−05 | 0.000466147 | 0.989449741 | 0.642913797 |
| 201302_at | ANXA4 | 7.73652E−05 | 0.001190689 | 0.989449741 | 0.476997399 |
| 202655_at | ARMET | 9.26164E−05 | 0.00048625 | 0.989449741 | 0.278663 |
| 201897_s_at | CKS1B | 9.26164E−05 | 0.000390432 | 0.999875537 | 0.295834243 |
| 211645_x_at | 0 | 9.30977E−05 | 0.001190689 | 0.989449741 | 0.465162816 |
| 200846_s_at | PPP1CA | 9.30977E−05 | 0.000689198 | 0.989449741 | 0.324223639 |
| 204279_at | PSMB9 | 9.30977E−05 | 0.001922581 | 0.989449741 | 0.359917688 |
| 200789_at | ECH1 | 0.000107598 | 0.001922581 | 0.989449741 | 0.250183945 |
| 203466_at | MPV17 | 0.000113201 | 0.000713756 | 0.999875537 | 0.691493338 |
| 205488_at | GZMA | 0.000120505 | 0.00029751 | 0.994317186 | 0.673401211 |
| 218026_at | CCDC56 | 0.000122403 | 0.001424129 | 0.989449741 | 0.279565664 |
| 204839_at | POP5 | 0.000122403 | 0.001190689 | 0.989449741 | 0.418721294 |
| 212569_at | SMCHD1 | 0.000122403 | 0.00325328 | 0.999875537 | −0.331795389 |
| 218746_at | TAPBPL | 0.000122403 | 0.001765977 | 0.989449741 | 0.321120498 |
| 204563_at | SELL | 0.000133444 | 0.001424129 | 0.989449741 | 0.307941715 |
| 209539_at | ARHGEF6 | 0.000137065 | 0.001827858 | 0.999170597 | 0.247520218 |
| 38241_at | BTN3A3 | 0.000137065 | 0.001771005 | 0.999875537 | 0.303236768 |
| 205081_at | CRIP1 | 0.000137065 | 0.002477145 | 0.999170597 | 0.257126487 |
| 221081_s_at | DENND2D | 0.000137065 | 0.001930541 | 0.989449741 | 0.464622601 |
| 204834_at | FGL2 | 0.000137065 | 0.002525693 | 0.989449741 | 0.648945004 |
| 213357_at | GTF2H5 | 0.000137065 | 0.001999151 | 0.989449741 | 0.39183635 |
| 210502_s_at | PPIE | 0.000137065 | 0.001490239 | 0.989449741 | 0.271785205 |
| 218458_at | GMCL1 | 0.000137359 | 0.00634272 | 0.989449741 | −0.226475248 |
| 211963_s_at | ARPC5 | 0.000140872 | 0.001415219 | 0.989449741 | 0.303544959 |
| 204232_at | FCER1G | 0.000141945 | 0.001930541 | 0.989449741 | 0.277069582 |
| 202529_at | PRPSAP1 | 0.00015003 | 0.002461314 | 0.989449741 | 0.245784948 |
| 209702_at | FTO | 0.000154806 | 0.001537436 | 0.989449741 | 0.264580463 |
| 203096_s_at | RAPGEF2 | 0.000154806 | 0.001775858 | 0.989449741 | −0.326225528 |
| 212136_at | ATP2B4 | 0.00016222 | 0.001632865 | 0.989449741 | 0.384076656 |
| 221671_x_at | IGKC | 0.00016222 | 0.001424129 | 0.989449741 | 0.365217597 |
| 219409_at | SNIP1 | 0.00016222 | 0.002442598 | 0.989449741 | −0.253702874 |
| 201850_at | CAPG | 0.000164212 | 0.00291282 | 0.989449741 | 0.319326031 |
| 204820_s_at | BTN3A3 | 0.000173406 | 0.001765977 | 0.989449741 | 0.359117268 |
| 212829_at | PIP5K2A | 0.000173406 | 0.002437481 | 0.999875537 | 0.263400444 |
| 201284_s_at | APEH | 0.000174099 | 0.003894405 | 0.989449741 | 0.34306097 |
| 218563_at | NDUFA3 | 0.000176874 | 0.002748357 | 0.989449741 | 0.235476452 |
| 204079_at | TPST2 | 0.000176874 | 0.001930541 | 0.989449741 | 0.293849427 |
| 203800_s_at | MRPS14 | 0.000180029 | 0.002229882 | 0.989449741 | 0.258068001 |
| 208998_at | UCP2 | 0.000193043 | 0.002692241 | 0.989449741 | 0.423766127 |
| 212613_at | BTN3A2 | 0.000199037 | 0.002705123 | 0.989449741 | 0.747702388 |
| 217286_s_at | NDRG3 | 0.000203249 | 0.001392062 | 0.999875537 | 0.368294346 |
| 200791_s_at | IQGAP1 | 0.0002203 | 0.00331601 | 0.989449741 | 0.357197839 |
| 207270_x_at | CD300C | 0.000223695 | 0.003114079 | 0.989449741 | 0.329820875 |
| 212484_at | FAM89B | 0.000227557 | 0.000807266 | 0.989449741 | 0.408397813 |
| 209879_at | SELPLG | 0.000241035 | 0.00325328 | 0.989449741 | 0.393555874 |
| 202333_s_at | UBE2B | 0.000241517 | 0.001763941 | 0.989449741 | −0.276830195 |
| 1729_at | TRADD | 0.000241723 | 0.008502132 | 0.996902938 | 0.296784617 |
| 202808_at | C10ORF26 | 0.000246633 | 0.003032572 | 0.999775135 | 0.371386316 |
| 203252_at | CDK2AP2 | 0.000248397 | 0.002477145 | 0.989449741 | 0.333028031 |
| 207108_s_at | NIPBL | 0.000263483 | 0.007499776 | 0.999170597 | −0.312838282 |
| 201548_s_at | JARID1B | 0.000275345 | 0.001632865 | 0.989449741 | −0.276402213 |
| 202659_at | PSMB10 | 0.000307326 | 0.00331601 | 0.989449741 | 0.272106446 |
| 200719_at | SKP1A | 0.000311347 | 0.007670592 | 0.989449741 | −0.23130029 |
| 213566_at | RNASE6 | 0.000312669 | 0.009062127 | 0.989449741 | 0.317555781 |
| 205718_at | ITGB7 | 0.000313372 | 0.001190689 | 0.992633412 | 0.2770242 |
| 204890_s_at | LCK | 0.000313372 | 0.001922581 | 0.989449741 | 0.262915413 |
| 211637_x_at | LOC90925 | 0.000313372 | 0.0095183 | 0.989449741 | 0.494634585 |
| 218061_at | MEA1 | 0.000313372 | 0.003450279 | 0.989449741 | 0.274379136 |
| 212299_at | NEK9 | 0.000313372 | 0.001601914 | 0.989449741 | 0.361947717 |
| 206150_at | CD27 | 0.000314872 | 0.005418639 | 0.989449741 | 0.25560753 |
| 202033_s_at | RB1CC1 | 0.000314872 | 0.003927436 | 0.989449741 | −0.283068359 |
| 204233_s_at | CHKA | 0.000327081 | 0.001415219 | 0.989449741 | −0.587980802 |
| 203790_s_at | HRSP12 | 0.000327081 | 0.002727981 | 0.989449741 | 0.342224821 |
| 201413_at | HSD17B4 | 0.000327081 | 0.006359435 | 0.989449741 | 0.333084834 |
| 214677_x_at | IGL@ | 0.000327081 | 0.002692241 | 0.989449741 | 0.423884783 |
| 213918_s_at | NIPBL | 0.000327081 | 0.00325328 | 0.995081416 | −0.27071854 |
| 211005_at | LAT | 0.000331555 | 0.000689198 | 0.992633412 | 0.312999176 |
| 205292_s_at | HNRPA2B1 | 0.000334082 | 0.004144142 | 0.989449741 | 0.315879927 |
| 206342_x_at | IDS | 0.000336065 | 0.00372096 | 0.989449741 | −0.245720985 |
| 203336_s_at | ITGB1BP1 | 0.000336065 | 0.001922581 | 0.989449741 | 0.304012724 |
| 221651_x_at | IGKC | 0.00033855 | 0.001930541 | 0.989449741 | 0.352132457 |
| 218175_at | CCDC92 | 0.000346126 | 0.006359435 | 0.989449741 | 0.238586642 |
| 201331_s_at | STAT6 | 0.000346126 | 0.002727981 | 0.989449741 | 0.308582369 |
| 219032_x_at | OPN3 | 0.000357036 | 0.002748357 | 0.989449741 | 0.315457222 |
| 211986_at | AHNAK | 0.000358691 | 0.003787898 | 0.989449741 | 0.319716188 |
| 205898_at | CX3CR1 | 0.000363049 | 0.002727981 | 0.989449741 | 0.64892969 |
| 201082_s_at | DCTN1 | 0.000380411 | 0.00325328 | 0.999875537 | 0.257862257 |
| 202845_s_at | RALBP1 | 0.0003886 | 0.007361396 | 0.989449741 | 0.387015183 |
| 39729_at | PRDX2 | 0.000390562 | 0.00457769 | 0.989449741 | 0.27918459 |
| 208018_s_at | HCK | 0.000392544 | 0.006148992 | 0.989449741 | 0.371305637 |
| 200982_s_at | ANXA6 | 0.000410744 | 0.004431603 | 0.999875537 | 0.463842527 |
| 207419_s_at | RAC2 | 0.000414077 | 0.002933255 | 0.989449741 | 0.302920265 |
| 205297_s_at | CD79B | 0.00042883 | 0.002441036 | 0.989449741 | 0.436193739 |
| 214259_s_at | AKR7A2 | 0.000435187 | 0.004726659 | 0.989449741 | 0.251578601 |
| 203341_at | CEBPZ | 0.000435187 | 0.004992461 | 0.989449741 | −0.307645978 |
| 220086_at | IKZF5 | 0.000435187 | 0.005091358 | 0.989449741 | −0.253415901 |
| 205664_at | KIN | 0.000435187 | 0.005952778 | 0.989449741 | −0.240921176 |
| 200634_at | PFN1 | 0.000435187 | 0.009369724 | 0.999875537 | 0.232632614 |
| 204243_at | RLF | 0.000435187 | 0.006968826 | 0.989449741 | −0.254361688 |
| 218491_s_at | THYN1 | 0.000435187 | 0.002154763 | 0.989449741 | 0.328772135 |
| 221708_s_at | UNC45A | 0.000435187 | 0.002727981 | 0.989449741 | 0.294145498 |
| 203990_s_at | UTX | 0.000435187 | 0.001490239 | 0.989449741 | −0.331952866 |
| 209138_x_at | IGL@ | 0.000443553 | 0.002810556 | 0.989449741 | 0.423688903 |
| 212315_s_at | NUP210 | 0.000443553 | 0.001632865 | 0.989449741 | 0.338828184 |
| 219014_at | PLAC8 | 0.000443553 | 0.003534516 | 0.989449741 | 0.255992476 |
| 202139_at | AKR7A2 | 0.000478035 | 0.003674925 | 0.989449741 | 0.257221263 |
| 201998_at | ST6GAL1 | 0.000478035 | 0.006229039 | 0.989449741 | 0.280024885 |
| 200615_s_at | AP2B1 | 0.000481603 | 0.004726659 | 0.989449741 | 0.308163531 |
| 203104_at | CSF1R | 0.000481603 | 0.006746971 | 0.989449741 | 0.389448679 |
| 217179_x_at | LOC96610 | 0.000481603 | 0.007048137 | 0.989449741 | 0.375568002 |
| 201214_s_at | PPP1R7 | 0.000483398 | 0.004412192 | 0.989449741 | 0.270884223 |
| 221666_s_at | PYCARD | 0.000485769 | 0.006739032 | 0.989449741 | 0.288863922 |
| 200703_at | DYNLL1 | 0.000492836 | 0.004306658 | 0.989449741 | 0.323659455 |
| 217157_x_at | 0 | 0.000499201 | 0.001679791 | 0.989449741 | 0.300769926 |
| 201954_at | ARPC1B | 0.000499201 | 0.01354814 | 0.989449741 | 0.214105409 |
| 209824_s_at | ARNTL | 0.000512016 | 0.009096479 | 0.989449741 | −0.252731918 |
| 211458_s_at | GABARAPL1 | 0.000512016 | 0.004749386 | 0.989449741 | −0.337349799 |
| 201762_s_at | PSME2 | 0.000536775 | 0.003894405 | 0.989449741 | 0.275608632 |
| 200684_s_at | UBE2L3 | 0.000536775 | 0.007624822 | 0.989449741 | 0.388182939 |
| 218201_at | NDUFB2 | 0.000537945 | 0.002525693 | 0.989449741 | 0.279688772 |
| 203079_s_at | CUL2 | 0.000544377 | 0.002930881 | 0.989449741 | −0.293647521 |
| 201903_at | UQCRC1 | 0.000544814 | 0.00511929 | 0.989449741 | 0.270111589 |
| 202013_s_at | EXT2 | 0.000556834 | 0.012460713 | 0.989449741 | 0.221371121 |
| 211048_s_at | PDIA4 | 0.000565589 | 0.002746411 | 0.989449741 | 0.273878867 |
| 210971_s_at | ARNTL | 0.000565656 | 0.003114079 | 0.989449741 | −0.31915569 |
| 216105_x_at | PPP2R4 | 0.000574375 | 0.002249 | 0.989449741 | 0.320742428 |
| 219594_at | NINJ2 | 0.000575274 | 0.011688155 | 0.989449741 | 0.231783025 |
| 220964_s_at | RAB1B | 0.000575274 | 0.006930342 | 0.989449741 | 0.241663222 |
| 212514_x_at | DDX3X | 0.000587289 | 0.00706957 | 0.989449741 | −0.25519555 |
| 220485_s_at | SIRPG | 0.000587289 | 0.00331601 | 0.989449741 | 0.285246281 |
| 218268_at | TBC1D15 | 0.000599965 | 0.005225298 | 0.989449741 | −0.281136062 |
| 202539_s_at | HMGCR | 0.000636419 | 0.00331601 | 0.989449741 | −0.284457554 |
| 214617_at | PRF1 | 0.000636419 | 0.001415219 | 0.999170597 | 0.47924031 |
| 200851_s_at | KIAA0174 | 0.000647237 | 0.009929511 | 0.989449741 | −0.233420403 |
| 207088_s_at | SLC25A11 | 0.000649551 | 0.004374833 | 0.990401573 | 0.290426005 |
| 208868_s_at | GABARAPL1 | 0.000650709 | 0.006968826 | 0.989449741 | −0.34883537 |
| 202944_at | NAGA | 0.000653612 | 0.007402147 | 0.989449741 | 0.324428948 |
| 221763_at | JMJD1C | 0.000665011 | 0.004622991 | 0.989449741 | −0.262994291 |
| 204858_s_at | ECGF1 | 0.000680268 | 0.014138091 | 0.989449741 | 0.274391085 |
| 215273_s_at | TADA3L | 0.000680268 | 0.014655112 | 0.989449741 | 0.431022902 |
| 214224_s_at | PIN4 | 0.000682813 | 0.005768649 | 0.989449741 | 0.250168816 |
| 202297_s_at | RER1 | 0.000682813 | 0.004749386 | 0.992633412 | 0.3028154 |
| 214437_s_at | SHMT2 | 0.000684445 | 0.009369724 | 0.989449741 | 0.270877435 |
| 212203_x_at | IFITM3 | 0.000685694 | 0.002727981 | 0.989449741 | 0.304891056 |
| 217143_s_at | TRA@ | 0.00069156 | 0.004726659 | 0.989449741 | 0.320170922 |
| 218256_s_at | NUP54 | 0.000700841 | 0.008087703 | 0.989449741 | −0.290309592 |
| 213720_s_at | SMARCA4 | 0.000702585 | 0.014810987 | 0.999875537 | 0.226058265 |
| 206978_at | CCR2 | 0.000707287 | 0.016490882 | 0.989449741 | 0.240810193 |
| 201543_s_at | SAR1A | 0.000707287 | 0.011710089 | 0.989449741 | −0.2625788 |
| 214768_x_at | 0 | 0.000712203 | 0.004306658 | 0.989449741 | 0.343460208 |
| 206383_s_at | G3BP2 | 0.000712203 | 0.002748357 | 0.989449741 | −0.313723775 |
| 215176_x_at | NTN2L | 0.000727566 | 0.004726659 | 0.989449741 | 0.339751138 |
| 209177_at | C3ORF60 | 0.000727566 | 0.008506404 | 0.989449741 | 0.257293634 |
| 204618_s_at | GABPB2 | 0.000727566 | 0.006148992 | 0.989449741 | −0.367502426 |
| 218773_s_at | MSRB2 | 0.000727566 | 0.005418639 | 0.989449741 | 0.357615708 |
| 221918_at | PCTK2 | 0.000728806 | 0.021079092 | 0.989449741 | −0.237531914 |
| 204821_at | BTN3A3 | 0.000739347 | 0.002155997 | 0.989449741 | 0.399235041 |
| 212135_s_at | ATP2B4 | 0.000762738 | 0.003518947 | 0.989449741 | 0.318713047 |
| 219343_at | CDC37L1 | 0.000762738 | 0.007309057 | 0.999860944 | −0.29606778 |
| 207945_s_at | CSNK1D | 0.000762738 | 0.016364469 | 0.990463252 | −0.290788077 |
| 204091_at | PDE6D | 0.000762738 | 0.004374833 | 0.999775135 | 0.617568961 |
| 212632_at | STX7 | 0.000762738 | 0.004756217 | 0.989449741 | 0.324204972 |
| 201100_s_at | USP9X | 0.000782045 | 0.007048137 | 0.989449741 | −0.248495758 |
| 200765_x_at | CTNNA1 | 0.000787612 | 0.00555987 | 0.989449741 | 0.246901887 |
| 214669_x_at | IGKC | 0.000818503 | 0.006608275 | 0.989449741 | 0.338664468 |
| 213475_s_at | ITGAL | 0.000818503 | 0.001775858 | 0.992633412 | 0.319906726 |
| 202447_at | DECR1 | 0.000822295 | 0.00331601 | 0.999875537 | 0.269322531 |
| 200762_at | DPYSL2 | 0.000822295 | 0.012331531 | 0.989449741 | 0.359599434 |
| 215121_x_at | IGL@ | 0.000822295 | 0.00724686 | 0.989449741 | 0.449199148 |
| 200658_s_at | PHB (includes | 0.000822295 | 0.011178107 | 0.989449741 | 0.271153703 |
| | EG: 5245) | | | | |
| 217148_x_at | 0 | 0.000824507 | 0.004728268 | 0.989449741 | 0.33805833 |
| 200812_at | CCT7 | 0.000824507 | 0.016482999 | 0.989449741 | 0.219696813 |
| 207224_s_at | SIGLEC7 | 0.000824507 | 0.013385865 | 0.989449741 | 0.305830409 |
| 219403_s_at | HPSE | 0.000830105 | 0.009732612 | 0.999875537 | 0.265302417 |
| 221532_s_at | WDR61 | 0.00084731 | 0.008087703 | 0.989449741 | 0.235336728 |
| 202502_at | ACADM | 0.000864933 | 0.002979665 | 0.989449741 | 0.401564746 |
| 213698_at | ZMYM6 | 0.000864933 | 0.01021407 | 0.989449741 | 0.270123556 |
| 204071_s_at | TOPORS | 0.000940098 | 0.001999151 | 0.989449741 | −0.321902977 |
| 218130_at | C17ORF62 | 0.000959138 | 0.019509876 | 0.989449741 | 0.441340283 |
| 220477_s_at | C20ORF30 | 0.000959138 | 0.008590251 | 0.999875537 | 0.245868211 |
| 204759_at | RCBTB2 | 0.000959138 | 0.011688155 | 0.989449741 | 0.268283287 |
| 205671_s_at | HLA-DOB | 0.000983379 | 0.006596765 | 0.989449741 | 0.332615313 |
| 219679_s_at | WAC | 0.00099235 | 0.00390025 | 0.989449741 | −0.265005602 |
| 205996_s_at | AK2 | 0.000994104 | 0.014655112 | 0.989449741 | 0.240184345 |
| 204205_at | APOBEC3G | 0.000995041 | 0.001601914 | 0.992633412 | 0.304346622 |
| 204369_at | PIK3CA | 0.001002085 | 0.009244488 | 0.989449741 | −0.306938779 |
| 213888_s_at | TRAF3IP3 | 0.00101061 | 0.002748357 | 0.989449741 | 0.298506161 |
| 203350_at | AP1G1 | 0.001012017 | 0.01779807 | 0.989449741 | −0.290224501 |
| 206666_at | GZMK | 0.001032512 | 0.006148992 | 0.989449741 | 0.381124515 |
| 217973_at | DCXR | 0.001041384 | 0.005901401 | 0.989449741 | 0.256480374 |
| 202034_x_at | RB1CC1 | 0.001060851 | 0.008506404 | 0.989449741 | −0.330899098 |
| 203645_s_at | CD163 | 0.001069412 | 0.011620845 | 0.996310208 | 0.400019376 |
| 205552_s_at | OAS1 | 0.001076471 | 0.00789891 | 0.989449741 | 0.488844142 |
| 207831_x_at | DHPS | 0.001091307 | 0.005361731 | 0.989449741 | 0.475729022 |
| 207655_s_at | BLNK | 0.001106608 | 0.010317256 | 0.989449741 | 0.349842564 |
| 205001_s_at | DDX3Y | 0.001120007 | 0.001930541 | 0.989449741 | −0.666861961 |
| | (includes | | | | |
| | EG: 8653) | | | | |
| 218571_s_at | CHMP4A | 0.00114921 | 0.001458279 | 0.989449741 | 0.318205226 |
| 200678_x_at | GRN | 0.001185216 | 0.015004176 | 0.989449741 | 0.235273089 |
| 202352_s_at | PSMD12 | 0.001185216 | 0.005848736 | 0.989449741 | −0.266721911 |
| 202589_at | TYMS | 0.001199952 | 0.006148992 | 0.999875537 | 0.375833885 |
| 203148_s_at | TRIM14 | 0.001202227 | 0.010834794 | 0.989449741 | 0.392683893 |
| 206748_s_at | SPAG9 | 0.001221534 | 0.008326908 | 0.989449741 | −0.323085644 |
| 216260_at | DICER1 | 0.001224234 | 0.005411025 | 0.989449741 | −0.312582444 |
| 209171_at | ITPA | 0.001224234 | 0.005361731 | 0.989449741 | 0.26696903 |
| 215512_at | MARCH6 | 0.001225556 | 0.007137669 | 0.989449741 | −0.391768817 |
| 203685_at | BCL2 | 0.001241664 | 0.014752724 | 0.989449741 | 0.316753201 |
| 202727_s_at | IFNGR1 | 0.001244945 | 0.00511929 | 0.992633412 | −0.304448541 |
| 215118_s_at | IGHG1 | 0.001252379 | 0.002727981 | 0.989449741 | 0.342881272 |
| 221044_s_at | TRIM34 | 0.00125746 | 0.01779807 | 0.989449741 | 0.298921393 |
| 211595_s_at | MRPS11 | 0.001301438 | 0.005411025 | 0.989449741 | 0.327142963 |
| 217752_s_at | CNDP2 | 0.001344373 | 0.009379981 | 0.989449741 | 0.281082748 |
| 222217_s_at | SLC27A3 | 0.001344798 | 0.006968826 | 0.999170597 | 0.288456759 |
| 205251_at | PER2 | 0.001364923 | 0.014531661 | 0.989449741 | −0.273999133 |
| 201688_s_at | TPD52 | 0.001403687 | 0.009425377 | 0.9946638 | 0.253835591 |
| 204891_s_at | LCK | 0.001419862 | 0.003894405 | 0.992633412 | 0.280545008 |
| 218487_at | ALAD | 0.001439968 | 0.006968826 | 0.989449741 | 0.326607698 |
| 201301_s_at | ANXA4 | 0.00144365 | 0.007639163 | 0.989449741 | 0.746644814 |
| 202201_at | BLVRB | 0.001573278 | 0.01584915 | 0.994317186 | 0.288007441 |
| 201195_s_at | SLC7A5 | 0.001573278 | 0.007106724 | 0.989449741 | −0.361964776 |
| 205260_s_at | ACYP1 | 0.001594725 | 0.014138091 | 0.999170597 | 0.486885421 |
| 39318_at | TCL1A | 0.001625883 | 0.015894187 | 0.989449741 | 0.292387585 |
| 218102_at | DERA | 0.001649114 | 0.011523846 | 0.989449741 | 0.248811393 |
| 216933_x_at | APC | 0.001673247 | 0.01779807 | 0.989449741 | −0.328628065 |
| 215535_s_at | AGPAT1 | 0.001690954 | 0.013903896 | 0.989449741 | 0.244873423 |
| 210793_s_at | NUP98 | 0.001706911 | 0.016373934 | 0.989449741 | −0.265306242 |
| 210875_s_at | ZEB1 | 0.001731676 | 0.006454233 | 0.999860944 | −0.536203945 |
| 203720_s_at | ERCC1 | 0.001744503 | 0.013206152 | 0.989449741 | 0.293891701 |
| 200660_at | S100A11 | 0.001744503 | 0.037352667 | 0.989449741 | 0.279874352 |
| 200900_s_at | M6PR | 0.00175887 | 0.009379981 | 0.989449741 | 0.355503452 |
| 215946_x_at | CTA-246H3.1 | 0.001776637 | 0.00325328 | 0.989449741 | 0.341566401 |
| 201989_s_at | CREBL2 | 0.001778721 | 0.016222394 | 0.989449741 | 0.239353485 |
| 210222_s_at | RTN1 | 0.001778721 | 0.014655112 | 0.989449741 | 0.4388518 |
| 221511_x_at | CCPG1 | 0.001837478 | 0.026823359 | 0.989449741 | 0.27677713 |
| 202503_s_at | KIAA0101 | 0.001837478 | 0.005361731 | 0.989449741 | 0.344399133 |
| 209422_at | PHF20 | 0.001890809 | 0.014996942 | 0.992633412 | −0.260712578 |
| 213370_s_at | SFMBT1 | 0.001925515 | 0.016250748 | 0.999875537 | −0.252680392 |
| 216041_x_at | GRN | 0.001937213 | 0.018875106 | 0.989449741 | 0.254505572 |
| 201990_s_at | CREBL2 | 0.001947681 | 0.016435548 | 0.989449741 | 0.27904165 |
| 216191_s_at | TRA@ | 0.001947681 | 0.011133735 | 0.999875537 | 0.381662453 |
| 211138_s_at | KMO | 0.001954903 | 0.013214901 | 0.989449741 | 0.261798831 |
| 201711_x_at | RANBP2 | 0.001954903 | 0.006608275 | 0.989449741 | −0.359000131 |
| 209268_at | VPS45 | 0.001954903 | 0.01867368 | 0.989449741 | 0.28924038 |
| 211684_s_at | DYNC1I2 | 0.001957198 | 0.014096459 | 0.989449741 | 0.268152837 |
| 219666_at | MS4A6A | 0.00202555 | 0.024230535 | 0.989449741 | 0.298775239 |
| 209123_at | QDPR | 0.00202555 | 0.007624822 | 0.989449741 | 0.291374348 |
| 210981_s_at | GRK6 | 0.002108686 | 0.008590251 | 0.989449741 | 0.322221965 |
| 218248_at | FAM111A | 0.002177781 | 0.011757556 | 0.989449741 | 0.256785004 |
| 205639_at | AOAH | 0.002200811 | 0.018063019 | 0.989449741 | 0.233983537 |
| 218432_at | FBXO3 | 0.002271966 | 0.019841242 | 0.989449741 | −0.243052502 |
| 217893_s_at | C1ORF108 | 0.002327952 | 0.010968324 | 0.989449741 | −0.283241076 |
| 211052_s_at | TBCD | 0.002327952 | 0.029443759 | 0.989449741 | 0.458961294 |
| 204552_at | 0 | 0.0023375 | 0.010703847 | 0.999860944 | 0.259989457 |
| 214230_at | CDC42 | 0.00235967 | 0.007366046 | 0.989449741 | −0.28391366 |
| 217957_at | C16ORF80 | 0.002360504 | 0.014531661 | 0.989449741 | −0.323672759 |
| 208923_at | CYFIP1 | 0.002403669 | 0.053507143 | 0.999860944 | 0.20010478 |
| 204960_at | PTPRCAP | 0.002439639 | 0.014324902 | 0.989449741 | 0.259927296 |
| 203741_s_at | ADCY7 | 0.002441506 | 0.007348671 | 0.999875537 | 0.301320352 |
| 204222_s_at | GLIPR1 | 0.002441506 | 0.0182804 | 0.989449741 | 0.372323325 |
| 203814_s_at | NQO2 | 0.002467859 | 0.020807539 | 0.989449741 | 0.309002396 |
| 202857_at | TMEM4 | 0.002467859 | 0.016111032 | 0.989449741 | 0.262027938 |
| 206697_s_at | HP | 0.002480813 | 0.033802633 | 0.989449741 | 0.318610166 |
| 217234_s_at | VIL2 | 0.002480813 | 0.008087703 | 0.999875537 | −0.323455207 |
| 220068_at | VPREB3 | 0.002508189 | 0.008087703 | 0.989449741 | 0.36430699 |
| 212239_at | PIK3R1 | 0.002512151 | 0.008240887 | 0.989449741 | −0.351698742 |
| 211430_s_at | IGHM | 0.002515933 | 0.011946681 | 0.989449741 | 0.502359564 |
| 212890_at | MGC15523 | 0.002544105 | 0.019765771 | 0.989449741 | 0.4407365 |
| 201830_s_at | NET1 | 0.00255705 | 0.02191709 | 0.989449741 | −0.365628151 |
| 204258_at | CHD1 | 0.00259005 | 0.020697692 | 0.989449741 | −0.263374998 |
| 222309_at | C6ORF62 | 0.002613143 | 0.020025632 | 0.989449741 | −0.309369764 |
| 204950_at | CARD8 | 0.002613143 | 0.025474368 | 0.989449741 | 0.244398697 |
| 209555_s_at | CD36 | 0.002664317 | 0.027150542 | 0.989449741 | 0.313914898 |
| 203334_at | DHX8 | 0.002739188 | 0.031758827 | 0.989449741 | −0.242178793 |
| 221210_s_at | NPL | 0.002757438 | 0.027569971 | 0.989449741 | 0.334476885 |
| 208930_s_at | ILF3 | 0.002778072 | 0.014379033 | 0.999875537 | −0.731713347 |
| 214700_x_at | RIF1 | 0.002804775 | 0.022785018 | 0.999170597 | −0.245875298 |
| 204512_at | HIVEP1 | 0.002808884 | 0.048702813 | 0.999875537 | −0.208486404 |
| 214836_x_at | IGKC | 0.0028273 | 0.007361396 | 0.989449741 | 0.307170528 |
| 202869_at | OAS1 | 0.002836989 | 0.061278571 | 0.989449741 | 0.355521465 |
| 202411_at | IFI27 | 0.002867417 | 0.005765311 | 0.999860944 | 0.827033954 |
| 219061_s_at | LAGE3 | 0.002889493 | 0.008673311 | 0.989449741 | 0.351213669 |
| 217118_s_at | C22ORF9 | 0.002895987 | 0.015722075 | 0.989449741 | 0.252802103 |
| 209341_s_at | IKBKB | 0.002895987 | 0.013059559 | 0.989449741 | 0.275617947 |
| 212647_at | RRAS | 0.002956805 | 0.020254751 | 0.989449741 | 0.437888582 |
| 207540_s_at | SYK | 0.003080047 | 0.043081212 | 0.999875537 | 0.51488359 |
| 201878_at | ARIH1 | 0.003149179 | 0.024529617 | 0.999875537 | −0.247120857 |
| 211798_x_at | IGLJ3 | 0.003180983 | 0.015772918 | 0.989449741 | 0.284322069 |
| 210438_x_at | TROVE2 | 0.003183382 | 0.017675986 | 0.989449741 | −0.270741307 |
| 220104_at | ZC3HAV1 | 0.003215554 | 0.019097439 | 0.989449741 | −0.242782025 |
| 209681_at | SLC19A2 | 0.003218487 | 0.00724686 | 0.999875537 | −0.340654718 |
| 204435_at | NUPL1 | 0.003225429 | 0.01847557 | 0.999860944 | −0.278092387 |
| 204236_at | FLI1 | 0.003235464 | 0.027092973 | 0.989449741 | 0.291791808 |
| 206011_at | CASP1 | 0.003252713 | 0.02180297 | 0.989449741 | 0.257280453 |
| 209930_s_at | NFE2 | 0.003252713 | 0.036706981 | 0.989449741 | 0.252075493 |
| 201679_at | ARS2 | 0.003269014 | 0.006695499 | 0.989449741 | −0.342030018 |
| 219529_at | CLIC3 | 0.003309914 | 0.026541622 | 0.999875537 | 0.501837283 |
| 201798_s_at | FER1L3 | 0.003374801 | 0.010834794 | 0.989449741 | 0.32575619 |
| 211865_s_at | FZR1 | 0.003416575 | 0.008537732 | 0.992633412 | −0.402156995 |
| 200796_s_at | MCL1 | 0.00343314 | 0.004367608 | 0.989449741 | −0.379899638 |
| 212368_at | ZNF292 | 0.003471202 | 0.015153914 | 0.989449741 | −0.326664906 |
| 208898_at | ATP6V1D | 0.003659949 | 0.03422722 | 0.989449741 | 0.226487959 |
| 206934_at | SIRPB1 | 0.00366028 | 0.043217608 | 0.999875537 | 0.343668306 |
| 213024_at | TMF1 | 0.003682604 | 0.009369724 | 0.989449741 | −0.283458269 |
| 221136_at | GDF2 | 0.003730435 | 0.026915366 | 0.999860944 | 0.41393192 |
| 206398_s_at | CD19 | 0.003756272 | 0.009014956 | 0.989449741 | 0.267618155 |
| 220386_s_at | EML4 | 0.003756272 | 0.027580386 | 0.989449741 | −0.249592455 |
| 219191_s_at | BIN2 | 0.003812976 | 0.008087703 | 0.998443527 | 0.28972134 |
| 201369_s_at | ZFP36L2 | 0.00382513 | 0.008087703 | 0.989449741 | −0.396936859 |
| 209674_at | CRY1 | 0.00383637 | 0.04338212 | 0.989449741 | −0.294783202 |
| 212631_at | STX7 | 0.003894289 | 0.010518011 | 0.999875537 | 0.284992549 |
| 213300_at | ATG2A | 0.003971598 | 0.042915787 | 0.989449741 | −0.291057792 |
| 210837_s_at | PDE4D | 0.003992387 | 0.030850293 | 0.989449741 | −0.238971281 |
| 201459_at | RUVBL2 | 0.003997839 | 0.020048456 | 0.996310208 | 0.420997172 |
| 200849_s_at | AHCYL1 | 0.004152031 | 0.026336476 | 0.989449741 | 0.239521439 |
| 216510_x_at | IGHM | 0.004152031 | 0.022115542 | 0.989449741 | 0.267384564 |
| 218673_s_at | ATG7 | 0.004181766 | 0.031980345 | 0.990401573 | 0.242400077 |
| 209448_at | HTATIP2 | 0.004294431 | 0.016998728 | 0.989449741 | 0.289392365 |
| 209657_s_at | HSF2 | 0.004295648 | 0.038492458 | 0.989449741 | −0.250387579 |
| 210104_at | MED6 | 0.004362687 | 0.059705599 | 0.989449741 | −0.206476793 |
| 209199_s_at | MEF2C | 0.004366742 | 0.033848804 | 0.989449741 | 0.277362016 |
| 208901_s_at | TOP1 | 0.004371954 | 0.01845181 | 0.989449741 | −0.244197725 |
| 212420_at | ELF1 | 0.004409498 | 0.011736904 | 0.989449741 | −0.421075125 |
| 207001_x_at | TSC22D3 | 0.00441375 | 0.008087703 | 0.989449741 | −0.385669354 |
| 204565_at | THEM2 | 0.004447673 | 0.034175352 | 0.989449741 | 0.261898479 |
| 201713_s_at | RANBP2 | 0.004486641 | 0.025862737 | 0.989449741 | −0.290946919 |
| 201101_s_at | BCLAF1 | 0.004493632 | 0.015623198 | 0.989449741 | −0.33563532 |
| 209773_s_at | RRM2 | 0.004538428 | 0.009062127 | 0.989449741 | 0.317141015 |
| 204244_s_at | DBF4 | 0.004667713 | 0.018925816 | 0.989449741 | −0.276178697 |
| 218562_s_at | TMEM57 | 0.004667713 | 0.02031709 | 0.992633412 | −0.337272049 |
| 206743_s_at | ASGR1 | 0.0047202 | 0.048329949 | 0.989449741 | 0.262995524 |
| 211368_s_at | CASP1 | 0.004724575 | 0.029689301 | 0.989449741 | 0.248072171 |
| 215049_x_at | CD163 | 0.004724575 | 0.029653891 | 0.989449741 | 0.343903258 |
| 214777_at | 0 | 0.00474319 | 0.030851645 | 0.999875537 | 0.470253868 |
| 213853_at | DPH4 | 0.00474319 | 0.032504947 | 0.989449741 | 0.284103318 |
| 202684_s_at | RNMT | 0.004827006 | 0.020195849 | 0.989449741 | −0.339233164 |
| 212189_s_at | COG4 | 0.004915846 | 0.021664295 | 0.989449741 | 0.304605563 |
| 214766_s_at | AHCTF1 | 0.005045322 | 0.031035179 | 0.989449741 | −0.2389356 |
| 202643_s_at | TNFAIP3 | 0.005049625 | 0.026107956 | 0.989449741 | −0.256706151 |
| 204520_x_at | BRD1 | 0.005056176 | 0.037489214 | 0.989449741 | −0.236422395 |
| 218160_at | NDUFA8 | 0.005104732 | 0.02725912 | 0.989449741 | 0.265540404 |
| 41577_at | PPP1R16B | 0.005114154 | 0.03758103 | 0.989449741 | −0.266716491 |
| 202802_at | DHPS | 0.005153729 | 0.012341023 | 0.989449741 | 0.411326347 |
| 212579_at | SMCHD1 | 0.005242671 | 0.020195849 | 0.989449741 | −0.270301779 |
| 213138_at | ARID5A | 0.005374749 | 0.025862737 | 0.999875537 | −0.257003315 |
| 205202_at | PCMT1 | 0.005419642 | 0.031014488 | 0.989449741 | 0.274242936 |
| 206474_at | PCTK2 | 0.005420029 | 0.009425377 | 0.989449741 | −0.280266936 |
| 222142_at | CYLD | 0.005433797 | 0.056829739 | 0.989449741 | −0.221781393 |
| 208325_s_at | AKAP13 | 0.005479037 | 0.015178539 | 0.989449741 | −0.338103785 |
| 204354_at | POT1 | 0.005501641 | 0.007361396 | 0.989449741 | 0.291545231 |
| 207686_s_at | CASP8 | 0.005510098 | 0.022693611 | 0.992633412 | −0.262343036 |
| 207791_s_at | RAB1A | 0.005532348 | 0.016970375 | 0.989449741 | −0.260040425 |
| 205819_at | MARCO | 0.0055492 | 0.022798957 | 0.989449741 | 0.556337817 |
| 210776_x_at | TCF3 | 0.005580048 | 0.005135308 | 0.999860944 | 0.343587607 |
| 201448_at | TIA1 | 0.005592773 | 0.008753175 | 0.989449741 | 0.384888382 |
| 210314_x_at | TNFSF13 | 0.005631239 | 0.030050096 | 0.989449741 | 0.251922246 |
| 213830_at | TRA@ | 0.005631239 | 0.061278571 | 0.999875537 | 0.313025901 |
| 202771_at | FAM38A | 0.005764725 | 0.019176407 | 0.999860944 | 0.277325037 |
| 218232_at | C1QA | 0.005843574 | 0.022998906 | 0.989449741 | 0.294564228 |
| 211634_x_at | IGHM | 0.005874475 | 0.003209262 | 0.989449741 | 0.401747885 |
| 211676_s_at | IFNGR1 | 0.005895325 | 0.017278892 | 0.992633412 | −0.26708949 |
| 211658_at | PRDX2 | 0.00592062 | 0.046710505 | 0.989449741 | 0.300257179 |
| 218660_at | DYSF | 0.005988683 | 0.038845427 | 0.989449741 | 0.26516736 |
| 210321_at | GZMH | 0.00599779 | 0.014324902 | 0.989449741 | 0.385606971 |
| 212842_x_at | RGPD5 | 0.006040184 | 0.029375171 | 0.989449741 | −0.256742246 |
| 213238_at | ATP10D | 0.006148056 | 0.014752724 | 0.989449741 | 0.396761691 |
| 218598_at | RINT-1 | 0.006309451 | 0.031494602 | 0.989449741 | −0.23835499 |
| 208130_s_at | TBXAS1 | 0.006360222 | 0.048745041 | 0.989449741 | 0.238746065 |
| 207630_s_at | CREM | 0.006433074 | 0.02936887 | 0.989449741 | −0.346620151 |
| 206761_at | CD96 | 0.006452047 | 0.010509546 | 0.999875537 | 0.305052027 |
| 209581_at | HRASLS3 | 0.006522732 | 0.037537936 | 0.989449741 | 0.273100246 |
| 209099_x_at | JAG1 | 0.0065548 | 0.043881101 | 0.989449741 | −0.237884195 |
| 218321_x_at | STYXL1 | 0.006594919 | 0.014655112 | 0.989449741 | 0.377829635 |
| 220560_at | C11ORF21 | 0.006888885 | 0.006785178 | 0.999875537 | 0.310692295 |
| 218400_at | OAS3 | 0.006916555 | 0.033848804 | 0.999875537 | 0.291024811 |
| 204630_s_at | GOSR1 | 0.007048224 | 0.055725696 | 0.989449741 | −0.370355173 |
| 209480_at | HLA-DQB1 | 0.007072014 | 0.012627228 | 0.989449741 | 0.583368519 |
| 208810_at | DNAJB6 | 0.007115524 | 0.026083865 | 0.989449741 | −0.248474131 |
| 213674_x_at | IGHD | 0.007117464 | 0.042140113 | 0.989449741 | 0.314831791 |
| 218504_at | FAHD2A | 0.007261903 | 0.027569971 | 0.989449741 | 0.275918665 |
| 201218_at | 0 | 0.007297492 | 0.027150542 | 0.989449741 | 0.319848628 |
| 217236_x_at | IGHG1 | 0.007312652 | 0.01466556 | 0.989449741 | 0.277945656 |
| 207190_at | ZZEF1 | 0.007312652 | 0.061503008 | 0.989449741 | −0.231303663 |
| 200683_s_at | UBE2L3 | 0.007490607 | 0.06144415 | 0.989449741 | 0.318072482 |
| 212070_at | GPR56 | 0.007499462 | 0.016998728 | 0.989449741 | 0.352222843 |
| 208499_s_at | DNAJC3 | 0.00794364 | 0.026676031 | 0.989449741 | −0.377832728 |
| 203454_s_at | ATOX1 | 0.007964188 | 0.05950856 | 0.989449741 | 0.228088671 |
| 210660_at | LILRA1 | 0.008046967 | 0.038992611 | 0.989449741 | 0.293688016 |
| 45714_at | HCFC1R1 | 0.008346281 | 0.016208342 | 0.989449741 | 0.321583436 |
| 218628_at | CCDC53 | 0.008370392 | 0.026336476 | 0.989449741 | 0.308291932 |
| 207104_x_at | LILRB1 | 0.008370392 | 0.028390504 | 0.989449741 | 0.276273653 |
| 202906_s_at | NBN | 0.008412904 | 0.061710728 | 0.989449741 | −0.228774539 |
| 212841_s_at | PPFIBP2 | 0.008431717 | 0.070499722 | 0.999875537 | −0.308356194 |
| 202820_at | AHR | 0.008543591 | 0.023346042 | 0.989449741 | −0.333964081 |
| 218793_s_at | SCML1 | 0.008724804 | 0.019699152 | 0.989449741 | −0.302915244 |
| 211644_x_at | IGKC | 0.008836995 | 0.04251771 | 0.989449741 | 0.368362779 |
| 215460_x_at | BRD1 | 0.008856719 | 0.043881101 | 0.989449741 | −0.240914782 |
| 216100_s_at | TOR1AIP1 | 0.008935905 | 0.067754828 | 0.989449741 | −0.251357762 |
| 201841_s_at | HSPB1 | 0.008945424 | 0.026676031 | 0.989449741 | 0.322182392 |
| 201906_s_at | CTDSPL | 0.008949709 | 0.115094717 | 0.989449741 | 0.229704349 |
| 205483_s_at | ISG15 | 0.009046991 | 0.030070903 | 0.999775135 | 0.3301337 |
| 209771_x_at | CD24 | 0.009174971 | 0.068140197 | 0.989449741 | 0.401443846 |
| 219184_x_at | TIMM22 | 0.009174971 | 0.006968826 | 0.989449741 | −0.626545634 |
| 211133_x_at | LILRB2 | 0.009264483 | 0.031360675 | 0.989449741 | 0.246592831 |
| 208621_s_at | VIL2 | 0.009378043 | 0.015178539 | 0.989449741 | −0.292897213 |
| 218968_s_at | ZFP64 | 0.009405491 | 0.02878907 | 0.989449741 | 0.278043195 |
| 203542_s_at | KLF9 | 0.009421114 | 0.014138091 | 0.989449741 | −0.303213811 |
| 213844_at | HOXA5 | 0.009456431 | 0.019884878 | 0.989449741 | −0.336925595 |
| 220532_s_at | TMEM176B | 0.009456431 | 0.072462443 | 0.989449741 | 0.253768337 |
| 203197_s_at | C1ORF123 | 0.009475056 | 0.025967346 | 0.989449741 | 0.316251039 |
| 220646_s_at | KLRF1 | 0.009842474 | 0.026676031 | 0.998443527 | 0.307337492 |
| 202932_at | YES1 | 0.009845258 | 0.063320125 | 0.989449741 | −0.260259979 |
| 212240_s_at | PIK3R1 | 0.010102748 | 0.044571978 | 0.989449741 | −0.280321363 |
| 214719_at | SLC46A3 | 0.010385639 | 0.08498821 | 0.989449741 | 0.234249392 |
| 203227_s_at | TSPAN31 | 0.010385639 | 0.081869754 | 0.989449741 | 0.269839505 |
| 217230_at | VIL2 | 0.010484209 | 0.030850293 | 0.999875537 | −0.606267812 |
| 212830_at | MEGF9 | 0.010642378 | 0.05302726 | 0.989449741 | 0.310775116 |
| 207840_at | CD160 | 0.010813258 | 0.03869558 | 0.999875537 | 0.248985995 |
| 205239_at | AREG | 0.010892256 | 0.046710505 | 0.992633412 | −0.447684123 |
| 203485_at | RTN1 | 0.011091972 | 0.079062359 | 0.989449741 | 0.352208833 |
| 215925_s_at | CD72 | 0.011114129 | 0.045839519 | 0.989449741 | 0.426533268 |
| 218877_s_at | TRMT11 | 0.011114129 | 0.052857992 | 0.989449741 | −0.243831432 |
| 209184_s_at | IRS2 | 0.011377086 | 0.025854059 | 0.989449741 | −0.370878022 |
| 206770_s_at | SLC35A3 | 0.01152795 | 0.081874529 | 0.989449741 | −0.210129814 |
| 202275_at | G6PD | 0.011571849 | 0.076810207 | 0.989449741 | 0.223272707 |
| 220370_s_at | USP36 | 0.011657632 | 0.017396935 | 0.989449741 | −0.316048827 |
| 219854_at | ZNF14 | 0.011657632 | 0.071210572 | 0.989449741 | −0.216348273 |
| 207983_s_at | STAG2 | 0.011784294 | 0.029443759 | 0.989449741 | −0.283436906 |
| 209460_at | ABAT | 0.011814732 | 0.077338625 | 0.989449741 | 0.332456127 |
| 202729_s_at | LTBP1 | 0.012045807 | 0.060192971 | 0.989449741 | 0.259651211 |
| 215716_s_at | ATP2B1 | 0.012329558 | 0.087788247 | 0.999775135 | −0.234367527 |
| 202382_s_at | GNPDA1 | 0.012329558 | 0.074482324 | 0.999875537 | 0.226427543 |
| 211135_x_at | LILRB2 | 0.012329558 | 0.04720123 | 0.989449741 | 0.290183832 |
| 217022_s_at | IGHA1 | 0.012330644 | 0.071279406 | 0.989449741 | 0.37940291 |
| 204771_s_at | TTF1 | 0.012349011 | 0.026541622 | 0.992633412 | −0.612140469 |
| 206881_s_at | LILRA3 | 0.012382773 | 0.146996117 | 0.989449741 | 0.205018265 |
| 208993_s_at | PPIG | 0.012478202 | 0.046710505 | 0.989449741 | −0.495377578 |
| 209385_s_at | PROSC | 0.012685741 | 0.086929712 | 0.989449741 | 0.281793793 |
| 213241_at | PLXNC1 | 0.012797271 | 0.06201157 | 0.989449741 | 0.245225098 |
| 209417_s_at | IFI35 | 0.01288426 | 0.052543489 | 0.989449741 | 0.245500095 |
| 202996_at | POLD4 | 0.01288426 | 0.013904233 | 0.989449741 | 0.504213833 |
| 208661_s_at | TTC3 | 0.013577437 | 0.020067307 | 0.998443527 | −0.558621455 |
| 203504_s_at | ABCA1 | 0.013691899 | 0.064956405 | 0.999875537 | −0.270427783 |
| 202723_s_at | FOXO1 | 0.013812872 | 0.111956122 | 0.989449741 | −0.219524825 |
| 211199_s_at | ICOSLG | 0.013899311 | 0.084773153 | 0.989449741 | −0.418583364 |
| 211576_s_at | SLC19A1 | 0.013947132 | 0.052781693 | 0.989449741 | 0.282972566 |
| 203791_at | DMXL1 | 0.013982542 | 0.102658371 | 0.999875537 | 0.254810714 |
| 215379_x_at | IGL@ | 0.014228373 | 0.029729359 | 0.989449741 | 0.299774111 |
| 202027_at | TMEM184B | 0.01463017 | 0.15080112 | 0.989449741 | −0.238216082 |
| 214995_s_at | APOBEC3F | 0.015429877 | 0.023623476 | 0.989449741 | 0.30052076 |
| 216379_x_at | CD24 | 0.01546115 | 0.10927203 | 0.989449741 | 0.337940524 |
| 211840_s_at | PDE4D | 0.015530692 | 0.077171886 | 0.989449741 | −0.315843621 |
| 212764_at | 0 | 0.015730425 | 0.093235833 | 0.999875537 | −0.302079268 |
| 208983_s_at | PECAM1 | 0.015730425 | 0.058827879 | 0.989449741 | 0.222235435 |
| 214508_x_at | CREM | 0.015876726 | 0.046275245 | 0.989449741 | −0.276762529 |
| 214273_x_at | C16ORF35 | 0.016155203 | 0.029776129 | 0.989449741 | 0.274342013 |
| 206829_x_at | ZNF430 | 0.01628046 | 0.031084112 | 0.999875537 | −0.334510066 |
| 219228_at | ZNF331 | 0.016758668 | 0.038989413 | 0.989449741 | −0.265651975 |
| 209281_s_at | ATP2B1 | 0.016778489 | 0.094912682 | 0.999875537 | −0.216021467 |
| 219210_s_at | RAB8B | 0.016810155 | 0.080902359 | 0.999860944 | −0.249121329 |
| 210154_at | ME2 | 0.016929959 | 0.058473604 | 0.989449741 | 0.231317327 |
| 206707_x_at | C6ORF32 | 0.017069796 | 0.061278571 | 0.989449741 | 0.245248804 |
| 210784_x_at | LILRB2 | 0.017430703 | 0.074383254 | 0.999875537 | 0.234595745 |
| 204181_s_at | ZBTB43 | 0.018118096 | 0.038102953 | 0.989449741 | −0.359978259 |
| 208450_at | LGALS2 | 0.018840047 | 0.151080324 | 0.999875537 | 0.231199005 |
| 213397_x_at | RNASE4 | 0.018840047 | 0.050336409 | 0.989449741 | 0.246937949 |
| 208541_x_at | TFAM | 0.01888466 | 0.043301094 | 0.999875537 | −0.36578751 |
| 220363_s_at | ELMO2 | 0.019035865 | 0.04251771 | 0.989449741 | −0.336672576 |
| 209127_s_at | SART3 | 0.019084727 | 0.01867368 | 0.989449741 | −0.579425607 |
| 211423_s_at | SC5DL | 0.019738942 | 0.054658281 | 0.999170597 | −0.26933491 |
| 203203_s_at | KRR1 | 0.019761987 | 0.081365496 | 0.989449741 | −0.273574762 |
| 217977_at | SEPX1 | 0.019798679 | 0.095742497 | 0.989449741 | 0.30918947 |
| 204833_at | ATG12 | 0.020092997 | 0.060143693 | 0.999860944 | −0.29732725 |
| 216576_x_at | 0 | 0.020504101 | 0.033500356 | 0.989449741 | 0.356263226 |
| 215621_s_at | IGHD | 0.020537251 | 0.049535517 | 0.989449741 | 0.469561382 |
| 205936_s_at | HK3 | 0.020626319 | 0.077265558 | 0.989449741 | 0.370492283 |
| 219497_s_at | BCL11A | 0.020630202 | 0.031084112 | 0.989449741 | 0.277512558 |
| 218856_at | TNFRSF21 | 0.020646727 | 0.258615288 | 0.989449741 | −0.365340192 |
| 212229_s_at | FBXO21 | 0.020966502 | 0.048891302 | 0.999875537 | −0.27937857 |
| 212531_at | LCN2 | 0.021154912 | 0.094912682 | 0.989449741 | 0.299904534 |
| 213672_at | MARS | 0.022676752 | 0.16378389 | 0.989449741 | −0.190080228 |
| | (includes | | | | |
| | EG: 4141) | | | | |
| 216915_s_at | PTPN12 | 0.023085786 | 0.028802905 | 0.989449741 | −0.322773907 |
| 208991_at | STAT3 | 0.023105118 | 0.04251771 | 0.989449741 | −0.429258343 |
| 207808_s_at | PROS1 | 0.024454572 | 0.097813067 | 0.989449741 | 0.277242815 |
| 213979_s_at | 0 | 0.02446421 | 0.057863327 | 0.989449741 | 0.265547674 |
| 204619_s_at | VCAN | 0.024551356 | 0.154020939 | 0.989449741 | 0.189540592 |
| 201367_s_at | ZFP36L2 | 0.024691689 | 0.042874833 | 0.999875537 | −0.499101398 |
| 220330_s_at | SAMSN1 | 0.025070537 | 0.062360925 | 0.999775135 | −0.263647206 |
| 216491_x_at | IGHM | 0.0254179 | 0.04328448 | 0.989449741 | 0.501481085 |
| 201110_s_at | THBS1 | 0.025706246 | 0.039755163 | 0.989449741 | −0.554321888 |
| 207269_at | DEFA4 | 0.025927783 | 0.143378347 | 0.989449741 | 0.328951536 |
| 207978_s_at | NR4A3 | 0.025927783 | 0.136476074 | 0.989449741 | −0.289082826 |
| 202208_s_at | ARL4C | 0.026525797 | 0.117920787 | 0.989449741 | −0.250545565 |
| 202988_s_at | RGS1 | 0.0275444 | 0.072842185 | 0.989449741 | −0.386389145 |
| 201939_at | PLK2 | 0.027574591 | 0.07697782 | 0.989449741 | −0.318660078 |
| 202458_at | PRSS23 | 0.027728103 | 0.162974212 | 0.999875537 | 0.262008375 |
| 209829_at | C6ORF32 | 0.028448903 | 0.083382181 | 0.989449741 | 0.242964416 |
| 202933_s_at | YES1 | 0.028448903 | 0.110632076 | 0.989449741 | −0.302993319 |
| 210367_s_at | PTGES | 0.02861839 | 0.09725281 | 0.999875537 | −0.231156235 |
| 208772_at | ANKHD1 | 0.028686587 | 0.112218548 | 0.989449741 | −0.44588397 |
| 207735_at | RNF125 | 0.028919122 | 0.098198128 | 0.989449741 | −0.306157663 |
| 214091_s_at | GPX3 | 0.029127031 | 0.076316804 | 0.989449741 | −0.251707391 |
| 204959_at | MNDA | 0.029463273 | 0.162352719 | 0.989449741 | 0.217286616 |
| 210139_s_at | PMP22 | 0.030423473 | 0.039148997 | 0.989449741 | −0.439210039 |
| 209967_s_at | CREM | 0.03081361 | 0.128158563 | 0.989449741 | −0.398455681 |
| 220001_at | PADI4 | 0.030881449 | 0.11007471 | 0.989449741 | 0.248581247 |
| 208651_x_at | CD24 | 0.031022644 | 0.101596709 | 0.989449741 | 0.380097925 |
| 212720_at | PAPOLA | 0.031022644 | 0.159495805 | 0.999875537 | 0.209683615 |
| 204006_s_at | FCGR3A | 0.031040754 | 0.044398381 | 0.999170597 | 0.376554 |
| 204198_s_at | RUNX3 | 0.031235279 | 0.11007471 | 0.989449741 | −0.263762479 |
| 201631_s_at | IER3 | 0.031367646 | 0.040272223 | 0.989449741 | −0.332792024 |
| 201534_s_at | UBL3 | 0.031682917 | 0.054438594 | 0.989449741 | −0.267888461 |
| 211635_x_at | 0 | 0.03230983 | 0.015674081 | 0.989449741 | 0.340412689 |
| 200895_s_at | FKBP4 | 0.03273201 | 0.11357739 | 0.989449741 | 0.217516851 |
| 209153_s_at | TCF3 | 0.033314149 | 0.051510117 | 0.999170597 | 0.281960028 |
| 208960_s_at | KLF6 | 0.033465179 | 0.091784194 | 0.989449741 | −0.691636254 |
| 206488_s_at | CD36 | 0.033527437 | 0.129528475 | 0.989449741 | 0.221187201 |
| 210178_x_at | FUSIP1 | 0.033527437 | 0.074158718 | 0.989449741 | −0.415118743 |
| 209504_s_at | PLEKHB1 | 0.033661797 | 0.031758827 | 0.989449741 | 0.318126329 |
| 203913_s_at | HPGD | 0.033684873 | 0.008087703 | 0.989449741 | −0.458826569 |
| 205033_s_at | DEFA1 | 0.033802918 | 0.20812861 | 0.989449741 | 0.338694932 |
| 218723_s_at | C13ORF15 | 0.033903606 | 0.07876279 | 0.989449741 | −0.249750457 |
| 205070_at | ING3 | 0.034660832 | 0.079120731 | 0.999875537 | −0.310353631 |
| 220751_s_at | C5ORF4 | 0.034700433 | 0.172103477 | 0.999860944 | 0.202019036 |
| 208987_s_at | FBXL11 | 0.034788285 | 0.048580539 | 0.989449741 | −0.447799698 |
| 208992_s_at | STAT3 | 0.034896789 | 0.031994065 | 0.989449741 | −0.338569068 |
| 208942_s_at | TLOC1 | 0.034896789 | 0.104374493 | 0.989449741 | −0.386927768 |
| 209604_s_at | GATA3 | 0.034927254 | 0.098912567 | 0.989449741 | −0.253801443 |
| 202871_at | TRAF4 | 0.03542462 | 0.08416156 | 0.989449741 | −0.274481617 |
| 219392_x_at | PRR11 | 0.035621799 | 0.075234284 | 0.989449741 | −0.402711374 |
| 205027_s_at | MAP3K8 | 0.035676625 | 0.062623254 | 0.989449741 | −0.265793388 |
| 204567_s_at | ABCG1 | 0.035736869 | 0.092341417 | 0.996080789 | −0.251947095 |
| 218449_at | C4ORF20 | 0.035948991 | 0.127321099 | 0.999875537 | 0.225420003 |
| 203505_at | ABCA1 | 0.036437022 | 0.132615325 | 0.992633412 | −0.278556795 |
| 211560_s_at | ALAS2 | 0.036875551 | 0.051998922 | 0.999860944 | 0.531623369 |
| 214446_at | ELL2 | 0.037411336 | 0.051163635 | 0.989449741 | −0.527512602 |
| 212954_at | DYRK4 | 0.03744809 | 0.245711527 | 0.989449741 | 0.184060875 |
| 202927_at | PIN1 | 0.037481626 | 0.076508004 | 0.999875537 | 0.263650358 |
| 212592_at | IGJ | 0.037485741 | 0.058827879 | 0.989449741 | 0.331582846 |
| 218401_s_at | ZNF281 | 0.037632567 | 0.061278571 | 0.989449741 | −0.289322273 |
| 214786_at | MAP3K1 | 0.039034036 | 0.04512331 | 0.989449741 | −0.348471489 |
| 210653_s_at | BCKDHB | 0.039877111 | 0.067715814 | 0.999875537 | 0.286175627 |
| 200878_at | EPAS1 | 0.039920199 | 0.101126257 | 0.989449741 | −0.305545648 |
| 220319_s_at | MYLIP | 0.040709639 | 0.130484533 | 0.989449741 | −0.272540562 |
| 209959_at | NR4A3 | 0.04271719 | 0.146996117 | 0.989449741 | −0.384959402 |
| 207414_s_at | PCSK6 | 0.042954608 | 0.130824771 | 0.989449741 | 0.266397191 |
| 210244_at | CAMP | 0.043058783 | 0.15724045 | 0.989449741 | 0.370996496 |
| 201109_s_at | THBS1 | 0.043727365 | 0.076171233 | 0.989449741 | −0.468029423 |
| 211285_s_at | UBE3A | 0.04463363 | 0.158570058 | 0.999775135 | −0.262984362 |
| 204961_s_at | NCF1 | 0.046220251 | 0.180186992 | 0.989449741 | 0.218415324 |
| 209023_s_at | STAG2 | 0.04635073 | 0.043778052 | 0.989449741 | −0.334015439 |
| 213653_at | METTL3 | 0.047100971 | 0.043065116 | 0.999860944 | 0.304049124 |
| 218237_s_at | SLC38A1 | 0.047158302 | 0.077497989 | 0.989449741 | −0.332756015 |
| 206871_at | ELA2 | 0.047306295 | 0.151483987 | 0.989449741 | 0.375756567 |
| 211506_s_at | IL8 | 0.047319227 | 0.110267486 | 0.989449741 | −0.422779419 |
| 212224_at | ALDH1A1 | 0.04837586 | 0.168321646 | 0.989449741 | 0.231824985 |
| 212005_at | 0 | 0.049589112 | 0.051163635 | 0.992633412 | −0.367774805 |

TABLE 6
Subgroup X-IL-15 Regulated Genes and Metrics

| | | | | Entrez | Entrez | | |
|---|---|---|---|---|---|---|---|
| | | | False | Gene ID | Gene ID | Entrez | Exemplar |
| | | | Discovery | for | for | Gene ID | SEQ ID |
| Gene Name | Gene Description | Log Ratio | Rate | Human | Mouse | for Rat | NO. |
| BCL2 | B-cell CLL/lymphoma 2 | 0.336331 | 0.0076092 | 596 | 12043 | 24224 | 1 |
| CALM1 | calmodulin 1 (phosphorylase | 0.317008 | 2.80E−05 | 801 | 12313 | 24242 | 2 |
| | kinase, delta) | | | | | | |
| CCL2 | chemokine (C-C motif) ligand 2 | −3.197736 | 7.90E−12 | 6347 | 20293 | 287562 | 3 |
| CCR1 | chemokine (C-C motif) | −1.080562 | 3.03E−04 | 1230 | 12768 | 57301 | 4 |
| | receptor 1 | | | | | | |
| CCR2 | chemokine (C-C motif) | −0.327232 | 0.0080358 | 1231 | 12772 | 60463 | 5 |
| | receptor 2 | | | | | | |
| CD40 | CD40 molecule, TNF receptor | −0.500496 | 4.45E−04 | 958 | 21939 | 171369 | 6 |
| | superfamily member 5 | | | | | | |
| CD44 | CD44 molecule (Indian blood | 0.390189 | 5.38E−04 | 960 | 12505 | 25406 | 7 |
| | group) | | | | | | |
| CD53 | CD53 molecule | −0.324923 | 8.30E−05 | 963 | 12508 | 24251 | 8 |
| CD86 | CD86 molecule | −0.375037 | 1.94E−04 | 942 | 12524 | 56822 | 9 |
| CD8B | CD8b molecule | 0.529394 | 0.0041036 | 926 | 12526 | 24931 | 10 |
| CEACAM1 | carcinoembryonic antigen- | −0.332004 | 0.0126034 | 634 | 26365 | 81613 | 11 |
| (includes | related cell adhesion molecule | | | | | | |
| EG: 634) | 1 (biliary glycoprotein) | | | | | | |
| CKS1B | CDC28 protein kinase | −0.272981 | 0.0053558 | 1163 | 54124 | 499655 | 12 |
| | regulatory subunit 1B | | | | | | |
| CX3CR1 | chemokine (C—X3—C motif) | −0.921081 | 4.55E−04 | 1524 | 13051 | 171056 | 13 |
| | receptor 1 | | | | | | |
| CXCR4 | chemokine (C—X—C motif) | 0.435521 | 8.49E−05 | 7852 | 12767 | 60628 | 14 |
| | receptor 4 | | | | | | |
| DUSP11 | dual specificity phosphatase | 0.263793 | 0.0052424 | 8446 | 72102 | 297412 | 15 |
| | 11 (RNA/RNP complex 1- | | | | | | |
| | interacting) | | | | | | |
| FAS | Fas (TNF receptor | −0.452922 | 5.24E−05 | 355 | 14102 | 246097 | 16 |
| | superfamily, member 6) | | | | | | |
| GABPB2 | GA binding protein | 0.359036 | 0.0375917 | 2553 | 14391 | 364738 | 17 |
| | transcription factor, beta | | | | | | |
| | subunit 2 | | | | | | |
| GDI2 | GDP dissociation inhibitor 2 | 0.316008 | 4.30E−05 | 2665 | 14569 | 29662 | 18 |
| (includes | | | | | | | |
| EG: 2665) | | | | | | | |
| GNAS | GNAS complex locus | 0.281187 | 0.0067831 | 2778 | 14683 | 24896 | 19 |
| (includes | | | | | | | |
| EG: 2778) | | | | | | | |
| GZMB | granzyme B (granzyme 2, | −0.353436 | 0.0469344 | 3002 | 14939 | 171528 | 20 |
| | cytotoxic T-lymphocyte- | | | | | | |
| | associated serine esterase 1) | | | | | | |
| HIST2H2AA3 | histone cluster 2, H2aa3 | −0.491629 | 6.69E−06 | 8337 | 15267 | 690131, | 21 |
| | | | | | | 365877 | |
| HNRPA2B1 | heterogeneous nuclear | −0.31297 | 0.0199395 | 3181 | 53379 | 362361 | 22 |
| | ribonucleoprotein A2/B1 | | | | | | |
| ICAM2 | intercellular adhesion molecule 2 | −0.411062 | 1.03E−04 | 3384 | 15896 | 360647 | 23 |
| IFI35 | interferon-induced protein 35 | −1.539826 | 1.00E−14 | 3430 | 70110 | 287719 | 24 |
| IFIT1 | interferon-induced protein with | −3.348328 | 1.00E−14 | 3434 | 112419 | 294090 | 25 |
| | tetratricopeptide repeats 1 | | | | | | |
| IFITM1 | interferon induced | −1.137174 | 1.00E−14 | 8S19 | 68713 | 293618 | 26 |
| | transmembrane protein 1 (9-27) | | | | | | |
| IL15 | interleukin 15 | −0.721076 | 3.65E−08 | 3600 | 16168 | 25670 | 27 |
| IL2RG | interleukin 2 receptor, gamma | −0.345266 | 0.0395431 | 3561 | 16186 | 140924 | 28 |
| | (severe combined | | | | | | |
| | immunodeficiency) | | | | | | |
| KLRK1 | killer cell lectin-like receptor | 0.309414 | 0.0062409 | 22914 | 27007 | 24934 | 29 |
| | subfamily K, member 1 | | | | | | |
| LEF1 | lymphoid enhancer-binding | 0.382174 | 0.0058507 | 51176 | 16842 | 161452 | 30 |
| | factor 1 | | | | | | |
| LYN | v-yes-1 Yamaguchi sarcoma | −0.286898 | 0.0067434 | 4067 | 17096 | 81515 | 31 |
| | viral related oncogene | | | | | | |
| | homolog | | | | | | |
| MT1G | metallothionein 1G | −0.486602 | 6.25E−05 | 4495 | | | 32 |
| MT1H | metallothionein 1H | −0.703151 | 4.92E−04 | 4496 | | | 33 |
| MX1 | myxovirus (influenza virus) | −2.060167 | 1.00E−14 | 4599 | 17858 | 286918 | 34 |
| | resistance 1, interferon- | | | | | | |
| | inducible protein p78 (mouse) | | | | | | |
| MYD88 | myeloid differentiation primary | −0.405963 | 2.54E−09 | 4615 | 17874 | 301059 | 35 |
| | response gene (88) | | | | | | |
| NAGA | N-acetylgalactosaminidase, | −0.278443 | 0.0181778 | 4668 | 17939 | 315165 | 36 |
| | alpha- | | | | | | |
| NP | nucleoside phosphorylase | −0.283364 | 0.0153042 | 4860 | 18950 | 290029 | 37 |
| PIM1 | pim-1 oncogene | −0.436049 | 1.78E−08 | 5292 | 18712 | 24649 | 38 |
| PLEK | pleckstrin | −0.538329 | 4.27E−04 | 5341 | 56193 | 364206 | 39 |
| PSMB10 | proteasome (prosome, | −0.673245 | 1.32E−09 | 5699 | 19171 | 291983 | 40 |
| | macropain) subunit, beta type, | | | | | | |
| | 10 | | | | | | |
| PSMB9 | proteasome (prosome, | −0.859797 | 5.56E−10 | 5698 | 16912 | 24967 | 41 |
| | macropain) subunit, beta type, | | | | | | |
| | 9 (large multifunctional | | | | | | |
| | peptidase 2) | | | | | | |
| RAB8A | RAB8A, member RAS | −0.364174 | 3.01E−06 | 4218 | 17274 | 117103 | 42 |
| | oncogene family | | | | | | |
| RAC2 | ras-related C3 botulinum toxin | −0.313038 | 0.012755 | 5880 | 19354 | 366957 | 43 |
| | substrate 2 (rho family, small | | | | | | |
| | GTP binding protein Rac2) | | | | | | |
| S100A11 | S100 calcium binding protein | −0.854907 | 6.19E−08 | 6282 | 277089 | 445415 | 44 |
| | A11 | | | | | | |
| SELL | selectin L (lymphocyte | −0.574007 | 8.36E−07 | 6402 | 20343 | 29259 | 45 |
| | adhesion molecule 1) | | | | | | |
| SFRS7 | splicing factor, arginine/serine- | 0.277934 | 0.042304 | 6432 | 225027 | 362687 | 46 |
| | rich 7, 35 kDa | | | | | | |
| SP100 | SP100 nuclear antigen | −0.433437 | 4.59E−11 | 6672 | 20684 | 363269 | 47 |
| TFDP2 | transcription factor Dp-2 (E2F | 0.282919 | 0.0456667 | 7029 | 211586 | 300947 | 48 |
| | dimerization partner 2) | | | | | | |
| TJP2 | tight junction protein 2 (zona | −0.341516 | 0.0078942 | 9414 | 21873 | 115769 | 49 |
| | occludens 2) | | | | | | |
| TLR2 | toll-like receptor 2 | −0.523644 | 7.64E−04 | 7097 | 24088 | 310553 | 50 |
| HLX | Homeobox-like gene | −0.301298 | 0.0105950 | 3142 | 15284 | 364069 | 61 |
| IL15RA | interleukin 15 receptor, alpha | | | 3601 | 16169 | 364775 | 62 |
| PDIA4 | protein disulfide isomerase | −0.3346159 | 0.00237448 | 9601 | 12304 | 116598 | 63 |
| | family A, member 4 | | | | | | |
| SORL1 | sortilin-related receptor, L(DLR | 0.3383286 | 9.76E−05 | 6653 | 20660 | 300652 | 64 |
| | class) A repeats-containing | | | | | | |
| TNFSF10 | tumor necrosis factor (ligand) | −1.3477080 | 3.11E−10 | 8743 | 22035 | 246775 | 65 |
| | superfamily, member 10 | | | | | | |

TABLE 7
Stringency Conditions

| | Poly- | Hybrid | Hybridization | |
|---|---|---|---|---|
| Stringency | nucleotide | Length | Temperature and | Wash Temp. |
| Condition | Hybrid | (bp)¹ | Buffer^{H} | and Buffer^{H} |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}*; 1xSSC | T_{B}*; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}*; 1xSSC | T_{D}*; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}*; 2xSSC | T_{L}*; 2xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1x SSPE is 0.15M NaCl, 10 mM NaH₂PO₄, and 1.25 mM EDTA, pH 7.4) can be substituted for SSC (1x SSC is 0.15M NaCl and 15 mM sodium citrate) in the hybridization and wash buffers.
T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length,Tₘ(° C.) = 81.5 + 16.6(1og₁₀[Na⁺]) + 0.41 (% G + C) − (600/N), where N is the number of bases in the hybrid, and [Na⁺] is the molar concentration of sodium ions in the hybridization buffer ([Na⁺] for 1x SSC = 0.165 M).

TABLE 8
Serum Markers of Exacerbation

| | P value: | P value: | P value: | Mean | Mean | Mean | | | |
|---|---|---|---|---|---|---|---|---|---|
| Serum | Exacerb v | Asthma | Quiet v | ρg/ml | ρg/ml | ρg/ml | N | N | N |
| Biomarker | Quiet | v Healthy | Healthy | Exacerb | Quiet | Healthy | Exacerb | Quiet | Healthy |
| ST2 | 0.017 | 0.006 | 0.152 | 90.2 | 61.1 | 55.4 | 69 | 85 | 43 |
| CHI3L1 | | 0.003 | | *64,750.0 | | 43,800 | *82 | | 52 |
| (YKL-40) | | | | | | | | | |
| IL5 | 0.028 | 0.001 | 0.018 | 0.5 | 0.4 | 0.1 | 37 | 37 | 45 |
| Eotaxin | 0.803 | 0.188 | 0.098 | 578.2 | 525.1 | 626.4 | 37 | 37 | 45 |
| TNFa | 0.017 | 0.200 | 0.953 | 1.7 | 1.4 | 1.4 | 37 | 37 | 45 |
| IL8 | 0.110 | 0.140 | 0.502 | 6.0 | 4.1 | 3.5 | 37 | 37 | 45 |
| IL13 | 0.711 | 0.126 | 0.140 | 5.0 | 5.6 | 3.0 | 13 | 13 | 7 |
| MCP-1 | 0.096 | 0.139 | 0.753 | 240.8 | 201.4 | 196.8 | 37 | 37 | 45 |
| TARC | 0.690 | 0.583 | 0.486 | 42.2 | 44.9 | 40.6 | 37 | 37 | 45 |

*Represents total asthma (quiet and exacerbation)

TABLE 9
Cluster X Biomarkers Having Low Intra-subject Variability

| | | | | Q v. E |
|---|---|---|---|---|
| | | AOS v HVOS | Quiet v. | followup 1 |
| Gene Name | GenBank No. | FDR all visits | Exacerb FDR | FDR |
| interferon induced transmembrane | NM_003641.1 | 0 | 0 | 0.932963506 |
| protein 1 (IFITM1) | | | | |
| transcriptional coactivator Sp110b | AF280094.1 | 0 | 7.81859E−11 | 0.838299238 |
| (SP110) | | | | |
| tumor necrosis factor (ligand) | NM_003810.1 | 0 | 1.72681E−10 | 0.971768475 |
| superfamily, member 10 (TNFSF10) | | | | |
| interferon-induced protein 41, 30 kD | NM_004509.1 | 0 | 6.0713E−10 | 0.663534687 |
| (IFI41) | | | | |
| interferon induced transmembrane | AA749101 | 6.87162E−15 | 0 | 0.977627636 |
| protein 1 (IFITM1) | | | | |
| interferon induced transmembrane | AL121994 | 5.45762E−12 | 0 | 0.764947366 |
| protein pseudogene (SEQ ID NO: 58) | | | | |
| Homo sapiens hypothetical protein | NM_016619.1 | 2.67267E−11 | 0 | 0.547460786 |
| (LOC51316) (SEQ ID NO: 59) | | | | |
| ubiquitin-conjugating enzyme E2L 6 | NM_004223.1 | 2.80205E−11 | 0 | 0.804966785 |
| (UBE2L6) | | | | |
| interferon induced transmembrane | BF338947 | 4.70201E−11 | 0 | 0.804966785 |
| protein 3 (IFITM3) | | | | |
| Fc-gamma receptor I B1 (FCGR1A) | L03419.1 | 1.45092E−09 | 1.03296E−09 | 0.713883189 |
| myxovirus (influenza) resistance 2, | NM_002463.1 | 7.20437E−09 | 0 | 0.823670503 |
| homolog of murine (MX2) | | | | |
| 2-5oligoadenylate synthetase 2 | NM_016817.1 | 1.39899E−08 | 1.23025E−13 | 0.994302012 |
| (OAS2) | | | | |
| high affinity Fc receptor (FcRI) b form | X14355.1 | 2.84849E−08 | 4.37509E−11 | 0.543105461 |
| (FCGR1A) | | | | |
| signal transducer and activator of | NM_007315.1 | 7.50544E−08 | 8.19311E−10 | 0.806943262 |
| transcription 1, 91 kD (STAT1) | | | | |
| myxovirus (influenza) resistance 1 | NM_002462.1 | 1.25342E−06 | 0 | 0.92335789 |
| (interferon-inducible protein p78) | | | | |
| (MX1) | | | | |
| interferon, alpha-inducible protein | NM_022873.1 | 2.7602E−06 | 0 | 0.911573276 |
| (clone IFI-6-16) (G1P3) (IFI6) | | | | |
| bone marrow stromal cell antigen 2 | NM_004335.2 | 1.61277E−05 | 3.80484E−11 | 0.947786651 |
| (BST2) | | | | |
| hypothetical protein FLJ22693 | NM_022750.1 | 2.68573E−05 | 0 | 0.906722412 |
| (FLJ22693) (PARP12) | | | | |
| similar to interferon-induced protein | BC001356.1 | 4.13047E−05 | 0 | 0.844024603 |
| 35, clone MGC: 2935 (IFI35) | | | | |
| proteasome (prosome, macropain) | NM_002818.1 | 5.5437E−05 | 4.12266E−12 | 0.576624724 |
| activator subunit 2 (PA28 beta) | | | | |
| (PSME2) | | | | |

TABLE 10
Cluster Y Biomarkers Having Low Intra-subject Variability

| | | AOS v HVOS | Quiet v. | Q v. E |
|---|---|---|---|---|
| Gene Name | GenBank No. | FDR all visits | Exacerb FDR | followup FDR |
| CDC28 protein kinase 1 (CKS1) | NM_001826.1 | 0.000741 | 0.00039 | 0.999876 |
| defender against cell death 1 (DAD1) | NM_001344.1 | 0.000526 | 0.000469 | 0.98945 |
| hypothetical protein FLJ10143 | NM_018009.1 | 0.000144 | 0.000269 | 0.98945 |
| (FLJ10143) (TAPBPL) | | | | |
| immunoglobulin (mAb59) light chain V | D84143.1 | 9.65E−05 | 0.000466 | 0.98945 |
| region (IGL2) | | | | |
| intercellular adhesion molecule 2 | AA126728 | 6.25E−05 | 0.000308 | 0.98945 |
| (ICAM2) | | | | |
| ELAV (embryonic lethal, abnormal | BC003376.1 | 5.9E−05 | 0.000298 | 0.98945 |
| vision, Drosophila)-like 1 (Hu antigen | | | | |
| R) | | | | |
| interleukin 2 receptor, gamma (severe | NM_000206.1 | 5.64E−05 | 0.000206 | 0.98945 |
| combined immunodeficiency) (IL2RG) | | | | |
| vesicle-associated membrane protein | NM_003761.1 | 1.28E−07 | 0.000469 | 0.98945 |
| 8 (endobrevin) (VAMP8) | | | | |
| annexin A2 (ANXA2) | BC001388.1 | 6.08E−08 | 0.000938 | 0.98945 |
| intercellular adhesion molecule 2 | NM_000873.2 | 1.45E−08 | 0.000298 | 0.98945 |
| (ICAM2) | | | | |
| cat eye syndrome chromosome | NM_017424.1 | 7.13E−09 | 0.000714 | 0.98945 |
| region, candidate 1 (CECR1) | | | | |
| natural killer cell transcript 4 (NK4) | NM_004221.1 | 2.02E−09 | 0.000635 | 0.98945 |
| (IL32) | | | | |
| leukocyte-associated Ig-like receptor | AF109683.1 | 8.04E−10 | 0.000689 | 0.98945 |
| 1b (LAIR1) | | | | |
| arginine-rich, mutated in early stage | NM_006010.1 | 6.4E−10 | 0.000486 | 0.98945 |
| tumors (ARMET) | | | | |
| adenylate kinase 2 (AK2) | AL513611 | 1.33E−11 | 0.000469 | 0.98945 |
| natural killer cell enhancing factor | L19184.1 | 4.38E−14 | 0.00039 | 0.98945 |
| (NKEFA) (PRDX1) | | | | |
| KIAA0102 gene product (KIAA0102) | NM_014752.1 | 6.87E−15 | 0.000228 | 0.98945 |
| (SPCS2) | | | | |
| HSPC022 protein (RAC2) | BE138888 | 0 | 0.000269 | 0.98945 |
| Hs.11774 protein (peptidyl-prolyl | BE797213 | 0 | 0.000269 | 0.98945 |
| cistrans isomerase) NIMA-interacting, | | | | |
| 4 (parvulin) (PIN4) | | | | |
| ribonuclease, RNase A family, 2 | NM_002934.1 | 0 | 0.000931 | 0.98945 |
| (liver, eosinophil-derived neurotoxin) | | | | |
| (RNASE2) | | | | |

TABLE 11
Exacerbation plus Infection Sample Biomarkers

| | | | | | FDR | |
|---|---|---|---|---|---|---|
| | FDR: | Δlog2: | FDR: | FDR: | Quiet asthma | |
| | Exacer only | Exacer only | Exacer/Infec | All Exacer | v. | |
| | v. | v. | v. | v. | Healthy non- | |
| Gene | Quiet | Quiet | Quiet | Quiet | asthma | SEQ ID NO |
| IFITM3 | 0.001 | −0.333 | 0.003 | 5.17094E−06 | 4.70201E−11 | |
| G1P2 | 0.027 | −0.408 | 0.001 | 7.23786E−05 | 0.000782438 | |
| IF127 | 0.025 | −0.731 | 0.002 | 8.16669E−05 | #N/A | |
| TCN2 | 0.022 | −0.209 | 0.010 | 0.000100698 | 1.11604E−05 | |
| G1P3 | 0.022 | −0.365 | 0.016 | 0.000146777 | 2.7602E−06 | |
| SN | 0.027 | −0.324 | 0.007 | 0.000165414 | 0.391303978 | |
| IFI44 | 0.045 | −0.333 | 0.002 | 0.000165414 | 0.129278459 | |
| EIF4B | 0.025 | 0.142 | 0.023 | 0.000253558 | 0.007556601 | |
| SERPING1 | 0.091 | −0.420 | 0.003 | 0.000539881 | 0.003395405 | |
| APOBEC3A | 0.045 | −0.287 | 0.023 | 0.000587634 | 0.002173562 | |
| LY6E | 0.067 | −0.357 | 0.018 | 0.000762438 | 0.235837992 | |
| RPL22 | 0.067 | 0.119 | 0.035 | 0.001291265 | 0.058418958 | |
| MX1 | 0.171 | −0.281 | 0.007 | 0.001557619 | 1.25342E−06 | |
| OASL | 0.175 | −0.246 | 0.012 | 0.002382976 | 0.869662043 | |
| IFIT3 | 0.151 | −0.344 | 0.028 | 0.002974539 | 0.226938617 | |
| PPGB | 0.370 | −0.114 | 0.002 | 0.002974539 | 2.09308E−06 | |
| UNK_AF063612 | 0.154 | −0.225 | 0.041 | 0.004087944 | 0.495985855 | |
| OAS3 | 0.265 | −0.277 | 0.050 | 0.009513928 | 0.000250457 | |
| PSME2 | 0.293 | −0.174 | 0.041 | 0.009513928 | 5.5437E−05 | |
| CD44 | 0.492 | 0.071 | 0.011 | 0.015400702 | 0.1315576 | |
| IFITM2 | 0.445 | −0.164 | 0.032 | 0.017938992 | 2.59456E−14 | |
| ECGF1 | 0.531 | −0.170 | 0.012 | 0.020179897 | 0.058038605 | |
| OAS1 | 0.445 | −0.311 | 0.042 | 0.022950993 | 0.002188974 | |
| PLAC8 | 0.492 | −0.137 | 0.028 | 0.02429545 | 2.67267E−11 | |
| IRF7 | 0.492 | −0.202 | 0.034 | 0.026794854 | 0.174138096 | |
| IFI35 | 0.506 | −0.200 | 0.032 | 0.027466143 | 4.13047E−05 | |
| TYMS | 0.514 | −0.172 | 0.030 | 0.027466143 | 2.20758E−07 | |
| RNASE2 | 0.706 | −0.142 | 0.002 | 0.027466143 | 0 | |
| FLJ38348 | 0.561 | −0.129 | 0.041 | 0.042413043 | 0.039377722 | |
| KIAA0101 | 0.572 | −0.147 | 0.036 | 0.043812874 | 4.44916E−07 | |
| UBE2L6 | 0.579 | −0.133 | 0.032 | 0.043812874 | 2.80205E−11 | |
| PIAS2 | 0.560 | 0.088 | 0.050 | 0.045371225 | 6.69202E−05 | |
| IFIT1 | 0.588 | −0.438 | 0.035 | 0.046057322 | 0.001371158 | |
| FCGR1A | 0.570 | −0.141 | 0.048 | 0.046330608 | 2.84849E−08 | |
| PSMA6 | 0.562 | −0.074 | 0.042 | 0.047062283 | 0 | |
| PSMB3 | 0.644 | −0.068 | 0.022 | 0.050202891 | 0 | |
| RRM2 | 0.591 | −0.142 | 0.038 | 0.050259485 | 7.24762E−05 | |
| FCER1G | 0.638 | −0.109 | 0.030 | 0.055052737 | 6.78321E−11 | |
| S100A11 | 0.616 | −0.162 | 0.042 | 0.063397546 | 0 | |
| DYSF | 0.773 | −0.120 | 0.028 | 0.12030689 | 2.2082E−06 | |
| PSMD8 | 0.856 | −0.046 | 0.041 | 0.217968184 | 2.75751E−12 | |
| CECR1 | 0.899 | −0.071 | 0.022 | 0.232280881 | 7.13012E−09 | |
| BLVRA | 0.886 | −0.043 | 0.030 | 0.232506885 | 0 | |
| HP | 0.898 | −0.083 | 0.030 | 0.244441709 | 2.18019E−05 | |
| BLVRA | 0.886 | −0.065 | 0.036 | 0.255095135 | 0 | |
| PSMB2 | 0.930 | −0.030 | 0.023 | 0.283759498 | 2.0947E−11 | |
| UNK_AW514267 | 0.925 | 0.052 | 0.042 | 0.304446425 | 8.82495E−08 | |
| SEPHS1 | 0.959 | 0.018 | 0.022 | 0.336856388 | 0.293094414 | |
| FLJ10726 | 0.957 | 0.017 | 0.038 | 0.367468887 | 0.9215285 | |
| PDXK | 0.974 | −0.017 | 0.023 | 0.385945685 | 2.17167E−05 | |
| TSPYL5 | 0.982 | 0.014 | 0.041 | 0.45192057 | 0.016253975 | |
| ARIH2 | 0.983 | 0.007 | 0.042 | 0.474810162 | 0.008139129 | |
| TRIM10 | 0.990 | −0.012 | 0.007 | 0.484966296 | 0.690008027 | |
| SMARCB1 | 0.966 | −0.023 | 0.050 | 0.718001203 | 0.869116514 | |

TABLE 12
Exacerbation with Infection Biomarkers

| | | | | | FDR | |
|---|---|---|---|---|---|---|
| | FDR: | Δlog2: | FDR: | FDR: | Quiet asthma | |
| | Exacer only | Exacer only | Exacer/Infec | All Exacer | v. | |
| | v. | v. | v. | v. | Healthy non- | |
| Gene | Quiet | Quiet | Quiet | Quiet | asthma | SEQ ID NO |
| SMARCB1 | 0.966 | −0.023 | 0.050 | 0.718001203 | 0.869116514 | 63 |
| TRIM10 | 0.990 | −0.012 | 0.007 | 0.484966296 | 0.690008027 | 64 |
| ARIH2 | 0.983 | 0.007 | 0.042 | 0.474810162 | 0.008139129 | 65 |
| TSPYL5 | 0.982 | 0.014 | 0.041 | 0.45192057 | 0.016253975 | 66 |
| PDXK | 0.974 | −0.017 | 0.023 | 0.385945685 | 2.17167E−05 | 67 |
| FLJ10726 | 0.957 | 0.017 | 0.038 | 0.367468887 | 0.9215285 | 68 |
| SEPHS1 | 0.959 | 0.018 | 0.022 | 0.336856388 | 0.293094414 | 69 |
| UNK_AW514267 | 0.925 | 0.052 | 0.042 | 0.304446425 | 8.82495E−08 | 70 |
| PSMB2 | 0.930 | −0.030 | 0.023 | 0.283759498 | 2.0947E−11 | 71 |
| BLVRA | 0.886 | −0.065 | 0.036 | 0.255095135 | 0 | 72 |
| HP | 0.898 | −0.083 | 0.030 | 0.244441709 | 2.18019E−05 | 73 |
| BLVRA | 0.886 | −0.043 | 0.030 | 0.232506885 | 0 | 74 |
| CECR1 | 0.899 | −0.071 | 0.022 | 0.232280881 | 7.13012E−09 | 75 |
| PSMD8 | 0.856 | −0.046 | 0.041 | 0.217968184 | 2.75751E−12 | 76 |
| DYSF | 0.773 | −0.120 | 0.028 | 0.12030689 | 2.2082E−06 | 77 |
| S100A11 | 0.616 | −0.162 | 0.042 | 0.063397546 | 0 | 44 |

What is claimed is:

1. A method of determining a molecular signature of asthma exacerbation of a patient with asthma, comprising:
(a) obtaining a sample from the patient;
(b) measuring the levels of two or more products in a sample obtained from the patient, wherein each product is produced from a gene which is differentially expressed during asthma exacerbation;
and
(c) comparing said levels of step (b) to reference levels of said two or more products,
wherein a difference between said levels of step (b) and the reference levels indicates the molecular signature of asthma exacerbation for the individual,
wherein the molecular signature indicates a type of asthma exacerbation selected from the group comprising exacerbation associated with innate immunity, exacerbation associated with cognate immunity, exacerbation associated with concomitant airway infection and exacerbation associated with no airway infection.

2. The method of claim 1, wherein the reference level is the level of said product in a sample obtained from the individual during an asthma quiet period.

3. The method of claim 1, wherein the reference level is the average of the level of said product in samples obtained from individuals who are not undergoing an asthma attack or asthma exacerbation.

4. The method of claim 1, wherein the sample comprises peripheral blood mononuclear cells.

5. The method of claim 1, wherein the type of asthma exacerbation comprises exacerbation associated with innate immunity and the gene is selected from the group comprising genes set forth in Table 4, Table 6 and Table 9.

6. The method of claim 5, wherein the gene is selected from the group consisting of IFI35, IFIT1, IFITM1, MX1, CCL2, SP100, PSMB9, PSMB10, MYD88 chemokine C-C motif receptor 1 (CCR1), chemokine C-X3-C motif receptor 1 (CX3CR1), S100 calcium binding protein A11 (S100A11), interleukin 15 (IL15) and PIM1.

7. The method of claim 1, wherein the type of asthma exacerbation comprises exacerbation associated with cognate immunity and the gene is selected from the group comprising genes set forth in Table 5 and Table 10.

8. The method of claim 1, wherein the type of asthma exacerbation comprises exacerbation associated with concomitant airway infection and the gene is selected from the group comprising genes set forth in Table 11 and Table 12.

9. The method of claim 1, wherein the type of asthma exacerbation comprises exacerbation not associated with airway infection and the gene is selected from the group comprising interferon induced with helicase C domain 1 (IFIH1), leukotriene A4 hydrolase (LTA4H) and open reading frame number 25 of human chromosome 6 (C6ORF25).

10. The method of claim 1, wherein the product is a protein.

11. The method of claim 1, wherein the product is a mRNA.

12. A method for assessing the effectiveness of a therapy, comprising:
(a) administering a therapy to a patient;
(b) measuring the level of at least one product in a sample obtained from the individual, wherein the product is produced from a gene which is differentially expressed during asthma exacerbation;
(c) comparing said level of step (a) to a reference level of said product, wherein a difference between said level of step (a) and the reference level indicates that the therapy is effective,
wherein the therapy is either an asthma therapy or an investigational asthma therapy.

13. The method of claim 12, wherein the reference level is the level of said product in a sample obtained from the individual prior to the administration of the therapy.

14. The method of claim 12, wherein the reference level is the average of the level of said product in samples obtained from individuals who are undergoing an asthma attack or asthma exacerbation.

15. The method of claim 12, wherein the sample comprises peripheral blood mononuclear cells.

16. The method of claim 12, wherein said gene is selected from the group consisting the genes set forth in Table 2, Table 3, Table 4, Table 5, Table 6, Table 8, Table 9, Table 10, Table 11 and Table 12.

17. The method of claim 16, wherein the gene is selected from the group consisting of interferon-induced protein 35 (IFI35), interferon-induced protein with tetratricopeptide repeats 1 (IFIT1), interferon-induced protein 44-like (IFI44L), interferon-induced protein 27 (IFI27), interferon-stimulated gene 15 (ISG15), serpin peptidase inhibitor clade G member 1 (SERPING1), interferon-induced protein with tetratricopeptide repeats 3 (IFIT3), interferon-induced protein 44 (IFI44), lymphocyte antigen 6 complex locus E (LY6E), interferon induced transmembrane protein 1 (IFITM1), interferon-inducible protein p78 (MX1), chemokine C-C motif ligand 2 (CCL2), SP100 nuclear antigen (SP100), proteasome subunit beta type 9 (PSMB9), chemokine C-C motif receptor 1 (CCR1), chemokine C-X3-C motif receptor 1 (CX3CR1), proteasome subunit beta type 10 (PSMB10), myeloid differentiation primary response gene 88 (MYD88), interleukin 15 (IL15), calcium binding protein A11 (S100A11) and pim-1 oncogene (PIM1).

18. The method of claim 12, wherein the product is a protein.

19. The method of claim 12, wherein the product is an mRNA.

20. A method of identifying a compound that is effective for treating asthma exacerbation, comprising: providing a candidate compound to a cell and determining whether said compound inhibits IL-15 activity in the cell, wherein inhibition of IL-15 activity indicates that said compound is effective for treating acute exacerbation of asthma.

21. The method according to claim 20, wherein said IL-15 activity is (a) binding of IL-15 to a cognate receptor, (b) a downstream IL-15 signaling event, or (c) both.

22. The method according to claim 20, wherein the cell is a peripheral blood mononuclear cell (PBMC).

23. An array for use in diagnosing asthma exacerbation in a patient, comprising a plurality of discrete regions on a substrate, each of which comprises a probe disposed thereon, wherein at least 15% of the plurality of discrete regions has disposed thereon probes that specifically detect a marker of asthma exacerbation in PBMCs or other tissues.

24. The array of claim 23, wherein the marker of asthma exacerbation comprises at least one marker selected from the group consisting of the markers set forth in Tables 2, 3, 4, 5, 6, 8, 9, 10, 11 and 12.

25. The array of claim 24, wherein the marker of asthma exacerbation has an FDR for exacerbation versus quiet of less than or equal to 0.00001.

26. The array of claim 23, wherein each probe is a polynucleotide.

27. The array of claim 23, wherein each probe is an antibody or fragment thereof.

28. The array of claim 23, wherein each probe is an aptamer.

29. The array of claim 26 comprising a polynucleotide probe for each of IFIT1, MX1 and CCL2; and optionally comprising a polynucleotide probe for any one or more of IFI35, IFITM1, SP100, PSMB9, PSMB10, MYD88, PIM1, CCR1, CX3CR1, S100A11, IL15, IFI27, ISG15 SERPING1, IFIT3, IFI44 and LY6E; wherein each of said polynucleotide probe is a single-stranded polynucleotide comprising at least 22 contiguous nucleotides.

30. A kit comprising a detection reagent which binds to the gene product of any one of a plurality of genes that are differentially expressed in a sample obtained from an individual having an asthma exacerbation versus a sample obtained from an individual having an asthma quiet period.

31. The kit of claim 30, wherein the plurality of genes is selected from the group consisting of the genes set forth in Tables 1-6 and 8-12.

32. The kit of claim 30, wherein the gene product comprises a polypeptide and the detection reagent comprises an antibody, a fragment of an antibody, or an aptamer.

33. The kit of any one of claims 30, wherein the gene product comprises a polynucleotide and the detection reagent comprises an oligonucleotide that hybridizes to the polynucleotide.
